(19) United States
(12) Patent Application Publication
(10) Pub. No.: US 2019/0127313 A1
(43) Pub. Date: May 2, 2019

(54) ANTIMICROBIAL AGENTS

(71) Applicant: THE UNIVERSITY OF WARWICK, Coventry, Warwickshire (GB)

(72) Inventors: Gregory Leonard CHALLIS, Coventry, Warwickshire (GB); Daniel John GRIFFITHS, Coventry, Warwickshire (GB); Joleen Solange Liesbet MASSCHELEIN, Coventry, Warwickshire (GB)

(21) Appl. No.: 16/094,617

(22) PCT Filed: Apr. 21, 2017
(86) PCT No.: PCT/GB2017/051124

§ 371 Date: Oct. 18, 2018

(30) Foreign Application Priority Data
Apr. 21, 2016 (GB) 1606933.8

(51) Int. Cl. C07C 69/732; C12P 7/64; C07C 69/738; C07C 235/28; A61K 31/232; C12P 13/02

(52) U.S. Cl. CPC C07C 69/732; C12P 7/6436; C07C 69/738; C07C 235/28; C07C 2601/16; C12P 13/02; C07C 2601/08; C07C 2601/14; A61K 31/232

(57) ABSTRACT

The invention provides novel compounds of formula (I) and their pharmaceutically acceptable salts, metabolites, isomers (e.g. stereoisomers) and prodrugs. Such compounds are effective in the treatment of infections caused by Gram-negative bacteria such as Acinetobacter baumannii. In formula (I), X is O, NR (where R is either H or C₁₋₃ alkyl, e.g. CH₃), or CH₂; R³ is H, F, CI, Br, I, or CH₃; R⁴ is H, or OH; R⁵ and R⁶ are independently selected from H and OH, or R⁵ and R⁶ together are ═O; R⁷ is H, F, CI, Br, I, or CH₃; R⁸ is H, OH, or —OC(O)NR′₂ (where each R′ is independently H or C₁₋₃ alkyl, e.g. CH₃), preferably R⁸ is H, OH or —OC(O)NH₂; R⁹ is a 5- or 6-membered, saturated or unsaturated, carbocyclic ring optionally substituted by one or more substituents, or R⁹ is an optionally substituted straight-chained or branched C₁₋₆ alkyl group (e.g. C₁₋₃ alkyl group); R¹⁰ is a straight-chained or branched C₁₋₈ alkyl group (e.g. C₁₋₆ alkyl group), a C₄₋₆ cycloalkyl group, or an optionally substituted aryl or heteroaryl group; and each --- independently represents an optional bond (i.e. each of C₂-C₃, C₄-C₅, C₆-C₇, C₈-C₉, C₁₀-C₁₁ and C₁₈-C₁₉ are independently either C—C (single) or C═C (double) bonds).

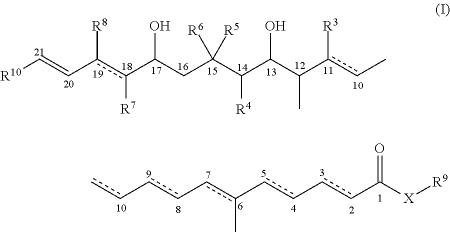

The present invention relates to novel polyketide compounds, their preparation, and their use as antimicrobial agents. The invention further relates to antimicrobial agents obtained from a Vibrio rhizosphaerae strain, or from a variant and/or mutant thereof.

Bacterial pathogens are prominent in many diseases and the treatment of bacterial infections has become increasingly difficult over recent years with the emergence of a number of antibiotic resistant bacterial strains. Examples include methicillin-resistant Staphylococcus aureus (MRSA), vancomycin-resistant Enterococci (VRE), and multidrug-resistant Gram-negative bacteria such as Acinetobacter baumannii. In addition to the emergence of antibiotic resistant strains, there are many bacterial infections that remain difficult to treat, for example, infections in immuno-compromised patients (e.g. those with AIDS).

There is therefore an ongoing need to identify new antimicrobial agents that can be used to treat microbial infections effectively, including those caused by drug resistant microbes, for example, infections caused by drug-resistant bacteria.

The inventors have identified a novel polyketide compound (herein referred to as “vibroxin”) as responsible for the anti-microbial activity of a Vibrio rhizosphaerae strain. This compound has been isolated and its structure determined as shown below:

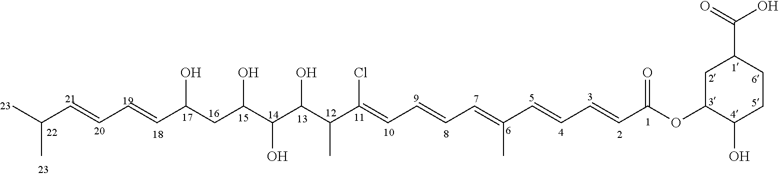 [SMILES: CC(/C=C/C=C/C(=O)OC1CC(C(=O)O)CCC1O)=C\C=C\C=C(/Cl)C(C)C(O)C(O)C(O)CC(O)/C=C/C=C/C(C)C]

(This structure is intended primarily to illustrate the connectivity of the molecule and is not necessarily intended as an accurate representation of the cis/trans stereochemistry of the double bonds in the molecule).

Vibroxin has a similar structure to that of enacyloxin IIa, a compound having antibiotic activity that is produced by strains of Burkholderia and Frateuria species. Enacyloxin IIa has the following structural formula:

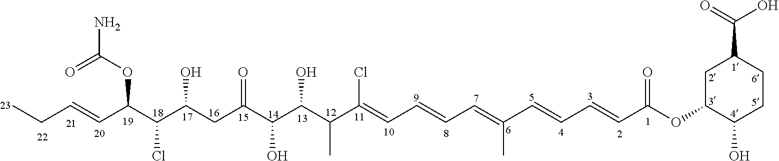

WO 2011/101631 describes the previously known compound enacyloxin IIa and derivatives having anti-bacterial activity. However, such compounds are unstable. The ester group is prone to rearrangement and hydrolysis. This chemical instability limits their potential for clinical use.

The present inventors have discovered that vibroxin, and the derivatives of vibroxin that are herein described, are structurally simpler, in some cases are more chemically stable, and have equal or superior potency against multidrug-resistant pathogenic bacteria such as Acinetobacter baumannii. This structural simplification, and improvement in stability and potency represents a significant advance over the earlier known compounds. Furthermore, based on what is known about the structure-activity relationship for enacyloxin IIa, it could not have been predicted that biological activity would be retained or enhanced following modification at the C18, C19 and/or C22 positions of the molecule.

The present invention also provides certain analogues of vibroxin. These may be produced using methods known in the art, for example techniques capable of modifying the genes responsible for vibroxin biosynthesis in order to produce recombinant microbes that biosynthesise the analogues. Such analogues are as herein described and may differ from vibroxin at key positions on the polyketide chain and/or in respect of modifications made to the dihydroxycyclohexane carboxylic acid (DHCCA) moiety. Specific methods which may be used to produce analogues of vibroxin may involve the use of a heterologous host for the expression of vibroxin biosynthetic genes, or a combination of vibroxin and enacyloxin biosynthetic genes, knock-out mutagenesis, mutasynthesis, semi-synthetic modification and/or total chemical synthesis.

Accordingly, the present invention provides novel polyketide compounds that are effective against a range of microbes, including bacteria and resistant bacteria, and in particular against multidrug-resistant Gram-negative bacteria such as Acinetobacter baumannii. The invention also provides recombinant microorganisms and hybrid vibroxin/enacyloxin gene clusters capable of producing such compounds.

The compounds of the invention, including but not limited to those specified in the examples herein, possess the ability to inhibit and/or prevent the growth of microbes. Such compounds may be useful in the treatment of a wide variety of microbial infections. The present invention further provides pharmaceutical compositions comprising one or more compounds according to the invention. In addition, compounds of the invention may be useful in the treatment of microbial infections described herein either when used alone or in combination with other therapeutic agents.

Further aspects of the present invention include: processes for the preparation of the compounds according to the invention; methods for the treatment of infections by microbes, including drug-resistant strains thereof, comprising administering a compound according to the present invention; and uses of the compounds according to the present invention.

Viewed from a first aspect the invention provides a compound of formula (I), or a pharmaceutically acceptable salt, metabolite, isomer (e.g. stereoisomer) or prodrug thereof:

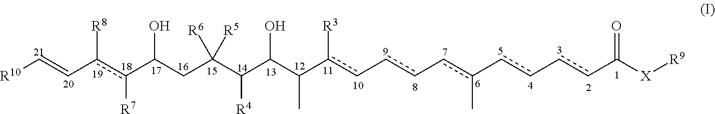

wherein:
X is O, NR (where R is either H or C₁₋₃ alkyl, e.g. CH₃), or CH₂;
R³ is H, F, Cl, Br, I, or CH₃;
R⁴ is H, or OH;
R⁵ and R⁶ are independently selected from H and OH, or R⁵ and R⁶ together are ═O;
R⁷ is H, F, Cl, Br, I, or CH₃;
R⁸ is H, OH, or —OC(O)NR′₂ (where each R′ is independently H or C₁₋₃ alkyl, e.g. CH₃), preferably R⁸ is H, OH or —OC(O)NH₂;
R⁹ is a 5- or 6-membered, saturated or unsaturated, carbocyclic ring optionally substituted by one or more substituents, or R⁹ is an optionally substituted straight-chained or branched C₁₋₆ alkyl group (e.g. C₁₋₃ alkyl group);
R¹⁰ is a straight-chained or branched C₁₋₈ alkyl group (e.g. C₁₋₆ alkyl group), a C₄₋₆ cycloalkyl group, or an optionally substituted aryl or heteroaryl group; and
each --- independently represents an optional bond (i.e. each of C₂-C₃, C₄-C₅, C₆-C₇, C₈-C₉, C₁₀-C₁₁ and C₁₈-C₁₉ are independently either C—C (single) or C═C (double) bonds).

In one embodiment, R⁹ is a cyclohexyl or cyclopentyl ring which is optionally substituted by one or more substituents.

In another embodiment, R⁹ is a cyclohexenyl ring which is optionally substituted by one or more substituents.

In another embodiment, R⁹ is a straight-chained or branched C₁₋₆ alkyl group (e.g. C₁₋₃ alkyl group) which may be substituted by one or more substituents. Preferably it is a straight-chained alkyl group.

Optional substituents which may be present in group R⁹ include one or more of the following: OH, NR^{a}₂ (where each R^{a} is independently H or C₁₋₃ alkyl, e.g. CH₃), SR^{b} (where R^{b} is H or C₁₋₃ alkyl, e.g. CH₃), halogen (e.g. F, Cl, Br, or I), C₁₋₃ alkyl (e.g. CH₃), CO₂H (or an ester thereof), PO₃H₂ (or an ester thereof) and SO₃H₂ (or an ester thereof). Suitable ester-forming groups include optionally substituted alkyl, alkenyl, cycloalkyl, cycloalkenyl, aryl and heteroaryl groups. Examples of such groups include optionally substituted C₁₋₁₂-alkyl, C₁₋₁₂-alkenyl, C₃₋₁₀-cycloalkyl, aryl and heteroaryl groups, wherein the aryl and heteroaryl groups may contain from 5 to 10 carbon atoms and the heteroaryl groups further contain one or more (e.g. 1, 2, 3 or 4) heteroatoms selected from N, O and S.

In one embodiment, the substituents which may be present in group R⁹ may be selected from any of the following: OH, NH₂, SH, F, Cl, Br, I, CH₃, CO₂H, PO₃H₂ and SO₃H₂.

In one embodiment, one, two or three (preferably one or two) substituents may be present in group R⁹. Where more than one substituent is present, these may be the same or different. Preferably, at least one of the substituents will be CO₂H or an ester thereof as herein defined.

In one embodiment, where R⁹ is substituted by more than one substituent (e.g. two or three substituents), the substituents may be selected from the group consisting of CO₂H (or an ester thereof), and OH. Preferably R⁹ may be substituted by one CO₂H group (or an ester thereof), and/or by one OH group, e.g. by one CO₂H group (or an ester thereof), and by one OH group.

In one embodiment, R⁹ is an optionally substituted cyclohexyl or cyclopentyl group. Substituents on these rings may be any of those herein described. Preferably, the substituents may be selected from CO₂H (or an ester thereof), and OH. In one embodiment, R⁹ is a cyclohexyl ring substituted by one CO₂H group (or an ester thereof), and by one OH group. These substituents may be present at any ring positions, but in one embodiment these may be para to one another.

Where R⁹ is a straight-chained or branched C₁₋₆ alkyl group (e.g. C₁₋₃ alkyl group), this is preferably substituted. Preferred substituents are selected from CO₂H (or an ester thereof), and OH. In one embodiment, R⁹ is a straight-chained or branched (preferably straight-chained) C₁₋₆ alkyl group (e.g. C₁₋₃ alkyl group) substituted by one CO₂H group (or an ester thereof), and/or by one OH group. Where present, any CO₂H group (or an ester thereof) will typically be provided at the terminal position of the alkyl group.

Examples of R⁹ groups include any of the following (in which * denotes the point of attachment of the substituent to the remainder of the molecule):

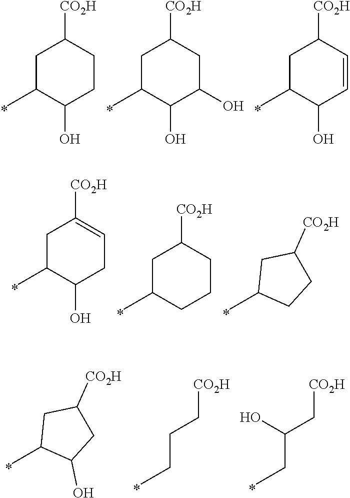

In one embodiment R¹⁰ is a straight-chained or branched C₁₋₈ alkyl (e.g. C₁₋₆ alkyl) group, preferably a straight-chained or branched C₁₋₅ alkyl, more preferably a straight-chained or branched C₁₋₄ alkyl. Examples of such groups include methyl, ethyl, isopropyl, and tert. butyl.

In another embodiment R¹⁰ is a C₄₋₆ cycloalkyl group, for example a cyclohexyl or cyclopentyl group. In one embodiment, R¹⁰ may be a cyclohexyl group.

In another embodiment R¹⁰ is an optionally substituted aryl group or heteroaryl group wherein the aryl and heteroaryl groups may contain from 5 to 10 carbon atoms and the heteroaryl groups further contain one or more (e.g. 1, 2, 3 or 4) heteroatoms selected from N, O and S. Examples of optional ring substituents include OH, NR^{a}₂ (where each R^{a} is independently H or C₁₋₃ alkyl, e.g. CH₃), SR^{b}(where R_{b} is H or C₁₋₃ alkyl, e.g. CH₃), halogen (e.g. F, Cl, Br, or I), and C₁₋₃ alkyl (e.g. CH₃). In one embodiment, R¹⁰ may be optionally substituted phenyl, e.g. unsubstituted phenyl.

In a preferred embodiment, R¹⁰ is an isopropyl group.

In one embodiment, R⁹ is a substituted cyclohexyl group and R¹⁰ is isopropyl.

In one embodiment, the invention provides a compound of formula (Ia) or a pharmaceutically acceptable salt, metabolite, isomer (e.g. stereoisomer) or prodrug thereof:

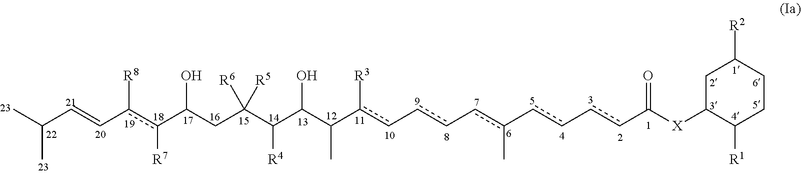

wherein:
R¹ is H, OH, NR^{a}₂ (where each R^{a} is independently H or C₁₋₃ alkyl, e.g. CH₃), SR^{b} (where R^{b} is H or C₁₋₃ alkyl, e.g. CH₃), halogen (e.g. F, Cl, Br, or I), or C₁₋₃ alkyl (e.g. CH₃), preferably wherein R¹ is H, OH, NH₂, SH, F, Cl, Br, I, or CH₃;
R² is H, CO₂H (or an ester thereof), PO₃H₂ (or an ester thereof) or SO₃H₂ (or an ester thereof), preferably wherein R² is H, CO₂H, PO₃H₂, or SO₃H₂;
X is as herein defined;
R³ to R⁸ are as herein defined; and
each [symbol] independently represents an optional bond (i.e. each of C₂-C₃, C₄-C₅, C₆-C₇, C₈-C₉, C₁₀-C₁₁ and C₁₈-C₁₉ are independently either C—C (single) or C═C (double) bonds).

In one embodiment the invention provides compounds of formula (Ia) and their pharmaceutically acceptable salts, metabolites, isomers (e.g. stereoisomers) and prodrugs, wherein:
R¹ is H, OH, NH₂, SH, F, Cl, Br, I, or CH₃;
R² is H, CO₂H, PO₃H₂, or SO₃H₂;
X═O, NH, or CH₂;
each of C₂-C₃, C₄-C₅, C₆-C₇, C₈-C₉, C₁₀-C₁₁ and C₁₈-C₁₉ are independently either C—C (single) or C═C (double) bonds;
R³ is H, F, Cl, Br, I, or CH₃;
R⁴ is H, or OH;
R⁵ is H and R⁶ is OH, or R⁵ and R⁶ are ═O;
R⁷ is H, F, Cl, Br, I, or CH₃; and
R⁸ is H, OH, or OC(O)NH₂.

In another embodiment, at least one of R⁷ and R⁸ in formula (I) or (Ia) is hydrogen. In another embodiment, both R⁷ and R⁸ are hydrogen.

In one embodiment, the invention provides a compound of formula (Ib), or a pharmaceutically acceptable salt, metabolite, isomer (e.g. stereoisomer) or prodrug thereof:

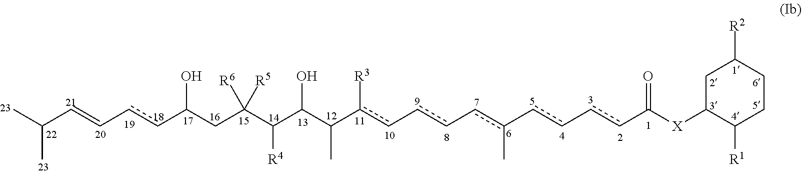

wherein: R¹ to R⁶ and X are as herein defined.

In certain embodiments of the invention, X is O or NR (where R is either H or C₁₋₃ alkyl, e.g. CH₃). Preferably X is O or NH. More preferably, X is NH.

In certain embodiments of the invention, R is H or Cl. Preferably, R³ is Cl.

In certain embodiments, R⁴ is OH.

In certain embodiments, R⁵ is H and R⁶ is OH.

In one embodiment of the invention C₂-C₃, C₄-C₅, C₆-C₇, C₈-C₉, C₁₀-C₁₁ and C₁₈-C₁₉ are C═C (double) bonds.

In one aspect, the invention provides a compound of formula (II), or a pharmaceutically acceptable salt, metabolite, isomer (e.g. stereoisomer) or prodrug thereof:

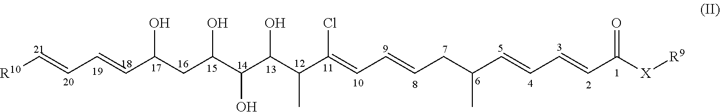

wherein X, R⁹ and R¹⁰ are as herein defined.

In one embodiment of formula (II), R¹⁰ is isopropyl. In a further embodiment of formula (II), R⁹ is a cyclohexyl ring substituted by one CO₂H group (or an ester thereof), and by one OH group.

In another embodiment, the invention provides a compound of formula (IIa), or a pharmaceutically acceptable salt, metabolite, isomer (e.g. stereoisomer) or prodrug thereof:

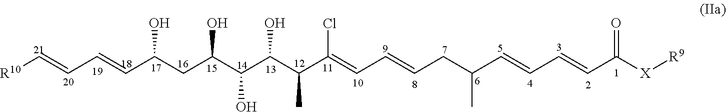

wherein X, R⁹ and R¹⁰ are as herein defined.

In one embodiment of formula (IIa), R¹⁰ is isopropyl. In a further embodiment of formula (IIa), R⁹ is a cyclohexyl ring substituted by one CO₂H group (or an ester thereof), and by one OH group.

Examples of compounds according to the invention include the following, and their pharmaceutically acceptable salts, metabolites, isomers (e.g. stereoisomers) and prodrugs:

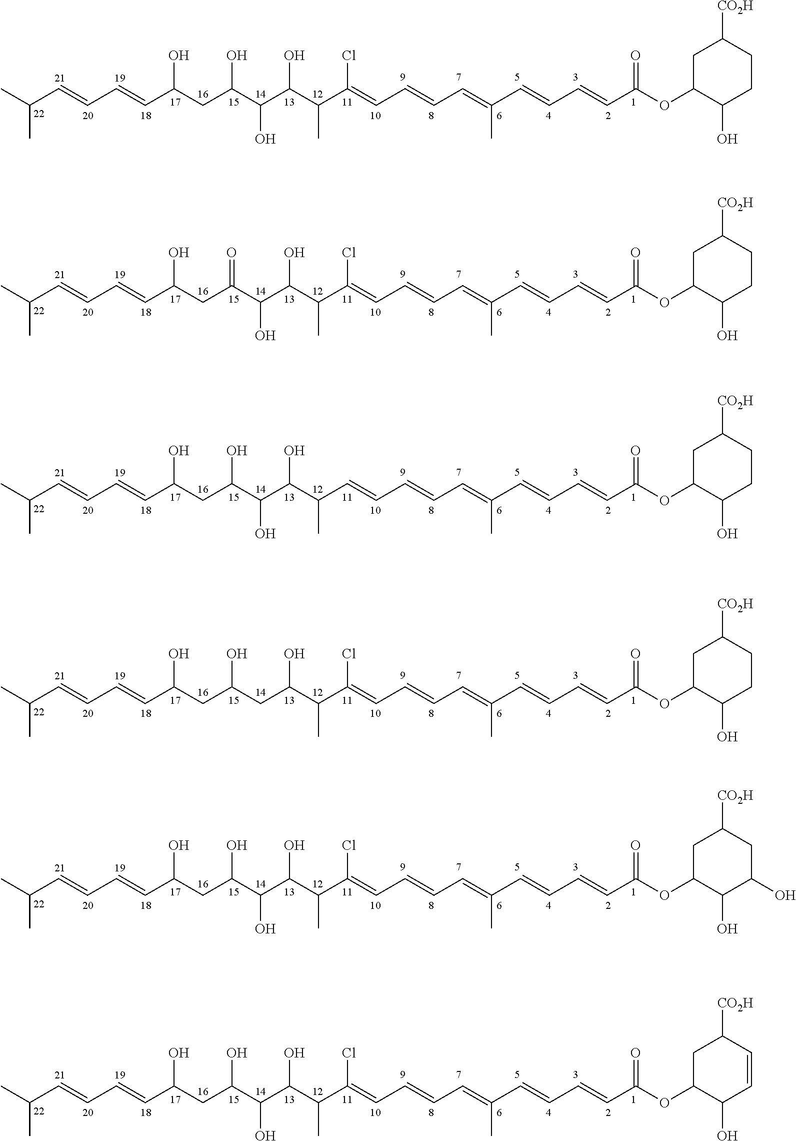

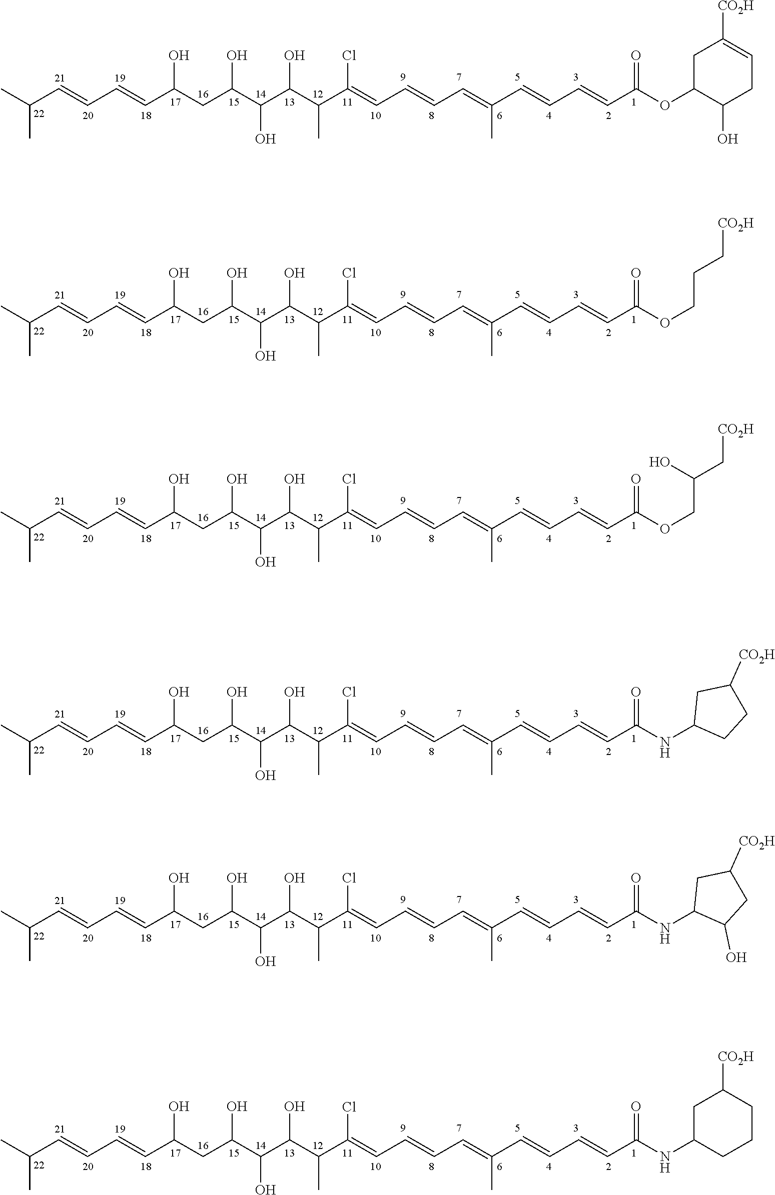

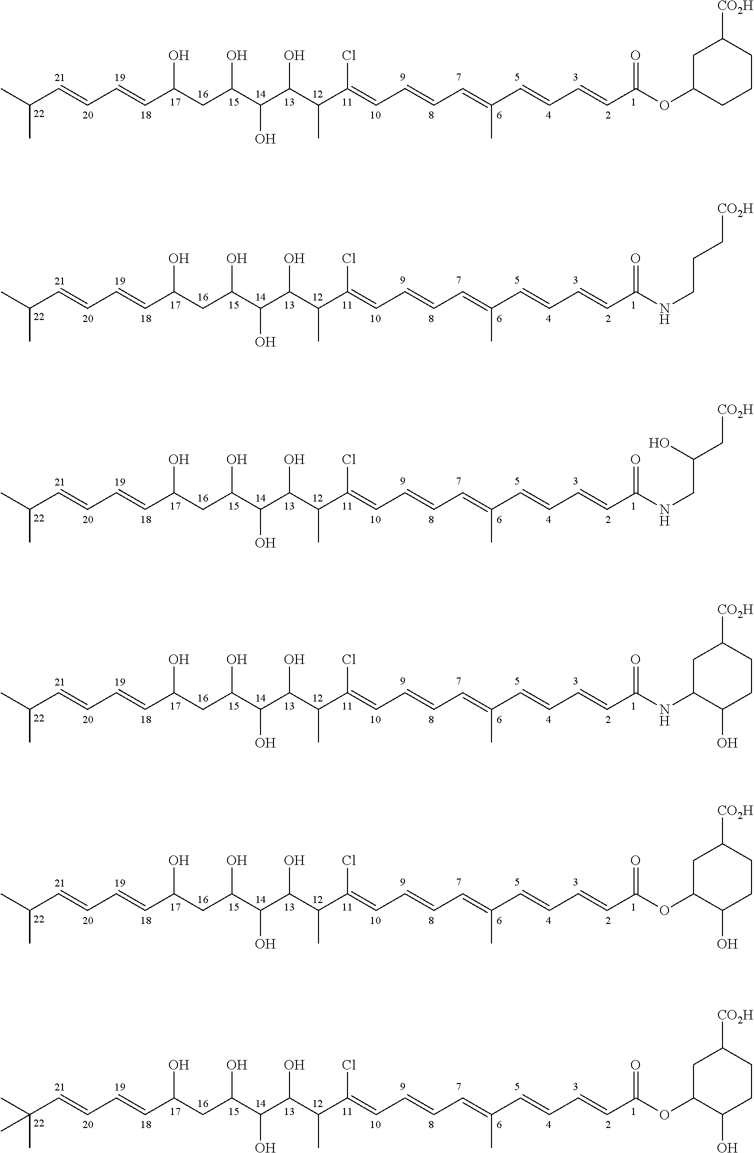

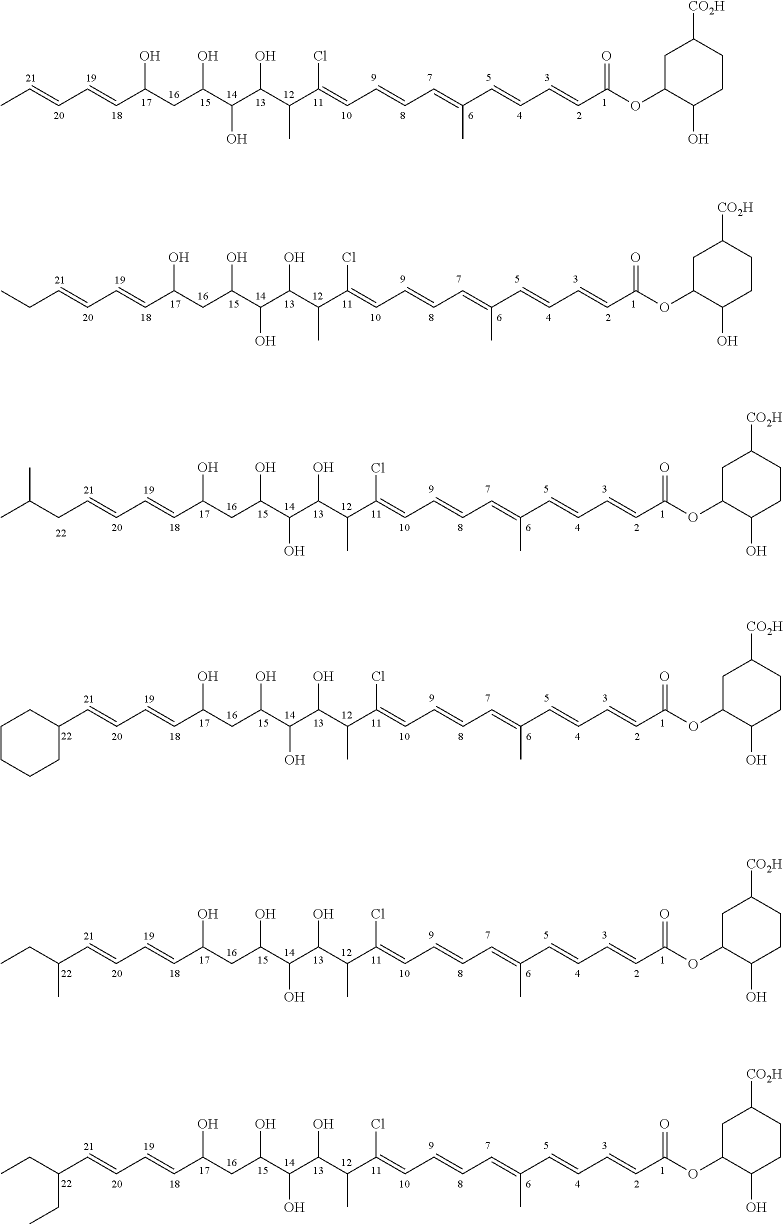

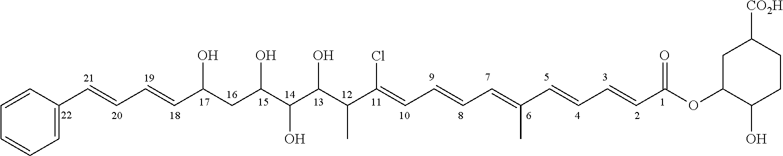 [SMILES: CC(/C=C/C=C/C(=O)OC1CC(C(=O)O)CCC1O)=C\C=C\C=C(/Cl)C(C)C(O)C(O)C(O)CC(O)/C=C/C=C/c1ccccc1]

Further examples of compounds according to the invention include the following, and their pharmaceutically acceptable salts, metabolites, and prodrugs:

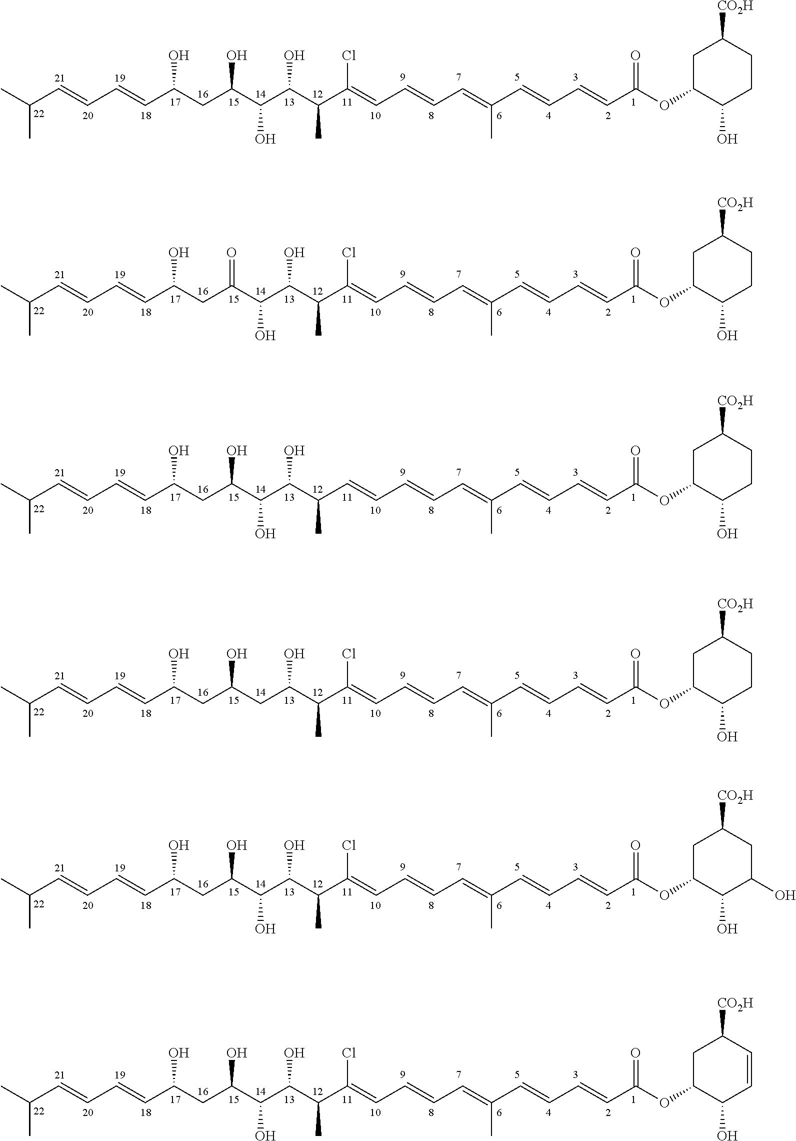

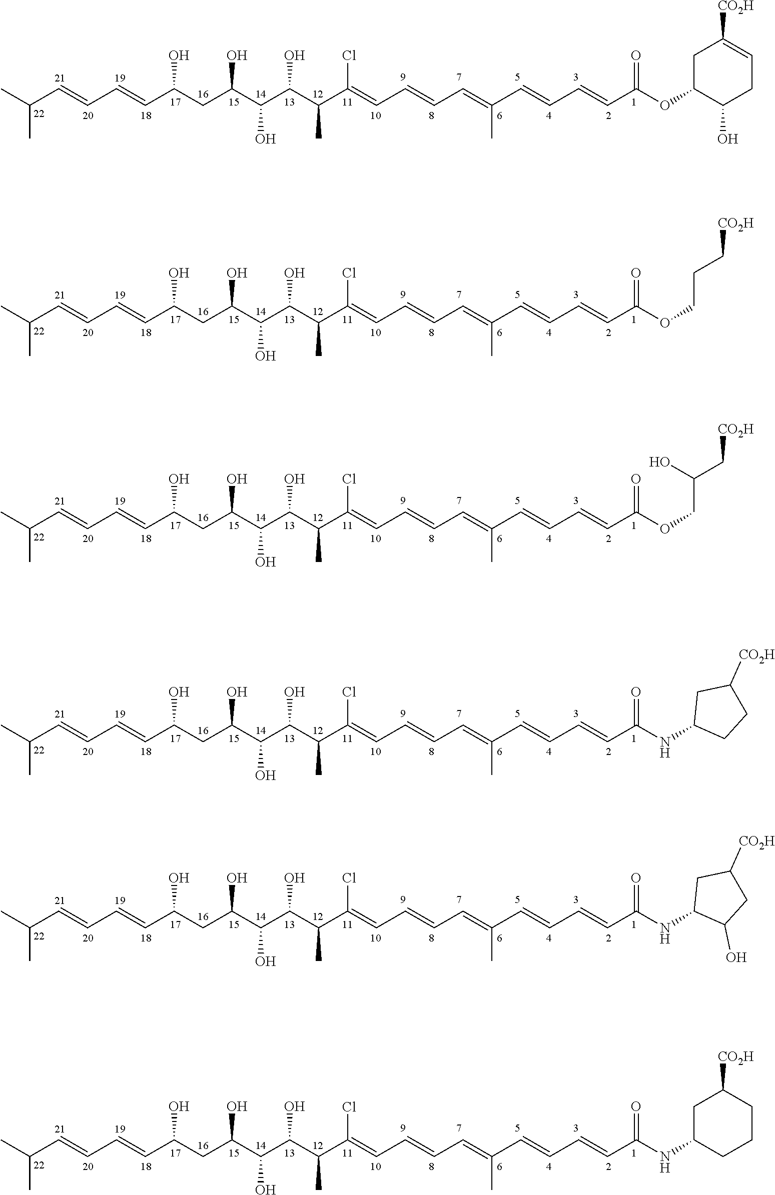

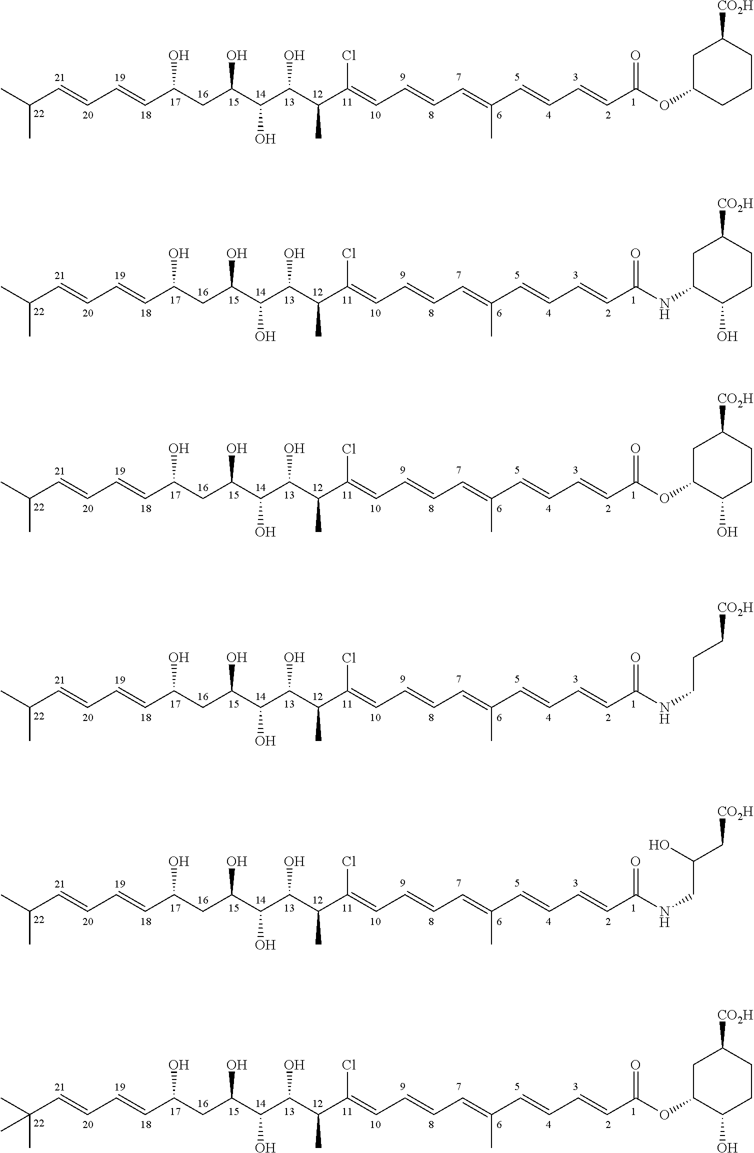

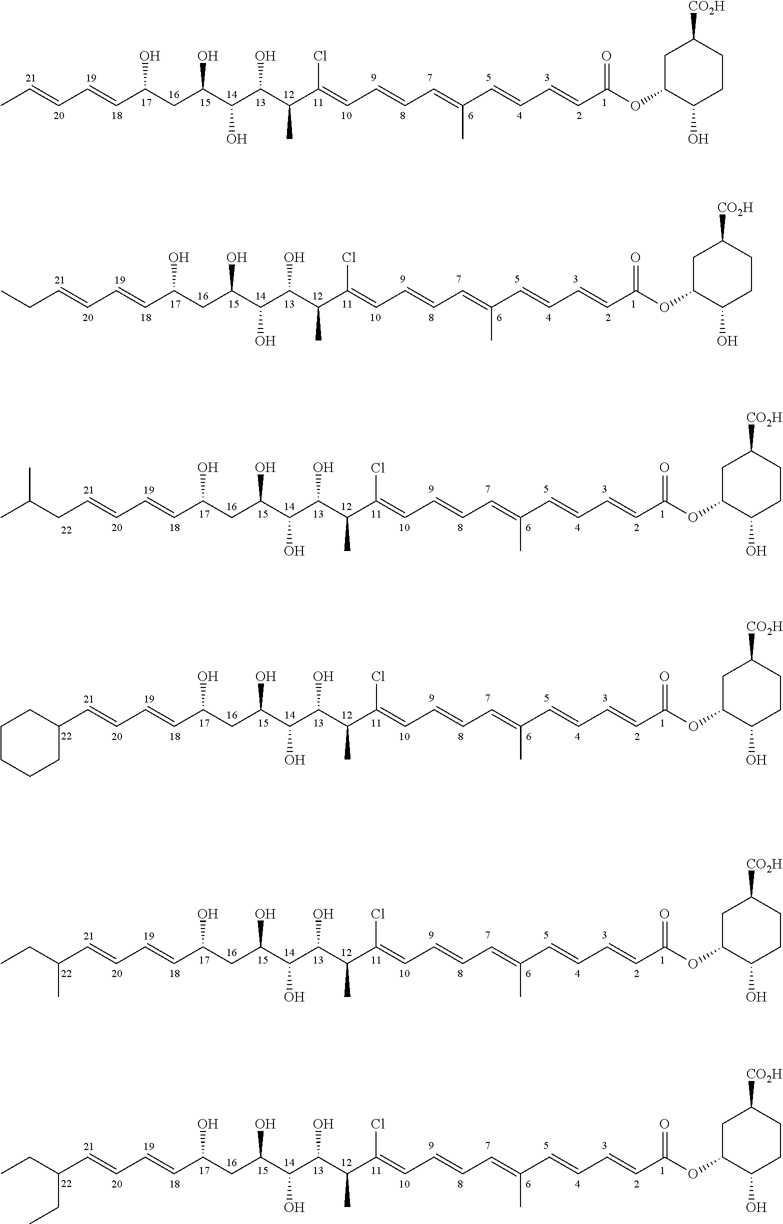

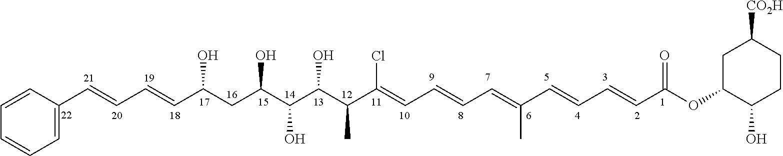 [SMILES: CC(/C=C/C=C/C(=O)O[C@@H]1C[C@@H](C(=O)O)CC[C@@H]1O)=C\C=C\C=C(/Cl)[C@@H](C)[C@@H](O)[C@H](O)[C@H](O)C[C@@H](O)/C=C/C=C/c1ccccc1]

The compounds of the invention are suitable for pharmaceutical and medical uses, in particular they are useful as antimicrobial agents. More specifically, the compounds of the present invention provide new agents for application against bacteria, multidrug-resistant bacteria and combinations thereof thus offering both separate and combination treatment potential. The compounds of the present invention have application for the treatment of various infections, for example including infections of the skin and skin structure, infections of the respiratory system, endocarditis, hospital acquired infections, infections of the digestive system, urinary system, nervous system, blood infection, soft tissue infection, nasal canal infections and infection associated with cystic fibrosis. The compounds of the present invention also find application in relation to or for animal/veterinary illnesses.

Thus, in another aspect, the invention provides a pharmaceutical composition comprising a compound according to the invention or a pharmaceutically acceptable salt, metabolite, isomer (e.g. stereoisomer) or prodrug thereof along with one or more physiologically acceptable carriers, excipients or diluents.

Also provided are methods of treating infections (such as those listed above) comprising administration of one or more compounds of the invention, optionally in combination with one or more further active agents.

In a related aspect, the invention provides a compound as defined herein for use as a medicament or in therapy, e.g. for use in the treatment of infections such as those listed above. In one embodiment, the compounds of the invention may be used to treat infections caused by a microbe which is resistant to known antimicrobial agents.

In another aspect, the invention provides a variant and/or mutant of the microorganism Vibrio rhizosphaerae, e.g. a variant and/or mutant of Vibrio rhizosphaerae MSSRF3 (DSM 18581).

In this aspect the term “variant” includes, but is not limited to, a bacterial strain that differs from the specified bacterial strain but which is able to produce vibroxin or any of the derivatives thereof as described herein, e.g. according to the methods described herein. This term can also mean a bacterial strain that differs from the specified bacterial strain but which retains sufficient genotypic or phenotypic characteristics to maintain a taxonomic similarity.

In this aspect the term “mutant” includes, but is not limited to, a bacterial strain that has arisen as a result of mutation in, or gene editing of, the specified bacterial strain provided said mutant strain is able to produce vibroxin or any of the derivatives thereof as described herein, e.g. according to the methods described herein. This term can also mean a bacterial strain that differs from the specified bacterial strain as a result of mutation, or gene editing, which for example results in an altered gene, DNA sequence, enzyme, cell structure, etc.

Such mutants can be produced in a manner known in the art, for example by physical means such as irradiation (for example UV), by exposure to chemical mutagens or by genetic manipulation of DNA of the bacterium. Methods for screening for mutants and isolating mutants will be known to a person skilled in the art.

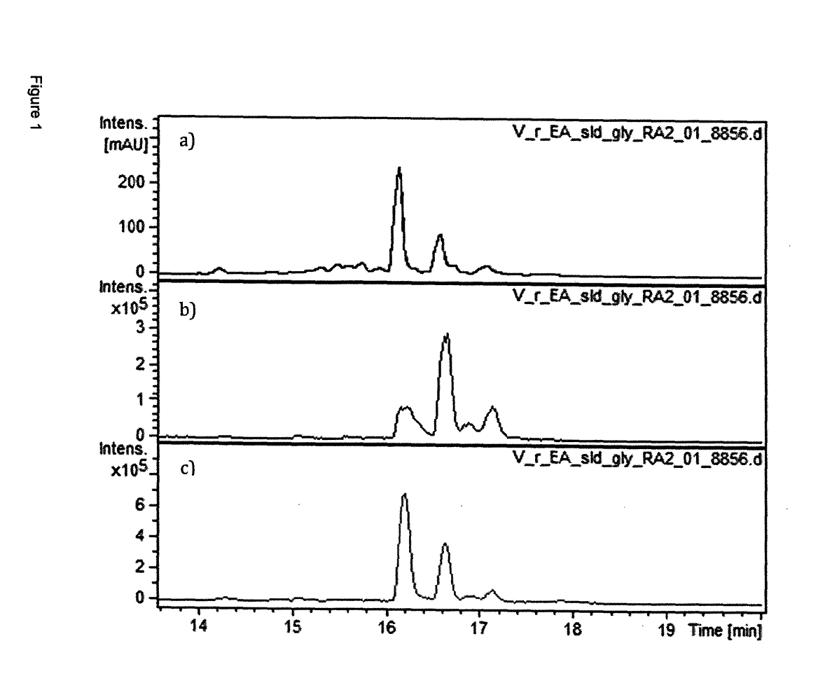

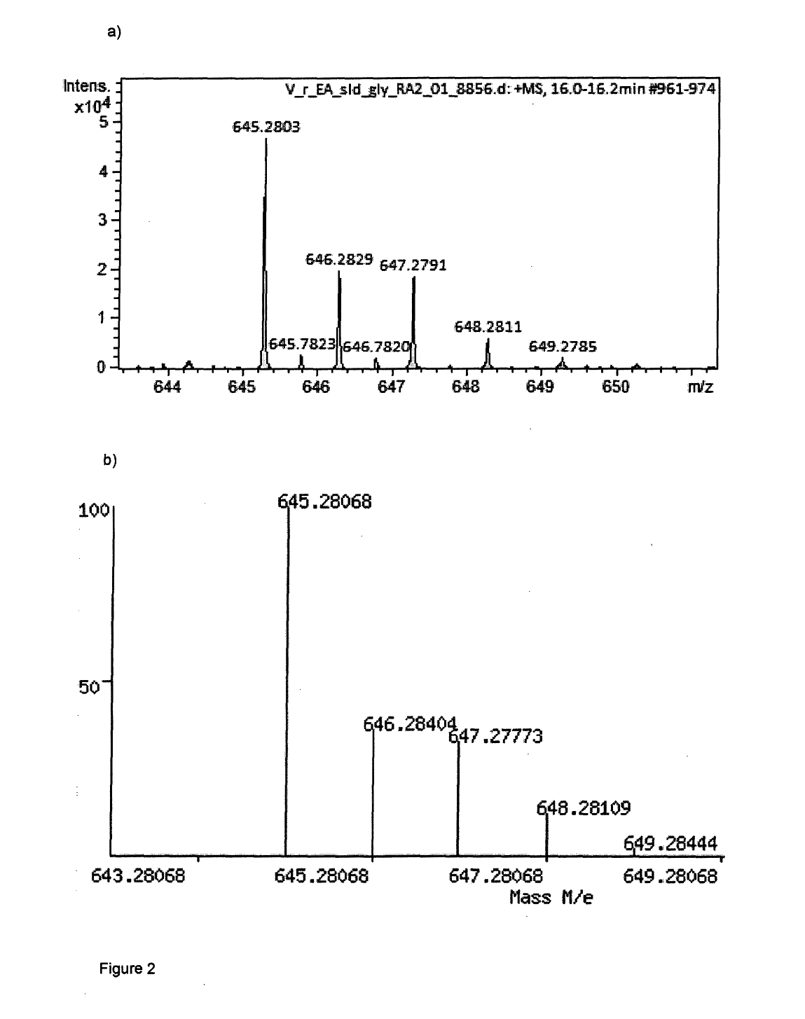

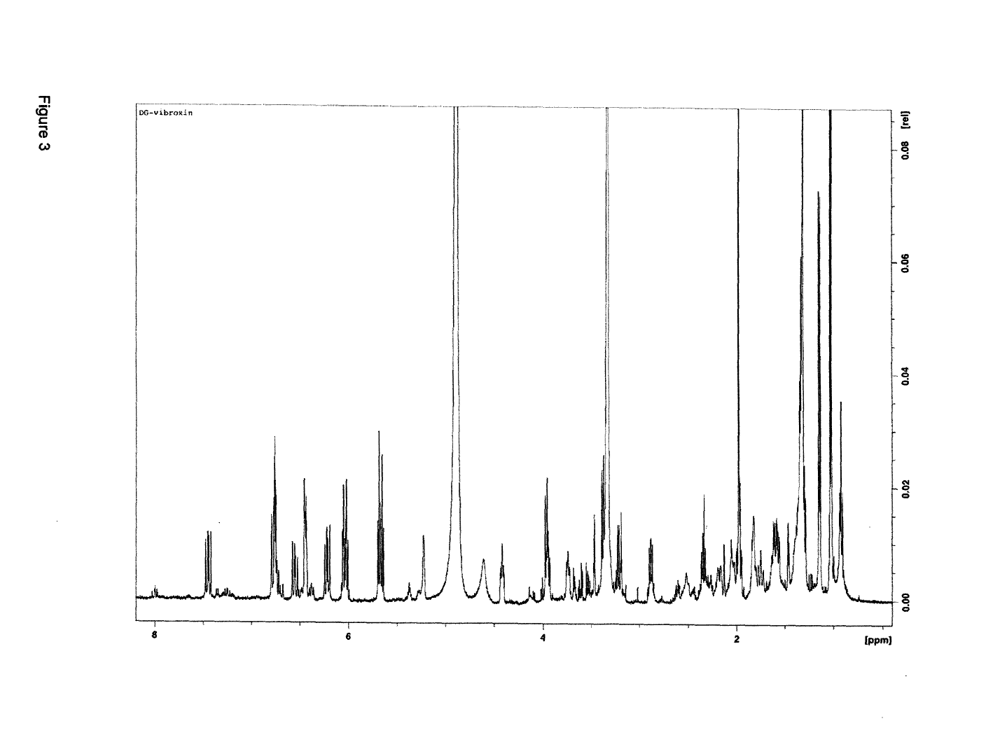

Accordingly, the invention also provides an active agent, especially an antimicrobial agent, obtained or obtainable from Vibrio rhizosphaerae, e.g. from Vibrio rhizosphaerae MSSRF3 (DSM 18581). Preferably, the active agent is a polyketide compound, especially a compound having one or more of the characteristics identified below:
a molecular formula C₃₃H₄₇ClO₉;
carbon (13C) and hydrogen (1H) NMR spectral signals substantially in accordance with Table 3 and/or one or more of FIGS. 3 to 7; and
a mass spectral signal substantially in accordance with FIG. 1 and/or FIG. 2.

Where compounds of the invention are used in the treatment of an infection caused by a microbe, the microbe may be a Gram-negative bacterium. Such infectious Gram-negative bacteria are preferably selected from Acinetobacter species, Burkholderia species, Ralstonia species and Stenotrophomonas species. The bacterium may, for example, be Acinetobacter baumannii, Enterococcus faecium, Staphylococcus aureus, Klebsiella pneumoniae, Pseudomonas aeruginosa, or Enterobacter cloacae.

Preferably, the compounds according to the present invention are for use in the treatment of an infection caused by more than one type of microbe, for example, two or more different bacterial species.

Preferably, the compounds according to the present invention are for use in the treatment of an infection caused by a microbe that is resistant to at least one antimicrobial drug, for example an antimicrobial drug known in the art. The infection may be caused by one or more bacteria that show resistance to common antimicrobial drugs. The bacterium may be multidrug-resistant. For example, the infection may be caused by carbapenem-resistant Acinetobacter baumannii, or by MRSA or VRE.

The antimicrobial drug against which the microbe has become resistant may be an antibacterial drug. The antibacterial drug may be selected from, but is not limited to: drugs of the carbapenem family, drugs of the penicillin family, drugs of the vancomycin family, drugs of the aminoglycoside family, drugs of the quinolone family, drugs of the daptomycin family, drugs of the cephalosporin family, drugs of the macrolide family and combinations thereof. Examples of such antibacterial drugs include carbapenems, penicillin, ampicillin, methicillin, vancomycin, gentamycin, ofloxacin, ciprofloxacin, daptomycin, cefdimir, erythromycin, equivalents thereof, and combinations thereof.

Preferably, the compounds according to the present invention are for use in the treatment of an infection in an animal, preferably a mammal, more preferably a human. Preferably, the compounds according to the present invention are for use in the treatment of an infection in a non-human mammal, such as a dog, cat, horse, etc. The compounds according to the present invention therefore have application in both human and veterinary medicine.

Preferably, the compounds according to the present invention are for use in the treatment of an infection of the respiratory system, digestive system, urinary system, nervous system, a blood infection, a soft tissue infection, a skin infection, a nasal canal infection, or combinations thereof.

Preferably, the compounds according to the present invention are for use in the treatment of a bacterial infection of the respiratory system or a portion thereof, for example, the upper respiratory system.

Preferably, the compounds according to the present invention are for use in the treatment of an infection associated with immuno-compromised individuals, for example in the treatment of elderly or paediatric patients.

Preferably, the compounds and methods of the present invention are for use in treating a variety of infections that comprise different types of Gram-negative bacteria, including aerobic or anaerobic bacteria. These types of infections include intra-abdominal infections, pneumonia, bone and joint infections, and obstetrical/gynaecological infections and urinary tract infections.

The compounds and methods of the invention may also be used to treat an infection including, without limitation, endocarditis, nephritis, septic arthritis and osteomyelitis.

According to a further aspect of the present invention, there is provided a pharmaceutical composition comprising a compound according to the present invention, or a pharmaceutically acceptable salt, metabolite, or prodrug thereof, in combination with a pharmaceutically acceptable carrier. The pharmaceutical composition may further comprise one or more other therapeutic agents, for example selected from an anti-inflammatory agent, anti-cancer agent or immuno-modulatory agent, or different types of antibacterial and/or antifungal agents.

Preferably, a therapeutic agent, other than a compound of the present invention, may be administered concurrently with a compound of the present invention. In a preferred embodiment, an antibacterial and/or antifungal agent may be administered concurrently with a compound of the present invention. Co-administration of an antifungal agent and/or an antibacterial agent, other than a compound of the present invention, may be useful for mixed infections such as those caused by different types of bacteria, or those caused by both bacteria and fungi. The different therapeutic agents may be administered sequentially, separately or simultaneously.

Antibacterial agents and classes thereof that may be co-administered with a compound of the present invention preferably include, without limitation, penicillins and related drugs, carbapenems, cephalosporins and related drugs, aminoglycosides, ceftriaxone, daptomycins and macrolides.

Antifungal agents that may be co administered with a compound according to the present invention preferably include, without limitation, caspofungen, polyenes, such as amphotericin, nystatin and pimaricin; azoles, such as fluconazole, itraconazole, ketoconazole, voriconazole and sertaconazole; and allylamines, such as naftifine and terbinafine.

Another aspect of the present invention relates to the use of a compound according to the present invention for inhibiting the growth or survival of a microbe. The microbe may be resistant to at least one antimicrobial agent. The microbe is preferably a bacterium, for example at least one bacterium selected from Acinetobacter species, Mycobacterium species, Burkholderia species, Pseudomonas species, Ralstonia species, and Stenotrophomonas species.

According to another aspect of the present invention, there is provided a process for the preparation of a compound according to the present invention. Preferably, the process comprises cultivating a microorganism capable of producing a compound as herein described, such as Vibrio rhizosphaerae MSSRF3 (DSM 18581), or a mutant or variant thereof, optionally in the presence of any appropriate precursor compound such as those which are herein described. Vibrio rhizosphaerae MSSRF3 (DSM 18581) is a red-pigmented strain originally isolated from the rhizosphere of mangrove-associated wild rice (see Kumar et al., Vibrio rhizosphaerae sp. nov., a red-pigmented bacterium that antagonizes phytopathogenic bacteria. int. J. Syst. Evol. Microbiol. 57(Pt10): 2241-6, 2007). It is commercially available from a number of sources, including the Leibniz Institute DSMZ (German Collection of Microorganisms and Cell Cultures).

Cultivation of the microorganism may be carried out in a culture or nutrient medium comprising a source of assimilable carbon, nitrogen, and inorganic salts, thereby producing a cultivation medium comprising the desired compound. Preferred nutrient media are agar-based (e.g. a BSM-agar supplemented with NaCl and glycerol). Where a precursor compound is supplied to a blocked mutant in order to produce certain vibroxin analogues as herein described, this compound will typically be added to the nutrient medium. Where agar is used, for example, the precursor compound may be applied to the agar (e.g. at a concentration of about 10 mM) before spreading the chosen microorganism on top.

Optionally, the desired compound may be recovered from the cultivation medium or fermentation broth. The process may further comprise converting any compound obtained into an alternative compound according to the invention by known chemical syntheses. The process may also comprise converting the compound obtained into a pharmaceutically acceptable salt.

Conversion of any —COOH group to an ester derivative may be effected using methods which are known in the art (see, for example, March, J., Advanced Organic Chemistry, John Wiley & Sons, 4th edition, 1992). For example, vibroxin may be reacted with an optionally activated alkyl compound, such as a diazoalkane, to form the respective alkyl ester.

Vibroxin and its derivatives can be isolated and purified from the culture medium using known methods and taking account of the chemical, physical and biological properties of the natural substances. For the isolation, vibroxin may be extracted from an agar culture or liquid culture using an organic solvent, such as methanol or ethyl acetate, and may be subjected to further purification. The further purification of vibroxin may be effected by chromatography on suitable materials, for example on reverse phase HPLC resins.

Insofar as the vibroxin and its derivatives are present as stereoisomers, they can be separated using known methods, for example by means of separation using a chiral column.

Preferably, the producer microorganism is Vibrio rhizosphaerae, e.g. Vibrio rhizosphaerae MSSRF3 (DSM 18581), or a mutant or variant thereof as herein described. Other microorganisms, in particular bacteria, engineered to carry the appropriate biosynthetic genes may also be used.

Vibrio rhizosphaerae MSSRF3 (DSM 18581) produces the novel compound vibroxin, which has the following chemical structure:

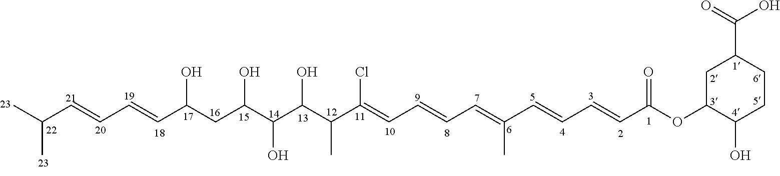 [SMILES: CC(/C=C/C=C/C(=O)OC1CC(C(=O)O)CCC1O)=C\C=C\C=C(/Cl)C(C)C(O)C(O)C(O)CC(O)/C=C/C=C/C(C)C]

Preferably, the nutrient medium in the process for the preparation of the compounds according to the present invention comprises glycerol as the sole carbon source. The glycerol may be present in an amount of between about 2 g/L and about 12 g/L, or between about 4 g/L and about 10 g/L, such as about 5 g/L.

The nutrient or minimal media may comprise yeast extract. In a preferred embodiment, the yeast extract is present in an amount of between about 0.01% w/v and about 0.1% w/v, such as between about 0.025% w/v and about 0.075% w/v, or about 0.05% w/v.

The nutrient or minimal media may comprise casamino acids. In a preferred embodiment, the casamino acids are present in an amount of between about 0.01% w/v and about 0.1% w/v, such as between about 0.025% w/v and about 0.075% w/v, or about 0.05% w/v.

Preferably, the bacterium is incubated at a temperature of between about 20° C. and about 37° C., such as between about 28° C. and about 32° C., or about 30° C. In some embodiments, the bacterium is incubated at a temperature of less than about 30° C.

Preferably, the method comprises incubating the bacterium on nutrient or minimal media up to and including at least part of the stationary phase. In preferred embodiments, the method comprises incubating the bacterium on minimal media for between about 16 hours and about 120 hours, or for between about 48 hours and about 96 hours, or for between about 48 hours and about 72 hours. In further preferred embodiments, the method comprises incubating the bacterium on minimal media for at least about 16 hours, or at least about 48 hours, or about 48 hours.

Preferably, the nutrient or minimal medium comprises a basal salts medium (BSM). Preferably, the basal salts medium comprises the formulation originally described by Hareland et al. (“Metabolic function and properties of 4-hydroxyphenylacetic acid 1-hydroxylase from Pseudomonas acidovorans”, J. Bacteriol. (1975) 121: 272-285).

Preferably, the production of Vibrio rhizosphaerae antibiotics and the extraction thereof are carried out using a solid surface growth medium such as BSM (basal salts medium) agar. Preferably, the recovery of a compound according to the present invention from the growth medium comprises extraction of the compound with a solvent, preferably an organic solvent such as an alcohol (e.g. methanol) or ethyl acetate.

Preferably, the step of recovering the antimicrobial agent from agar-grown cultures comprises breaking up the nutrient or minimal media, preferably by cutting up the agar, prior to extraction of the antimicrobial agent using ethyl acetate. The microorganisms are grown on the agar surface, and the agar cut into blocks after growth. The antimicrobial agents are then extracted from the agar blocks using a solvent, preferably an organic solvent such as ethyl acetate.

Vibroxin and analogues thereof in accordance with the invention may also be prepared from recombinant (genetically modified) or hybrid microbial systems, conveniently bacterial systems.

The vibroxin biosynthetic gene cluster has been identified by the inventors from within the genome of Vibrio rhizosphaerae MSSRF3 (DSM 18581) by homology to the gene cluster responsible for enacyloxin biosynthesis in B. ambifaria AMMD (Mahenthiralingam et at, Enacyloxins are products of an unusual hybrid modular polyketide synthase encoded by a cryptic Burkholderia ambifaria Genomic Island. Chem Biol. 18, 665, 2011) and B. gladioli pv. cocovenenans HKI 10521 (DSM 11318) (Netzker et al., Microbial communication leading to the activation of silent fungal secondary metabolite gene clusters. Front. Microbiol. 6, 299, 2015). The cluster is defined from the scaffold referenced as BS26DRAFT_scaffold00001.1 (GenBank: KL543967.1) between the coding sequences BS26_RS12130 and BS26_RS12145. The vibroxin biosynthetic genes were annotated on the basis of sequence similarity to those from the enacyloxin biosynthetic gene cluster in B. ambifaria AMMD.

The vibroxin biosynthetic gene cluster (SEQ ID NO: 1) has been determined by the inventors to have approximately 20 predicted genes based on homology to the enacyloxin biosynthetic gene cluster of B. ambifaria AMMD. The vibroxin genes have been designated vbx A to T (SEQ ID NOs: 2, 4, 6, 8, 10, 12, 14, 16, 18, 20, 22, 24, 26, 28, 30, 32, 34, 36, 38 and 40, respectively; and encode polypeptides of SEQ ID NOs: 3, 5, 7, 9, 11, 13, 15, 17, 19, 21, 23, 25, 27, 29, 31, 33, 35, 37, 39 and 41, respectively). Table 1 below discloses the putative function of each vbx gene, the homologous B. ambifaria gene and the percentage sequence identity between the corresponding gene sequences.

TABLE 1
Proposed functions of genes in the vibroxin biosynthetic gene cluster based on sequence
similarity to genes in the cluster directing enacyloxin biosynthesis in B. ambifaria AMMD

| | | | | | Percentage | Percentage |
|---|---|---|---|---|---|---|
| | SEQ ID NO of | SEQ ID NO of | | | identity in | identity in |
| | nucleotide | amino acid | | Homologous | nucleotide | amino acid |
| Gene | sequence | sequence | Putative function | gene | sequence | sequences |
| vbxA | 2 | 3 | FAD-dependent halogenase | bamb_5928 | 68 | 68 |
| vbxB | 4 | 5 | α-Ketoglutarate and non-haem iron-dependent | bamb_5927 | 71 | 71 |
| | | | hydroxylase | | | |
| vbxC | 6 | 7 | Type II thioesterase | bamb_5926 | 45 | 44 |
| vbxD | 8 | 9 | Polyketide synthase | bamb_5925 | 48 | 48 |
| vbxE | 10 | 11 | Polyketide synthase | bamb_5924 | 49 | 50 |
| vbxF | 12 | 13 | Polyketide synthase | bamb_5923 | 46 | 46 |
| vbxG | 14 | 15 | Polyketide synthase | bamb_5922 | 46 | 46 |
| vbxH | 16 | 17 | Polyketide synthase | bamb_5921 | 50 | 50 |
| vbxI | 18 | 19 | Polyketide synthase | bamb_5920 | 47 | 47 |
| vbxJ | 20 | 21 | Polyketide synthase | bamb_5919 | 51 | 50 |
| vbxK | 22 | 23 | Enoyl reductase involved in dihydroxycyclohexane | bamb_5918 | 71 | 72 |
| | | | carboxylic acid biosynthesis | | | |
| vbxL | 24 | 25 | Isomerase involved in dihydroxycyclohexane | bamb_5912 | 59 | 56 |
| | | | carboxylic acid biosynthesis | | | |
| vbxM | 26 | 27 | Shikimate-5-dehydrogenase involved in | bamb_5913 | 53 | 53 |
| | | | dihydroxycyclohexane carboxylic acid biosynthesis | | | |
| vbxN | 28 | 29 | Enoyl reductase involved in dihydroxycyclohexane | bamb_5914 | 57 | 56 |
| | | | carboxylic acid biosynthesis | | | |
| vbxO | 30 | 31 | Nonribsomal peptide synthetase condensation | bamb_5915 | 49 | 49 |
| | | | domain | | | |
| vbxP | 32 | 33 | Dehydratase involved in dihydroxycyclohexane | bamb_5916 | 67 | 68 |
| | | | carboxylic acid biosynthesis | | | |
| vbxQ | 34 | 35 | Peptidyl Carrier Protein | bamb_5917 | 36 | 35 |
| vbxR | 36 | 37 | LuxR transcriptional family regulator | bamb_5911 | 54 | 53 |
| vbxS | 38 | 39 | Hypothetical protein | bamb_5929 | 51 | 51 |
| vbxT | 40 | 41 | MATE family efflux protein | bamb_5933 | 55 | 54 |

The entire cluster, or any of the component genes thereof, including any of vbxA to vbxT, may be used with recombinant techniques to prepare genetically modified (“recombinant”) microorganisms capable of producing vibroxin and the vibroxin analogues according to the invention. Such microorganisms may be bacteria, in particular those which have, or are engineered to have, some or all components of another polyketide biosynthetic system (e.g. the enacyloxin biosynthetic system). Selective and/or over expression of the individual gene components of the vibroxin gene cluster, i.e. the vbx genes, and/or the mutation (sequence modification/editing) thereof, allows the design of tailored vibroxin compounds, e.g. the vibroxin analogues of the invention, and/or increased production of the target vibroxin molecule(s). Hybrid systems in which functionally complementary genes from other polyketide biosynthetic systems are expressed together with some, or all, of the vibroxin biosynthetic gene cluster of the invention provides for further control of the design of vibroxin analogues.

More specifically, analogues that lack the C-11 chlorine atom and C-14 hydroxyl group, or have modifications to the DHCCA moiety can be produced by constructing in frame deletions in vbxA, vbxB, vbxL, vbxP, vbxN and vbxK. To create vibroxin analogues in which the moderately labile ester linkage is replaced by a more stable amide bond, a mutasynthesis approach can be employed involving feeding of 3-amino-4-hydroxycyclohexane carboxylic acid (AHCCA) to mutants blocked in DHCCA biosynthesis. Such mutants can be prepared by deleting one or more genes within the vibroxin gene cluster responsible for the biosynthesis of DHCCA (i.e. vbxP, vbxN, vbxM, vbxL and vbxK). (Cis, cis) and (trans, trans)-3-amino-4-hydroxycyclohexane carboxylic acid can be produced in racemic form by high-pressure hydrogenation of commercially available 3-amino-4-hydroxybenzene carboxylic acid with rhodium on alumina as described, for example, by Wang et al., in Bioorganic and Medicinal Chemistry 14: 2242-2252, 2006, the entire contents of which are incorporated herein by reference.

Vibroxin analogues with other modifications to the DHCCA-derived moiety (for example, those in which the DHCCA-derived moiety is replaced by one of the acyclic groups herein described) can be produced via a similar mutasynthesis strategy in which one or more genes within the vibroxin gene cluster responsible for the biosynthesis of DHCCA are blocked. Suitable precursors for the acyclic moiety include compounds such as 4-amino butyric acid, 4-amino-3-hydroxy butyric acid, 4-hydroxy butyric acid, 3,4-dihydroxybutyric acid, and other similar such compounds. Such precursor compounds are either commercially available or may readily be prepared using known chemical synthetic methods.

In another embodiment, a vibroxin derivative in which the C-15 hydroxyl group is replaced with a keto group can be produced by co-expressing the PQQ-dependent oxidase encoded by bamb_5932 with the vibroxin BGC.

The vbxR gene is predicted to encode a LuxR-like transcriptional activator that induces expression of the vibroxin BGC in response to homoserine lactone (HSL) signalling molecules. Improved levels of vibroxin production in a heterologous host may be obtained by adding a cocktail of commercially-available HSLs to the culture medium or by co-cultivation with an appropriate HSL-producing organism.

Thus, in a further aspect there is provided a nucleic acid molecule comprising:

(a) the nucleotide sequence set forth in SEQ ID NO: 1;

(b) a nucleotide sequence which has at least 70% sequence identity with the nucleotide sequence set forth in SEQ ID NO: 1 and which encodes a functional vibroxin biosynthetic gene cluster;

(c) a nucleotide sequence degenerate with the nucleotide sequence set forth in SEQ ID NO: 1;

(d) a nucleotide sequence encoding a functional component of the vibroxin biosynthetic gene cluster encoded by the nucleotide sequence set forth in SEQ ID NO: 1;

(e) a nucleotide sequence which has at least 75% sequence identity with the nucleotide sequence of (d) and which encodes a functional component of the vibroxin biosynthetic gene cluster encoded by the nucleotide sequence set forth in SEQ ID NO: 1; or

(f) the complement of any one of (a)-(e),

preferably operably associated with one or more regulatory elements.

The invention further provides a nucleic acid molecule comprising:

(a′) the nucleotide sequence set forth in any one of SEQ ID NOs: 2, 4, 6, 8, 10, 12, 14, 16, 18, 20, 22, 24, 26, 28, 30, 32, 34, 36, 38 or 40;

(b′) a nucleotide sequence which has at least 75% sequence identity with the nucleotide sequence set forth in any one of SEQ ID NOs: 2, 4, 6, 8, 10, 12, 14, 16, 18, 20, 22, 24, 26, 28, 30, 32, 34, 36, 38 or 40 and which encodes a functional equivalent of vbx A to vbx T, respectively;

(c′) a nucleotide sequence which is a fragment of the nucleotide sequence set forth in any one of SEQ ID NOs: 2, 4, 6, 8, 10, 12, 14, 16, 18, 20, 22, 24, 26, 28, 30, 32, 34, 36, 38 or 40 and which encodes a functional equivalent of vbx A to vbx T, respectively;

(d′) a nucleotide sequence degenerate with the nucleotide sequence set forth in any one of SEQ ID NOs: 2, 4, 6, 8, 10, 12, 14, 16, 18, 20, 22, 24, 26, 28, 30, 32, 34, 36, 38 or 40; or

(e′) the complement of (a′), (b′), (c′) or (d′),

preferably operably associated with one or more regulatory elements.

In a further aspect the invention provides a polypeptide, wherein the amino acid sequence of the polypeptide:

(a) comprises the amino acid sequence set forth in any one of SEQ ID NOs: 3, 5, 7, 9, 11, 13, 15, 17, 19, 21, 23, 25, 27, 29, 31, 33, 35, 37, 39 or 41;

(b) comprises an amino acid sequence which has at least 75% sequence identity with any one of SEQ ID NOs: 3, 5, 7, 9, 11, 13, 15, 17, 19, 21, 23, 25, 27, 29, 31, 33, 35, 37, 39 or 41 and which is a functional equivalent of vbx A to vbx T, respectively;

(c) comprises an enzymatically active fragment of any one of SEQ ID NOs: 3, 5, 7, 9, 11, 13, 15, 17, 19, 21, 23, 25, 27, 29, 31, 33, 35, 37, 39 or 41;

(d) comprises the amino acid sequence encoded by the nucleotide sequence set forth in any one of SEQ ID NOs: 2, 4, 6, 8, 10, 12, 14, 16, 18, 20, 22, 24, 26, 28, 30, 32, 34, 36, 38 or 40;

(e) comprises the amino acid sequence encoded by a nucleotide sequence which has at least 75% sequence identity with the nucleotide sequence set forth in any one of SEQ ID NOs: 2, 4, 6, 8, 10, 12, 14, 16, 18, 20, 22, 24, 26, 28, 30, 32, 34, 36, 38 or 40 and which encodes a functional equivalent of vbx A to vbx T, respectively; or

(f) comprises the amino acid sequence of a functional component of the vibroxin biosynthetic gene cluster encoded by the nucleotide sequence set forth in SEQ ID NO: 1.

The invention also provides a nucleic acid vector comprising a nucleic acid molecule of the invention. Also provided is a host cell comprising a nucleic acid molecule of the invention or a nucleic acid vector of the invention.

As used herein, the term “nucleic acid molecule” refers to a DNA or RNA molecule, which might be single- or double-stranded. Preferably, the nucleic acid molecule is a DNA molecule, most preferably a double-stranded DNA molecule. In certain embodiments the nucleic acid molecule may be genomic DNA or cDNA. In other embodiments the nucleic is a single stranded RNA molecule carrying an above mentioned complementary sequence and said nucleic acid may be used in RNA interference methods or techniques.

The nucleic acid molecule of the invention is preferably isolated or purified. As used herein, the term “isolated nucleic acid” means that the nucleic acid molecule is not contiguous with other genes or nucleotide sequences with which it is normally associated in the natural source of the polypeptide-encoding nucleic acid. For example, an isolated nucleic acid of the invention comprising a vbx gene of interest will not carry said vbx gene contiguously with a directly neighbouring nucleotide sequence, e.g. a nucleic acid encoding its directly neighbouring gene(s), in the vibroxin biosynthetic gene cluster. The references to a nucleic acid comprising the vibroxin biosynthetic gene cluster should be construed accordingly. Thus, the isolated nucleic acid molecule is not a wild type genome of a bacterium.

As used herein, the term “purified nucleic acid” means a nucleic acid molecule which is free or substantially free from other non-contiguous nucleic acids and/or is free or substantially free from one or more of the following: bacteria, agar, yeast extract, tryptone.

In some embodiments of the invention, the nucleic acid molecule is a recombinant nucleic acid or produced by other artificial means, i.e. not obtained from a natural source.

In preferred embodiments the nucleic acids of the invention defined in terms of percentage sequence identity to another nucleotide sequence have at least 80%, 85%, 86%, 87%, 88%, 89%, 90%, 91%, 92%, 93%, 94% 95%, 96%, 97%, 98% or 99% sequence identity to their reference sequences, e.g. with SEQ ID NOs: 1, 2, 4, 6, 8, 10, 12, 14, 16, 18, 20, 22, 24, 26, 28, 30, 32, 34, 36, 38 or 40, preferably as calculated by the BLASTN method of alignment.

Percentage sequence identity, for both nucleic acids and proteins, according to the invention can be also be calculated using any of the widely available algorithms, e.g. the BLAST methods of alignment (Altschul et al. (1997), “Gapped BLAST and PSI-BLAST: a new generation of protein database search programs”, Nucleic Acids Res. 25:3389-3402; and http://www.ncbi.nlm.nih.gov/BLAST) using default parameters, or the Clustal W2 multiple sequence alignment program (http://www.ebi.ac.uk/Tools/clustalW2) using default parameters (DNA Gap Open Penalty=15.0; DNA Gap Extension Penalty=6.66; DNA Matrix=Identity; Protein Gap Open Penalty=10.0; Protein Gap Extension Penalty=0.2; Protein matrix=Gonnet; Protein/DNA ENDGAP=−1; Protein/DNA GAPDIST=4).

With regard to nucleotide sequence comparisons, MEGABLAST, discontiguous-megablast, and BLASTN may be also used to accomplish this goal. Preferably the standard or default alignment parameters are used. MEGABLAST is specifically designed to efficiently find long alignments between very similar sequences. Discontiguous MEGABLAST may be used to find nucleotide sequences which are similar, but not identical, to the nucleic acids of the invention. In some embodiments, the BLAST nucleotide searches can be performed with the BLASTN program, score=100, wordlength=12. The word size is adjustable in BLASTN and can be reduced from the default value to a minimum of 7 to increase search sensitivity. In other embodiments the discontiguous megablast page (www.ncbi.nlm.nih.gov/Web/Newsltr/FallWinter02/blastlab.html) is used. This page uses an algorithm which is similar to that reported by Ma et al. (Bioinformatics. 2002 March; 18(3): 440-5). Parameters unique for discontiguous megablast are: word size: 11 or 12; template: 16, 18, or 21; template type: coding (0), non-coding (1), or both (2).

The nucleic acid of the invention is preferably operably associated with one or more regulatory elements, e.g. a promoter and/or a terminator element. In certain embodiments such elements are not from Vibrio, or more particularly Vibrio rhizosphaerae (e.g. Vibrio rhizosphaerae MSSRF3). Preferably such elements will, in general, be functional in microbial, e.g. bacterial cells, but the skilled person would be able to select or design such elements to be compatible with the specific context in which the nucleic acids of the invention are being employed.

As used herein the term “operably associated” or “operably linked” with a promoter means that the polypeptide-encoding region may be transcribed from that promoter. The polypeptide-encoding region may, for example, be immediately 3′ to the promoter, in which case the promoter will direct the transcription of the coding sequence. Alternatively, the polypeptide-encoding region may be part of an operon or cluster in which case the associated or linked promoter will direct the transcription of all of the polypeptide-encoding regions within that operon/cluster.

The promoter or promoters are preferably ones which are operable in bacterial cells. More preferably, the promoters are bacterial promoters. Suitable promoters include inducible promoters, such as those that are inducible with specific sugars or sugar analogues, e.g. arabinose (e.g. lac, ara), those inducible with antibiotics (e.g. tetracycline, tet), those inducible with IPTG (e.g. trp, tac, Pspac), those inducible with heat (e.g. hsp70), those inducible with anaerobic induction (e.g. nisA, pfl, trc, IPL, IPR, T7), P11, Idh, sec (secDF), SV40 promoter, those inducible with xylose (e.g. Pxyl promoter), those inducible with osmotic shock, cell density (quorum sensing), anaerobicity, antibiotics, or growth phase. In some embodiments, the promoter is a constitutive promoter, e.g. the promoters for the thiolase gene (thl) or the permease operon (hfuC). In other embodiments, the promoter is one from Burkholderia, e.g. Burkholderia gladioli (e.g. Burkholderia gladioli pv. cocovenenans), Burkholderia ambifaria (e.g. Burkholderia ambifaria AMMD), or from Frateuria. In other embodiments, the promoter is one from a polyketide (e.g. enacyloxin) biosynthetic gene cluster, e.g. a Burkholderia or Frateuria polyketide biosynthetic gene cluster. In still further embodiments the promoter, e.g. as described above, is one from Vibrio, e.g. Vibrio rhizosphaerae (e.g. Vibro rhizosphaerae MSSRF3).

The nucleic acid molecule of the invention, with or without operable association with a regulatory element, will preferably be in the form of a nucleic acid vector, particularly an expression vector, or a plasmid. The vector or plasmid may comprise one or more selectable markers and/or other genetic elements. Preferably, the vector or plasmid is less than 100 Kb, more preferably less than 90, 80, 70, 60, 50, 40, 30 or 20 Kb. Preferably, the vector or plasmid additionally comprises one or more antibiotic resistance genes. Examples of such genes include genes conferring resistance to ampicillin, erythromycin, neomycin/kanamycin, tetracycline, chloramphenicol, spectinomycin, bleomycin and puromycin. In some embodiments, the vector or plasmid also comprises one or more genes conferring tolerance to one or more heavy metals, e.g. mercury. Other selectable markers include auxotrophy genes, e.g. genes for essential amino acids.

The vector or plasmid may also comprise an origin of replication, for example a Gram positive and/or a Gram negative bacterial origin of replication. The vector or plasmid may also comprise one or more insertion sequences, e.g. Tn10, Tn5, Tn1545, Tn916 and/or ISCb.

The nucleic acid molecule of the invention, or the plasmid or vector, may be introduced into a host cell, e.g. a microorganism, preferably a yeast or bacterial cell. In certain embodiments the host cell will not be a human cell. The bacterial cell may, for example, be a Gram-positive or Gram-negative bacterium. In some embodiments, the bacterium will be a bacterium that has, or has been engineered to have, some or all components of another polyketide biosynthetic system (e.g. the enacyloxin biosynthetic system). In other embodiments the bacterium may be from the genus Vibrio, Burkholderia, Frateuria, Sorangium (e.g. Sorangium cellulosum) or Pseudomonas. In other embodiments standard experimental bacteria may be used as host, e.g. E. coli or Streptomyces. In some embodiments of the invention, the host cell is from the genus Burkholderia, e.g. Burkholderia gladioli, in particular Burkholderia gladioli strain LMG-P 26202 or Burkholderia gladioli pv. cocovenenans, or Burkholderia ambifaria, in particular Burkholderia ambifaria AMMD. In other embodiments the host cell is not from the genus Vibrio, more particularly Vibrio rhizosphaerae (e.g. Vibrio rhizosphaerae MSSRF3).

The invention further provides a process for making a recombinant host cell, e.g. bacterial host cell, comprising introducing a nucleic acid molecule of the invention, or a nucleic acid vector or plasmid of the invention, into a host cell. Methods of introducing nucleic acid molecules, plasmids and vectors into host cells are well known in the art. These include transformation, transfection and electroporation techniques.

The invention also provides a recombinant (genetically modified/engineered) host cell, e.g. those disclosed above, comprising a nucleic acid molecule of the invention, or a vector or plasmid of the invention. The nucleic acid molecule or vector or plasmid may be present in the cytoplasm of the host, or it may be integrated in the host genome. Preferably the host cell containing the nucleic acid molecule or vector of the invention produces vibroxin or a vibroxin analogue of the invention under conducive conditions.

The invention therefore provides a cell, preferably microorganism, e.g. a bacterium, e.g. those disclosed above, comprising a nucleic acid molecule, a vector or plasmid of the invention, wherein the nucleic acid molecule, vector or plasmid is present in the cytoplasm of the cell.

The invention also provides a cell, preferably a microorganism, e.g. a bacterium, e.g. those disclosed above, comprising a nucleic acid molecule of the invention or an operon or vector or plasmid of the invention, wherein the nucleic acid molecule, operon, vector or plasmid is present in (e.g. stably integrated into) the genome of the cell.

The polypeptides of the invention may be isolated and/or purified. In particular, the polypeptides of the invention may be in a form which is isolated from one or more of the following: bacteria, yeast extract, tryptone, agar, other enzymes or other polypeptides, in particular polypeptides that are not vbx polypeptides (e.g. encoded by the vibroxin gene cluster).

The polypeptides of the invention may be purified, i.e. the polypeptide may be substantially pure. In particular, the polypeptides may be at least 90%, preferably at least 95% and more preferably at least 99% pure. Purity may be assessed using SDS-PAGE or any other appropriate method.

The invention also provides variants or derivatives of any of the polypeptides of SEQ ID NOs: 3, 5, 7, 9, 11, 13, 15, 17, 19, 21, 23, 25, 27, 29, 31, 33, 35, 37, 39 or 41. The polypeptides of the invention may be altered in various ways including substitutions, deletions, truncations, and/or insertions of one or more (e.g. 2-5, 2-10) amino acids, preferably in a manner which does not substantially alter the biological activity of the polypeptides of the invention. Guidance as to appropriate amino acid changes that do not affect biological activity of the protein of interest may be found in the model of Dayhoff et al. (1978) Atlas of Protein Sequence and Structure (Nat'l. Biomed. Res. Found., Washington, D.C.), herein incorporated by reference. Conservative substitutions, such as exchanging one amino acid with another having similar properties, may also be made. In particular, substitution of one hydrophobic amino acid such as isoleucine, valine, leucine or methionine for another may be made; or the substitution of one polar amino acid residue for another, such as arginine for lysine, glutamic for aspartic acid, or glutamine for asparagine, may be made.

One or more (e.g. 1-5, 1-10) amino acids in the polypeptides of the invention may be substituted by their corresponding D-amino acids, preferably amino acids at the N- and/or C-terminus.

In particular, the invention provides variants of the any of the polypeptides of SEQ ID NOs: 3, 5, 7, 9, 11, 13, 15, 17, 19, 21, 23, 25, 27, 29, 31, 33, 35, 37, 39 or 41, wherein the amino acid sequence of the variants comprise or consist of an amino acid sequence having at least 80%, preferably at least 85%, 86%, 87%, 88%, 89%, 90%, 91%, 92%, 93%, 94% 95%, 96%, 97%, 98% or 99% sequence identity with the reference sequence, preferably using the blastp method of alignment.

Like other BLAST programs, blastp is designed to find local regions of similarity. When sequence similarity spans the whole sequence, blastp will also report a global alignment, which is the preferred result for protein identification purposes. Preferably the standard or default alignment parameters are used. In some instances, the “low complexity filter” may be taken off. BLAST protein searches may also be performed with the BLASTX program, score=50, wordlength=3. To obtain gapped alignments for comparison purposes, Gapped BLAST (in BLAST 2.0) can be utilized as described in Altschul et al. (1997) Nucleic Acids Res. 25: 3389. Alternatively, PSI-BLAST (in BLAST 2.0) can be used to perform an iterated search that detects distant relationships between molecules (see Altschul et al. (1997) supra). When utilizing BLAST, Gapped BLAST, PSI-BLAST, the default parameters of the respective programs may be used.

“Functional” in the context of the polypeptides of the invention refers to a role in the synthesis, transport or transfer of a polyketide moiety, preferably vibroxin or a vibroxin derivative or vibroxin-related molecule. Specifically in relation to vbxA proteins, it refers to the activities recited in Table 1.

The Vbx proteins of the invention are polypeptides involved in the biosynthesis of vibroxin in Vibrio rhizosphaerae (e.g. Vibrio rhizosphaerae MSSRF3). A functionally equivalent fragment or variant of a Vbx protein (Vbx A to Vbx T) will therefore display the same activity (e.g. catalytic activity, transcriptional activity) and preferably the same or substantially the same levels of said activity as the full length Vbx protein from which it derives, e.g. those as defined by SEQ ID NOs: 3, 5, 7, 9, 11, 13, 15, 17, 19, 21, 23, 25, 27, 29, 31, 33, 35, 37, 39 and 41. In these embodiments “substantially the same” may be expressed as at least 50% of the activity of the full length Vbx protein, e.g. at least 60%, 70%, 80%, 90, or 95% of the activity of the full length Vbx protein.

The polypeptides of the invention may be used in methods for the preparation of vibroxin or the vibroxin derivatives of the invention, e.g. in cell-free methods or in methods involving cell factories, or to modify vibroxin produced in cell factories.

In a further aspect of the invention there is provided a method for expressing a vibroxin biosynthetic gene cluster, e.g. that is encoded by the nucleic acids of the invention described above, or a functional component thereof in a host cell, said method comprising introducing a nucleic acid molecule, or a plasmid or vector comprising said nucleic acid molecule, into a host cell and subsequently culturing said host cell under conditions conducive to the expression of said cluster or component thereof from said nucleic acid, said nucleic acid comprising:

(a) the nucleotide sequence set forth in SEQ ID NO: 1;

(b) a nucleotide sequence which has at least 70% sequence identity with the nucleotide sequence set forth in SEQ ID NO:1 and which encodes a functional vibroxin biosynthetic gene cluster;

(c) a nucleotide sequence degenerate with the nucleotide sequence set forth in SEQ ID NO: 1;

(d) a nucleotide sequence encoding a functional component of the vibroxin biosynthetic gene cluster encoded by the nucleotide sequence set forth in SEQ ID NO: 1; or

(e) a nucleotide sequence which has at least 75% sequence identity with the nucleotide sequence of (d) and which encodes a functional component of the vibroxin biosynthetic gene cluster encoded by the nucleotide sequence set forth in SEQ ID NO: 1.

In a further aspect of the invention there is provided a method for expressing a Vbx protein of the invention as described above in a host cell, said method comprising introducing a nucleic acid molecule, or a plasmid or vector comprising said nucleic acid molecule, into a host cell and subsequently culturing said host cell under conditions conducive to the expression of said vbx protein from said nucleic acid, said nucleic acid comprising:

(a′) the nucleotide sequence set forth in any one of SEQ ID NOs: 2, 4, 6, 8, 10, 12, 14, 16, 18, 20, 22, 24, 26, 28, 30, 32, 34, 36, 38 or 40;

(b′) a nucleotide sequence which has at least 75% sequence identity with the nucleotide sequence set forth in any one of SEQ ID NOs: 2, 4, 6, 8, 10, 12, 14, 16, 18, 20, 22, 24, 26, 28, 30, 32, 34, 36, 38 or 40 and which encodes a functional equivalent of Vbx A to Vbx T, respectively;

(c′) a nucleotide sequence which is a fragment of the nucleotide sequence set forth in any one of SEQ ID NOs: 2, 4, 6, 8, 10, 12, 14, 16, 18, 20, 22, 24, 26, 28, 30, 32, 34, 36, 38 or 40 and which encodes a functional equivalent of Vbx A to Vbx T, respectively; or

(d′) a nucleotide sequence degenerate with the nucleotide sequence set forth in any one of SEQ ID NOs: 2, 4, 6, 8, 10, 12, 14, 16, 18, 20, 22, 24, 26, 28, 30, 32, 34, 36, 38 or 40.

Culture conditions may be those as described above. The expression products or a portion thereof may be subsequently separated or isolated from said host cells and/or the media in which said cells have been cultured with any of the purification techniques for protein known in the art and widely described in the literature or any combination thereof. Such techniques may include, for example, precipitation, ultrafiltration, dialysis, various chromatographic techniques, e.g. gel filtration, ion-exchange chromatography, affinity chromatography, electrophoresis, centrifugation, etc. Likewise an extract of host cells may also be prepared using techniques well known in the art, e.g. homogenisation, freeze-thawing, etc. and from this extract the polypeptides of the invention can be purified. The host cells may be those described above.

It may be desirable to modify said nucleic acids, e.g. to mutate or edit, for instance as described above, in order to obtain modified versions of the vbx polypeptides or the vibroxin gene cluster. Such modifications may alter the selectivity, specificity, efficiency and function of the individual components of the cluster thereby altering the structure of the vibroxin product or its efficiency or yield of production.

In a further aspect of the invention there is provided a method for the preparation of vibroxin or a vibroxin analogue, e.g. those of the invention described above, said method comprising introducing at least one nucleic acid molecule of the invention, or a plasmid or vector comprising said at least one nucleic acid molecule, into a host cell and subsequently culturing said host cell under conditions conducive to the production of vibroxin or a vibroxin analogue of the invention. Alternatively the method may comprise culturing a recombinant (genetically modified) host cell of the invention as defined above under conditions conducive to the production of vibroxin or a vibroxin analogue of the invention. Culture conditions may be those as described above for Vibrio rhizosphaerae. Recovery of the vibroxin or a vibroxin analogue or a portion thereof may conveniently be achieved as described above for Vibrio rhizosphaerae. The host cells may be those described above. Vibroxin analogues obtained from such methods form a further aspect of the invention.

It may be desirable to modify, e.g. mutate or edit, for instance as described above, said nucleic acids and/or said recombinant host cells prior to the culture step in order to obtain further vibroxin analogues. Mutation of host cells/nucleic acids can be achieved by routine means, e.g. exposure to radiation (e.g. UV) and/or chemical mutagens. Gene editing technologies, e.g. CRISPR/Cas 9 may also be used. Vibroxin analogues obtained from such methods form a further aspect of the invention.

The compounds of the present invention may be used in therapy. As such, according to another aspect of the present invention, there is provided a method for the treatment of an infection, the method comprising administering to a subject in need thereof, a compound according to the present invention, or a pharmaceutically acceptable salt, metabolite, or prodrug thereof, wherein the infection is caused by a microbe, optionally wherein the microbe is resistant to an antimicrobial drug.

According to another aspect of the present invention, there is provided a method for the treatment of an infection, the method comprising administering to a subject in need thereof, a compound according to the present invention, or a pharmaceutically acceptable salt, metabolite, or prodrug thereof, wherein the infection is caused by at least one pathogenic bacterium that is susceptible to vibroxin or an analogue thereof as herein described, for example at least one bacterium selected from Acinetobacter species, Burkholderia species, Ralstonia species, and Stenotrophomonas species.

According to another aspect of the present invention, there is provided the use of a compound according to the present invention, or a pharmaceutically acceptable salt, metabolite, or prodrug thereof, preferably a therapeutically acceptable amount thereof, in the manufacture of a medicament for the treatment of a microbial infection.

Also provided is a method for inhibiting the growth of a microbe, the method comprising contacting the microbe with a compound according to the present invention, or a pharmaceutically acceptable salt, metabolite, or prodrug thereof, or with a bacterium capable of producing the compound. The method may be performed in vitro or in vivo. In the case of contact with a bacterium capable of producing the compound, suitable conditions, such as those identified above, may be provided in order that the antimicrobial compound is produced.

It is preferred that a therapeutically effective amount of a compound as herein described, in any stereochemical form, or a mixture of any stereochemical forms in any ratios, or a pharmaceutically acceptable salt, metabolite, or prodrug thereof, is present or is used in the above aspects of the invention.

The following definitions shall apply throughout the specification and the appended claims.

Within the context of the present application, the terms “comprises” and “comprising” are interpreted to mean “includes, among other things”. These terms are not intended to be construed as “consists of only”.

Unless otherwise stated or indicated, the term “alkyl” means a monovalent saturated, linear or branched, carbon chain, such as C₁₋₈, C₁₋₆ or C₁₋₄, which may be unsubstituted or substituted. The group may be partially or completely substituted with substituents independently selected from one or more of halogen (F, Cl, Br or I), hydroxy, nitro and amino. Non-limiting examples of alkyl groups methyl, ethyl, n-propyl, iso-propyl, n-butyl, iso-butyl, sec-butyl, tert-butyl, n-pentyl, iso-pentyl, n-pentyl, n-hexyl, etc. An alkyl group preferably contains from 1-6 carbon atoms, e.g. 1-4 carbon atoms.

Unless otherwise stated or indicated, the term “cycloalkyl” refers to a monovalent, saturated cyclic carbon system. Unless otherwise specified, any cycloalkyl group may be substituted in one or more positions with a suitable substituent. Where more than one substituent group is present, these may be the same or different. Suitable substituents include halogen (F, Cl, Br or I), hydroxy, nitro and amino.

Unless otherwise stated or indicated, the term “alkenyl” means a straight-chained or branched unsaturated hydrocarbon chain of 2-20 carbon atoms, such as C₂₋₁₀, C₂₋₈, C₂₋₆ or C₂₋₄, which may be unsubstituted or substituted, and containing at least one double bond. The group may be partially or completely substituted with substituents independently selected from one or more of halogen (F, Cl, Br or I), hydroxy, nitro and amino. Non-limiting examples of alkenyl groups include ethenyl, 1-propenyl, 2-propenyl, 2-methyl-1-propenyl, 1-butenyl, 2-butenyl-pentenyl, 3-pentenyl, 3-methyl-2-butenyl, 3-methyl-but-2-enyl, 3-hexenyl, 1,1-dimethyl-but-2-enyl, and the like.

Unless otherwise stated or indicated, the term “aryl” is intended to cover aromatic ring systems. Such ring systems may be monocyclic or polycyclic (e.g. bicyclic) and contain at least one unsaturated aromatic ring. Where these contain polycyclic rings, these may be fused. Preferably such systems contain from 6-20 carbon atoms, e.g. either 6 or 10 carbon atoms. Examples of such groups include phenyl, 1-napthyl, 2-napthyl and indenyl. A preferred aryl group is phenyl. Unless stated otherwise, any “aryl” group may be substituted by one or more substituents, which may be identical or different, for example halogen (F, Cl, Br or I), hydroxy, nitro and amino.

As used herein, the term “heteroaryl” is intended to cover heterocyclic aromatic groups. Such groups may be monocyclic or bicyclic and contain at least one unsaturated heteroaromatic ring system. Where these are monocyclic, these comprise 5- or 6-membered rings which contain at least one heteroatom selected from nitrogen, oxygen and sulphur and contain sufficient conjugated bonds to form an aromatic system. Where these are bicyclic, these may contain from 9-11 ring atoms. Examples of heteroaryl groups include thiophene, thienyl, pyridyl, thiazolyl, furyl, pyrrolyl, triazolyl, imidazolyl, oxadiazolyl, oxazolyl, pyrazolyl, imidazolonyl, oxazolonyl, thiazolonyl, tetrazolyl, thiadiazolyl, benzimidazolyl, benzooxazolyl, benzofuryl, indolyl, isoindolyl, pyridonyl, pyridazinyl, pyrimidinyl, imidazopyridyl, oxazopyridyl, thiazolopyridyl, imidazopyridazinyl, oxazolopyridazinyl, thiazolopyridazinyl and purinyl. Preferred heteroaryl groups include pyrrole, indole, thiazole, triazole or pyridine. Unless stated otherwise, any “heteroaryl” may be substituted by one or more substituents, which may be identical or different, for example halogen (F, Cl, Br or I), hydroxy, nitro and amino

The term “antimicrobial” includes antibiotics and chemicals capable of inhibiting or preventing the growth of, or capable of killing, microbes, especially bacteria. An example of an antimicrobial chemical is a disinfectant.

The term “antibiotic” means an agent produced by a living organism, such as a bacterium, that is capable of inhibiting the growth of another living organism, for example another bacterium, or is capable of killing another living organism, for example another bacterium.

The term “therapeutically effective amount” means an amount of an agent or compound which provides a therapeutic benefit in the treatment of a microbial infection.

The term “treatment” includes prevention, reduction, amelioration or elimination or the disorder or condition.

The term “pharmaceutically acceptable” means being useful in preparing a pharmaceutical composition that is generally safe, non-toxic and neither biologically nor otherwise undesirable and includes being useful for veterinary use as well as human pharmaceutical use.

Suitable pharmaceutically acceptable salts may include acid addition salts which may, for example, be formed by mixing a solution of the antimicrobial agent with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the antimicrobial agents of the invention carry an acidic moiety, suitable pharmaceutically acceptable salts thereof may include alkali metal salts (e.g. sodium or potassium salts); alkaline earth metal salts (e.g. calcium or magnesium salts); and salts formed with suitable organic ligands (e.g. ammonium, quaternary ammonium and amine cations formed using counter-anions such as halide, hydroxide, carboxylate, sulfate, phosphate, nitrate, alkyl sulfonate and aryl sulfonate). Illustrative examples of pharmaceutically acceptable salts include but are not limited to acetate, adipate, alginate, ascorbate, aspartate, benzenesulfonate, benzoate, bicarbonate, bisulfate, bitartrate, borate, bromide, butyrate, calcium edetate, camphorate, camphorsulfonate, camsylate, carbonate, chloride, citrate, clavulanate, cyclopentanepropionate, digluconate, dihydrochloride, dodecylsulfate, edetate, edisylate, estolate, esylate, ethanesulfonate, formate, fumarate, gluceptate, glucoheptonate, gluconate, glutamate, glycerophosphate, glycolylarsanilate, hemisulfate, heptanoate, hexanoate, hexylresorcinate, hydrabamine, hydrobromide, hydrochloride, hydroiodide, 2-hydroxy-ethanesulfonate, hydroxynaphthoate, iodide, isothionate, lactate, lactobionate, laurate, lauryl sulfate, malate, maleate, malonate, mandelate, mesylate, methanesulfonate, methylsulfate, mucate, 2-naphthalenesulfonate, napsylate, nicotinate, nitrate, N-methylglucamine ammonium salt, oleate, oxalate, pamoate (embonate), pahnitate, pantothenate, pectinate, persulfate, 3-phenylpropionate, phosphate/diphosphate, picrate, pivalate, polygalacturonate, propionate, salicylate, stearate, sulfate, subacetate, succinate, tannate, tartrate, teoclate, tosylate, triethiodide, undecanoate, valerate, and the like.

The term “metabolite” means any intermediate or product resulting from metabolism of a compound according to the present invention.

The term “prodrug” means a functional derivative of a compound according to the present invention, such as an ester or an amide, that is biotransformed in the body to form the active drug. Reference is made to Goodman and Gilman's, The Pharmacological basis of Therapeutics, 8th ed., Mc-Graw-Hill, Int. Ed. 1992, “Biotransformation of Drugs”, p. 13-15.

The term “isomer” used herein refers to all forms of structural and spatial isomers. In particular, the term “isomer” is intended to encompass stereoisomers. Unless otherwise indicated, the structures presented herein are not intended as an accurate representation of the cis-trans stereochemistry of any of the double bonds in the molecule. All potential combinations of cis and trans stereochemistries are considered to be encompassed by the invention.

With regard to stereoisomers, the compounds of formulae (I), (Ia), (Ib), (II) and (IIa) herein described may have one or more asymmetric carbon atoms and may occur as racemates, racemic mixtures and as individual enantiomers or diastereomers. All such isomeric forms are included within the present invention, including mixtures thereof. Cis (E) and trans (Z) isomerism may also occur. The present invention includes the individual stereoisomers of the compounds of the invention, together with mixtures thereof. Separation of diastereoisomers or cis and trans isomers may be achieved by conventional techniques, e.g. by fractional crystallisation, chromatography or HPLC. A stereoisomeric mixture of the compounds may also be prepared from a corresponding optically pure intermediate or by resolution, such as by HPLC of the corresponding racemate using a suitable chiral support or by fractional crystallisation of the diastereoisomeric salts formed by reaction of the corresponding racemate with a suitable optically active acid or base, as appropriate.

The term “potentially susceptible bacteria” or means bacteria which have the potential for their growth to be inhibited or destroyed by an antimicrobial agent produced by an antimicrobial producing bacterium. Examples include those listed herein whose growth may be inhibited by the antimicrobial agents of the present invention.

Antimicrobial agents of the invention can be incorporated into pharmaceutical compositions suitable for administration. Such compositions typically comprise at least one antimicrobial of the invention and at least one pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). In all cases, the composition must be sterile and should be fluid to the extent that syringability exists. It must be stable under the conditions of manufacture, transfer and storage. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminium mono stearate and gelatin.

Sterile injectable solutions can be prepared by incorporating the active compound (e.g., an antimicrobial according to an embodiment of the invention) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavouring agent such as peppermint, methyl salicylate, or orange flavouring.

For administration by inhalation, the compounds can be delivered in the form of an aerosol spray of liquid, or powdered or formulated antibiotic (e.g. within liposomes as stated below) from pressured container or dispenser which contains a suitable propellant, e.g. a gas such as carbon dioxide, or a nebulizer.

For topical administration, the compounds may be delivered in the form of gels, creams, ointments, sprays, lotions, salves, powders, aerosols, drops, solutions and any of the other conventional pharmaceutical forms in the art. Ointments, gels and creams may, for example, be formulated with an aqueous or oily base with the addition of suitable thickening and/or gelling agents. Any thickening or gelling agents used should be non-toxic and non-irritant. Formulations for topical treatment, e.g. treatment of bacterial infected wounds, may be based on gel formulations, e.g. hydrogels. The compounds of the invention may be incorporated into such hydrogel formulations. Lotions may be formulated with an aqueous or oily base and will, in general, also contain one or more emulsifying, dispersing, suspending, thickening or colouring agents. Powders may be formed with the aid of any suitable powder base. Drops (e.g. eye drops), sprays (e.g. nasal sprays) and solutions may be formulated with an aqueous or non-aqueous base also comprising one or more dispersing, solubilising or suspending agents. Aerosol sprays are conveniently delivered from pressurised packs, with the use of a suitable propellant.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays, pessaries or suppositories. For transdermal administration, the active compounds can be formulated into ointments, salves, gels, or creams as generally known in the art.

The compounds can also be prepared in the form of suppositories (e.g. with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The pharmaceutical compositions can be included in a container, pack, or dispenser together with instructions for administration. According to this aspect, the invention provides a kit comprising at least one compound according to the invention or a pharmaceutical composition of the invention, optionally in addition to one or more further active agents as defined herein, preferably with instructions for the administration thereof in the therapeutic treatment of the human or animal body, e.g. the treatment of infection by one or more infectious organisms as hereinbefore defined.

When using the compounds according to the present invention, the dose can vary within wide limits and, as is customary and is known to the physician, is to be suited to the individual conditions in each individual case. It depends, for example, on the nature and severity of the disease to be treated, on the mode of administration, or on whether an acute or chronic condition is treated or whether prophylaxis is carried out. An appropriate dosage can be established using clinical approaches well known in the medical art. In general, the daily dosage for achieving the desired results in an adult weighing about 75 kg is from about 0.01 to about 100 mg/kg, preferably from about 0.1 to about 50 mg/kg, in particular from about 0.1 to about 10 mg/kg.

Within this specification embodiments have been described in a way which enables a clear and concise specification to be written, but it is intended and will be appreciated that embodiments may be variously combined or separated without departing from the invention.

The invention will now be further illustrated by the following non-limiting examples and figures, in which:

FIG. 1 shows the LC-MS analysis of ethyl acetate extracts from V. rhizosphaerae cultures grown on BSM2S-glycerol-agar. a) UV-Vis chromatogram (380 nm); b) Extracted ion chromatogram at m/z 645.2806; and c) Extracted ion chromatogram at m/z 605.2881.

FIG. 2 shows the high-resolution LC-MS analysis of vibroxin. a) Observed mass spectrum for vibroxin ([M+Na]⁺); and b) simulated mass spectrum for C₃₃H₄₇ClNaO₉⁺.

FIG. 3 shows the ¹H NMR spectrum of vibroxin (d₄-MeOH, 500 MHz).

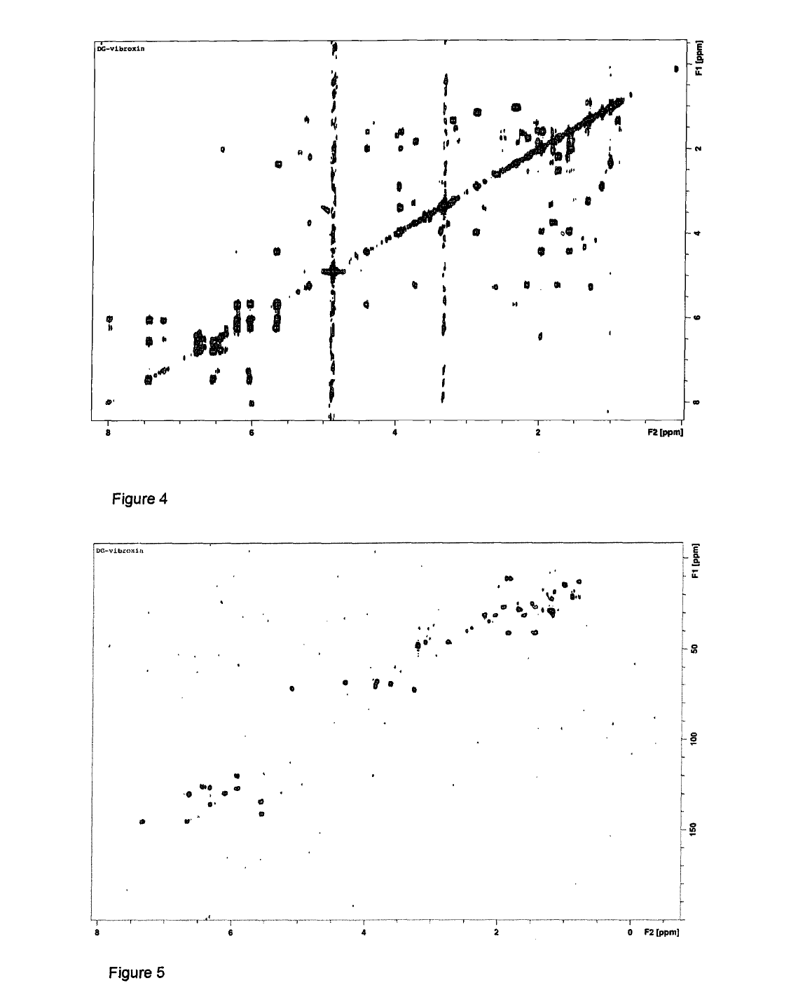

FIG. 4 shows the COSY NMR spectrum of vibroxin (d₄-MeOH, 500 MHz).

FIG. 5 shows the ¹H-¹³C HSQC spectrum of vibroxin (d₄-MeOH, 500/125 MHz).

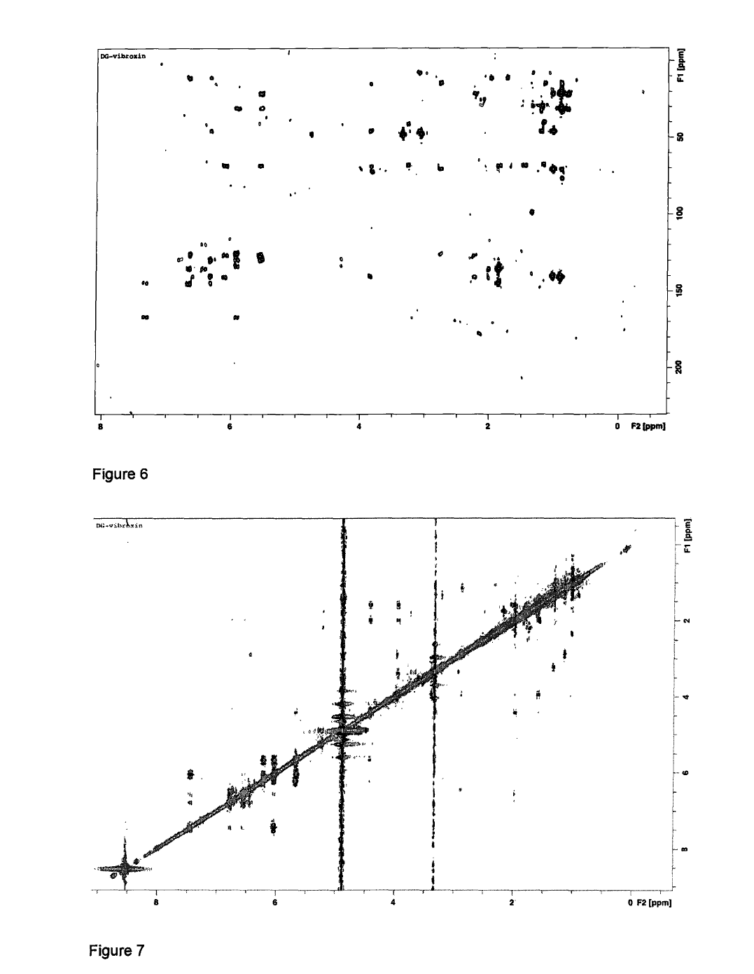

FIG. 6 shows the ¹H-¹³C HMBC NMR spectrum of vibroxin (d₄-MeOH, 500/125 MHz).

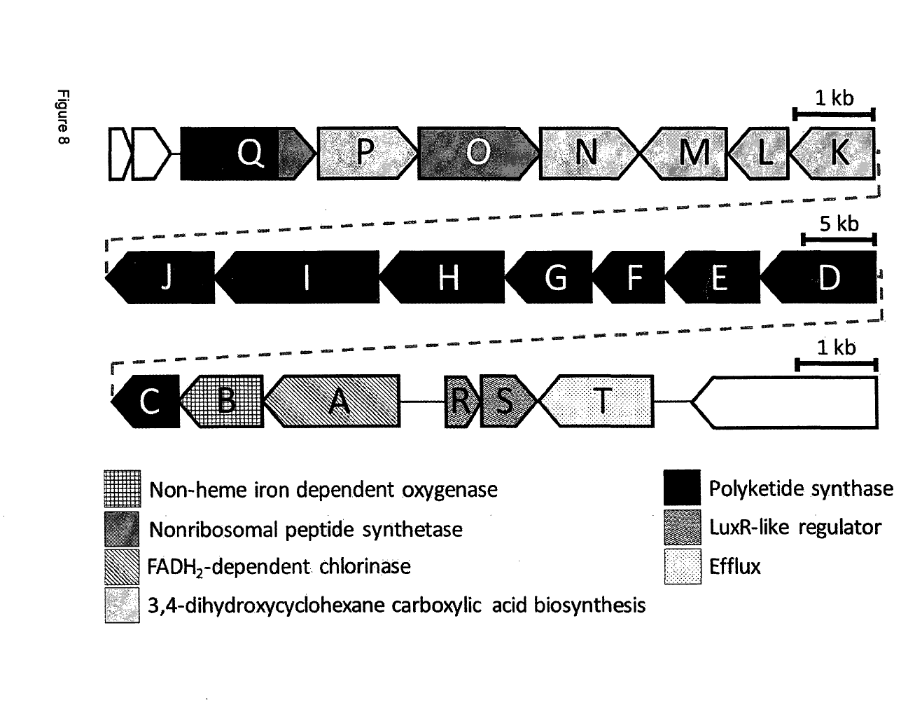

FIG. 7 shows the NOESY spectrum for vibroxin with a mixing time of 100 ms (d₄-MeOH, 500 MHz).

FIG. 8 shows the vibroxin biosynthetic gene cluster.

### EXAMPLES

### Example 1—Isolation and Structure Elucidation of Vibroxin

Vibrio rhizosphaerae MSSRF3 (DSM 18581) was acquired from the Leibniz Institute DSMZ (German Collection of Microorganisms and Cell Cultures). For vibroxin production and isolation, large scale cultures of V. rhizosphaerae were grown on BSM2S-agar (basal salt medium¹ supplemented with 2% NaCl, and glycerol to a final concentration of 4 g/L) at 30° C. (see Hareland et al., Metabolic function and properties of 4-hydroxyphenylacetic acid 1-hydroxylase from Pseudomonas acidovorans. J. Bacterioal. 121: 272, 1975). After 65 hours, the biomass was scraped off and the agar was extracted twice with an equal volume of ethyl acetate. After filtration, the solvent was removed by rotary evaporation in vacuo and the residue was re-dissolved in acetonitrile for high-resolution LC-ESI-MS analysis. Separation was performed on a Zorbax Eclipse Plus C₁₈ column (1.8 μm, 2.1×100 mm, Agilent) using the elution profile shown in Table 2 with a flow rate of 0.2 ml/min and monitoring absorbance at 380 nm (FIG. 1a). The molecular formula of vibroxin was shown to be C₃₃H₄₇ClO₉ (m/z calculated for C₃₃H₄₇ClNaO₉⁺: 645.2806, found: 645.2803) (FIG. 1b). Additionally, in-source fragmentation of the [M+H]⁺ ion yielded a daughter ion with m/z 605.2881, corresponding to dehydration of the parent ion (FIG. 1c). The high-resolution mass spectrum observed for vibroxin is consistent with the predicted isotopic distribution for a C₃₃H₄₇ClNaO₉⁺ species (FIG. 2).

TABLE 2
LC-MS elution profile for extracts

| Time | Acetonitrile % | Water % |
|---|---|---|
| (minutes) | (0.1% FA) | (0.1% FA) |
| 0 | 30 | 70 |
| 5.3 | 30 | 70 |
| 17.3 | 100 | 0 |
| 22.6 | 100 | 0 |
| 25.3 | 30 | 70 |
| 34 | 30 | 70 |

Vibroxin was purified from the ethyl acetate extracts of the agar by semi-preparative HPLC using a ZORBAX-SB C₁₈ column (21.2×100 mm, 5 μm) using the elution profile in Table 3 while monitoring absorbance at 380 nm. Fractions from multiple runs were pooled, concentrated in vacuo, and subsequently lyophilized to give 0.3 mg purified vibroxin as a yellow/brownish solid.

TABLE 3
HPLC elution profile for vibroxin purification

| Time | Methanol % | Water % |
|---|---|---|
| (minutes) | (0.1% FA) | (0.1% FA) |
| 0 | 70 | 30 |
| 2 | 70 | 30 |
| 5 | 85 | 15 |
| 30 | 100 | 0 |
| 32 | 100 | 0 |
| 36 | 70 | 30 |
| 40 | 70 | 30 |

For structure elucidation, purified vibroxin was dissolved in 150 μL of d₄-MeOH. Experiments were taken from the Bruker suite of pulse sequences (including ¹H, COSY, HMBC, HSQC, NOESY) and run on a Bruker Avance III HD 500 MHz spectrometer equipped with a DCH cryoprobe. NOESY mixing times were 30 ms and 100 ms. Spectra are shown in FIGS. 3-7. The resulting chemical shift assignments for vibroxin are listed in Table 4.

TABLE 4
NMR assignments for vibroxin isolated from
V. rhizosphaerae MSSRF3

| | δ_{H}/ppm (no. of protons, | |
|---|---|---|
| position | multiplicity, J/Hz) | δ_{C}/ppm |
| 1′-COOH | | 178.1 |
| 1′ | 2.52 (1H, m) | 38.5 |
| 2′ | 2.17 (1H, m) | 31.5 |
| | 1.74 (1H, m) | |
| 3′ | 5.22 (1H, m) | 72.2 |
| 4′ | 3.73 (1H, m) | 69.6 |
| 5′ | 1.82 (2H, m) | 28.3 |
| 6′ | 2.05 (1H, m) | 26.8 |
| | 1.57 (1H, m) | |
| 1 | | 167.4 |
| 2 | 6.03 (1H, d, 15.0) | 120.2 |
| 3 | 7.45 (1H, dd, 15.0, 11.0) | 145.1 |
| 4 | 6.55 (1H, dd, 15.0, 11.0) | 125.9 |
| 5 | 6.78 (1H, br d 15.0) | 145.3 |
| 6 | | 135.8 |
| 6-Me | 1.97 (3H, br s) | 11.2 |
| 7 | 6.44 (1H, br d, 10.0) | 135.6 |
| 8 | 6.77 (1H, m) | 130.1 |
| 9 | 6.77 (1H, m) | 130.1 |
| 10 | 6.44 (1H, br d, 9.5) | 126.4 |
| 11 | | 140.7 |
| 12 | 2.88 (1H, dq, 10.0, 7.0) | 46.0 |
| 12-Me | 1.13 (3H, d, 7.0) | 14.8 |
| 13 | 3.96 (1H, br, d, 10.0) | 70.8 |
| 14 | 3.37 (1H, m) | 72.8 |
| 15 | 3.94 (1H, m) | 68.2 |
| 16 | 2.05 (1H, dd, 17.0, 4.5) | 41.1 |
| | 1.57 (1H, m) | |
| 17 | 4.41 (1H, m) | 68.8 |
| 18 | 5.67 (1H, dd, 15.0, 6.5) | 134.2 |
| 19 | 6.21 (1H, dd, 15.0, 10.0) | 129.7 |
| 20 | 6.03 (1H, dd, 15.0, 10.0) | 126.9 |
| 21 | 5.65 (1H, dd, 15.0, 6.5) | 141.1 |
| 22 | 2.33 (1H, sept, 6.5) | 31.3 |
| 23 | 1.02 (6H, d, 6.5) | 21.4 |

The NMR spectroscopic data for vibroxin were similar to those reported for enacyloxin. However, several structural differences between vibroxin and enacyloxin were observed. Firstly, a doublet at 1.02 ppm, integrating for six protons, suggested the presence of an isopropyl group. The location of this isopropyl group at C-22 was established on the basis of HMBC correlations between the protons of the C-22 methyl groups and C-22 and C-21. The C-15 carbonyl group in enacyloxin is substituted by a hydroxyl group in vibroxin. This was deduced on the basis of the C-15 chemical shift in the ¹³C NMR spectrum (68.2 ppm) and COSY correlations between the proton attached to C-15 and those attached to C-14 and C-16. Vibroxin further lacks the C-19 carbamoyloxy and C-18 chloro groups, and the C-18/C-19 single bond is replaced by a double bond. 3JH,H coupling constants of 15 Hz for H-2/H-3, H-4/H-5, H-18/H-19 and H-20/H-21 and NOESY correlations between the C-6 methyl group and H-4 indicated that the C-2/C-3, C-4/C-5, C-18/C-19 and C-20/C-21 double bonds all have the E-configuration. Similarly, a correlation between the C-12 methyl group and H-10 in the NOESY spectrum is consistent with an E configuration for the C-10/C-11 double bond.

The relative stereochemistry of the dihydroxycyclohexane carboxylic acid (DHCCA) moiety in vibroxin was determined by hydrolyzing the ester linkage in vibroxin under basic conditions. LC-MS comparisons with purified enacyloxin that was hydrolysed similarly and authentic standards of (1S,3R,4S)-DHCCA and (1R,3R,4S)-DHCCA revealed that the DHCCA moiety in vibroxin has the same relative stereochemistry as the corresponding portion of enacyloxin. The polyketide chain resulting from alkaline hydrolysis of the ester linkage in vibroxin could also be detected by LC-MS. The same was not true for the corresponding portion of enacyloxin, which had degraded. This indicates that vibroxin has greater chemical stability than enacyloxin.

The alkaline hydrolysis reaction was carried out by combining 15 μl of a 5 mg/ml solution of vibroxin/enacyloxin in methanol with 185 μl 0.4M KOH. Following incubation at 37° C. for 4h, the mixture was acidified (<pH 4) with 35% HCl. The samples were analysed by UHPLC-ESI-QTOF-MS analysis using a Dionex UltiMate 3000 RS UHPLC connected to a Zorbax Eclipse Plus column (C18, 100×2.1 mm, 1.8 μm) coupled to a Bruker MaXis IMPACT mass spectrometer. The elution profile is shown in Table 5. The mass spectrometer was operated in positive ion mode with a scan range of 50-3000 m/z.

TABLE 5
LC-MS elution profile for analysis of the alkaline hydrolysis reaction

| | Acetonitrile % | Water % |
|---|---|---|
| Time | (+0.1% formic | (+0.1% formic |
| (minutes) | acid) | acid) |
| 0 | 5 | 95 |
| 5.3 | 5 | 95 |
| 17.3 | 100 | 0 |
| 22.3 | 100 | 0 |
| 25.3 | 5 | 95 |
| 34 | 5 | 95 |

### Example 2—Minimum Inhibitory Concentration Measurements

Minimal inhibitory concentrations (MICs) were determined by the CLSI broth microdilution method. Briefly, representative members of the ESKAPE panel of pathogens were grown overnight in Mueller-Hinton (MH) broth at 30° C. Each organism was diluted to a final concentration of 5×10⁵ colony-forming units/μL using McFarland turbidity standards. Concentrations of vibroxin followed twofold dilutions starting at 32 μg/mL. Assays were incubated for 18h at 30° C. The resulting MICs were determined (defined as the lowest concentrations that visibly inhibited bacterial growth).

Cell suspensions without visible growth were then plated out on LB agar plates to determine the minimal bactericidal concentration (MBC). The MBC was set as the lowest concentration required to kill 99.9% of the originally inoculated 5.10⁵ CFU/ml. All MIC and MBC determinations were performed in triplicate.

The deduced MIC and MBC values for vibroxin are listed in Table 6. Despite the significant structural differences between vibroxin and enacyloxin, vibroxin was found to possess the same potency as enacyloxin against multidrug-resistant A. baumannii strains (MIC=2 μg/ml). This is unexpected because vibroxin lacks the C-18 chloro group shown to be essential for bioactivity in enacyloxin (MIC of C-18 deschloro-enacyloxin³ against A. baumannii strains=>32 μg/ml) (see Furukawa et al., Complete structural and configurational assignment of the enacyloxin family, a series of antibiotics from Frateuria sp. W-315. Chem Biodivers. 4(7):1601-4, 2007).

TABLE 6
Activity of vibroxin against a panel of ESKAPE pathogens

| | MIC | |
|---|---|---|
| Organism | (μg/mL) | MBC(μg/mL) |
| Acinetobacter baumannii DSM25645 | 2 | 8 |
| Acinetobacter baumannii ATCC17978 | 2 | 8 |
| Acinetobacter baumannii AYE | 2 | 4 |
| Acinetobacter baumannii S1 | 1 | 8 |
| Acinetobacter baumannii AB5075 | 2 | 4 |
| Enterococcus faecium DSM25390 | >32 | |
| Staphylococcus aureus DSM21979 | >32 | |
| Klebsiella pneumoniae DSM26371 | >32 | |
| Pseudomonas aeruginosa DSM29239 | >32 | |
| Enterobacter cloacae DSM16690 | >32 | |

### Example 3—Vibroxin Biosynthetic Gene Cluster

The vibroxin biosynthetic gene cluster in V. rhizosphaerae MSSRF3 (FIG. 8) was identified by homology to the gene cluster responsible for enacyloxin biosynthesis in B. ambifaria AMMD (Mahenthiralingam et al., Enacyloxins are products of an unusual hybrid modular polyketide synthase encoded by a cryptic Burkholderia ambifaria Genomic Island. Chem Biol. 18: 665, 2011) and B. gladioli pv. cocovenenans HKI 10521 (DSM 11318) (Netzker et al., Microbial communication leading to the activation of silent fungal secondary metabolite gene clusters. Front. Microbiol. 6: 299, 2015). The cluster is defined from the scaffold referenced as BS26DRAFT_scaffold00001.1 between the coding sequences BS26_RS12130 and BS26_RS12145. The vibroxin biosynthetic genes were annotated on the basis of sequence similarity to those from the enacyloxin biosynthetic gene cluster in B. ambifaria AMMD (Table 7).

TABLE 7
Proposed functions of genes in the vibroxin biosynthetic gene cluster
based on sequence similarity to genes in the cluster directing
enacyloxin biosynthesis in B. ambifaria AMMD

| | | Homologous | Percent |
|---|---|---|---|
| Gene | Putative function | gene | identity |
| vbxA | FAD-dependent chlorinase | bamb_5928 | 68 |
| vbxB | α-Ketoglutarate and non-heme | bamb_5927 | 71 |
| | iron-dependent | | |
| | hydroxylase | | |
| vbxC | Thioesterase | bamb_5926 | 45 |
| vbxD | Polyketide synthase | bamb_5925 | 48 |
| vbxE | Polyketide synthase | bamb_5924 | 49 |
| vbxF | Polyketide synthase | bamb_5923 | 46 |
| vbxG | Polyketide synthase | bamb_5922 | 46 |
| vbxH | Polyketide synthase | bamb_5921 | 50 |
| vbxI | Polyketide synthase | bamb_5920 | 47 |
| vbxJ | Polyketide synthase | bamb_5919 | 51 |
| vbxK | Dihydroxycyclohexane | bamb_5918 | 71 |
| | carboxylic acid | | |
| | biosynthesis | | |
| vbxL | Dihydroxycyclohexane | bamb_5912 | 59 |
| | carboxylic acid | | |
| | biosynthesis | | |
| vbxM | Dihydroxycyclohexane | bamb_5913 | 53 |
| | carboxylic acid | | |
| | biosynthesis | | |
| vbxN | Dihydroxycyclohexane | bamb_5914 | 57 |
| | carboxylic acid | | |
| | biosynthesis | | |
| vbxO | Nonribosomal peptide | bamb_5915 | 49 |
| | synthetase condensation | | |
| | domain | | |
| vbxP | Dihydroxycyclohexane | bamb_5916 | 67 |
| | carboxylic acid | | |
| | biosynthesis | | |
| vbxQ | Hybrid polyketide | bamb_5917 | 36 |
| | synthase-nonribosomal peptide | | |
| | synthetase | | |
| vbxR | LuxR-like transcriptional regulator | bamb_5911 | 54 |
| vbxS | Hypothetical protein | bamb_5929 | 51 |
| vbxT | MATE family efflux protein | bamb_5933 | 55 |

### Example 4—Activity of Vibroxin Against Human Ovarian Cancer Cells

Vibroxin was found to have an IC₅₀ between 50 and 100 μM against A2780 human ovarian cancer cells. The cells were obtained from the European Collection of Animal Cell Culture and grown as single monolayers in Roswell Park Memorial Institute medium (RPMI-1640) supplemented with 10% (v/v) fetal calf serum, 1% (v/v) 2 mM L-glutamine and 1% (v/v) penicillin (10 k units/mL)/streptomycin (10 mg/mL). Cells were kept at 310 K in a humidified atmosphere containing 5% CO₂ and maintenance passages were done at ca. 80% confluency.

For cytotoxicity measurements, 96-well plates were used to seed 5000 A2780 cells per well. These were left to pre-incubate in drug-free media at 310 K for 48 h before adding various concentrations of vibroxin. A stock solution of vibroxin was prepared in 5% v/v DMSO and 95% v/v cell culture medium. Then, serial dilutions with culture medium were carried out to achieve working concentrations. The cells were exposed to various concentrations of vibroxin for a period of 24 h, the culture supernatants were removed by suction and each well was washed with PBS. A further 72 h were allowed for the cells to recover in drug-free medium at 310 K. A modified version of the SRB assay was used to determine cell viability (Skehan et al., New colorimetric cytotoxicity assay for anticancer-drug screening. J. Natl. Cancer Inst. 82(13):1107-12, 1990; and Vichai et al., Sulforhodamine B colorimetric assay for cytotoxicity screening. Nat. Protoc. 1: 1112-16, 2006). In this assay, sulforhodamine B binds to basic amino acid residues of proteins in fixed cells. The percentage of viable cells resulting from exposure to vibroxin was determined by measuring the absorbance due to soluble sulforohodamine relative to an untreated control. The absorbance measurements were carried out using a BioRad iMark microplate reader with a 470 nm filter. Mean percentage cell viability values+/−1 standard deviation were calculated from duplicates of triplicates in two independent sets of experiments.

### Example 5—Preparation of Vibroxin Analogues

Vibroxin analogues can be prepared by mutasynthesis and genetic manipulation of the vibroxin biosynthetic gene cluster. Genetic manipulation of the vibroxin gene cluster can be done by cloning the entire vibroxin BGC in an expression vector using transformation associated recombination (TAR) in yeast and expressing it in a suitable heterologous host (Yamanaka et al., Direct cloning and refactoring of a silent lipopeptide biosynthetic gene cluster yields the antibiotic taromycin A. Proc Natl Acad Sci. USA 111(5):1957-62, 2014). Standard genetic engineering techniques analogous to those reported previously (Liu et al., In vitro CRISPR/Cas9 system for efficient targeted DNA editing. mrBIO. 6:e01714-15, 2015) can be used to introduce mutations and gene deletions. The vbxR gene is predicted to encode a LuxR-like transcriptional activator that induces expression of the vibroxin BGC in response to homoserine lactone (HSL) signalling molecules. Improved levels of vibroxin production in a heterologous host may be obtained by adding a cocktail of commercially-available HSLs to the culture medium or by co-cultivation with an appropriate HSL-producing organism.

Analogues that lack the C-11 chlorine atom and C-14 hydroxyl group, or have modifications to the DHCCA moiety can be produced by constructing in frame deletions in vbxA, vbxB, vbxL, vbxP, vbxN and vbxK. A vibroxin derivative in which the C-15 hydroxyl group is replaced with a keto group can be produced by co-expressing the PQQ-dependent oxidase encoded by bamb_5932 with the vibroxin BGC. To create vibroxin analogues in which the moderately labile ester linkage is replaced by a more stable amide bond, a mutasynthesis approach can be employed involving feeding of 3-amino-4-hydroxycyclohexane carboxylic acid (AHCCA) to mutants blocked in DHCCA biosynthesis. Such mutants can be prepared by deleting one or more genes within the vibroxin gene cluster responsible for the biosynthesis of DHCCA (i.e. vbxP, vbxN, vbxM, vbxL and vbxK). Vibroxin analogues with other modifications to the DHCCA-derived moiety can be produced via a similar mutasynthesis strategy.

To produce analogues with modifications to the isopropyl group, again mutasynthesis can be employed. The conserved Ser residue that undergoes phosphopantetheinylation in the ACP domain of the VbxD loading module is mutated to Ala, and the N-acetylcysteamine (NAC) thioesters of isobutyric acid and a range of analogues are fed to the resulting mutant.

1. A compound of formula (I), or a pharmaceutically acceptable salt, metabolite, isomer (e.g. stereoisomer) or prodrug thereof: 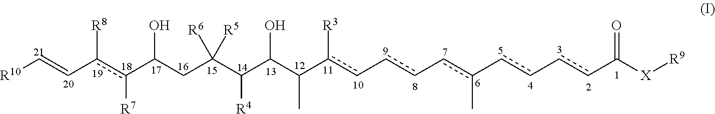
wherein:
X is O, NR (where R is either H or C₁₋₃ alkyl, e.g. CH₃), or CH₂;
R³ is H, F, Cl, Br, I, or CH₃;
R⁴ is H, or OH;
R⁵ and R⁶ are independently selected from H and OH, or R⁵ and R⁶ together are ═O;
R⁷ is H, F, Cl, Br, I, or CH₃;
R⁸ is H, OH, or —OC(O)NR′₂ (where each R′ is independently H or C₁₋₃ alkyl, e.g. CH₃), preferably R⁸ is H, OH or —OC(O)NH₂;
R⁹ is a 5- or 6-membered, saturated or unsaturated, carbocyclic ring optionally substituted by one or more substituents, or R⁹ is an optionally substituted straight-chained or branched C₁₋₆ alkyl group (e.g. C₁₋₃ alkyl group);
R¹⁰ is a straight-chained or branched C₁₋₈ alkyl group (e.g. C₁₋₆ alkyl group), a C₄₋₆ cycloalkyl group, or an optionally substituted aryl or heteroaryl group; and
each [symbol] independently represents an optional bond (i.e. each of C₂-C₃, C₄-C₅, C₆-C₇, C₈-C₉, C₁₀-C₁₁ and C₁₈-C₁₉ are independently either C—C (single) or C═C (double) bonds).

2. A compound as claimed in claim 1, wherein R⁹ is an optionally substituted cyclohexyl or cyclopentyl ring, an optionally substituted cyclohexenyl ring, or an optionally substituted, straight-chained C₁₋₆ alkyl group.

3. A compound as claimed in claim 1 or claim 2, wherein R⁹ is substituted by one or more of the following groups: OH, NR^{a}₂ (where each R^{a} is independently H or C₁₋₃ alkyl, e.g. CH₃), SR^{b} (where R_{b} is H or C₁₋₃ alkyl, e.g. CH₃), halogen (e.g. F, Cl, Br, or I), C₁₋₃ alkyl (e.g. CH₃), CO₂H (or an ester thereof), PO₃H₂ (or an ester thereof) and SO₃H₂ (or an ester thereof).

4. A compound as claimed in any one of claims 1 to 3, wherein R¹⁰ is a straight-chained or branched C₁₋₈ alkyl (e.g. C₁₋₆ alkyl) group, preferably a straight-chained or branched C₁₋₅ alkyl, more preferably a straight-chained or branched C₁₋₄ alkyl, e.g. methyl, ethyl, isopropyl, or tert.butyl.

5. A compound as claimed in claim 1 of formula (Ia), or a pharmaceutically acceptable salt, metabolite, isomer (e.g. stereoisomer) or prodrug thereof: 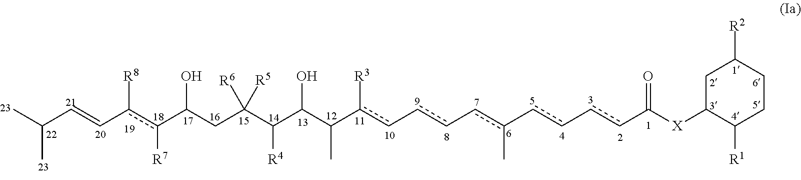
wherein:
R¹ is H, OH, NR^{a}₂ (where each R^{a} is independently H or C₁₋₃ alkyl, e.g. CH₃), SR^{b} (where R_{b} is H or C₁₋₃ alkyl, e.g. CH₃), halogen (e.g. F, Cl, Br, or I), or C₁₋₃ alkyl (e.g. CH₃), preferably wherein R¹ is H, OH, NH₂, SH, F, Cl, Br, I, or CH₃;
R² is H, CO₂H (or an ester thereof), PO₃H₂ (or an ester thereof) or SO₃H₂ (or an ester thereof), preferably wherein R² is H, CO₂H, PO₃H₂, or SO₃H₂;
X is as defined in claim 1;
R³ to R⁸ are as defined in claim 1; and [symbol]
represents an optional bond (i.e. C₂-C₃, C₄-C₅, C₆-C₇, C₈-C₉, C₁₀-C₁₁ and C₁₈-C₁₉ are either C—C (single) or C═C (double) bonds).

6. A compound as claimed in claim 5, wherein:
R¹ is H, OH, NH₂, SH, F, Cl, Br, I, or CH₃;
R² is H, CO₂H, PO₃H₂, or SO₃H₂;
X═O, NH, or CH₂;
C₂-C₃, C₄-C₅, C₆-C₇, C₈-C₉, C₁₀-C₁₁ and C₁₈-C₁₉ are either C—C(single) or C═C (double) bonds;
R³ is H, F, Cl, Br, I, or CH₃;
R⁴ is H, or OH;
R⁵ is H and R⁶ is OH, or R⁵ and R⁶ are ═O;
R⁷ is H, F, Cl, Br, I, or CH₃; and
R⁸ is H, OH, or OC(O)NH₂.

7. A compound as claimed in any one of the preceding claims, wherein at least one of R⁷ and R⁸ in formula (I) or (Ia) is hydrogen, preferably wherein both R⁷ and R⁸ are hydrogen.

8. A compound as claimed in claim 1 of formula (Ib), or a pharmaceutically acceptable salt, metabolite, isomer (e.g. stereoisomer) or prodrug thereof: 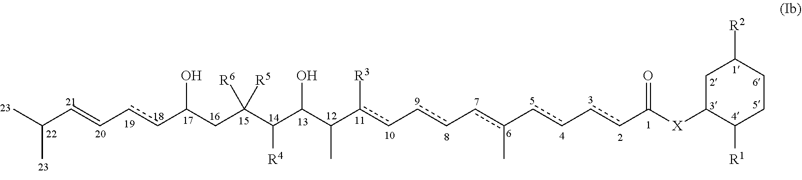
wherein: R¹ to R⁶ and X are as defined in any one of claims 1 to 6.

9. A compound as claimed in any one of the preceding claims, wherein X is O or NR (where R is either H or C₁₋₃ alkyl, e.g. CH₃), preferably wherein X is O or NH, e.g. wherein X is NH.

10. A compound as claimed in any one of the preceding claims, wherein R³ is H or Cl, preferably Cl.

11. A compound as claimed in any one of the preceding claims, wherein R⁴ is OH.

12. A compound as claimed in any one of the preceding claims, wherein R⁵ is H and R⁶ is OH.

13. A compound as claimed in any one of the preceding claims, wherein C₂-C₃, C₄-C₅, C₆-C₇, C₈-C₉, C₁₀-C₁₁ and C₁₈-C₁₉ are C═C (double) bonds.

14. A compound as claimed in claim 1 of formula (II), or a pharmaceutically acceptable salt, metabolite, isomer (e.g. stereoisomer) or prodrug thereof: 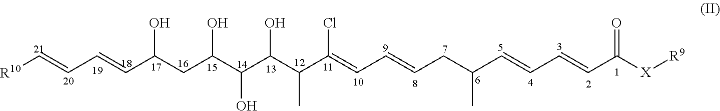
wherein X, R⁹ and R¹⁰ are as defined in any one of claims 1 to 4 and 9.

15. A compound as claimed in claim 1 of formula (IIa), or a pharmaceutically acceptable salt, metabolite, isomer (e.g. stereoisomer) or prodrug thereof: 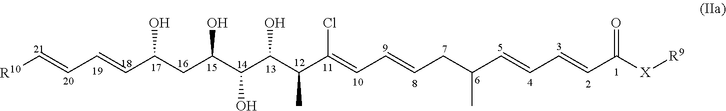
wherein X, R⁹ and R¹⁰ are as defined in any one of claims 1 to 4 and 9.

16. A compound as claimed in claim 1 selected from any of the following compounds, or a pharmaceutically acceptable salt, metabolite, isomer (e.g. stereoisomer) or prodrug thereof:

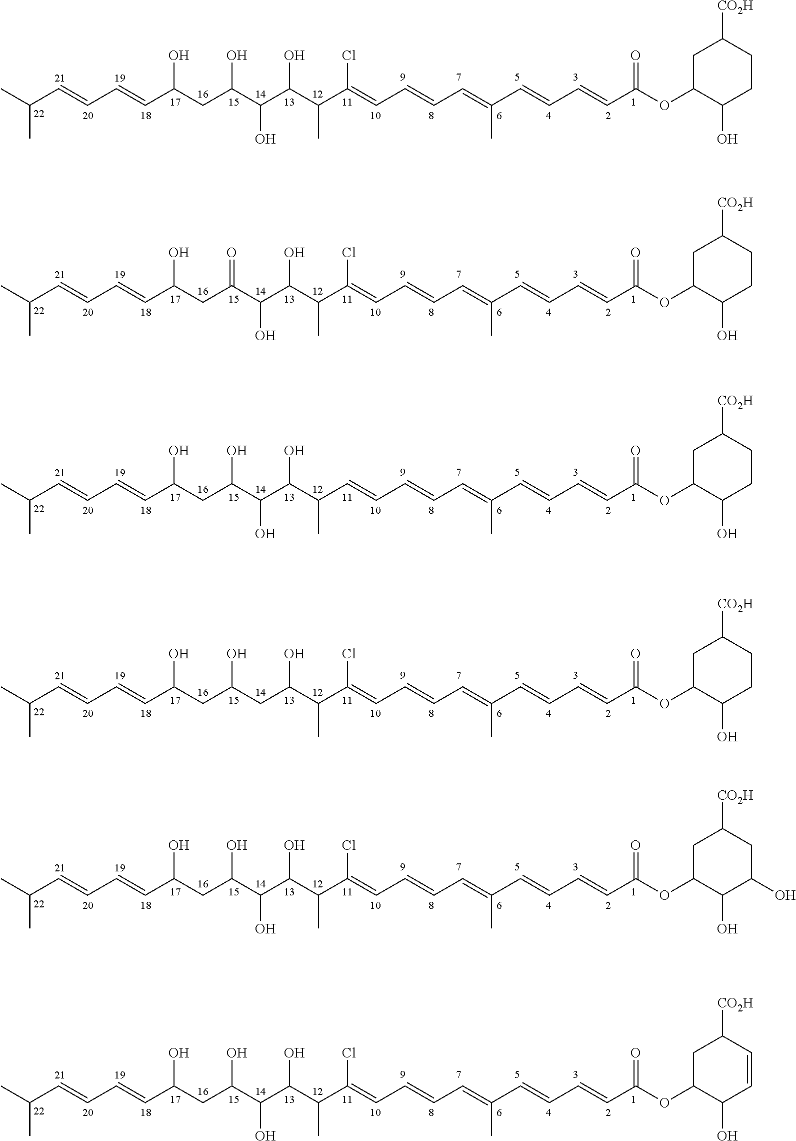

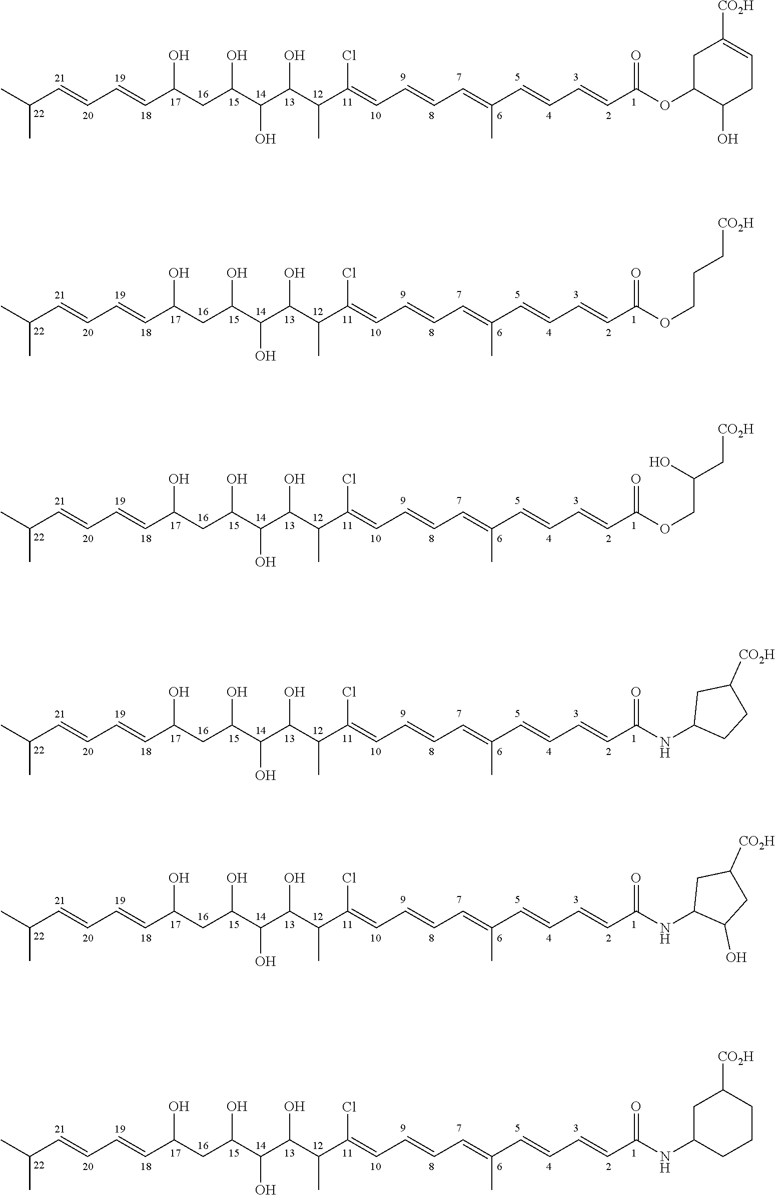

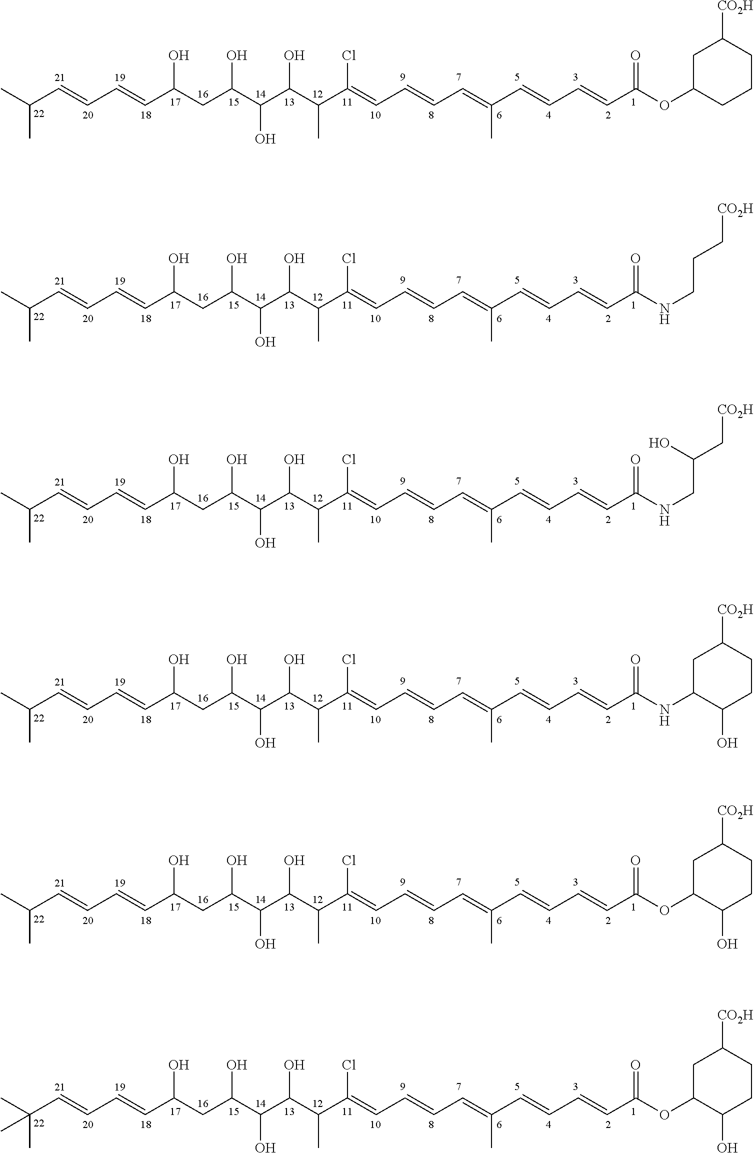

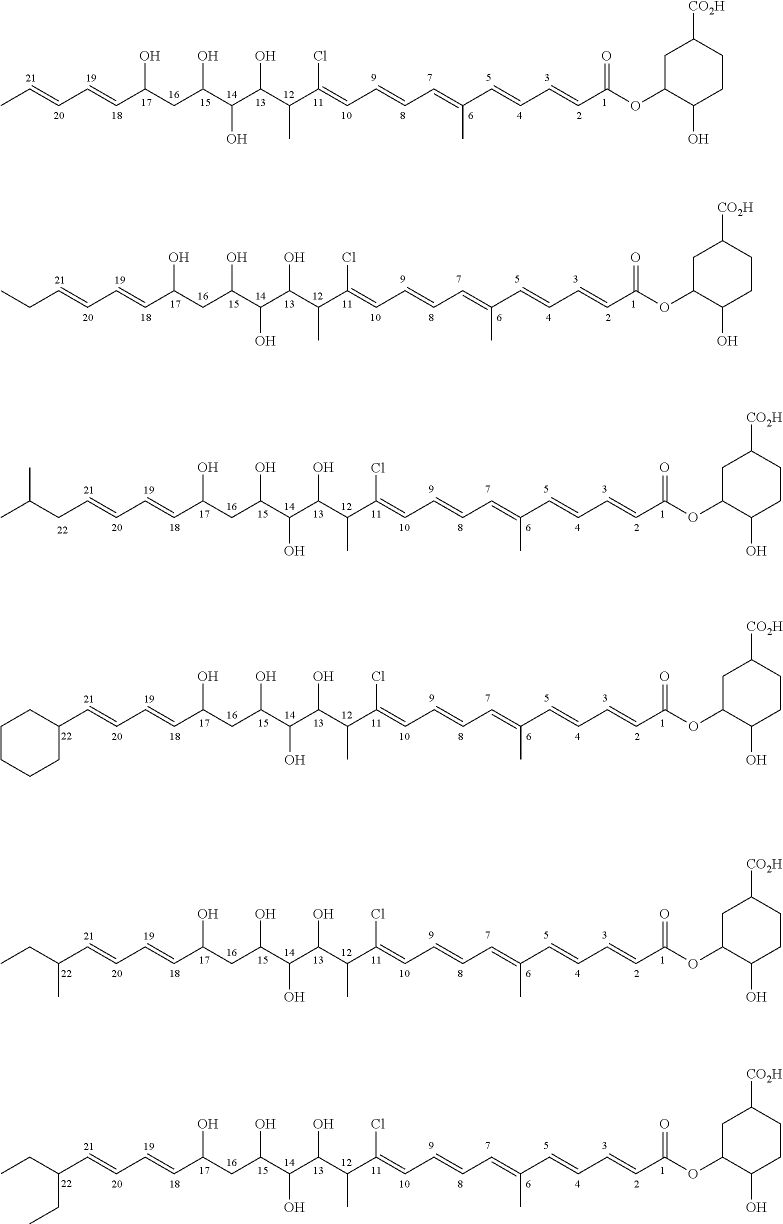

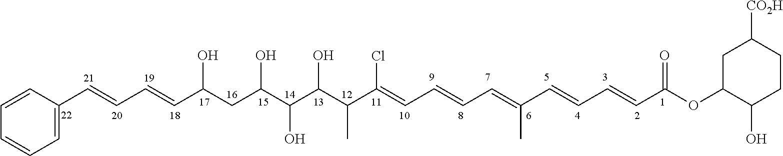 [SMILES: CC(/C=C/C=C/C(=O)OC1CC(C(=O)O)CCC1O)=C\C=C\C=C(/Cl)C(C)C(O)C(O)C(O)CC(O)/C=C/C=C/c1ccccc1]

17. A compound as claimed in any one of claims 1 to 16 for use as a medicament.

18. A compound as claimed in any one of claims 1 to 16 for use as an antimicrobial agent.

19. A compound as claimed in any one of claims 1 to 16 for use in the treatment of an infection caused by a microbe which is a bacterium.

20. A compound for use as claimed in claim 19 in the treatment of an infection caused by a microbe which is a Gram-negative bacterium, e.g. selected from Acinetobacter species, Burkholderia species, Ralstonia species and Stenotrophomonas species.

21. A compound as claimed in any one of claims 1 to 16 for use in the treatment of an infection caused by at least one microbe which is resistant to at least one antimicrobial drug.

22. A compound for use as claimed in claim 21 in the treatment of an infection, wherein the antimicrobial drug is selected from drugs of the carbapenem family, drugs of the penicillin family, drugs of the vancomycin family, drugs of the aminoglycoside family, drugs of the quinolone family, drugs of the daptomycin family, drugs of the cephalosporin family, drugs of the macrolide family, and combinations thereof.

23. A compound for use as claimed in claim 22 in the treatment of an infection, wherein the antimicrobial drug is selected from penicillin, ampicillin, methicillin, vancomycin, gentamycin, ofloxacin, ciprofloxacin, daptomycin, cefdimir, erythromycin, equivalents thereof, and combinations thereof.

24. Use of a compound as claimed in any one of claims 1 to 16 in the manufacture of a medicament for use in treating an infection caused by at least one microbe as defined in any one of claims 19 to 23.

25. A compound for use as claimed in any one of claims 19 to 23 in the treatment of infection, or a use as claimed in claim 24, wherein the infection is an infection of the respiratory system, digestive system, urinary system, nervous system, a blood infection, a soft tissue infection, a skin infection, a nasal canal infection, or combinations thereof.

26. A pharmaceutical composition comprising a compound as claimed in any one of claims 1 to 16 and a pharmaceutically acceptable carrier.

27. A pharmaceutical composition as claimed in claim 26, further comprising at least one other therapeutically active agent.

28. A pharmaceutical composition as claimed in claim 27, wherein the compound according to any one of claims 1 to 16 and the other therapeutically active agent are adapted for sequential, separate or simultaneous administration.

29. A variant or mutant of the microorganism Vibrio rhizosphaerae, e.g. of Vibrio rhizosphaerae MSSF3 (DSM 18581).

30. An active agent, especially an antimicrobial agent, obtained or obtainable from a microorganism as defined in claim 29.

31. The active agent of claim 30 having mass spectral and/or NMR spectroscopic properties substantially according to one or more of FIGS. 1 to 7 and/or Table 3.

32. A process for the preparation of a compound as claimed in any one of claims 1 to 16, comprising cultivating a microorganism capable of producing said compound, in a culture medium comprising a source of assimilable carbon, nitrogen, and inorganic salts and, optionally, recovering said compound from the culture medium and, optionally, further converting the compound into a pharmaceutically acceptable salt thereof.

33. A process as claimed in claim 32, wherein the microorganism is Vibrio rhizosphaerae or a strain of Vibrio rhizosphaerae as defined in claim 29.

34. A process as claimed in claim 32 or claim 33, further comprising converting the compound into another compound of formula (I) by chemical synthesis and, optionally, further converting the resultant compound into a pharmaceutically acceptable salt thereof.

35. A method for the treatment of an infection, the method comprising administering to a subject in need thereof a compound as claimed in any one of claims 1 to 16, wherein the infection is caused by at least one microbe, optionally wherein the microbe is resistant to an antimicrobial drug.


SEQUENCE LISTING (41 sequences)

SEQ ID NO: 1 - DNA, 69117 bp, Vibrio rhizosphaerae MSSRF3
atgtctacagccccgcctctagccaataccagcccgagcgctcacaaaagttattttaaagaagcacttaaactgtcattaccattagttatttcacagtttctcatcatggcaaatggatttatttgtaatctaatgttgtctcgggtcagtgaatcagcttttgcagctggtttatttattaatacgatccaaatgattcttgtcaccgttgtttttggactgctttcatctctcagcccattgattggtattgtcacaggtcagggaaagcctgaacgtgtaggccagttatttatcgctgggtgcattatttcactgatactgagtttctctagcattgctatattgatctatattgagccaattatgttgctgttagggcagcctgagaatttggctcatctctgcgcagagtattttcgaatttacttatggtctgtacctgctgctggtttaattacagtttttatacagctttttttaggaacatttaaacaggttgttgtatttctatatagcataggaaatttactcttatcatctgtcctcagttatatccttatatttggtaaatttgggtttccagcgatggggttggaggggctagcttgggctatctcaataacttcttgcgtggcagtgcttagtctaggttgctttatatttagaagctctgattataaagataagaaattaactaatctgaaatggcagtttctcaaacaagattcaggtcgtattattcgacttggtttccctatttcgttacatgtagggaatgagatgttttcattcctattcattattataatggttggttggattagtatagaagcactcaatatgcagcaggttgtcactagatacttaatgatgctagccatccctatatttggcttatctcaggcggttactgttgcaattagcaagcagtttggagaggaaaagtttggtgaagtcataaatatagggcgagcttatatccgaatgggagttatctattcttctgttgttctattattatttgcaaccattcctgatgtgtttattcgagtgtttattgataacaccccccaaaatacagctatttataaaactctctccattatattaattttagttgcaattgggcaggttttcgatgcgataaaaaatattgttacagggtcgcttagagggcttcatgatactaaatttccgatgctacttagttttattactgtgtggccgataggcgtgcctcttgcttatttaatgggatttacctttgaatggggattgattggcattacgattgcccatagtttggcaatcgcattttcttgtatcttcttgtattggcgttggattcagaaatccagaaatatggccaaaaggtagtattcagctattttgaacgccctccctttgttttcgcgcttgttgtaatttttctcgcaagcgtgatttcgcctctggatctttcggttttacccagtctcgacctattttttcccagtcaggatcttgaggattgagacctaaaccttcaatgtgtggtgcttgagccagaaaacattgccctttaaattgtagtaagtagaaacccttgtgagtatctaagtacgcctttgcatcatcataagttgggcaccagacatttaagttgggctcactgacacctgaacgatataactcagtggcttcagtatcatcaatgtgatcttccactttgatcacctgtgccattgttgaccaatcgatatatcctgcttcttttgcaagtagctccaaatacttgtttatggaaaaagtattaagacgctttaactgtattgattcatcatctgatcggcttttcaaaattcctgatgttttaaatctttcaagtatcgccttgatatcctcttcgacgctctctttttctaagcctaacttgaccatttttcttaggcgatttgcctgaactctacacttatcgattctacttatagcttccatctttcatatcctaggtttgacttactttttattgttgtagagtttgtcaggcgtataggcatcttgtgccgatattatctccatgagctcttctaaatcatcgtctgggttatagtctgggtcgtccaaaggttcgatgagttcaatgatttcgaaagtaaatgcttcttcgcctaaacggttccagtccgcctgaattgctttatttcgatggctaccaagtttaagttcggtcttatgtcggttcagtctcgcttgaacgtctttacaggttcctatcagtgatagacgatcttcttgattgtaaacccgataagcacccatcggtcgtggtgtttctttatactgtttctttagtttttttcttctatccatcggtttatccacattaattattcgaaaatatatgacagcttgttaaacagtttggtttaggttgttgatattgaggttctttgaacctaattgatcgaccctaaagtagatatttaacgaaactcattctctctaatgaaagtatctccgtcaatgacgaagtgcatgttcctgatactgtaactattcctagtctcctttcatttttatagcgacttgttttgtatttttagatgtttcctgaaaatatcaatctttatataaataatactgtcattaattggggtggcggtgagtctattaaatgatattttatttggtaaaattatcatttaataatgagtgcaagtaacatatacataataaatatacataataaatatacatcatgtataaaaagttatttttgatatatagagaaaggttatttctatattatccttgatattcaatatattaggatattcattttttgattgatgtatgtcacataaatggttttttataatgttataaataaaattaaaggtttatttgtaaaaaatgggtgacaataaataaaaactagttaatataatgtacatattaatgattatttgttgttttttttgttttctgattaatgaggtgtatcttttgacctgttgaggatgttgttatttattaaatagctctctgttaaataaccctctgctttattgggtggtaaaaatagagtgtgaatttctattctcgttttctgaaattattatgatttctatgtttattcgtagattcaaataatattaaaatgatcgtaaatatatattacgattttcgcctatggaatattaaggagtggcctagatatgggacaccaacaaacggatgttttaattttaggtggcggactggcaggattaaccttagcacgtcagttgcgaatggaaatgcctgagctggatattatagttcttgagcgaagtaaaaggccggctcctgaggcttgttataaggttggtgaatcgagtgtagaagttgctgcgcattattttgcagaacgtctagagttgagagaacacatcgataatgagcaactgccaaagcttgggttgagatacttctttcctcagggcgagaatcgagatgtcacgaaacgctttgaaattggaccaagcgagtttccagttactcctagttatcagttggatcgaggccgctttgaaaattatctgcatgaattgaatgtggataatggcattgaaattcatactaaagcaaaagttcaacaggtaacgctaaattctgaagaaatgcatgaagtaaaatatatgcatggatccgaggtgaagtctattcagagtaagtggttggttgatgccaccggacgtaatcagttaattaagcgtaaactgaatttggagaaagatagcaaccttcataatgatgccgtctggtttagattggacgcagaggttgatattggcagtttaagtgatgttccccaatggcaggatcgtgctgttgaaccgcgtcgtttaagtacgaatcacttcatgggaccaggttattggttatggtttattcctttgggttcaggtgcaaccagtgttgggattgtctttgataatcaaatgcataatttcaaagatatgaacaactttgagaaagctttaaattggttgtatgaacatgaaccgcaatgtgcagagttagttgaaagttaccgaagccaagtgatggattttattggatatcggaattattcatatagttgtaagcaagtattttcagcggatcgctggtgtattactggtgaatccggagtatttatcgatcctttctattctcctggtagtgattttattgcttatagcaacgattttgtgactgaactaattgtacaagacttcaaaggtaaagatattagacaattggcatcttcttttaacgatacctttatgaagttagctgatgtggtctcaatgctatatgaaggcttatatccaaaatttgggaatggttttgtgatgaagcagaaaattctttgggatagcgttatttatctcggtgtcacatgtctgttgtactttaatcgaaagtttactgatcctgattttttagcgttggttgattcagagttagcacattacaactatttattacgttctgtagctgaacactttaaacgtcaacccattactcatgatgtacatctcgatggcgaatatgtcgatttaactaaaacctttttgtttggtagaaatctgaacgaagagttgaccattgaatataaagatgacgacgcgctgattgaaaaactaagagagaatatcgaactgctcgataaaatatccgtaagtattttcaataatgttgatattgcgtggaattttcaacctaaaaaattagcagatacaagtgaagagcaagtcgtaaacagctaggttttaataactctggagcattcaatagataccgaataagtaaatccagaaatatcaagagtgggagaattggttgtgtcaccatttgagcagaatttagaaattcgtaaagctgtgattaaggtaatgaaagaaggcgggtattaccaaaaaatcatggatggggattctgaattagaccgtgtcaaagaattcttaattcggatggagaaagcccaacacggagaacgtttatataagcagcatcctacatttttaccgattttcccaggcctcgataatcaaccgatgcgctcatctaaaggcgatcctgttgctgactacttacgtgaagcaacaccagcaatcaaagaagacgcgctgcgtttaagagatcgcacactgtcttttacggggggaattgtgacagatggggcatggaatattttcccactttggtacatgggagttaatctcccgtttatgtctgcacactgtcccaacttatcccgaattgctgccgagatacctcgttgtggcatgttgcacccattttctgaagcgctattgtcttggcaggatcctcacacccatctcgctgcgcattgtagtgttgattcgttgaggctgcgatatagcgttggcattattgttgagtccgattgtagtctacgtgttggtgaggtaaggaagcagtgggagaccggagagtcaatcatattcgaagattgttttgaacatgaagcatggaatggggataaaagtcgtttagtgttcatcattgatacttggcatccagaccttaccgccattgagtgtgaagccttactcgcagggtttcggaagaaagaagttcgccatatcctctatgagtaccgtatgtcggaggcgatgagacctttcctgctgaaccgatttgaaatagaagaaaaagagcctttgattcaacagtactgggagcatgaaggtgaattatatgttcctaagccaaaaaattgggggatatgggggacgacccctgtgtttacctgagtccctgaacgggctcatatagagccctaatgacattcgcggtattttcatcacaaggaataatgagaatagccgcttaaaacaatcatcatggataaggatatatcattttgttcggcgtcgattttgaacaatgctttagctcgccagtaaaggagcgtactaatgcaaatgtgatattgttttgttttccctacgctggtgcgggggtctcttcttatcacacactttgtaatttattacctttggaagtcattccatttgttgccagattacccggtcgtgaaaccacacatcaacaaccgccatttactgaaatgtcagatattgtttcgtacttgtcaaaggcaattcgccctgctttaggaaagtcgtcaatttttttttggggtcacagtatgggggctcttcttgcgtttgaaatcgcgagaattgtgcagaaagattacaccttagatggtttgattgtttcaggacatgtcgctcctcatttacctgttaagccaaagcctgtttcaattaaggaaatggatgacgctcagttccttgaaattgtcagaagctacggtggtatgcctgatgccattctccagaatcccgatatcctccaattagttttaccgcaaatcagagctgatttaaccgcgctagagggatatcaattcgttcagggagaaaaacttgagagtgatattttttgtattaatggtcagtccgatcatatggtcgatccacataacgtgatgctttggcaagaattaacgtcagcgaaattctccagtcaatggctaccagggtcgcatttcttcattaatgaacaacgccatggggtggtgaagcgcattagcaatatcattcgtcatcaacttgttcaaatagatatcatctaacaaaaggacacatagcatgctggaagtcgttaatacctaccttcatggctacgcagcaattccgatgctaatggtttgtcaagagacaggtatcatgactgcgttggaaaagggacctgtaagcgtacaggaaataacaaaagagctgtcggccaacattggttatagccgactgatttttcgaaccctgaatgcactgggttatgtcacaacgacagaccgggaaaaatatacactgagtccggatacaaatcatgactggcgtctacctgacacaatgattgaagtttataaaatcgatttctatgattattttttgaatggtactcataaaaacaaggtgagagcttggctcgatttgtcagtcgatgattggggtaaaactgataaagaaacgcagcagctcatggatggcgtgatcatggtgccgtttttattggcgctcgctgaattaggactcggtcaggacgaaaacatccagtccagtttacttgaacagtatattcacgaaagtatttggtctgatgtacagtattttctgactcggaaaaacctgttagaagaagacaaactagtccttaatcaggctggttggcacttatgtaatcgtgcgttgaccatggctactatggtgtcataccggccaatgttattgaattttaaagaactactttgtggtgacccaaagcaagtgtttgaccgtgatgaattgggacatgaagtccatgtcgatcgaagccttaatgtgattgggagcggcttccaacatggtaagtattttttcgatttggcagagtttatcgttgagattttcaatcgaacacctgtctcagagcaacctaattatatcgtcgatatggggtgtggcgatggtgcgttgttaaaaacactttatgaggaaatcactaaaaatagtttaagaggacagtatctagaacaataccctctgcttttggttggtgctgattataacgagaaatccttgctggaatcagcaaagacattggatggattccctcatctgttgttacctggggatattggtaagcctcaggatttgatgacagatctacggtcatacggcattacagatgttgatcaaattatacatgtacgttcatttttagatcatgatagacccatcaatattgaggcagcaccgacagacatgatccccagagatgatcatattcttgtcaatggtagaggtgattggattgactctgatacggtgttcaaagacctctgtgagtatttgacacggtggtcaagtattttggggaaacatggactaattctgctcgaagttttttctttacccattagcttaacgcgcgaatattttaatcagacagagagtttccatttcgatttctatcacgctatgtccggacaagttttagtcgatgctggaatgttccatcaagcactcgcttgtgcaggtttgtatcctaaccgagagacattgtcgagatatcccaaaacaacacctttttctcgtattgtattgcagcagctcttgcctaagcaggtcattgtaaggccttttgtcttagatgatatgccggctgtgctcgccattgatcgggcttgttggcctgagcacatgagactttcaacaaccgagattgagcgacgtcatcgcacctttcctaaaggtcaactcgtcgtggaatatgaagggcgtgttgttagtgtactttattctcaacgcattgacgaaatcgagccgatattttctctgggttatgcaaattatttgaatgctcataataatgatggtcgatattggcaactcttggggcttagttcagatcctgattatcaatttttgtcgctgggggatcatcttttagagcatacgcttgatttagcgtcactcaccccgaaagtcgaagcagtgtatggggtaacacggtgcgtggcatatgctcaacaatctgtgccattggaagaatacatttttaagaaagatgctcagggttatccaattgaaccgttattgcgctttcattttagccatggggcttcgatagagggtttagtgcctgagacacgtccagaggatgaagagaatgaaggtgcaggggttctgatccgctatgacctgagcaaacgttttcaaagaccaaatcaaactcaaccgagagcaacaagagatatagagcgggcaattactatcgtgtcagattgtattcgccagataatgaaaacaccaagtgattttcgagatgatttgccgttaaaggagctagggttggattccatgggattgatggaactccgtcttttacttaatcgtgcatttggtattgagtttgaacctgcgtatttcttcaattatccaacggcagtcgcgatcgctgactacatcgataaattaaacaaatcttacgtacagcctgaagcagagctgagtcatgcatcgcttgtgggacgacaggcagtgcctaaccgcgtgtctgtatctgatgtcggacgctctgactccgaagttcaatcgtcgcaagagactgacgttgccattgttggcatgtcattacgtttccctcaagggattgaaacacttgaggaattctggtcggtcttaacacaaggagagtgcgtaattagtgagcgtccggagacccgatggcgagaatttcaatccgaattggagacattagatacacgttggcaacacatccatcgtggcggttttttgaaagacatagataagtttgatgcggaattttttcatattacccctgcggaagccgcatccttagacccacaacaacgtatcttgctagagcttgcttgggaagcttttgaaaatgccggtatggatgttgaacaatttgcaagtcacccagtcggtatttttttaggcgcttatacacaagattatcaaacgctgaccttacgacaaggtgttgatgcggtggatgcctattttggtggtagtaatgctttatcgacagcagcaggacggttggcgtacttttttgacttccgtgggccaacgctcacacttgataccgcatgttcttcttcgagtagtgcgatttatacagcatgtcagaatcttcttgaaggcaacgcagaaatagcactggcaggcgcgatcaatacgatgatttccccaaatttgagtttagcttatgcaaaagcaggaatgttgtctgtcgatgggctttgtaaaacgtttgatgctgaggccaatggttatgtgcgaagtgaaggtggggctttcctactgttaaaaagaaaagccgatgcaatccgggatggggattttattcacgcagtgattaaatcggccgcattgatgcaggatggacgcaccaatggtcttacggcaccgaatggcctcgctcaggaagatgtgattcgtcgagctgtgcagttgtccggatacaaagctgaggatgtcagttatatcgaagcgcatgggacggggacttatctaggggatcctgtagagatgcaggcactcagacaggttttctgtgatggtacggcacgtcggaacacattaaaagttggttcagtgaaaaccaatttaggccatacagaagcggcttcagggatggctggtatcatcaaagtcgctttatcgatgcaacatcaatggattccaccacatctgcatcttcaaaatatgaatacattgatgaatattgcgcaggctgacattgatgtaccgacgcaaggtgagttctgggaagtgaccgaaggccaaccaagattggctgggattagctcttttggtttcagcggtagtaacactcatattgtactagaagagtttcacatgcctgactctgagcagtggcatggacgtagcccattacccgctgttatctctgccaaatcgattcaatcgttacggcagaatatccttgctttgctgacgtatatcgatcagcacggggcaacactcaatcttgctgcactatcgaaaacactgacacaaggacgtacgcagcattgttaccgaatctcttttccttttgattcgcttgagacactgaaaacgtctctgcaagcgcagttaaaaaatttatcctcagagaatccgcctgttacttcatcggttcaacccagaatcgcatttatgtttactggtcagggggctcagtataaggggatggcttctcgcctggatcgttttagtccggttttccatcaacacctgtcagagtgtgccgctttcgttcagacgtatggtgaatttgatttatttgagcttctttgggggaatgatgagactctgatccaccaacctcgctatactcaagtcgcgttattttgtatcgaatatagtctctctgaaatgttacgggagtcagggattgaagctgaagcggttcttggacatagcgtcggagagtatgcggcagcttgttataccaaagtcattagcctcgaaaccgcgatccaattgctcttttatcgcggtaagttgatggatgaacaaactgcaatgggtaagatggttgcagttcttgcaccattgagcgatgttgagtgcttagttgaaggctatgaacttgtctctattgcagcgaagaatgggccttcaaatcaggtaatatcgggggatccagaacagatcgagcgcattgttcaaaatgctgaacaactgaagatgaaaacatttccactcaccgtggaaagagctttccattctccgctgatggcccctattctcgaaccattcaaagcgatagcggaaccactgacttatcaggcaccacagtcagatcttttttcaaacttaactggtgatcgatataccgaggtgatggatagccaatattggactgatcatatcagctcacccgtgaactttgagcaatctgtacgaagtcttctgaagcatggggtcgatatggtggtcgaaattggcccccgtccgattctgacgaatatgggtttactgattgatgcagataaaaatacgctttggctttctactcttgtcgatgctgagaaccataatctagcgagtctctttactcggttgagtcagtctggtgtagacattgattggcgtttttatcctcatgaaagcgtgacgagactctctgacgtaccactttatcagtttgatcgaagcaaatactggctcactgagtcttcgaaagagcacgctcttacatctgtctccgtcgatgatagcgcagaacaggatacaaggcgcgatatagacaaagagaccgaatcacaaccgcagtctgctacgcctaacttagttggtatctttccgaaccgtatcgaaccaaagcgtgaccgtgatatatgggcgcatgcgattggcagcgagagtctatttcctgccagtggttacattgggttggcgttagaggctgggatgtgttgtttgaagcgagacggttacgtagagcttagagatatgcatctcgaacagatgttgacactgtctgatgaacaagaaacctatattcaagtgagagtttcagaaggtgattcgggcaatatgctttctatacaattagaagcatggcaggcggatcagaaggctccccgatccttgtatgcaagaacagcaatgaaagcactgacgtttggtccggaggatgcttgggaaaaggaagtcacaactgttgatctgccattaaccagtgacgctgtcatgcagggagaaacgttctataaacgaatttcagcattaggctatgcctatcggcctccgttccaagggattcggaatgttcggcgcgctggaaatactttggtcacgacactcagattttctaatacaacgccaaatgcaggctatgcagcgacgccatggagtctggatgggtgtttccaaaccattctcgctgtcgtcatcgatgatttagaagctaaccatgaccaacttttactcccagtcattattgagcagtttacttggttgtcgtcacttccgtctgaagtgaatgtcgcatgtgtgtgtcagtcgacgacccgtggcatatcagccaacatgtcaatttatgacattgcaggtcaattattagttaatattgttgggttgaactgcgtgtgggtgaatcgccaaagtctaggtttgcaagccattccgagcagtacaccggagatttatcgtcccgattggatggcatctgaggtatcagctgagcttttatcacttgtctcactcaaagaacaaatccctgcttggttggtacttactgatgataatggtcaggggcaggcaattatcgatgtgctgacctcggcagggatgcgagttgaagtgctgcaatgggagaaggctcagaagccatccatatttaccagacagttaatgaatttctcgacatcacaagagacaccatttggactcgttcatagttggtggttaactcaaccgactaatggtgctcatgctagggggcggtatgaacttgatttagctgtggctcagtttgtagcctcaacagatacagagcgtttatcgcgggctattttcctaacatcacatgcctatccggttttgaaaactgacgagatggatgttgaccaaggtgcggggctttgggggttggtgaatacactacgagccgaagtcgctacgcatgaactcacccttgttgatgttgcaccgacacaaactgaggaagaagcacgtcgtcaggctgagcaaattttgctgacgtctaatgcgacactcaaggctacagaagccacggattggcctgaacattttgcgttacgacagggcgctttatttcaacgtgtgcttcaaccagtaacaagctcgactgaatctatcagaatctcgggtgatgccagctatattgtcaccggtgggctcgggggaattgggcaatacattactacatttcttcataaagaaggcgcagggcagattgtactgttgggacggtcatttcctgaagaattacccatttgggtgcaacaccttaatcaaaaacgtcaagcgattattctgcttcgctgtgatgtgacagagccgcacgatattaccaatgtgatgaagcagttagagcaaggtcccacggtccgaggactgattcacgcagccggagttgtggaagatgcgttgctcgtcaatcagactcaggaaatgctttctcgggtttatgcccctaaggttgaggggacgaaacatcttgtggatgcgtttgcaacagagtctcttgatttcgtttggttattctcttctatcgtcggtattttgggggctgtagggcaggcgaattatgcaacagccaatgcggtgatggacagttatgcgcattatctacggcaacaaggtattagggcaacatcgattaactggggcccctggaaagagacgggaatgctgtctgatttgtctcagatggcgctagagcgtgcgagttattttatgacggggattgatccgagcgcttgtagtgatgatttgtttactgctttgctcaattcggatgaagcacaatgttgtgttgttcagtggcatattgaacacatcaatcaggaaaaatatctgccgcaaattttgtcttcattgcgtacccaaccgatacaagagcaggtctctgtctcgttggcatcggttttagaaggggcattaggtgaagaacaggtgttacgtacccgtcgttttgtggcagaaaaaatcgcccaattaacgggtctatctcccgacgtgattgaaccgtccaaaccgttctctgattatggtgttacttccattgttagtgttgaactcagttcaatgttaaccaccagtgtcggtgcaacttttgcgacgaccatgatgtttgactatccaacgctcagtttgttggcggaatacgtattggaacggagtgcgttgagagaccagagcaatgatagtgttcccatcaatgatgacagtttgtcccatgaatctttctttgagatagacaaggatttagatgaaatcgagaatcttgatgacgaactacttactgctttattgagtgaggagatagatgatgagtgaatccagttcgtggaaagggagttacctaaaaagactgaatcagttgtcaaaaaatcagttgttagctctggcacttcggcagagagaacaactacttgatgtcaatcagcaggttatgcaaccggatgacacagttgcgatagttggtattggatgtcgatttcctggcggtgtggtggacaccgatagctattggaaactcttgtgttcatcggaaagtgcaatcactcagatgactgatgaacgatggcacatgcaggggatttatgatcccaatcctgaggtggctgggaaaattcatacacgttctgtcggtttgttggatcaggtggataagtttgatgcgagcttcttttctattacgccgcgtgaagctgaatctatggatccacagcaacgacttttgttagaggtgacatgggaagccattgaaaatagtggtcatgcctgttcagaatttgaaggaaaatcggttggtttgttcatcgggatgatgaataaagattatcttcacttgaatgcgccagacatgatgggtgtcaatgcaaagcattccccttattacgcatctggtgaagcatttagtattggtgctggtcggttagcatacattctaggacttcgcgggccgtgtatgacgttggatacggcgtgttcttcttctctagtgggggtgcatctggcgtgtcaaagcttgctcaaggatgaatgtgattatgcaatcgctggtggtagttcacttatcctttctccagaagcgtctatcgtcagttctaatgctcgtatgttatcgccaacaggtcaatgctggacctttgatgagaaagcagatggttatgtgcgcagtgaagggtgtgccgttgtcgcactcaaacgtttgtctgatgccctggcagatggtgatccagtggtggccacaattgcagctacagctgtcaatcataatgggcggagtcagggattaactgcacctagttcaacggcacaagctgagttgatgcaaactgccctcaaaaaagcgcggattcaaccatcgcaagttcgtttcattgaggcgcatgggaccggtacgccattgggtgatccgattgagatgaattcaatccagtctgtttatggtcaggagcggagtgaagataattcattgaaaattggatcagtgaaagcattgattggacataccgaggcgagtgcgggtgttgcggggcttattaaactggctttatgtgtgtcaaaaaacagaatcattccacagcgtaatttcactcgtttaaatcctcatatccgattactggatggcgtagaaatcgcgacagaaacacagtcattttccccttcagaatccgagtctgaaccattccaattgcactatggcgctgtgaattcttttggtttcagtggatcaaatgcgcacgcaattgtgagcaatgtacagctcacatcaccaatagtgacatggcgaagtccacaactgttaaccatttccgccgaaactcccaaagcgctgttggatttgatgctgcgttatcgtgattatatttccgatgagagcctagatttacaggcgttagcctataccagtcaagtgggacgagatcatttcccggtacggatcgctttggatggagaggcgctaagtacgctaagagaatctttagatagggcgatttccagcttttctcatgatagatatgcgcccaaagtcgatcatgatggtattgtgttgattttgagtgatttaggggaagatgcacagagactacaacagaaattttctaaatctagctcagtttttgctcagcatttcaaaacaatatttgaagcatcgtttgctgatacccacgttgatttacacggtctaattgatttagataaatatgcgctaggtaaagaagaagcttcttatcttgtgctatgtgccctctgccaaacattacggacagcgggtgttgcatttactgcgattactgggactgatttgtgtagtaaattggctgctgccagtgctgttggcatgattcagcatcaggatggtttgcgttatctcagagtatgtctgcacaaagaagcaatgataggttggcaacttggcatcccatcggtttctctgattgatatatggcatggtgggaactgttcagcactttatcaggacatcgaaagcttcaggggttggttagaagagccaaatcaaggaacgctgttaaatagcggcgtagatagaaaaacgctgtttctcagtgttacggatggttttcaaacagcagaaggctcatcagtgcttcccctattgggcgagcatgaggatgcaaaagattgggacactatttgggctaccttgtatgaagcgggtctaaaacttcattgggaagggctatatagtggtactcgtccccgacgtctcgtgctgcccaattatgtttttcaacgtcgccattattggcctgagaatgccaaaacgaaacaattacatgaagtcagtcctgaagattgccatcgatttaaggtgttatggcaagaggaaacagtaacgctggcagccgtgtctgacattaaaccgtgtgttattctgattgaagggccgatagatgacttaccgatggagaacttaccatcagaggtaacgttgggggttctgcccaaagggtggcaagaaagagctgtgatcgacacggtattgactgaataccatcttgatcgttccacgcctgtagagctactcgtctggtttagacctcatgatagcttttctacgggatggcatagtgacagtcagtcccatgaaaccatcgtgaccgcggttgaacaacgtgctaaagcgttattaaacctgacacaggcgcttctagaacgcacaaattatagtcagttccggtttggggttgttaccgaaggggttgaaagtatttctgagcggactgcagtgaatgtggttgatgggctggtcaatggatttatacagacgttgtgcatggaacaaccgcagttgcgtccaaggtgtatcgatctggatcctaatagcttagcggccgataaattcgctcagattgtgactgtattatactcgaaggatgatgaaaatcatttggcatttcgtcagggacgcagatatattcatcgcttggcaaaagctgaccgagtgctctcaaaatcggtgaccgtacgtcatgatcgagcctacttaattacagggggattgggtggcatcgggctctcggtagcgagatggttatcccgtgccggggctggtcaaatattgcttgtgtcacgtcgaggaagtgaagcgcccgatgtcgcagagagattggcgacgctcaaacgggaaagaatgtccgtgacactggttcaggctgatgtgcaagattatgatgccttacgtggtgccgttgaatcgtcagccaattttcccttagctggttttattcatagtgctggtcaactagccgatgcatcattacagaatctgactcatacacaatttcaacaagtttttcagtcaaaggttgtaggggctttgaatctacatcgtctctcgcaagagatggtgtctggcactgatgtggatttattccttttattttcatcgatagccagtttggtagggtcggcagggcaggccaactataccagtgcgaatgggtttaccgctgcattggggcgttcgcgtcgtgccgtcggattgcccgctacggtcatacattggggaccctggtcagatgttgggatggcaagtgatgaacgattaaaccataagatcgagcgctctggtttggttctaattgaccccgatttagcgttgtcctctatgctagatgcgttgaccgagcatcagacagagtcagtcattgctcaatttaattggccgcgtatcaatgcttatttggcggaacgagtgccactgcctctattatctcaattcacacaaacgacgcttgaggcttcgcagtctgccaccattgatgcatcttcccatcatgaggataacaaccaccgtgatttagcaagcgcactgaaacgacaaaatgacacggttgccatacaccagttggcaaaatatgttgaaaccacgattcgtcaagtcttagctatcgacagcgaagattacattgatccgaaaacttcggtgctcgacatggggatggattctttgttgtcggtcgaattacgaaatcgttttgccaccgaattggatttgaaactaccggtttctctcatgtttgataagccgacgattgatggtgtgtcccgcttgcttctggacgaattgcgtcaacaacataaagcgagagctgaagaaacagaaactgtcggtatacactcgattcaccatgcagctcctatagttgctgaagaaagtgcttcgatggaggcagcgcagactctgtcgccaccagtgtcctcatcgccagtaaggatggataatcaagatattgcgattattggtgtcgggtgccgtttacctggcggttcggatgggattgaggctttctggaacaatcttatcggaggtgtggacttagtgagtcctttcgatggaagccgttgggatatcaatcgtttctattctgatgatgtgtcagcgaatgggcgtatgtattcgaatgatggcggacagattacaaatgtacacggatttgacaaccaacttttcggactgagtgatcgtgaagccgaatatatggatccacaacagcgtattgctctggaagttgcctgggagacgattgaatcagcggcctacacgccagaatctctcgcggatcagggaggcatcttcattggccccgggccttctgattttgcagatctatcacaacgacatactaaagcgattacggggttaatgagtcagggccaccatattagtgcgatccctggtcgtattgcgtatcactttgactggaaagggccatgtatggcgattgatacggcttgttcctcatctttagtggcggtgctaaccgcggcacaacatcttaatcagggagattgtcgggttgccttggctgggggagtgaatctgattctttcacccgccaataatatcgttcaatctaaagcaggtatgttgtctccaagcggtcgatgccggacttttgatagtgaggccgatggttatattcgctccgaggggtgtgctatggttttgctcaagcgtttgtccgatgcagagagagatggcgacgtgatttggggggtgataaaaggtggagcgaccaatcagaatggacaaggtcagagtatgactgcgccaagtgcccttcaacaggccaacctgattgagtcggtgctaaccaaagcgaatctacagccgtcaatgatccgttatgttgaggcacatggtactggcaccaagctgggtgatcctatcgaaatgtcagcgctgcaacaaacttatggaagtcaccatgatgctgagaatccactctatgtcggctcagtgaaaagtaacctaggacatacagagagtgctgcgggtgcggtggggctgctgaaagttttgttaatgcttaagcatcgacagattccgcccaacttacatctccatcaattaaacccgttattagaaatagacgaatcagtcgttcgtattccaactacacctgtcgcattaaaagacaaagaaaataatgagttaatgtgtgcagtgagttcattcgggtttagtggcacaaatgcacatttggtcgtggctgcctatgacaatgataaggctcagttatatagtaagaattctgatgatcgaattttctgtctgtcagctcgaacaccaacagcattactggccttgagtcaacgctatatttccgcattggaaagtttaccaacttcagaagacagggacacggttttggccaatagttgttattcatcacttgttggccgttgtcgccatcaacataccatgtgtttatacccagatagttatgaagatctgattgtgcaactgcgagcaatagaaacatctatgaatcaggataagttgccgttacctcagtccatagagcaagtcgctttttgtttaacattgagtacagatattccgccattgaaagcgcttgccatgaaccgacaacagcagtttgttgattcattacacatggcagaacaaagcatcctccagtacgcaagtatcgatcttcacaatgtcagtcaagctgatatcagtccagccttacgactgaaattatcctggttttgtgtgcattacgcgacaatccgatggctgatgtctgtgggtattcaaccggacaaagttttgttcagtgatgatatgtggttggttaccgcggtgttatggcaggaatgcacttgggagagtgcgatcgaacaattgctgcatggcgtttctgaggcaccatctcagttaggacggattccgttggagagaactagtctgagtggtggctctttacttttatcagattttgaaggcgtcggtcaccattgtctgaatgttgtgtcaagcaaagatgacagtttatggcgaaacgcgctagggacactttggtgcggtggacgaacagtgaattgggatgtgtatttcgaacatagtcactgccagcgtcgattgatgccaacttacccatttgaacgacgggactgttcaagaccacaaggtttggttcgtggtgaaaacccggtgaatcgtatgcttgaagagctgctcttgaattaattgaatcattggattgatggctgtcctacagggatggcgaaggagactgaattgccccattttcgagaccaacttaatcatctgaccaaagctcaattgagtgaattggtcgtagcattacgtgaggaagtgttacagctaaggccagatgaagttagtgaagtgcctatttatcagacccattggcaacaaatatctctaccgcaacttgaagacggagagatatctgctcggttgatgatcattatcccttcccatgagagtttccctgaaatatcgatggaggacagtggcattcaggttgagtacttcgtactggatacaactcaggggtggacgccacaaaacatatcaagacaactagaggggcgtctcgaatatattgatatattaatctttcttttgccaaatacttcatttgatatctcagcaccaaaggctgcgcagacgatgttagatatttggtcggcaacaatggctttggctgctgtgatcgcagagcgggtcagtcctcccagattatggttgttcacgcattgggggcagtcgttacccgaggatgaacatgaaattgatactgtcgcttcccctctgattgctttaggaaaatctttatcattagagtgtcctcggatgtgggggggatgcatcgatttagatacgtcaacattgtctctcacctctgcgatcgaagaagtcttgtgtgatgcgggggatgaagaggtcgcctaccgagcaggaatacgctatatcccggaacttcatcgctcgacgacggctacaaaatcgaccggtgatatggttcgtctaaaaccgaatgccacttatctggtcactgggggaatgggaggcattgggcgtcggttggtgtcgcatctactcaatcatggcgtatctcgagtcgttgtatttggccggagagggaaagaggtaccagagattgaacaaaccttgaatcattggcatcgaatgtacccgcatgctgagattgatttgatttcggtcgatttgggcaacttgtcagcagtgagagaagcgttagatgacttacgtcggcagggacccgcattaaaaggtatttttcatgtcgcgggcagtagccagcaagtcgctttaaatcatctggatatcgactcgattgatagcatgatgcgagcaaaagctgctggcgccttgttcctcgatgtgctgacaagagatgcggcccttgatttccagatttatttttcatccatatctggttgctggggaaccgcatcgttgattccttatgcgatggcgaatcgattcctagatgctttgagtacgtatcgtaatcatcagggacacttcacgaaaagtattgcttgggggccttgggcaaatatcggaatggttattgaccagcagcaggagtctttcagccatttaggattgggtttgattgaaccgaatgtcggtatggcgatgcttggccgtttaatgaatacggttggatctcaggttcaggtggttgatgtcgcgtggcaacgttacgctcagcatctaccgatggacaagcatttgcgttggtttcgacatttgattggtggtgctgagaccatataccaaccagttgtggaaaatacaagtcttgaaaatttctctgacccgcagcaaacgttggaggtcttgtgtagcatcgtgagtgagttactgggtgaggtgttgattgagaatgcggtttacaaaccgacgcaagagattggtttaacatcgattctcggtgttgagctaagtcagagaatccgtcagcggtttgaagtgccatgccgtccaactgtcatttacgattatccgaacctctacgaaatgtcagtcgatatctctgagcaatggtacaagctatatgctcagacgatgaaacaggaagcaaatgctcctaccgttgaacaaacgcattctacccatacaaacgcacagggaaatgaggctatcgctattgtggcaatggcttgtcagttacccagtgctgataacccagaatccttatggacgatgatggagcaagcgctgaatgatggcaaagatgtgattaatcgcacgcctgttcatcgttttgatctgtcccgtttttgttcgatgaatgatgaatcaggtaaggcatttaaccttgctgggggatatttggacgatatttcaggttttgaccatgcatgttttcaattgtctcataatgaagcgaagttcatggatccgcagcaacgactagcgattgaaacaacgtggcgggcctttgaagatgcaggtatagaaccgcatcatttgatcgatggcacatcgagtgagatggccgatacgggggtgttttttgggattggtcagaacgaatatggtccactgtgtcgatcagatataagtagtgagtacgcagggctgatgcctacggggcaatcgatgaacttgattgcagggagaatctctcatctgtttggactcaaggggccggcgattgcatatgatactgcctgttcatcttctcttgttgcgttagatgctgccgtacgtcaccttcgtcgtgaatcagggaaaatcgccattgttggtggtgtgaacacattaattgcaccagaaagttttgtgttgttgtctaaggctggtgctttgtcgaagagaggacgaggatctgcatttgatgcatcggcagatggttatgtcagagccgaaggttgtgttgtgatggtgctcaaacgtttgtctgatgctcaagcggccggagacagaattcatgcggtgattcacggcagtgcggtcaatcatgatgggcggagtagtggactgaccgctcccaacgggagggctcaagagcaggtgattcgtgccgcattaaaagatgctgaggtttcacccgaacaggtgactttggttgaagctcatgggacaggaacgccacttggcgacccgattgaatatcatgccttacgtgctgtctatacggatggaaagtcgaggtctgcgcctctgaaattagggaccatcaagtcattcatcggtcatacagagtctgcttctggtttagcgggattattaaaactggtattgaccctgcaaaagcgaacaatgcctggccagttacattttaatacgatcaatccttatattgatgcgacgcaggggatcgtcattcctcgtcaatctcaggtactagaggatacggggcgattattgggtgcggtcagttcttttgggtttaacgggaccaatgcacatgtaattgtcgagcgtggggaaggggggcattcccgcgccttgccagcccaacctttccatcgtgttcgctgttggtatagtgaccgcccactcaatgaaagtgtcggacttgcacaagcattttctcagcgccaggaacaagcaacccaatggtgtgttcctaccaagtattatgttaagcggtggaaagatgccgaggttgagtgccaccaagcagcgtcggaactgtcaacactttcccatgtgctacttgtctctcagaagggtgccgagcggttgtgtcagcaatggtatcaaacattggttgcacaaggtttgactattacggaagcaagtttcgaacaagttccttctttacaggggcgatttgatagaaccgtcgtttgtttgaatgatggagcagagtcgacagaagatatgtctaccgcttcttggcagaactcggccgcgtctgtttggcaagtcttacaagatcttgtgacagcacgttttgaaagtggccatctgcttgtgtgtgaatccagtcggttgaatcgtgcctccaccgggggcaattcctgctggcaggcagttttatcctgctatcgtaaagagcgagctgatttttcagcgacactggtttcatgcaatgctgaattgtctgactgcttaccacagtctatgacatggttgctcacgacaaaagaaacggagtgttacctctcagcaaatcaagcttatatcccacgactgacagagcttcctccgttttcagcgaattacatgccaattctgtcggcacaacatagttacattgtgacagggggaacggggggagcgggcgcaagtctcatccaatggctgttacgttgtggagcacgacatatcattaatctgaatcgacgtcctcctgatgaggcgcaaacagtattgttgcagcaatggtgtcgcgatttcaatgcaagcattcaaacggtcaggggggatgtctccaattttgctaccttacaaggtgtgctggagaacacactcgcaaaagcgcctccattgaatggtattttccattgtgcgggtgtgctggagcaaaatttgtttgaacagcatacctggtcacaagttgaagaaatgttgcatgcgaagcatcaagggactctcaatcttcatagagcgaccctcgatcaaccattacagtattttgtcgtgttctcttcattggtgtcattgctaggtcagagtggtcagtcaggttatgccttttcgaatgcgatggcagagaaggtcatcctacatcgccgtactcagaatttgcccggactggccattcaatgggggccttgggctggtggtggcatggccagtcgggcgggaaaaggattagcagcacactatcgacaaatgggtgtctcgatgttggaggaggagagctacctccacgcgctagggggggtattggcgagtgctaccgaacctgagtctttgccaccggttattgcgattggtgatattgactggcagcgttatttatcgaccacaatggcaccgctgccattgtgcgaagacttacattcagtcgagaccggactgttgcatgatcaaaccgaaaatgatgaaccggatgaaagtttatcatcttctctatcgaaggttgctcctgaacgccgcatgcgtttgctgaaagagcgccttaagcagattgtaagtgagtgtcttgaccagaatgcccctcaattaattgaggattttgaaggctttagtgcgctcgggatcgactcacttcattctatgattctccataaaaaactagaaaaagaactgggttgtccgttgccccaaaccatcgccttcgattatccgactattactgcactatcaagtttccttgccaaacatcaactgggttcgttgtttgtccatacaccttcaatggcgcaagaagagcatgagactgatgagatggtgaattatagcgaagatgagctggcacggattttacacgaagagattgaacgtctggaggaaacctcgtgacggcaaacaataaagatgtgctgctgcaatcgattaaaacgattaaagcgctcaaaaatcgacttgcacaatatgaaaaaaatcaccaaacagatattgcaatcattggtattggctgccgatttcccggaggggtccgtgatctctcgtcattctggaatctgttagatgccggtaaaagtggtgtcattgaagttggcaacgaccgatggagtaatcagcagtttgttgattcggattatgatgcggtcggtaaattagtctctccctatgccgggttactggaacgcatttatgactttgatgctgaattctttggtttgtcagcggttgaggcagaaaaccttgacccgcagcaacggctgttgctggttcaaagctgggaagcattagaagatgccggttatgatatacatgcattgcgggggagtgatacgggagtttttgtcggtattggtagccaagattatggcatggcactatcggctgatgttcaacatgcgaatgcttatattgcatcgggcaattcgccaagtatggctgccggacgtttatcatatttctatgactttactgggccaacgatgtcaatcgatacggcttgctcttcttctcttgttgctgtgaatgaagcttgtcggcgcttacaggatcatcattgtcaattggcgctggctgcgggcgtgaatgctgttctgaccccacattccggggtgaatttttcacgggcacgaatgttgacttcagaacgcgattgccatgtatttgatgcgcgtgcaaagggttatgttcgaggtgaaggctgcggtgttgtcgtactcaaacgattgtcagatgctttacaagatggagatcgtatccatgcggtgatcaaaagtgtcgcgatcaaccatgatggtcatagtagtggattgactgttccgaatggttcggctcaggaagcggtgattcaatcagcacttgctcaggccaatctcagagcagaggacatccaatatgttgaagctcacggcacaggaaccagtttgggtgacccgatagaggcacgtgcattggcatccgttttttctttaggacatgacgcgcagttgccaattcatgtcgggagtgtcaaagctaacttagggcatttggaagccgcctcgggcattgccagtctgattaaagccgctcttatcgtcagtcgttcagaagcccccccacaacgcggattcgagcaaattaacccgaagatcgactgggatagtgatgtttttcgtattcctctgacaaccgaaaaactgagctgccctgatggggcaagcgtgagtgctggtattagtaactttggttttagtggaaccaatgttcatgccattgtgtctgctgtatcggtcaataaagaggtacatgcggacaagacggatacatcgatgacggggccagtggtacttgccttgagtgcgaaaacatctggagcgctacatcgtcatgtcgaagatatgattcgctaccttgggagccagtcgacacaaaccttacccactgtgagttacacctcgacttgtcgacgggcagctttgtctgaacgaattgctgtttgtggggctgaccccgatgagttgatcaaagcattacaagaagcgatgcgaggaagagtgtcttctatgcaacgcaatcccatcgtgctggttttgtcaggagaggatgatccagaccaattacgtgaactcatttcaacagagaagcatgaagacatcgtgacaggctcttggtctcaggaaactgagcatgttcgattgcatcgtggtttactcgagcaactcgcacattatggtgtttaccctgatcagattttggcggaaggcattgaacctgcgtatattcatgcctggttgacagggacagaagcgcctacacaacaaccttatttgatacggtcgtcagcagcacctgatgtttggctgattgggccgaatgcgactctacaactggagcaaccagaagatatggctgcggcaatgtcgtcatctcttgtcttagggcaaagatgccctgattttctggacaaggataagtgtgagtttcaggtattggcgggtcagaaaacattatctcctgcactggctaaactattttccgcaggtgtggcgattcagtggtcatcttgttattcccaaaagtatgctgtgattgacgattttccgaagcgtcaatttgactataaaacattcaagtcccctcgggttgattgcatactacacaaaaaaaatatgctcaggtctgatattcatccaatggtcacgcataaactcagtcaaccgaatgagatgttgacctatgaattggatgcagagacaccatggctcgatttcattgaacagcatcgtgttcaggggcatcgaattttacctgcctcattattaatcgaactcatgcatcatttagcggcagatgcgctggatgttgctcagttacagttgtcagcaatcgagtttttccatcccgttgatcttgatttagctgataggtcataccttgctcaggttcaacgacacccctctgatgggggaacaccacaagtcgatatcgttttgtggagtcgtggttcgacagttcgagataaagattggattcgtcatgcaagtgcaaaatgtacccctcgaacagaatcagttgtgtccataattgacctagagtccttccacgttgctgatgagtatcgctgggatattctagatgttgatcaattgtatgcgcagcatcatgaagccggggtgatattaggggaacaattccggggcgtaaaacagttgaagtcaaataccaatgtgctggaaggtcaagttgtccttgaaaaagcagatctggccgaatcgacgacattatttaccctgttacttgatagttgtttccagattagcggtggccagccagaaacgaactctgacgttcatttgcttgcgtctctgggaaatgcagtatttgaaacggatttaccatcgagcttaattgtgcggatgttatcgaaaccaagtaatgatgggcgttgttttgatgtattgattacccagcaaaatgggcaagttgtgggggctttttctgatgtcttctttaaacgattgccgaaagcttcaacgcattcaacagcagttcagtcaacctctgaatggttgtctcctgctcaatgtttttattctcagacctggcaacgatcatcttggaaaacagaggggggggctgaacccactagctttttatctccttctgctttatttcagaatgttgaaacagctgatttttgggcagagcgatttggattggaaacttataatgcttatcggcaaggtgttgagcagttctgtttaaaagctgttgtcaaaacattccgagaattgggatatgttgacaacatttctcttccgaaagcattggcccaatgccggattcatgagtctcagcagaaactgttttctcacctttggcacttgttgaaaaagacaggccaagaggcctcggttgctgatgagctagcccatatagattggacacaactttccgtatcttatcccgagtacgactcggaaaccatattcatgcaacactgtgtctctgctttaagaggtgtactgctgggtgaacaagacccattggatatcctattccgggatacatcaatggttggtagtgatgctgtttatttgaattcccccatttctagggcgctcaatgcgcagcttgggcagatggtagcggccttgagtgcaaagcgtccgttaaggattttagaaattggagcaggaactggaggaacaacgaaaagtgtacttgaggctttgcatgacctccctgttgagacttactgctttacggatatctcatcattatttcttaaaagggcggaggagatattccagaaccattccttcatggaattccaaattttagatatcgaacaaccgattgccgagcaattatttgaagctgggacttatgatctcatcattgcagcaaatgtcctgcatgcaacacgctctatctctcagacactgggacacgttcgagagttgctcgcccctgatggatacttgttgttacgggaatgtatttcacaacagttggctgccgatttgagttttgggatgacagaaggttggtggcgttttgaagacacgatgctacgccctgattatgcggtgatgagtgctgaacaatgggagcagcaacttttagaacacgatttttcttacgctcagtctatcctgcctcatccaatgagcgctgaagcgctcattgtggctcaagctgaaaattgttccgaacaggaacaatggcttttaattcaagatggtcaggcgcaaatctgggtcgaagagcttaagcaacaaggcgctttgtgtgaatgtctttcttggaaagatatcatgagtggtgtcaagttacccgcacaatactcgccagattaccttgtttgttttcctccggatactgaagacagtccagacatcgttgaagcgagtatacagtgtgctgaatcaacgatgtcactgtgccgtcatttatttgaagacaaacgtttttatcaaacccgtgtctggggggtgacgacacaagccgaaaaagtgttgccgtcggatccactcacaggacttggccaatcgaccatgacgggagtgatgaaaagtgcagcattagaatacccaggtcggattggcggtgtgattgattgtgaaaaactgtgcggtgatcaagacagagtatcgatgattcaacaaatgttgtcccatattcggcagccaggacatttacgctatatcgcgatcagagctaatcaaccctatcagccattattgttgcctgacatagccaaagaagaaggggctatgtctaattcgatagttcaaagcctctgtgctggaggtgctgtgctcatcacgggtggttttggtggcattggaactgcgttgacgaatgctttaggggcatttatgaagtcaaatggaaattatcctacaggacaaaatacaaaaggtactttggtcttggttagccgtcatatcgaaggcagctctcaacaaactcaggtacatgctttggaaaacatgggttttagagtgctagcgattgaagcggatttgagtattcccgctcaagtcgatgatgtcttcgaacagatagaagcccaatctctccatcttgaacatttaattcataccgcgggtatcggtggtgatcagatgattcgtgaaagtcagtcgggtgatttaagagaggtggtgttatccaagctagcaagcacttggtatctgcatcaacgcgcaccaagggatcttaaaacattcttagttctctcttcgatggttgggctatggggggctaaagggaaagcacactacgtgtttgcgaatcattttgctgatcgtgtaatacagcttcgtcgttcgctagggcttgctggctctgttgtccagttgggacctgtggatgccggaatgttaaatcttgcgggtaaggaagcggcactgcgggttggtgttcgaagtttcaaagtgcaggacatcgcaaagatgctcattggtgaattgcgagctgctgagtgtgcgattttagacattgattggcgcatctttaaacctttctatcgaggaagttggttgcagtcctattttgagaaacttggtttgagaagttatcgcagtgatgcctctgttatgactcatgctgatgaatattcattctcaaatcgttttatggcacaaccagtcgcgaagcgtcgaaaatttgttgaggcagaattgttcagtattttgcgagatgtgttgggattaagcggtgacatcaaaagttatctggaaacagggtttcatgaactcgggatggattcgttattaacgatgtcttttgcagaaaaggttgcaggcatgtctggaaaagaagtgtcgtctattgatgttttggacaatgcaaatcttcaacgcttgagtcagtggttatttgaccaattcaaacatcaactagagaagaccgatgtgacacagaaaaatggacagcatatttcttctgtgttcaacgcgtctcaaccgcaggactccgttacatctggagtcactttggaacagatagagcatgaattaaaagccatgcaagaaacattggaggatatctgatggaccagacgcatgaattactcttagccagacagaatgcattgataaaacagagtcgtgagaagatccgtgctttgtcagcacagattaatcaaccgattgcgattgtagggatggggtgtcgtgttcccagtgcagataatgttgaagcattatgggaattgcttcaacaaggtgacgaggctatatcggaggtgccattgacgcgttgggacatcgatgattattacgatccgaaccctgggacaccagaaaaaacctatagccgtcgtgctggttatctgtcagatattgagcaatttgacgcgagattttttggtatttctccgcgagaagcccgtcaaatggatccacaacagcgtttattaatggaagtcagttatcatgcacttgagaatgcaatgatatctgctgagcgtttacggaatgaagccgtcggtgtttttgttgggattagctcgagtgaatacggtgcaatgaccttttcaggggatcgtgatgcgagccctgatgcctactcgattacaggtacatcgttgaactcagcggcagggcggctcgcttactactacggttttaatggtccggccttagcaattgatacggcgtgctcttcctctttggttgcaatttatcaggcttgtcgaagtctcgtgaatcaggaatgccatacagctttggctggcggggtgaatactttattgagtcccggaccaacggttgcattagcgcaaaataaagtactgagtgccagtggtcattgcagtccatttagtcatgatgctgatggattggtgcgaggtgaaggatgtggcgtgcttgtgctcaagagacttgaagatgccgtggcagagaaatgccaaatactagctgtcattcgttctgggcatgtcaaccaagatggtgccagtagtggcttaacggttccgaatggttatgcacaacaatctttaatcaagacggcattgaaggttgcccaagtggacccggcggcaattcagtatgttgaaacacatggtactgggacagagctaggggatccgattgaagcgaaggcactacgacaggcactgtgtcaagagcaggtaagacaatcgccacttttgattggctcaattaaagcgaatatcggtcaccttgaggccgcgagtggggtcgctggggtgataaaagtcgtattggccttgaaacatcacatgttgccaaaacaaatcaatgtggcacaaaaatccacattgatttattggggggatgatttagatattgtcaacaattcgaccccattgacctatgaccctgagcacccgttttatgcgggagtgagcagttttggttttagtggcaccaatgcccaccttattcttcaggacccggtcagtgctgttgttgatgtggaggctccgcaaacaatctctcctgttcttgaaaagtcgacctcagaatctcatgcttttcttggtatttcggctaaagatccacaagccttaaaattattgctgcaagactacagtgagtattttgcctcagaaagagactggcaaggggcttgtgctgctgttaacgcagggcgcagtcaatatccgtatcgtgccggttttgttgcgacagatcgtcaaacgctggtatctcaactgaaagctgccagccataatgaggatgtaagggaaccctcactgccaccgcttgttacatggctttttacggggcagggctcgcagtatgttcagatgggaaaggttctgtttgaaacccagccggattttcgacggtggttagaacacgctgacgcggctattcaacctcaccttggtcgttcaattctatctgtaatctggcatgaagctgctcaactcaatgacactcagtatactcagccggccatattctgtttgcagtatgctcttgctcaatttttgtcgacacgtgttgggatgccccaatatgtgttgggacacagtatcggcgaatatgccgcagcggttgtcgctggtatcttcgatttggatgatgctgctcggatgatcgttgctagaggacgattaatggttgaacgttgttcccccgggggaatgatggtcgtttttgcggatcgccaacaagtagaggaattgttaacgcatagccgtcaagatgtgagtttagccgttgtaaatggtcccaaaaaccacgtgctatctggttcaggggaggccattcagcatttggctcagattgctgggaaacgcgacattcggtgtgtttcattgccggtttctcatgcttttcattcatcgatgatgacccctatgttgcgtgaattcgaagccgttgtgcaacagactaattttaatcgtccgcagattagctttatttctactgcgttgggacgttccgtcgatgaagaattattagattcatcctattgggtctctcaggtcagcggggctgttctgtttgaacaggccattgcggccctgacggcagaaccggattggcaggatgcctcttctcgtttgtgtgtagagttagggccacatagacaactgattgatatggcaagaatgttacctgatacgtcagatgctcaatggttgaatatgctccaaagtgggaataaccatcatgtctgctatgaaacgatgcgttctctataccttgctggcctaccgtttcaatggccagaaatttccgcctataagagttcacagcctttgccgcagtatccgtttattcggcaaccatattggttagagggagaacgacgcagaaacgttgcttcagcaaacttagactacgatgtggtatggaatcatgtgttgttcagtcaggcaggagacacatatttaaaatcagagaatgagcagaataatttttgtttagtatgcacggaacaagagtgtcaattggctgcagctttatcgaattcaatggctcagattgggacagaatgtcaaattgtggcaccgaaagctttgattcaagacgggagtgcccttgatcattgtcattcagtgattttatatatgccagacggacaagctccgcctgaatcgcaaacatgttttgaaacattgtcgttgattgtacaaatgactcaagaccgagtggctcgtactcccaggttagtcattgtggttgatagtggcgaacaaggttataggccattggcttctggtcttgccggactttgccgcaccgtgcgtgaagaaacatcattcgatctgacgttgattggtttggatgatcgcttagacgtgacacgccgggctcagatgttagctgccgaaattcatacgcctcgttgtggcgattgggagagacgccttgtggatgatcaggttctggttccccggttggtctctgcatctcaaaccgatacgcatcagcgatcccgggtgaatgttcacccagaacggagctatctcgtgaccgggggaacggggactttaggacgcttatttgcggagatgcttctcgagcaaggtgcgaaagatctaatcctcttggttcgccagacagagcatcatgtactgaatgaattaagaacgaaatcgacgtctttgggggctcgtcttagtgtctatggtgttgatttgactgatggtgatgcaattcagaacgtttttgaagtgttgaagaatactcatctgcctttggccggcatcattcatgctgctgggcaattgtcggatgccgctttcaatcaattgaatgcagaatcgtttgcgcgtgcatttgatgttaaggctgtcggtgggtggtggcttgaccgactgagtgaagatatgccgctggacttttttgtcatgacatcctcgattgcatcggtgttgggggcggttggacaagccaattacgcatccgcgaatggttatcttgatggtctcgcattacaacgatatgagcaaggtaagcctgtcatgagcatccgtctgggggccgttgctgacactggtatgacagcacatgacgatgtgaaacgccagctccgtcgtatggggatcaagccgttgcctttgtctgtcattcgacaccggtgtgcagcatggttgggggcgtcatcaccattgtcgatgttagcagagattgattggcccagagttcgagcccgggaaggggagactgcacgcccattcttgacggagatgttgccgcccataaatggcgcaataacccagtcaatgtcctcatctctatccggaaataaggggaaatgccaacgcgaacagattgaaactatcgtgatcgatacgattgctgcgattctcggtatatctgagtctgaccagatcggaaaaatggacacgctcaatgcgctgggtatggactcagtcactttggttgaattacgtgaacaattgcaattgcaacttggggcggagattccgacaaggttcttgtttgactttccacaagtgggcaagcttattcggcatttacagattgaactccatgatcatgacaatcaacagcgtgcgacagaggggtatgatagtacgtctgagacgctgcccgcagagtctgatgagcaagcaagtcgtcgggatattgcgattgttggtatcgcttgtcgtttccctggtagtgtgaactcccctgaagcgttttggtcagcgttgaaggaagggagagatttagtcggggagattgatgcacttcgttgggatgttcgatcattgatgcgtcaagggcagctagtcacggcacgtgctggtattatcgatcacttggatgctttcgactgtggtttgtttggtattacgccgcgtgaagcgcagtgtatggatccacaacaacgattattgttggaggccgggtgggaagccctggagcgcgcggggtacgatttttcagccaccgatgtgctgggggggatattcattggtcctggtcctgatgattatgcgaagcgatttccgacggatgcagcgaccttatctcatcaccatagcacaggaaatgcattaagtgttactgctgggcgtttagccttcactctggattggcaagggccaacattggcggtggatacagcatgttcttcatcactaatggctgtacatctcgcaactcaggctttacgtcaaggtgaatgcagtattgcgcttgcggggggcgtgaatttgctgttgtcaccagaaacatctgttttactttctaaagggagtatgttagcgcctgatgggcgttgtaaaacatttgatgccaaggcgaatggatatgtccgcagtgaagggtgtggtcttgttgttcttaaatgcttaaaagatgccgttgctgatggggatgatgtcatcgcagtgattcgaggatctgcttccaatcaggatggtcacagtcaaggacttaccgcacccaatggacagtcgcaacaacgtgttttgcgtcgagctttacgggatgctgcggttgaccccacgcaggttgaactgttggaagcgcatgggacgggaacaccgttgggtgatcccattgaaatgtcagcagcgcaagctgtttatgcagagaggctgtctcgtcattccccgttatgggtatcgtctgtcaaaacaaatatgggacatgcggaagcagcggcaggcattgccggacttataaaaacagcgctttgtttgtaccatcagacgattgtgccccatctacatttggcgcaattgaatccagaaattcagctcgacgacacggttcgtatcccagacacaacacagccatggccacatgatggtactgagcgttatgcagccgtgagttcatttggtttcagtgggacaaatgtccacatgatattgcaatctgcacctgaaagagcctctatatctacaaagagggatacaccagatggtttacgaatctcagccgcgacaaaagttgcactcaacgacatgatgcatgcttatcttgatagattggttgtgctacctgcaaaacaatatgaagatttttgccagcagagttggcgtcgtgctgagttggcgcatacaaggatatttgcagctgcgccgcagggggagatgattgctgaacttcgctcaatattggaaagttcacacacggtaacaagtgtgggacaccaaacgtcggctctgtcgatgctgggcggatctttaccgtgttatccattccagcggcaacatttctggcaaccactctcagctgttcgaaaagaacgctccaacgcagttaatctgggcgtgagattgggccccagaagcgcaacgaaggtggtttatgccgttgattacctgaatcagccccctttcaatctcgaagaccatttggtccataacgaatttgtggtaccagccgctgcacatttagccctgattattggcatgctcagggatttgcgcgggagttgttgctgggaagttttagatgttttatgtgagggaacattcatccctgatgcagagacagaagtggccttgtatgagtttacgacgatcactgaagatgaaggcgaagcgttcagcattgaagttctaagtgaacactctgaggggatccggacacacttgcgagctagagcaaaatcgacggagactcaagctgcttctatcgtgattccagtgtcgaatccaccattaactgaaatcaatgcagccactttttatgatgaactttattcttcagaaattgccttgtctcatgaatttcgcagtattgtgtcgatccaacagtttgttggctactcgaagtcagaaatgcgttggtatgcaggtgattcagggccattagtgccgggagagctcgattcactattacaaacgatcgctttggcgacaaaggctgaacagccaggcactagtcatatgagcggggcgacaattcccgttactattgatagattacttgtggcatctctgggagatagagaggcggtcccaaacttgccatttatctgtcagacccgtctcaataaagaagatcgtggcgctgcttttgtccatgacatcgctttgtcggtcgaaggggaacaaccatttctgagtattgagggattatgtactcggtgggtgagtgcgcagcagatcaaaccgatcagagcttcttctgcttatctgaatgaagcgtgggtggctgaacctttggcgacgccattgacaccatcggaagagggggcgccaatttatgttttgttaaacacggtcgaagatgaaattgaccctgatttcgcatctgctttagcaccgctgaacatactgcatcaccctacgactcaggtggttgaacaaacgtgtcgtgaacgacatgaaagtcatgatgttgttttgatttactgtttatctcaatatgatccgcatggcagcgtagcaaacagcgacgggatgcaagggatgatggcgcttgtctctgccgtcagatacgctcataaccttagtgaaaccttgtttgactctggtatagcaacacgttttcaggtgctttcaaaagaacatgcccatccaattggcaaagtaagtggttctcctttgtttgggtttgcatcgggactttgtaaatcactgagccttgaatggtcaaattgctctgtcggaatgctcgatgtggatacgttatcattgcagcaaaacgcaacccttttggaaaatgaatggtgtcaaatgccattgagttgggtctgctatcatcaagaacaccgctatgttcgacggattggggaatttgctggcgaggagattgttgggaagaatgcttttcattgtcgtgacacggggctttacctgttgacaggaggattgggggcactcgctttggagacatgcaaatggcttctaagtcatggcgcaagacgcttcctcttgctaggacgacgtgctgaaaatgagcaaatcagtcaacgattgcaagaacttcatgacacatttaatgctcagttcgagtatagagcctgtgatgtctctgacaggacggctttagagacgttgcttgctgaggaacagatttctgactcgctgtgtggcattttccattgtgctgggacgctgtctgatggggctttctcttccttaactgatgatgacttcgctcatgttatgcgtagcaaggtcattggaagctggaatttacatacattaactcaggacatgtcgcttgattgttttgttctgtattcatcattggcttcgttactgggggcagcaggacaaagtaattatgccgctgcaaacggttttatggatcagcttgcgcattatcgacatgctttaggcttaccagcactggcgctcaattggtctggctgggacgaaatcggtatggcagcaggaaagacatcagagactgggctgcgtttattgaatccagcgagagcgattatggatttggaacgcttgatgtcatctgatgtgattcaagcgggggtcattgatgtggattggtccatctttagcgcgcttgggcatcgaccgatacccgggtttgttcaacattggatagaaagtcatcaggttgaccaaacggataccgacaatgagaaagaagacaatagcgttaatctgttggaagaacttcgagcgcttccatcaatggctcgacttgattctgttaaaagctatttacgtcaaattacaagaaagatcatgaagttggatgaagagagtgaactggatgaccaaaagccatttcaagactttggatttgattctttgatgtccattgaactgaagcaaaagctatataaaaagttgcgtataagagtacctgcaactctgatatttgactatcccaatgtagaaagtctagcaacctatattatgcaaacactagaaccggactgtgagtcaacagagaagccattgccggatacaaataagcatatggatgagctggatgaagagcaactcgcacacatactggaaacactgctgtaaaggagtattcacttgaatatgaatcaaacacagaaaagaagtgaaaattcttacaaggatttattgaaaaattccatcctgaagattcaggagaaagatcagcgaatacaagcgcttgaaggtgagcgagataagcctattgcggtgattggtttatcttgccgtttccctggtgcttcgaatccggaatcgttctggcaattgatccgaaatggagagaatgcggtcaccacgatgacagaccagcggtgggatatggctgaatacttttccccacttgtgggggaacccggaaaaataagcacatgtcattttgggttgttgaaagatattgatcaatttgacccttctgcttttggtatctcagaggaagaaacaccttatctggatccgcagcatcggttgctacttgaacaggcatggcattgttttgaacgcgcaggtttcgatatcatggcactgagaggttcagatacgggtgtttttgttggtcaaatgaacagtgattatacaaatttgatcaaaagtactgaggatttgaatgcctatgtaggacaaggtaacgcgttgagcgctgcttcaggtcggttgtcctatgtttttgggctgaatggtccgagtgtatctgtcgatacggcatgttcttcatcactggttgctgtgcatcttgcttgtcaaagtttacggatgggagagtgctctacggctttagccggaggggtaaatttattattaagccccgaagctgcgattggtgcttctgttgctcacatgctttcagtgcgtgggcgctgcaatacatttggtgacggggcggatggttatgttcgctctgaagggtgtggcctcgtcttactcaaaaccttggagcaagcggaaaaagacggtgacaagatactggcggtaattcgaggttcagcagtgaatcaggatggacgcagtcatggattgaatgctcccaatggacccgcccagatggacgttatccgtcaggctttgtctcaggcaaagctggatcctgccgaggtaaattatctcgaagcgcatgggactggcaccgccctcggagaccctgttgaggttcaggctgtggatcaggtctatggacaagcaaaagggcgtaccaaaccgttagtgatgggggcggtgaaagccaatatcggtcattgtgaatcggctgcaggggttgctgggttgattaagttaatccagatggtccaaggcgatgtactgccgccgattacgcatttggagcaactcaacccgcactttgatggtttgagcgagtcgcttgtttttccgaagggcgcgtcgaaagatgggacgtcttcgttcacatggcatgatgagcagtctcgtatcgctgctgtgagctcgtttggttataccggtacgaatgttcatatgatagtctctcagcactcgactgagaaagatgcgctgaacgttcaagcccatgctgaatcaagtaagacacagtcccaagctgtttatccattctatttttctgcgcatagcattacgtcattgcgccaacaactctctcaattccgtgattttttgaaagaaacgcctcacctttcactgcctcttatttccggagcggtgaatagtattcgtaatcattttgcttatcgttttggttgtcatgcggcaacgaggcaagaacttttatcttgtcttgtgtcagggattcaaggtgaatactcagagccgcttggaaaaccgactatggtcgcctgttttggtccttccaatagtcaatatcccataccacctcagatctttgcctctgacacatggtgctcgtattttcggtgtattgaaacccgagtcacacaagcgactgaattcagtatcgaagcgttgatgtcgtctcaggcatatcccaccctgcgccacttgatttcacaactttcttatgtcgagcacctcgtggaagaaggggccgagataagaaaggttcaggcatctggtacaggcacgattgctgccctcgtttttgctaaatctttgacacttgagcaagcagcagatttatgtgtgcgcttggatcaaatctcaggagctgatttaacagagcaagagcaacgagttgaagcggatatccgatatctgattcaggacatacacctaaaagaagcgcagcgtcagatctttgtcaaaggacatgactcgacgcgtcaggatgtcgattgggtaaaatggccttatcacaatgtctcagaagcaaagcaaagactgcttgaggcgttgctctcacaacagcatccgtatcactggtcaatgctagactcagagattggcattgatgtttcagctttgcagatttttcctcagaagactatcgaatccgagactcagccctcatggattaatttacgcatcaatttgtttaatcgtggggcgacgctgcgatgggactatacgacgataccgcaggataaatctcaatatcccgtcttgccggattatgtctttgcacgacgtcgctattggttacccccacaagtgacagagcgagtggcgtcactcccatcaatgttctcaactgtggggcacagtatctttacaacagttttagccgtgcctgatggtggaagcttatatgcgggtgaacttgccttatcacggttaccatttttaagagatcacctcgttgcaggagagattgttctacctgcaagtgtttatataaacttaattgcagaggtgtgtgtcggtcccgatggactgcctgcgaacattgttcaaatacagatcagccaagcgtgtattttggattctcaacccattggtgtgtattgtcgagtcgacccgactgagaatggcatagcgaaggttgaaatttatacaacatcagagcgtgggaaatggacgaaacatgtcagtgcgcaagtaaatgttggttttcgtgccgagttaaatatcaagccgcttgctgatgttcggaaacggtgctctcaggtcatctctgttgaacaacacttactcagagccaaacagtcgggtattgagtatggcccttcttttcaggccattcgtcagttatatagcggttcaggtgttgcattggccaaaatcgaatggccatctgatttgccgaattactggtcaggttgtggcttacatccggtgatgttggatgcttgctttcaggtcatcagtggggcaatggggacgcaaaattcagacagaaaactacctttgtatgtgccgacagagcttcatgaaatgcatcttcagggaaatagtgctgagagcttatggtgttttgtacaaattcttgcccctgacgaagtgtttcacaatgaagcaacgatgtatgactacttgagccaacaagaaaagctttccgttgctttatatgtatatgatgaacagggaaaagcgattctctcaattaagcgttttgaggcatcgaagtatcggccggagcttaaaggggcattatggcaagaatggttgctagaaaagcaatggataccctgtcggttttcttccgagaaattagatgtcgatgtgtctgcattgctgacgcaggcacaatctcggtttagtactgtaccttatacgatgtctgtggacatgatgcaggatctggatgcgctttctggacatttcattcatcgagcattgacgcagtttggcttcgatgagcagttttttacatcattacttactgctcatacgctgcccgatgagatgctcgaacaatacgggattctgccagattatcagcgattactaaaaaaattagtttccctgagtgagcacctccctgtttcagcactgcctgctgatgaaatcgaatcaaaactgagatctaagctaggggaagaaacgcatgagttggacttattcgttcgctgtggtaacgcactagctgaggtactgcaagggcaaacgaaagcggttgatctgctattcgaggcgggccattcacaagggaccgaggcgctttatcaagatagccctgggagtttgttgttaaatcaacggatagcaacactggttgctgaagctgtaagtcaaataccttcaggcagacgattaaggattttagaagttggtgcagggactggtgctacaacgcaacaagttttggagcaagtcgatggtaaaaacatcgattatgtctttaccgacatttcgccccattttttgatgcgtgcaaaagaaaaatttggtgcattcgagtcggtagactatcggatatttgatttgcagcaaaatccactagaacaagaattcactgctggggcatttgatgttgtgattgcagtaaatgtgctgcatgcaaccgctgatctgactcagaccctgaatcatttatctctctgtctagccgatggcggcctcttgttattacgtgaggtaacagagcagcaagcctggcttgacctcagctttgggatgacacctggctggtggagttatacggacacatcgttacgtcaaaatggaccattattaaataccgaagagtggcagactttgttacatgattcggggtttgagtcaacgcttgtgacggatgagctagagcgaaccgaaagtatatttattgcagagaaaattgcgagcaccactgatgtggtatctaagggatgctacttagttcttggtgatgaagacaccatcgaaacccaagggctaaacgccctgaaaaatgagttggcgcaacgtggattggattgggtgtttatttccctgaatgaattcatgcagcatcctcgctttcaagaattagatgcattttcaacattactgatgtcggtggaagcgcagcaaggcccgattatcggtttagtctatgcgtggtctatgagtccgtcagacctaaactgtgacgacctattggagaagtctgagcgttatctgaaatatccattactgctttgtcaaacgctgttggatgcgcgctggcgacacctttctcctagctttttgaccgcaggtgcccagccacgggatcacaaagttgcacagcctttacaggcgatggtttggggacacgtgttcacttatatcaacgaaaatgcagcatttgcacggcttattgatttagatggtacttctgtttcaggtgaatgcttgtatgaagcgttgacacaaaaagaagaatgtcagttggttgttcgagaggggcagttgtgtgctgcacgtcttaaacgggcaacgttatcggttccggaagcgccagcacagcggtgtaacattgtggctgatgggacctatgtgatcaccggtggatttggcgatttaggactgcaaacggcacaaaaacttgttgaacaaggggcccaaaacttgattctcattggccgtcgtgagcgtcgagatgtcatgaaaacattggatatgctccgtgaacaaggtgtgaatgtcgttcctctttatgttgatgtcagtgatggtgaggcactcaatactgcactgaaccaagtgttagatgagttacccatgattcgaggggttgtccattcggtgggggtcttagatgatggcgtaatagaaaaacaacgctgggaaagatatctgaaggtactaaaacccaaggtctttggtgcaattcacctctatgaggcagtatctggttacgacctggatttttttgtgatctattcttcagcagcagcggtaatgggaaatccaggccaggctaatcatgccgctgccaatgcatttttagacgcattcgcttggtatctgcgtggacaaggctgccctgggcttgcgattggctggggggcttggtctgaaataggtgctgcggctgcaagagggattacagcgcgtttgactcaaaatcaatccattgcaggtaccatctctcctgagcagggtgctgatttgattgcaaaacagtttgccagtaaaaatgtgcagttcactgtattacctatagacttcgaccgaaagatagatacacagtatttaccacaggttcaacgtttgttatcgagcctactgaagagtaacaaatcgggagtccagcaagcaggaacagcacctgataatcgagaaaaggaacatgcagatcagtttgtgcaagacttgctacaaatggatgaagctcaacgacaacgtcaagtcgaaaattatctacaaggtaccttagtcaaattactcaagcaacatgggaacattgaacatcaaaccagtttatttgaattggggcttgattctctgcttgccattgacttgcgaagcttgttggaaaatcaattccggcagaaatttgagtcaaccctattgtatgactatcccaccattcattcattgactgagtttcttctccaatgtgtcagtgacaatcatgggcagcaaaaaactccgttcactcatcatgcagaaacgctttccagtgatcctgcaacgaaaactgctgatgtcgaactgcaaggagatgatgcgattgcggtggttgggatgtcctgccgcttccctgggggggcaaattcgattggagagtattgggaattactgaaaaatggtgtagatgcggttcagccgattcctgaaacacgttgggatcattcgcgatattatgacccggacaaaacgcaatcggggaaaatctatgttgcagaaggatgttttattgatgaagtcgctcagttttgtccagagagatttggtattgctggtattgaagcagaccttatggacccgcaacagcggctacttttagatgtttgttatgaagctttagagagtgcaggacagaacccaatgggattaggcggttctgaaacgggcgtcttcatgggggttatgacgcaggactacttacatttgacgcaacatgtgcgtgagaatgcgttttatgtcggaactggttctgcgaatagtgtggtcgcaggacgagtgtctcatgtcttcggtctgatggggccgacaatgactcttgatacagcctgttcatcttccttagtcggggttcagatggcatgcactaatttgcggtctggtgcatgtgatatggcgatagcaggtggtgtaagtttacagctttctccagaacctctggttatcgagtgtgcaggtggcatgctttcaccaagcgggcactgtcgcacatttgatgccagtgctgatggttttgttcgtggggaaggttgtggtgtagtggtcttaaaacgcttggcggatgctaagcgggatcgggacccaatcgtgggtgtgattcgtggcggtgcagtgaaccatgatgggcgtgccggcggtttgaccgtaccgagtgggttatcgcagcaaaaggtgttggaaaaagcgcttcatgatgcccagttatccccttctgatatctcctatattgaagcacatggcacgggaactcacctcggggaccctctggaactaaatgcattacaggcggttttcagtgaatcacgcgataaggcgccattatatgtcggttcggttaaaaccaatattggtcatgctgaggctgcagcgggtgttgccgggttgatcaaggttctactctgtttgaaacacgaaaccttcgtccctcatttgcattttgaacagcctaaccccaattttgattgggaacagagcaatatacaggtaacgacttctcttcatgagtggacgtcgaaaacaaagcgtcgagctggagtgagttcttttgggttaagtggaacaaatgcgcatcttgtagtcgaagagtttcaggaaatgatgctccctcgaaaacaaccctcagagtttgtacctttggtggcgctctctcacatcaacccgacacaactaaagcgtgatgcggcccgataccatcagttactgactgaggatacgccattgctaatggatgcagcttatactttctgtatatctcgtactgggcaaagtgtgcaggcagtctttccggcgcaaacacaagaagcattgctcgctgggttaaagagattagccgatggtgagctggatattgtagatagaaagattaaacggccacggttggagtggcagatatcggtgaatcaaagagctgcatggatgaatcaggcctgtacttactatgacttgtatcatctgtttcgagggacgattgatgccagtgttgaagcgctcaagcagcgaggtttcgatgtattgtcctcgatagaactatgtacattgaaagacggttctgtagacgaaaatatcgcgctacctgagcaagttcgtttggccatcttaaatgtagcgtatggacgtttgctcttgtcactaggcattgcacccgagtgtatccaagttgaagggttatccttgctatgtgttgcaaccctcgtcggtgtgtcggatatcgaacagatgatcgatggtatgcttgcggacagtgatactgagcttaaagctattttagccgggatccatttttctcatggtcatatgaggatgagctttaaagctgatcgtcgtttgtcctctgctgtcaatgaacgctgcgactctatttgtccaaaaacgtccgttgattatgtttggggggacagtgatatttctctggatcttgttcgaagtgctgtcaatgggaaagcgtttactgaactccaccagttactgagcatgtgctcttctctgggattggatattgactggtatacttatttttctcccgttgaaccgcatcggatagccttgccttctagtcacttcccaacgcgacggtactgggttaatgaatcactcgagcaagaacaaacacgccaagtgccactgatcagtcaggacatcactgcagctgtagatgggtgtcgatatgttgacttccacttatctgttgaacatcaggttttcctcgacgaacatcgtttgagtaatacacggatattgcctgccgctgggagtctggcttttatccttcatgctcatcaatgttcgtatcctcttgaaatgaaaggggttcgcttggttcgccccgttcattttgaatcacagttgtccgttcagcttgcgatgaagccgacaggacggacggagctattttattgtgaaccaaccatgaaagagtggcatgtctttgcaacgatggaagccattgacatgagtgaatcatcgactgagagagctctcatcacggatgataatcaacttttgtcgacattccacgctgtactgaatgcgcctaggtacaccatggataaagagaccttttatcaggcgtctatccctcgagaactgaatcttggagagtcatatcgactgattgaggaagtgcaaggtgatggtctgtgtttggttgctagaattcggaatatttcatctgaatttgtattagatccgcgtgttttggatgcctgtatacagtctgtcaacggggtgggggacaagttagagagacaatcagacggactctttttgccttatgcgatacaaagtatttctattcatgactggccgtcaggctcacatttttggtgcatgacgcagtttttacccgagaacagtcatgctgatgaattggtctacgatattaccgttgtggatgaacatgaacgtatttgtgcgcggtttgaacatgccagcttcagaaaagtcaatgtccctatggttgacagcgccaagccagagacatcgattatccatcaattgcgatggcaaccctcgcctttacgaaatacagagtccgtgaattcaatcgcaattgatgcagttgaaaacttacaaaatgtattactaattggtaaagcaaatgctgttcaacaacggttgcaagagcaattaagtgattgtgcttgtcaccaagtggatgccgcagaagctgcacggcaatcagtcgatgaacttcgccttctattggatgaatgttcaaaggctggcgcgttcatctatactgagttgcttgaagcatctgatcgatcaccacacgatgttgaaatattgactcaagatatttggtatttgcagcaatggttagttgctgcaagcaaacaaggtaagccatttatcgttctgacccgaaccggacagtctgtctgtctgaatgatgtgcccttatctgcggcaagtgtcgcggctgcggctctggtgcaaactgctgttcaagaatttccgtcattcccggttctactgatcgatttggatgacctttcgacagaaagcgaggatggtatcggatggttagcggaaatcacaccgtcggtacaagactctgtggtcgcttaccgtgagggaaaacgttaccgaagcgttttggaagccgtacattctatagaagacgtgtcttgtgggaattccgtctctcctgtcaaaccgaaccggacttatataattacaggtgggttaggtgatattggcctactcacggctgaaaagctagtctcggaaggggcgacctcattggtactcatgagtcggaacgagcataccaatacgaatgaacgtattgaagcgctgcgaaatgttggatgtcaggtcgagatccgactgggtgatgtgtccgatatgacggtcctgcaagcacatattgagcagatgacgtcttgcatgccccctgttgctggtattttccatacggccgctatttcagccaatgccctcatgacacaacagacggcggccgactgggcggatgtgttgtcggttaaagtacagggggctctgaatttacttgaaatgagtcgagagatggaactggactgttttgtcctgttctcttccattgcatctattgggggatcaatcggtcaatctaattacgcagttgcgaatgcctttctagattatttcgcacaatcttggtcgcagttaggtgttccggtgatgagtatcaactggggcgcctgggaaaatacggggatggctggacgcgctgaagcgaaaggtattggattcgaacagcgtttgagtccagagcaggctctttcatctctctgcttcctgatgcaaaacccaagacctcaggtggtatgttttagagaaaatagggctttgaatacatctggtttggagctgtcgggtggtacttctctcaatgaagagctcaaacatcgtttgtttggagaggcgttcttcaatttatcggcagatgaacaaaacaatactgtcagccagacgattcggagcatattgagcgattttcttaaggttgatcccgaccacattaaggatgagcagccattttttgattgtgggatggattcaattaccgcagtcgatttcagtcaccaagtcggtgaagtctttggggttgagcttcacgttgatattgttttcgactatcccaatttgctgagccttgcagattatgtgctgcaactgttgttacaggatgtgccttcattcgatcctgttatcagcggggaagacggagcactcgtggatactttgtcccgtttatctgttgatgagaagcaacatcatatcaaacggacgattcggaacattttgtgtgagtttctaaaggttgatggaagccatattaaggatgaacagccattttttgacttagggatggactcaattactgcggttgagttcagtcgtcatgtgggagaagcatttgcactcgaactacatgtcgacattattttcgattaccccaatttatcaacgctcactacacaggtaatggaactattgtcattcgatgtatcacctcagccaatctcagaggacgtgacaattgagcagctctctaaaatgctggaacaggaattagaaaatgacgcatagaaatttgcaggaaacgacaccgatgcaagatacaaaggaattgttcagtaagagtctgaatgaaattaaacgactgaaagcgattaaccatcaactggaaacgatgcataaggagcccattgccattgtcggagcggcttgtcggtatccggggggaattggatcattggaacagttctgggaagctctggagcaaggacgtgattgcattgaaagaatgcaagatcagcgttggccgatgtcccgttttcttactgataatcctcataccgaaggtggtatttatagtgatgcgatggggttgcttgagaatgttgaccgttttgatcctgcacactttggtttatcggttgaagaagcaatacatttggatcctcagcatcggctcttgatggaaatggtttgggaaacgattgaagatgcaggatatgctgtagacagtttttcgggcacccggaccggtgtctatattgggttaatgagcgatgactatggtcagttgcagggccctttggaaaaagcaagctactatgtggggtcggggacggcacgtagttgtgctgctggtcgactctcttatacctatgggctggaagggcctgcgatgacgattgatacggcttgctcttcatccttagtgagtgtgcacctcgctgtgcaggcgatccgccgcggtgagtgtgataaggcaattgcgggcggagcgaatcttattctctcaccacaaggtagtgttgttgcttgtcgttctcaaatgttatcacgatcaggacactgtcagacttttgatatcaatgcggatgggtatgttcgttcagaggggtgcggtacggtcttgttgaaacgtctcagcagtgcgttgcaagatggggatcaaatttatgcattgattcgtgggtcagcgatcaatcacgatggccgtagccaagggttaacggcaccgagtggtcaagcccaacgcaaagtcattgctgccgctttagatgatgcaggggtttcacctgataaaattgaatttgttgaatgtcatggcacgggaacagcacttggtgatccaatcgaagttcgtgcgcttgaatcggcttatatcaagcaatcgcatcatcagcgaaaggcagatcagcctgtggataggctgcccttaaaactcggtgcactaaaatcgaatcttggtcatatggagtctgcggctggtattggtggattacacaaagccatggaagtgattcgtcaccgtaaggtgcctcaaaatcttcacttcaaaacattgaatccacaaatcaaagtggatcaaagtatgcttcatattgtcgatgaaccgacgcctttggctactgaagggcctgtatttgctggcgtgagctcatttggctttagtgggacgaatgcccacattattttggaatcatatacagaagcgaaagaaccgaaggatgaaaatgaagggaaaatatttcggttgtctgctcgttcccgcgctgctctgaatgattatgcaaaacaatatctcgaattcctgaatcagcattctgacatcgacttgtctgccttgtgttacaccgcggcaaccgggcgtgatgatagcgcgtatcgaatcgcgtttgcggttaacgatgtcaaagacctagaagcttgtctgaaagaatatattgaggtcacgggaggcgatgaatttttgccagaagcaggttccttccccttactttgggtgatcagtggccaaaccaatgtggactggcgcttagcgcatcacctataccagacgcgaagcacttatcgatcttcaattgagaaagcttacaaatatctagaagaaatggggtatccacactcgctcgctgactttgaatcaattttgacagggaccgagcagacagcggatgtattacctcatgcaatccatcgctgggcactggctgaactgttggttcatctaggcttatctccgacgtgtattacggggattggggttggggaatatgttgctgcgagtgtcgctggcttgattggttggcagtcgctgatggatattctatccacagggacggtcaaaaaaaccattgctttgggtcgcaagcgatatgactttgtcagtagtgccggcaaaaatgcagtattacccgaatcttggtttgggcatgagcctctggcacagaatgtgacactggaaaccctcggagccacatttagtatcgaatacgacacggtagattattgttgtatttccttggacgatacattgtcactgacgatgacggatacgcccgaggaaaatctgttccaatgggtatatggtcagacaaatcgggatgcaacaatgccgctccaaggatttctggcacagtgttatatgagtggttataccttgcattgggagcgagtctatgaggatagtcaatgtctgaaaatgagtttaccgacctatcctttccaacggcaaagcatatggactaagtggggttactcgtttgatgagacgcttccatcgacggttggtcgttccggaaaaatgttacagatgtcatcacgccatgagcgtcagggtgaagaggtcattgaacacccaatcttacagacggtctttgcctgtccttcagggattcgtaacttccaaggcgaactctcactggctcgaatgccttacctgggcgcacaccagatactcggtgaatccattgtgccggttagtttgtttgttgatattgttctcacggcaggtcggtggtgttggccagagcaaaacctatggattgaagacttgcagttgctgcataagtgtcctttaggtgaagcaccgattgacatttattgccatgtcaacccagatgctggaactgtttcagtgtatagcaaattacagcacgcaaaagagtggcagcaacatgtcagaggcagactatcgactagtgctcaggcgacggcaatgacgccacaggtcagtttgtctgaatttcagcatcaatgccatgaggcgatagctattcgtcatttctatcagggaattgcattaagtggcatgacttatggggaagactttcagggtatcttcgagatatatcggggcccccatacagcactggcaaaaattgctttaccaccgacggtcgagcagtctcttgttggatatagcacccatccaattctattggatgggtgcttgcaggcgattgtcgctgcatcagtgcagcgtcatgagcagggggttcgggtgccttcacaggttcgaggcattcgtctcttcaaaccgctcccggaaattttatggtgtgctgttgaggtctcttctgtgaactcgtctgatacggaggaaccggaggccggaaatagttacgcttctctgactgtgtttgattcgctcggtgaggtggtgatgaccattgagaggtttgaaacaacgcattatacggacactgaatctgttaaaactgccgcttatcatgaatggttgtatgacaagcaatggttacctgatgaaacggacggatccattcaggcaagagcggaaaacccagcatctgttgaacgtcattggttgctcttttctgatcagggcagtgtttgcaaggcactggaaacagcattgattgaccgtggtgatcgtgtttccgttctctgcctgaaccaacccgaggaacctcattatcggcaagtttcttttcaaagtatggcagatctaaatgaggtattcgacgcagtggagcatgatgtacatgctctaaccggtgtgatttatggctggtcagtcaccccatttgaagcggagagtcttggacatgagcaattatcgatttatgccaagtacccactttgggtatgtcaatcaattttggatcctcggcgaagacggttggcattgagctttctgacgtgtgacagtcagcctgtttatggacaagcgccttcacaaccgttggctgcgatgttatggggccacgtgacgagctacatcaatgaaaatgtagttcatgcaaatttgattgacttgttgtcaacagatcttatcaataatcatgaccttgaccatgtgctccacatactggatcaagtgaatgaatgtcaattcgctattcggaatggccaacgtttgattgctcgaattctcccaagtcagacagatttgtctcacaatgttcaattggatactacaggcacctatctgattactggtggattaggtgcacttggcttagaaacagcgaaagcgttaattcgtcaaggggctcgtcatctcattttagtcggccgtcgggctggtctcacagatgacagtcaagccgcgatacagtctcttgaggcgttgggcgcttctgtctatccattagttgcagatatttcgaaggcgcatgcttttgtcaacagcttaactgaacggttgattggcctacctcctttaaaaggagtcgtccactcagcgggtgtcttggccgatggcgttgtagcacagcagtcatgggaggattacctcaaagtatttgcgcctaaggttgcagggactttgtcgctctatcaagcggtgcgtgaacaagcactcgatttctttgtgatctactcgtctgcagcatcaatattaggaaaccctgggcaagcaaattacgcagctgcaaattcgtttgtggatagctttgtttggtatttaagaggtcaaggtgtcgctgcgacatcaattaattgggggggctggtcagaaattggcttggcagccaatatggcgcagcaagtacctgatagagaaaatgcgctacttggcttgattccacccagacagggcattcaggtgatagaggagcagctcatcagtgcgcatcctcaatttgctgtcttgcctttgaatcgtcaaatgacccttgatggcgataaaatgccgtatctacgtcaattattaagcaatgttctggacgatgatcgtaagcacctttcgacatccgcgatgcagaaacctgaagagcaatccacagctctgagcgttctggcaagtttaggacgtgttcatggtaatgatcgatgccgtctgctgaagcaatatttaaatcaagtgatcggtgagatgttaaaaattccgggtcagcttgatgagcgagccagtctgtttgatcttgggctcgattctttattaggtgttgatttgagactacggatagaaaaagatctggattgtgtcttggcatcgacacttttccatgactattcgacgatcgaaagtttaacaacctatctgttaaacgacgtgatcaaagacattgaagttgaagcgacgcaaaaggagcattcgccaacggctaatccgatgcatcatttgttaagcagtgctgatgtgaaggtgacaatctcacaaccagagaaaccacaagaaacgccacaggatcaagttgctggttcaggaaaaaatgaaagtcatagaaaatacacttctaacgctaagcagggtgatattgcgatcatcggtttatcgggacgttatcctggcgcatctgatctggagacgttttgggaaaatttacgggaaggttacgatggcatcactcgtgtaccagaagaacggtgggatgctgctgcttattatgatgaacgtaaaaatgtatccggtaaaagctatggggatatcgggggattcattgaaggtgtagatcagtttgaccctgagtttttcaatatcccacagcatatggcagcctacattgaccccaaagagcgcctgttccttgaaaccgtgtggaatttgttggaagacgcagcttatacccgggataaactgaaacagaattatcattctcgggttggtgtttttgtgggggcgatgtatcaactctatagtgcgtgtgccgggaatgttcatgagcagacggcaacgatgctgtcttcttataatgctattgctcaccgtgtgtcctatttcttcaatcttaaggggccgagtgttgccattgacaccatgtgttcatcttctcttacctccgtgcatttggcttgccagagtctatataacggagattgtgagattgcgattgcaggcggggtcaatctctctcttcatcctttgaaatatgtcggattaagtcaggctcacatcatgggcagtcatgcagggagccgaagtttcagcgatggagatgggtatctaccgtctgaaggggttggtgctgtactactcaaacctttagagagagctatcgaagatggtgaccgaatcgaagcggtgattaaagcttcaacgatcaatcatggcggtcattcaactgggttttatgcgccgaatcctgaagctcagaccgagttgatcgagacaaactttcagcgcgcgcagcttactcctgacagtattcagtatgttgaggcggcagccaatggggccagccttggagactccattgaatttaaagtattgaaccaagtattcatgaatgccggtgtgaaaaaaggcagttgtccgatggggacggttaagtcgaatatcggtcatgctgaagcggcttctggtatggcgcaaattgcgaaagttattttgcagatgaagcatcagacaatgatcccaacggtgaaagccgagcctctgaatccaaacattccactgttagagtctcccttccgtctgttgcatcagtgtcagcgttgggaacagccagagaatgatcaacgacgtgcgacggtcagttcatttggtgccggtggcgccaatgcgcacctgatcctagaagagtatgctccgaccgagagaagtacggcactgaaatcatcaattgttcctcatggagacgagatgatcgtgctttctgcgcgctcaaaaactcagttgcaacagatggtgaagaatatgctgatgtatttggacaaacatcaatcaaatgaggttgagacacaatcgaaatggttggctgatattgcacatactttgcagactggcagggaagaaatggattgtcgtttgtcattgctggttagcagtattgatgagttacgtcaggggttatctcattatctttcttctgaatctgttcttgatacggtttcagggaaggatatcgctcctgttcttatacaaacggggaacattcaagaccagctcgagctgcgaaacctccttttaggagtggccggagaagcgatggctcagtctttgattgctgaacggcagttggagaaactgatgctgcactgggtacagggagggcgtatctgttgggataaacttcgacaggggcagccggtgaagtgtatcagtttgccaacttatccctttgagcgacaacgttattggctctcaggtccgacaacgggcgaacagagacgacgggaaccccagatgggagaagaaataggtacaacctatgagtaaatagctaatgatttatttcattgcttttacttcaatctattacattaatattttggttatggagaatcagagtgtcgcttttttcatcatttgaacttggtaatgccacattaaaaaatcgaatagctatctcacccatgtgtatgtattgcgcgaaagatggcataccagatgattggcatttagttcatctcgggagtcgggcagtcggtggtgctggcttaatatttacagaagctacagcagtttcacctgagggacgtattacacatggttgtacagggttatggaatgaccagcaggtatctgcatgggcgcggattgtggactttttacatgcgcaagggactgtcccgggcattcagctcgctcatgccggacgtaaggcaagtaccgatttaccttggttaggtggtcatccacttcccccggatgcggggggatggacctcttatgcaccaagtccactggcgtttaatcaggggtacaatgtgccagttacattggataaaggcggtattgctaaagtgatagaagactttgcctcagcggctcggagggcgaagtctgccggctttagcgtgattgaaattcatgcagcgcatggctatttgttccatgaattcctttcccccttatcaaaccaaagagaagatgattatggcggctcccttgagaaccgggcgcggttgctgcgttcagtcattgcctctgtcagaagcgagtggccagctccctttccattggttgttcgtttatcggcaacagattgggcacccggtggctgggatattgacgagtgtgttcaattggcgatttggctgaaagaagatggtgtggatctgatagatacatcttctggcttgaatattgcggatgcaaaacctccttttgcacccgaatatcaagttaaattttctgcccagattcgtcgtgaggcaggcattgcaacaggtacagtcggcatgattactcctggtaaggaagcagatgacatcatttatcgaggtgatgcggatcttgtattgtttgccagagagtctttgcgtgacccttattttccttttcgtgcggctacggcgtgtggtgctgaggtttgtgtccccaaacaatatttacgggcttggtcgtctgccacttagatcatcaggataatttttatataaaggaagtgattgatttatgaatttatcaaatataccgagtttggcacttgctcatttgtcggttttaaatgtgccaccactcgaattggtcagcctggcagcaaaggtcggtttttctgatattggcttgcggttatatcctgctttttcgggttcaattttttatgaattacctgaaggttctgcccaatgtcgtgatatgcaacgtcgtttgagtgatgaaggcattggcgtcaacgatattgaatttattggtattggtgaaaattttaacccttcagatttgcaaggcctccttgataccgctggcgctctcggtgccaaacgtttatatgtttgtggcgatgatcctgatcaaactcgcttaattgataattatgcgcgtttgtgtgagttggcagcaccttatgatctttatgtggaactggaatatatggcttggcgggcggtgaaaaattttgaggatgcgtttgacgttgtaaccgccgctgggcaatcgaatggcggtatgttaattgacgcgcttcatttatttcgcactggagggacagtgcaagacatattacgtgcacctgacttcattcattctattcagttgtgtgatgcgatagctgaacaatgtccggcaacatctgaagatttactgcaagaagctcgttcagggcgtttagctccgggacaaggctctttacctttgaaatctttattaacaacattggctgatgggactgcaatctctttggaagtcccgacaaatagtgataagcctgctgcgcagcatgctcaagaaatctttcaatccacaataaatttgatccatcgggtagaggaaagtctatgtctttaaatgatttgcaagttcctgtaagcggcaatgtgatcgacactgagcagtcacctgtgtgtcaattagggttgattggttcagggattggacattcattgtcgccagcgctttataagtctgaagccactgcgcatcaaattcagtgtacttatacgctgtttgatttagatgagccagaaaccggagatttatcagaactattggcgcaggcagaacgctcccttttttcggggctcaacattacgattccctgtaagcaatccgtgattcccttgttagatacactgagtctggaagcaaaagcgattggagcagtcaataccgtttgttttcaaggccacaaacgggtgggatataacacggatgctgagggattccgtgaaagctttctcgcacagttgcagcatgtgaatctagagcgagtcatacagtttggagccggtggtgtcggtgcagcaaccgcatttagtttgttagagcttggggctcagaaactgacaattgtggatgtcatcgatgaccgcgcgcaccaactggttgatagacttaaggcctatttcccagagcgcgaaattacaatgacccatccttcagatgccgagataaaagcaacgatgggtatcgttaatgcgacgcctatcggctcagataagtattcaggttcagtagttgaactgaacctgctttcctcccatatgtgggttgcagatgtcgtctattccccagatgaaacagtgcttttaaaagcagcccgatcattaggatgtccaacattggatggtttgagcatgctgatccatcaggcggtgagggcatttgaattgtttacggggcagcaggctgatgtcactcgcatgttgaaacgttttagacaatcgcaagcaaatcatgcatagaatacttaactgatgcgtcgtcagaagaggctgaaaaattagtctttgtcgttgagaattatgagcttaaaatataaataaacactcaatcgatgccctaaagggcatcgattgagtgttttaatctatagattccattgtgacaacaactttgcccatgaccttacgatcctgtaagcgtttcatagctgttcctgactgagccagagggaaagtctctgaaatgaagggtttaatcttacctgcttgccaaagtgtgatgagttcttgcacatttttacgattggatactgtttcatcgttaacaaatttcccccaaaggacaccaaatacttggcaagctttgagtagaatcagattcattgggattttaggaataccggctgtaaatcctatcactaagtagcgtcctcgatgagctatagtccgtaatgctggctcgcaatagcaaccaccaacaggatcaacgatgacatttggaaggtgttgaccgcaggtggcctgaagttgatttttaaaatactttgatgcttcttgagttgatatatcttctggaccgatgatcccagcatgtgccccatagcgataagccagagagagcttttcttctgaagaggcgacagcaataacatgcgctcccatgaccttacctagctctacagcagcaagcccaacgcctcctgctgcgccaaggactaggagtgtttcccctgcttggagatcagcaaggttctttaaagtgtaataggctgttgcataagtgagcagtagactcgaagcttcttcaaaagactgttcatgaggaaacaaataacagtctttttcgcttaatgtaattttctctgcgaggccaccttgattacctgtggccgcaaatacatggtcaccgatttgcaaagttttaacttgggtacctatcgcttcgacaatgcctgctatttcagagcctggaatgaaggggcgttgtggtttatcctgatataaatcttgaataatcaagatatccgggtaattgattgcacaagatctaaccttgatcagcacttcattcggcgccaattcggggtctggcacttcacacagttcaaggctgtctggtcctcccgttgttttacttagtaaagctctcattattctttcctttttatgaagatagtcacttcaaattcacattattgaaacaaaaattctgctgttgttgcgaaaaacgtatcgagcaaagcttgttgttcttgagcggataactgttcatcattgagctctagagaaacatcgattgaatcgccaaaatcgagcagtcggaaataaatcgatggcaaaaggataggccaaggttcccctgaggaacgcgttactttgggtggccattcatatgtttccgtctcagacgtttgtttcgcgtcggattggcgaataatgaacatatttatccctgttgcaatatgtcttggatcttcaacgccttgtcctgtggtaccacttggtaacggaactctcagcatggtaggaattctgagttttatagcactgtggtattcaatctcattgtatttcagtatggattcgaaatcgtaatcggatggtatgtcaacttctacaaggtcactgcaaacaagataaccgatgatgggagtgactgagggggcagtacgtaaattccccacacaacggatcggaaattttgaaataccagaccaccgagacagagcaagtgcaaaagtggtaagaaatacattaaataatgaggttttgtgtgcaacagcatatgctctgatttgctgtagaaagtagccttttatgataaatttatagtctactttcgtgccagattgccaactgagaatttccttgctaactggggctttcagtggcgataaattttgcagacgctgattccagtaattgaataagggctgtcggtctgcgctgttcagccattgtttttcccatatgacataatcagtaaactgtatggcaggcgggagtgtttgtggaagagcgttggaaatcagagtatccaattcagacataattaatacgattgatcctgcatcgacaatgatatgattgaaaacgaacaataacaaaaagtcttctttggaaaggctcacaattttggctcggagcagccactgaccattgagtggaagttgcttttctgaaaatgcttttgctaattctttcagttctttttccgtttccagttctgagcctgtgggtgtataggtctgatattcaatcggaaacgaacccgcatcatttaaggtgatctttaaataaccgtcatcctcataaaatgatgctctaagtatgttatgtcgggagatgagttgtctaattgcagcttcgactctggctttgctggtattccgataaggcttaattaatggaagtcgttcctgttctagctgacgttctggtacgccataccaaaaccaccaaagtttctgtaacgcggagggttctatttcggtcgattcttcaaaagtcacaggtgtttcaagttgagtttctagcgctgataaataagtgatgatctcatctcggtgttcactgagcgtggttttatcttgatttgtgagggtgccttttggcgctctataagcgagcgcaccatccttcagcgttaaggctattcctctattttggaaattgaaaattaaatggggaatactttccattaaaatgttcttccttaatatcagttagtttcatctttaagggggacttgatataaaacaccatcattttccaatgccttaatttcttcatcggtgtagccgatactttcggcaatatcctgattatgttgaccaagatttggtgcgatatctaaccgttgaggttcaatattggagaaattaaaaggaacattgggcatcaaaatggtacctaattctggatgttcctgttcaatgatcattcgacgtgcatgaatctgtgggtctgccaatacttgtttgattgtctggactggggcgttaggcacattcgctttttcaagaagctcaatacagtttgcgactgttcggtgttttgcccaggatttgaccaagtcaacagcagcttgactatttgtattacgattttctgctgtctgaaagcgagtatcgttggcgagttcttcacccccaatgaggtgcgccaattgttgccaaacttcattcacttgtgcagcaatgacgatagcaccatcagaagcaggaaatacaccataaagggtggattgaggcaaatggttaccacttcgcttcaccacatcggtaccatcgctcagggtatggaactgtactgcaaaatcatgcattgaaaccatacagtcatataatgcaatatcaatatgttggccaataccggttgtatggcgactaaataaagctgcacataccgcagaaacaccatgactacccgcatacatatcagcgacaggcattctcatcagtggtggcacatcatttggttggccaatgagatccattgcaccactttttgcctctgcaattaaaccaaaacctgcacgctcggaatcaggtccagtttgtccgtaggctgagatagagcagtagattaagcccggattctcttctgctagtgtgtcgtatcccaatcctagtttttttagagcgccgggtctgtagttttcgataaagacatcagccgttttcgtcagctttttgatgaattccagaccacgaggatctttggcgtttacacatagtccttttttccccatattttgctgtagaaaatacccactgtgtcctttgataaaatagggatggtttcgtcctgcgtcgcctgctttgggacgctcaaccttaatgacttcggcacctaacgcggctaaacagcgtgaaacataaggacctgcgagaaagtgactgtaatcaatgactcggatcccttttagagggggcggttgttttagcatactgaattcctttcatttcggaaaaatactagacgatgaagccttcttcatattcgtcgtcttgagagtattcggatgatgagagcgcaagagtgagtacgatttgttcactcatcttggcaatggtttgtgcttcatagagacagttgagtggtaaatcagcgcctatggtgccacgtataatggagagcatatgcattcctaactgtgaatccccgccgatttcaaaaaagttttgatgtcgccctattttcggtctccctaagagctcttgccagatagtggttaagtcggattcgatgccactgatggtgatactcgaagacaaggcggtattttcttcggcattttgctgtgtatgtgtggatggctgataaaaaggataaacaggcaggtgaattttattcggtatgaccgatctttttattcttgtccatgaaatatcaccgccttggctccaatataacgcgagattactgagttctcctgtttcaatgagcgtgttgagcatcgtttccccactactaccggagaaaaggttttgaaattggttgttacgatttggatgaccacgatagagattcactggcacttgaggttgaaccctatttaatacattttgcacggggggggtggcctctccctcgtttgtgagaaattgagcgagaccgaagagaagttcatctaagcggttgacaaccattgctaatctatagcccatcgcctctctaccacattgcagagtataagctagatcttgtaaggacagcgttccggagttcgtgagcattgaactggcattattgatcttttcaaggggaataaaatcaaggatatgattgaatagttcggcagcttttatttgtagtgcttgagatgtgttggctgatagaacaataagttcagggtggttcagctcgggggctgtggctggtatttctactggagacaggatatgagaggaataattattgaatttattaggtctttcagaatcaggtgacaccatagccaaaatagggagttcatgatgctggctgcttctgggatccttaccaagtaagacgccccaaggtgttgcatccgggggcattgtactcaattgtaattgattggatgttttagataaggggacgccaagtattatagcaatctgagtatctccaaccaataaataatgggataactcaccaaatgaatggatagaggtaggtaaagctcgttcgtttaaaaaacaggaagagggtaaacccaataatttggagagatggttggtgatgctttcatatattgtttgatattcctgataattactgagtctagtgtcactaaaggttgtatctatcgcaaaatttggttcttgattgatataaacgacaatgttgtcactattgtttttattaataacgtctcttgtgacgcctgctgattcaagtagctgccagatcatttctagataacatttaacctgctcatcaaggtataattctggttgataaatcgtctcaaaaaaattcgtatccagagacgtagtgttgtgatttttattcgacttaatatcagttgaagcgtttgtatcaaataagaatggcttgctatcgttgtgaaaatggcttggtcttaaaggtgcttttgaggattggatagcagcgataatggcaatctctgacggtttgctcttacttaattccgattcagtcat

SEQ ID NO: 2 - DNA, 1578 bp, Vibrio rhizosphaerae MSSRF3
atgggacaccaacaaacggatgttttaattttaggtggcggactggcaggattaaccttagcacgtcagttgcgaatggaaatgcctgagctggatattatagttcttgagcgaagtaaaaggccggctcctgaggcttgttataaggttggtgaatcgagtgtagaagttgctgcgcattattttgcagaacgtctagagttgagagaacacatcgataatgagcaactgccaaagcttgggttgagatacttctttcctcagggcgagaatcgagatgtcacgaaacgctttgaaattggaccaagcgagtttccagttactcctagttatcagttggatcgaggccgctttgaaaattatctgcatgaattgaatgtggataatggcattgaaattcatactaaagcaaaagttcaacaggtaacgctaaattctgaagaaatgcatgaagtaaaatatatgcatggatccgaggtgaagtctattcagagtaagtggttggttgatgccaccggacgtaatcagttaattaagcgtaaactgaatttggagaaagatagcaaccttcataatgatgccgtctggtttagattggacgcagaggttgatattggcagtttaagtgatgttccccaatggcaggatcgtgctgttgaaccgcgtcgtttaagtacgaatcacttcatgggaccaggttattggttatggtttattcctttgggttcaggtgcaaccagtgttgggattgtctttgataatcaaatgcataatttcaaagatatgaacaactttgagaaagctttaaattggttgtatgaacatgaaccgcaatgtgcagagttagttgaaagttaccgaagccaagtgatggattttattggatatcggaattattcatatagttgtaagcaagtattttcagcggatcgctggtgtattactggtgaatccggagtatttatcgatcctttctattctcctggtagtgattttattgcttatagcaacgattttgtgactgaactaattgtacaagacttcaaaggtaaagatattagacaattggcatcttcttttaacgatacctttatgaagttagctgatgtggtctcaatgctatatgaaggcttatatccaaaatttgggaatggttttgtgatgaagcagaaaattctttgggatagcgttatttatctcggtgtcacatgtctgttgtactttaatcgaaagtttactgatcctgattttttagcgttggttgattcagagttagcacattacaactatttattacgttctgtagctgaacactttaaacgtcaacccattactcatgatgtacatctcgatggcgaatatgtcgatttaactaaaacctttttgtttggtagaaatctgaacgaagagttgaccattgaatataaagatgacgacgcgctgattgaaaaactaagagagaatatcgaactgctcgataaaatatccgtaagtattttcaataatgttgatattgcgtggaattttcaacctaaaaaattagcagatacaagtgaagagcaagtcgtaaacagctag

SEQ ID NO: 3 - protein, 525 aa, Vibrio rhizosphaerae MSSRF3
MGHQQTDVLILGGGLAGLTLARQLRMEMPELDIIVLERSKRPAPEACYKVGESSVEVAAHYFAERLELREHIDNEQLPKLGLRYFFPQGENRDVTKRFEIGPSEFPVTPSYQLDRGRFENYLHELNVDNGIEIHTKAKVQQVTLNSEEMHEVKYMHGSEVKSIQSKWLVDATGRNQLIKRKLNLEKDSNLHNDAVWFRLDAEVDIGSLSDVPQWQDRAVEPRRLSTNHFMGPGYWLWFIPLGSGATSVGIVFDNQMHNFKDMNNFEKALNWLYEHEPQCAELVESYRSQVMDFIGYRNYSYSCKQVFSADRWCITGESGVFIDPFYSPGSDFIAYSNDFVTELIVQDFKGKDIRQLASSFNDTFMKLADVVSMLYEGLYPKFGNGFVMKQKILWDSVIYLGVTCLLYFNRKFTDPDFLALVDSELAHYNYLLRSVAEHFKRQPITHDVHLDGEYVDLTKTFLFGRNLNEELTIEYKDDDALIEKLRENIELLDKISVSIFNNVDIAWNFQPKKLADTSEEQVVNS

SEQ ID NO: 4 - DNA, 936 bp, Vibrio rhizosphaerae MSSRF3
gtgtcaccatttgagcagaatttagaaattcgtaaagctgtgattaaggtaatgaaagaaggcgggtattaccaaaaaatcatggatggggattctgaattagaccgtgtcaaagaattcttaattcggatggagaaagcccaacacggagaacgtttatataagcagcatcctacatttttaccgattttcccaggcctcgataatcaaccgatgcgctcatctaaaggcgatcctgttgctgactacttacgtgaagcaacaccagcaatcaaagaagacgcgctgcgtttaagagatcgcacactgtcttttacggggggaattgtgacagatggggcatggaatattttcccactttggtacatgggagttaatctcccgtttatgtctgcacactgtcccaacttatcccgaattgctgccgagatacctcgttgtggcatgttgcacccattttctgaagcgctattgtcttggcaggatcctcacacccatctcgctgcgcattgtagtgttgattcgttgaggctgcgatatagcgttggcattattgttgagtccgattgtagtctacgtgttggtgaggtaaggaagcagtgggagaccggagagtcaatcatattcgaagattgttttgaacatgaagcatggaatggggataaaagtcgtttagtgttcatcattgatacttggcatccagaccttaccgccattgagtgtgaagccttactcgcagggtttcggaagaaagaagttcgccatatcctctatgagtaccgtatgtcggaggcgatgagacctttcctgctgaaccgatttgaaatagaagaaaaagagcctttgattcaacagtactgggagcatgaaggtgaattatatgttcctaagccaaaaaattgggggatatgggggacgacccctgtgtttacctga

SEQ ID NO: 5 - protein, 311 aa, Vibrio rhizosphaerae MSSRF3
MSPFEQNLEIRKAVIKVMKEGGYYQKIMDGDSELDRVKEFLIRMEKAQHGERLYKQHPTFLPIFPGLDNQPMRSSKGDPVADYLREATPAIKEDALRLRDRTLSFTGGIVTDGAWNIFPLWYMGVNLPFMSAHCPNLSRIAAEIPRCGMLHPFSEALLSWQDPHTHLAAHCSVDSLRLRYSVGIIVESDCSLRVGEVRKQWETGESIIFEDCFEHEAWNGDKSRLVFIIDTWHPDLTAIECEALLAGFRKKEVRHILYEYRMSEAMRPFLLNRFEIEEKEPLIQQYWEHEGELYVPKPKNWGIWGTTPVFT

SEQ ID NO: 6 - DNA, 780 bp, Vibrio rhizosphaerae MSSRF3
ttgttcggcgtcgattttgaacaatgctttagctcgccagtaaaggagcgtactaatgcaaatgtgatattgttttgttttccctacgctggtgcgggggtctcttcttatcacacactttgtaatttattacctttggaagtcattccatttgttgccagattacccggtcgtgaaaccacacatcaacaaccgccatttactgaaatgtcagatattgtttcgtacttgtcaaaggcaattcgccctgctttaggaaagtcgtcaatttttttttggggtcacagtatgggggctcttcttgcgtttgaaatcgcgagaattgtgcagaaagattacaccttagatggtttgattgtttcaggacatgtcgctcctcatttacctgttaagccaaagcctgtttcaattaaggaaatggatgacgctcagttccttgaaattgtcagaagctacggtggtatgcctgatgccattctccagaatcccgatatcctccaattagttttaccgcaaatcagagctgatttaaccgcgctagagggatatcaattcgttcagggagaaaaacttgagagtgatattttttgtattaatggtcagtccgatcatatggtcgatccacataacgtgatgctttggcaagaattaacgtcagcgaaattctccagtcaatggctaccagggtcgcatttcttcattaatgaacaacgccatggggtggtgaagcgcattagcaatatcattcgtcatcaacttgttcaaatagatatcatctaa

SEQ ID NO: 7 - protein, 259 aa, Vibrio rhizosphaerae MSSRF3
MFGVDFEQCFSSPVKERTNANVILFCFPYAGAGVSSYHTLCNLLPLEVIPFVARLPGRETTHQQPPFTEMSDIVSYLSKAIRPALGKSSIFFWGHSMGALLAFEIARIVQKDYTLDGLIVSGHVAPHLPVKPKPVSIKEMDDAQFLEIVRSYGGMPDAILQNPDILQLVLPQIRADLTALEGYQFVQGEKLESDIFCINGQSDHMVDPHNVMLWQELTSAKFSSQWLPGSHFFINEQRHGVVKRISNIIRHQLVQIDII

SEQ ID NO: 8 - DNA, 7959 bp, Vibrio rhizosphaerae MSSRF3
atgctggaagtcgttaatacctaccttcatggctacgcagcaattccgatgctaatggtttgtcaagagacaggtatcatgactgcgttggaaaagggacctgtaagcgtacaggaaataacaaaagagctgtcggccaacattggttatagccgactgatttttcgaaccctgaatgcactgggttatgtcacaacgacagaccgggaaaaatatacactgagtccggatacaaatcatgactggcgtctacctgacacaatgattgaagtttataaaatcgatttctatgattattttttgaatggtactcataaaaacaaggtgagagcttggctcgatttgtcagtcgatgattggggtaaaactgataaagaaacgcagcagctcatggatggcgtgatcatggtgccgtttttattggcgctcgctgaattaggactcggtcaggacgaaaacatccagtccagtttacttgaacagtatattcacgaaagtatttggtctgatgtacagtattttctgactcggaaaaacctgttagaagaagacaaactagtccttaatcaggctggttggcacttatgtaatcgtgcgttgaccatggctactatggtgtcataccggccaatgttattgaattttaaagaactactttgtggtgacccaaagcaagtgtttgaccgtgatgaattgggacatgaagtccatgtcgatcgaagccttaatgtgattgggagcggcttccaacatggtaagtattttttcgatttggcagagtttatcgttgagattttcaatcgaacacctgtctcagagcaacctaattatatcgtcgatatggggtgtggcgatggtgcgttgttaaaaacactttatgaggaaatcactaaaaatagtttaagaggacagtatctagaacaataccctctgcttttggttggtgctgattataacgagaaatccttgctggaatcagcaaagacattggatggattccctcatctgttgttacctggggatattggtaagcctcaggatttgatgacagatctacggtcatacggcattacagatgttgatcaaattatacatgtacgttcatttttagatcatgatagacccatcaatattgaggcagcaccgacagacatgatccccagagatgatcatattcttgtcaatggtagaggtgattggattgactctgatacggtgttcaaagacctctgtgagtatttgacacggtggtcaagtattttggggaaacatggactaattctgctcgaagttttttctttacccattagcttaacgcgcgaatattttaatcagacagagagtttccatttcgatttctatcacgctatgtccggacaagttttagtcgatgctggaatgttccatcaagcactcgcttgtgcaggtttgtatcctaaccgagagacattgtcgagatatcccaaaacaacacctttttctcgtattgtattgcagcagctcttgcctaagcaggtcattgtaaggccttttgtcttagatgatatgccggctgtgctcgccattgatcgggcttgttggcctgagcacatgagactttcaacaaccgagattgagcgacgtcatcgcacctttcctaaaggtcaactcgtcgtggaatatgaagggcgtgttgttagtgtactttattctcaacgcattgacgaaatcgagccgatattttctctgggttatgcaaattatttgaatgctcataataatgatggtcgatattggcaactcttggggcttagttcagatcctgattatcaatttttgtcgctgggggatcatcttttagagcatacgcttgatttagcgtcactcaccccgaaagtcgaagcagtgtatggggtaacacggtgcgtggcatatgctcaacaatctgtgccattggaagaatacatttttaagaaagatgctcagggttatccaattgaaccgttattgcgctttcattttagccatggggcttcgatagagggtttagtgcctgagacacgtccagaggatgaagagaatgaaggtgcaggggttctgatccgctatgacctgagcaaacgttttcaaagaccaaatcaaactcaaccgagagcaacaagagatatagagcgggcaattactatcgtgtcagattgtattcgccagataatgaaaacaccaagtgattttcgagatgatttgccgttaaaggagctagggttggattccatgggattgatggaactccgtcttttacttaatcgtgcatttggtattgagtttgaacctgcgtatttcttcaattatccaacggcagtcgcgatcgctgactacatcgataaattaaacaaatcttacgtacagcctgaagcagagctgagtcatgcatcgcttgtgggacgacaggcagtgcctaaccgcgtgtctgtatctgatgtcggacgctctgactccgaagttcaatcgtcgcaagagactgacgttgccattgttggcatgtcattacgtttccctcaagggattgaaacacttgaggaattctggtcggtcttaacacaaggagagtgcgtaattagtgagcgtccggagacccgatggcgagaatttcaatccgaattggagacattagatacacgttggcaacacatccatcgtggcggttttttgaaagacatagataagtttgatgcggaattttttcatattacccctgcggaagccgcatccttagacccacaacaacgtatcttgctagagcttgcttgggaagcttttgaaaatgccggtatggatgttgaacaatttgcaagtcacccagtcggtatttttttaggcgcttatacacaagattatcaaacgctgaccttacgacaaggtgttgatgcggtggatgcctattttggtggtagtaatgctttatcgacagcagcaggacggttggcgtacttttttgacttccgtgggccaacgctcacacttgataccgcatgttcttcttcgagtagtgcgatttatacagcatgtcagaatcttcttgaaggcaacgcagaaatagcactggcaggcgcgatcaatacgatgatttccccaaatttgagtttagcttatgcaaaagcaggaatgttgtctgtcgatgggctttgtaaaacgtttgatgctgaggccaatggttatgtgcgaagtgaaggtggggctttcctactgttaaaaagaaaagccgatgcaatccgggatggggattttattcacgcagtgattaaatcggccgcattgatgcaggatggacgcaccaatggtcttacggcaccgaatggcctcgctcaggaagatgtgattcgtcgagctgtgcagttgtccggatacaaagctgaggatgtcagttatatcgaagcgcatgggacggggacttatctaggggatcctgtagagatgcaggcactcagacaggttttctgtgatggtacggcacgtcggaacacattaaaagttggttcagtgaaaaccaatttaggccatacagaagcggcttcagggatggctggtatcatcaaagtcgctttatcgatgcaacatcaatggattccaccacatctgcatcttcaaaatatgaatacattgatgaatattgcgcaggctgacattgatgtaccgacgcaaggtgagttctgggaagtgaccgaaggccaaccaagattggctgggattagctcttttggtttcagcggtagtaacactcatattgtactagaagagtttcacatgcctgactctgagcagtggcatggacgtagcccattacccgctgttatctctgccaaatcgattcaatcgttacggcagaatatccttgctttgctgacgtatatcgatcagcacggggcaacactcaatcttgctgcactatcgaaaacactgacacaaggacgtacgcagcattgttaccgaatctcttttccttttgattcgcttgagacactgaaaacgtctctgcaagcgcagttaaaaaatttatcctcagagaatccgcctgttacttcatcggttcaacccagaatcgcatttatgtttactggtcagggggctcagtataaggggatggcttctcgcctggatcgttttagtccggttttccatcaacacctgtcagagtgtgccgctttcgttcagacgtatggtgaatttgatttatttgagcttctttgggggaatgatgagactctgatccaccaacctcgctatactcaagtcgcgttattttgtatcgaatatagtctctctgaaatgttacgggagtcagggattgaagctgaagcggttcttggacatagcgtcggagagtatgcggcagcttgttataccaaagtcattagcctcgaaaccgcgatccaattgctcttttatcgcggtaagttgatggatgaacaaactgcaatgggtaagatggttgcagttcttgcaccattgagcgatgttgagtgcttagttgaaggctatgaacttgtctctattgcagcgaagaatgggccttcaaatcaggtaatatcgggggatccagaacagatcgagcgcattgttcaaaatgctgaacaactgaagatgaaaacatttccactcaccgtggaaagagctttccattctccgctgatggcccctattctcgaaccattcaaagcgatagcggaaccactgacttatcaggcaccacagtcagatcttttttcaaacttaactggtgatcgatataccgaggtgatggatagccaatattggactgatcatatcagctcacccgtgaactttgagcaatctgtacgaagtcttctgaagcatggggtcgatatggtggtcgaaattggcccccgtccgattctgacgaatatgggtttactgattgatgcagataaaaatacgctttggctttctactcttgtcgatgctgagaaccataatctagcgagtctctttactcggttgagtcagtctggtgtagacattgattggcgtttttatcctcatgaaagcgtgacgagactctctgacgtaccactttatcagtttgatcgaagcaaatactggctcactgagtcttcgaaagagcacgctcttacatctgtctccgtcgatgatagcgcagaacaggatacaaggcgcgatatagacaaagagaccgaatcacaaccgcagtctgctacgcctaacttagttggtatctttccgaaccgtatcgaaccaaagcgtgaccgtgatatatgggcgcatgcgattggcagcgagagtctatttcctgccagtggttacattgggttggcgttagaggctgggatgtgttgtttgaagcgagacggttacgtagagcttagagatatgcatctcgaacagatgttgacactgtctgatgaacaagaaacctatattcaagtgagagtttcagaaggtgattcgggcaatatgctttctatacaattagaagcatggcaggcggatcagaaggctccccgatccttgtatgcaagaacagcaatgaaagcactgacgtttggtccggaggatgcttgggaaaaggaagtcacaactgttgatctgccattaaccagtgacgctgtcatgcagggagaaacgttctataaacgaatttcagcattaggctatgcctatcggcctccgttccaagggattcggaatgttcggcgcgctggaaatactttggtcacgacactcagattttctaatacaacgccaaatgcaggctatgcagcgacgccatggagtctggatgggtgtttccaaaccattctcgctgtcgtcatcgatgatttagaagctaaccatgaccaacttttactcccagtcattattgagcagtttacttggttgtcgtcacttccgtctgaagtgaatgtcgcatgtgtgtgtcagtcgacgacccgtggcatatcagccaacatgtcaatttatgacattgcaggtcaattattagttaatattgttgggttgaactgcgtgtgggtgaatcgccaaagtctaggtttgcaagccattccgagcagtacaccggagatttatcgtcccgattggatggcatctgaggtatcagctgagcttttatcacttgtctcactcaaagaacaaatccctgcttggttggtacttactgatgataatggtcaggggcaggcaattatcgatgtgctgacctcggcagggatgcgagttgaagtgctgcaatgggagaaggctcagaagccatccatatttaccagacagttaatgaatttctcgacatcacaagagacaccatttggactcgttcatagttggtggttaactcaaccgactaatggtgctcatgctagggggcggtatgaacttgatttagctgtggctcagtttgtagcctcaacagatacagagcgtttatcgcgggctattttcctaacatcacatgcctatccggttttgaaaactgacgagatggatgttgaccaaggtgcggggctttgggggttggtgaatacactacgagccgaagtcgctacgcatgaactcacccttgttgatgttgcaccgacacaaactgaggaagaagcacgtcgtcaggctgagcaaattttgctgacgtctaatgcgacactcaaggctacagaagccacggattggcctgaacattttgcgttacgacagggcgctttatttcaacgtgtgcttcaaccagtaacaagctcgactgaatctatcagaatctcgggtgatgccagctatattgtcaccggtgggctcgggggaattgggcaatacattactacatttcttcataaagaaggcgcagggcagattgtactgttgggacggtcatttcctgaagaattacccatttgggtgcaacaccttaatcaaaaacgtcaagcgattattctgcttcgctgtgatgtgacagagccgcacgatattaccaatgtgatgaagcagttagagcaaggtcccacggtccgaggactgattcacgcagccggagttgtggaagatgcgttgctcgtcaatcagactcaggaaatgctttctcgggtttatgcccctaaggttgaggggacgaaacatcttgtggatgcgtttgcaacagagtctcttgatttcgtttggttattctcttctatcgtcggtattttgggggctgtagggcaggcgaattatgcaacagccaatgcggtgatggacagttatgcgcattatctacggcaacaaggtattagggcaacatcgattaactggggcccctggaaagagacgggaatgctgtctgatttgtctcagatggcgctagagcgtgcgagttattttatgacggggattgatccgagcgcttgtagtgatgatttgtttactgctttgctcaattcggatgaagcacaatgttgtgttgttcagtggcatattgaacacatcaatcaggaaaaatatctgccgcaaattttgtcttcattgcgtacccaaccgatacaagagcaggtctctgtctcgttggcatcggttttagaaggggcattaggtgaagaacaggtgttacgtacccgtcgttttgtggcagaaaaaatcgcccaattaacgggtctatctcccgacgtgattgaaccgtccaaaccgttctctgattatggtgttacttccattgttagtgttgaactcagttcaatgttaaccaccagtgtcggtgcaacttttgcgacgaccatgatgtttgactatccaacgctcagtttgttggcggaatacgtattggaacggagtgcgttgagagaccagagcaatgatagtgttcccatcaatgatgacagtttgtcccatgaatctttctttgagatagacaaggatttagatgaaatcgagaatcttgatgacgaactacttactgctttattgagtgaggagatagatgatgagtga

SEQ ID NO: 9 - protein, 2652 aa, Vibrio rhizosphaerae MSSRF3
MLEVVNTYLHGYAAIPMLMVCQETGIMTALEKGPVSVQEITKELSANIGYSRLIFRTLNALGYVTTTDREKYTLSPDTNHDWRLPDTMIEVYKIDFYDYFLNGTHKNKVRAWLDLSVDDWGKTDKETQQLMDGVIMVPFLLALAELGLGQDENIQSSLLEQYIHESIWSDVQYFLTRKNLLEEDKLVLNQAGWHLCNRALTMATMVSYRPMLLNFKELLCGDPKQVFDRDELGHEVHVDRSLNVIGSGFQHGKYFFDLAEFIVEIFNRTPVSEQPNYIVDMGCGDGALLKTLYEEITKNSLRGQYLEQYPLLLVGADYNEKSLLESAKTLDGFPHLLLPGDIGKPQDLMTDLRSYGITDVDQIIHVRSFLDHDRPINIEAAPTDMIPRDDHILVNGRGDWIDSDTVFKDLCEYLTRWSSILGKHGLILLEVFSLPISLTREYFNQTESFHFDFYHAMSGQVLVDAGMFHQALACAGLYPNRETLSRYPKTTPFSRIVLQQLLPKQVIVRPFVLDDMPAVLAIDRACWPEHMRLSTTEIERRHRTFPKGQLVVEYEGRVVSVLYSQRIDEIEPIFSLGYANYLNAHNNDGRYWQLLGLSSDPDYQFLSLGDHLLEHTLDLASLTPKVEAVYGVTRCVAYAQQSVPLEEYIFKKDAQGYPIEPLLRFHFSHGASIEGLVPETRPEDEENEGAGVLIRYDLSKRFQRPNQTQPRATRDIERAITIVSDCIRQIMKTPSDFRDDLPLKELGLDSMGLMELRLLLNRAFGIEFEPAYFFNYPTAVAIADYIDKLNKSYVQPEAELSHASLVGRQAVPNRVSVSDVGRSDSEVQSSQETDVAIVGMSLRFPQGIETLEEFWSVLTQGECVISERPETRWREFQSELETLDTRWQHIHRGGFLKDIDKFDAEFFHITPAEAASLDPQQRILLELAWEAFENAGMDVEQFASHPVGIFLGAYTQDYQTLTLRQGVDAVDAYFGGSNALSTAAGRLAYFFDFRGPTLTLDTACSSSSSAIYTACQNLLEGNAEIALAGAINTMISPNLSLAYAKAGMLSVDGLCKTFDAEANGYVRSEGGAFLLLKRKADAIRDGDFIHAVIKSAALMQDGRTNGLTAPNGLAQEDVIRRAVQLSGYKAEDVSYIEAHGTGTYLGDPVEMQALRQVFCDGTARRNTLKVGSVKTNLGHTEAASGMAGIIKVALSMQHQWIPPHLHLQNMNTLMNIAQADIDVPTQGEFWEVTEGQPRLAGISSFGFSGSNTHIVLEEFHMPDSEQWHGRSPLPAVISAKSIQSLRQNILALLTYIDQHGATLNLAALSKTLTQGRTQHCYRISFPFDSLETLKTSLQAQLKNLSSENPPVTSSVQPRIAFMFTGQGAQYKGMASRLDRFSPVFHQHLSECAAFVQTYGEFDLFELLWGNDETLIHQPRYTQVALFCIEYSLSEMLRESGIEAEAVLGHSVGEYAAACYTKVISLETAIQLLFYRGKLMDEQTAMGKMVAVLAPLSDVECLVEGYELVSIAAKNGPSNQVISGDPEQIERIVQNAEQLKMKTFPLTVERAFHSPLMAPILEPFKAIAEPLTYQAPQSDLFSNLTGDRYTEVMDSQYWTDHISSPVNFEQSVRSLLKHGVDMVVEIGPRPILTNMGLLIDADKNTLWLSTLVDAENHNLASLFTRLSQSGVDIDWRFYPHESVTRLSDVPLYQFDRSKYWLTESSKEHALTSVSVDDSAEQDTRRDIDKETESQPQSATPNLVGIFPNRIEPKRDRDIWAHAIGSESLFPASGYIGLALEAGMCCLKRDGYVELRDMHLEQMLTLSDEQETYIQVRVSEGDSGNMLSIQLEAWQADQKAPRSLYARTAMKALTFGPEDAWEKEVTTVDLPLTSDAVMQGETFYKRISALGYAYRPPFQGIRNVRRAGNTLVTTLRFSNTTPNAGYAATPWSLDGCFQTILAVVIDDLEANHDQLLLPVIIEQFTWLSSLPSEVNVACVCQSTTRGISANMSIYDIAGQLLVNIVGLNCVWVNRQSLGLQAIPSSTPEIYRPDWMASEVSAELLSLVSLKEQIPAWLVLTDDNGQGQAIIDVLTSAGMRVEVLQWEKAQKPSIFTRQLMNFSTSQETPFGLVHSWWLTQPTNGAHARGRYELDLAVAQFVASTDTERLSRAIFLTSHAYPVLKTDEMDVDQGAGLWGLVNTLRAEVATHELTLVDVAPTQTEEEARRQAEQILLTSNATLKATEATDWPEHFALRQGALFQRVLQPVTSSTESIRISGDASYIVTGGLGGIGQYITTFLHKEGAGQIVLLGRSFPEELPIWVQHLNQKRQAIILLRCDVTEPHDITNVMKQLEQGPTVRGLIHAAGVVEDALLVNQTQEMLSRVYAPKVEGTKHLVDAFATESLDFVWLFSSIVGILGAVGQANYATANAVMDSYAHYLRQQGIRATSINWGPWKETGMLSDLSQMALERASYFMTGIDPSACSDDLFTALLNSDEAQCCVVQWHIEHINQEKYLPQILSSLRTQPIQEQVSVSLASVLEGALGEEQVLRTRRFVAEKIAQLTGLSPDVIEPSKPFSDYGVTSIVSVELSSMLTTSVGATFATTMMFDYPTLSLLAEYVLERSALRDQSNDSVPINDDSLSHESFFEIDKDLDEIENLDDELLTALLSEEIDDE

SEQ ID NO: 10 - DNA, 6687 bp, Vibrio rhizosphaerae MSSRF3
atgagtgaatccagttcgtggaaagggagttacctaaaaagactgaatcagttgtcaaaaaatcagttgttagctctggcacttcggcagagagaacaactacttgatgtcaatcagcaggttatgcaaccggatgacacagttgcgatagttggtattggatgtcgatttcctggcggtgtggtggacaccgatagctattggaaactcttgtgttcatcggaaagtgcaatcactcagatgactgatgaacgatggcacatgcaggggatttatgatcccaatcctgaggtggctgggaaaattcatacacgttctgtcggtttgttggatcaggtggataagtttgatgcgagcttcttttctattacgccgcgtgaagctgaatctatggatccacagcaacgacttttgttagaggtgacatgggaagccattgaaaatagtggtcatgcctgttcagaatttgaaggaaaatcggttggtttgttcatcgggatgatgaataaagattatcttcacttgaatgcgccagacatgatgggtgtcaatgcaaagcattccccttattacgcatctggtgaagcatttagtattggtgctggtcggttagcatacattctaggacttcgcgggccgtgtatgacgttggatacggcgtgttcttcttctctagtgggggtgcatctggcgtgtcaaagcttgctcaaggatgaatgtgattatgcaatcgctggtggtagttcacttatcctttctccagaagcgtctatcgtcagttctaatgctcgtatgttatcgccaacaggtcaatgctggacctttgatgagaaagcagatggttatgtgcgcagtgaagggtgtgccgttgtcgcactcaaacgtttgtctgatgccctggcagatggtgatccagtggtggccacaattgcagctacagctgtcaatcataatgggcggagtcagggattaactgcacctagttcaacggcacaagctgagttgatgcaaactgccctcaaaaaagcgcggattcaaccatcgcaagttcgtttcattgaggcgcatgggaccggtacgccattgggtgatccgattgagatgaattcaatccagtctgtttatggtcaggagcggagtgaagataattcattgaaaattggatcagtgaaagcattgattggacataccgaggcgagtgcgggtgttgcggggcttattaaactggctttatgtgtgtcaaaaaacagaatcattccacagcgtaatttcactcgtttaaatcctcatatccgattactggatggcgtagaaatcgcgacagaaacacagtcattttccccttcagaatccgagtctgaaccattccaattgcactatggcgctgtgaattcttttggtttcagtggatcaaatgcgcacgcaattgtgagcaatgtacagctcacatcaccaatagtgacatggcgaagtccacaactgttaaccatttccgccgaaactcccaaagcgctgttggatttgatgctgcgttatcgtgattatatttccgatgagagcctagatttacaggcgttagcctataccagtcaagtgggacgagatcatttcccggtacggatcgctttggatggagaggcgctaagtacgctaagagaatctttagatagggcgatttccagcttttctcatgatagatatgcgcccaaagtcgatcatgatggtattgtgttgattttgagtgatttaggggaagatgcacagagactacaacagaaattttctaaatctagctcagtttttgctcagcatttcaaaacaatatttgaagcatcgtttgctgatacccacgttgatttacacggtctaattgatttagataaatatgcgctaggtaaagaagaagcttcttatcttgtgctatgtgccctctgccaaacattacggacagcgggtgttgcatttactgcgattactgggactgatttgtgtagtaaattggctgctgccagtgctgttggcatgattcagcatcaggatggtttgcgttatctcagagtatgtctgcacaaagaagcaatgataggttggcaacttggcatcccatcggtttctctgattgatatatggcatggtgggaactgttcagcactttatcaggacatcgaaagcttcaggggttggttagaagagccaaatcaaggaacgctgttaaatagcggcgtagatagaaaaacgctgtttctcagtgttacggatggttttcaaacagcagaaggctcatcagtgcttcccctattgggcgagcatgaggatgcaaaagattgggacactatttgggctaccttgtatgaagcgggtctaaaacttcattgggaagggctatatagtggtactcgtccccgacgtctcgtgctgcccaattatgtttttcaacgtcgccattattggcctgagaatgccaaaacgaaacaattacatgaagtcagtcctgaagattgccatcgatttaaggtgttatggcaagaggaaacagtaacgctggcagccgtgtctgacattaaaccgtgtgttattctgattgaagggccgatagatgacttaccgatggagaacttaccatcagaggtaacgttgggggttctgcccaaagggtggcaagaaagagctgtgatcgacacggtattgactgaataccatcttgatcgttccacgcctgtagagctactcgtctggtttagacctcatgatagcttttctacgggatggcatagtgacagtcagtcccatgaaaccatcgtgaccgcggttgaacaacgtgctaaagcgttattaaacctgacacaggcgcttctagaacgcacaaattatagtcagttccggtttggggttgttaccgaaggggttgaaagtatttctgagcggactgcagtgaatgtggttgatgggctggtcaatggatttatacagacgttgtgcatggaacaaccgcagttgcgtccaaggtgtatcgatctggatcctaatagcttagcggccgataaattcgctcagattgtgactgtattatactcgaaggatgatgaaaatcatttggcatttcgtcagggacgcagatatattcatcgcttggcaaaagctgaccgagtgctctcaaaatcggtgaccgtacgtcatgatcgagcctacttaattacagggggattgggtggcatcgggctctcggtagcgagatggttatcccgtgccggggctggtcaaatattgcttgtgtcacgtcgaggaagtgaagcgcccgatgtcgcagagagattggcgacgctcaaacgggaaagaatgtccgtgacactggttcaggctgatgtgcaagattatgatgccttacgtggtgccgttgaatcgtcagccaattttcccttagctggttttattcatagtgctggtcaactagccgatgcatcattacagaatctgactcatacacaatttcaacaagtttttcagtcaaaggttgtaggggctttgaatctacatcgtctctcgcaagagatggtgtctggcactgatgtggatttattccttttattttcatcgatagccagtttggtagggtcggcagggcaggccaactataccagtgcgaatgggtttaccgctgcattggggcgttcgcgtcgtgccgtcggattgcccgctacggtcatacattggggaccctggtcagatgttgggatggcaagtgatgaacgattaaaccataagatcgagcgctctggtttggttctaattgaccccgatttagcgttgtcctctatgctagatgcgttgaccgagcatcagacagagtcagtcattgctcaatttaattggccgcgtatcaatgcttatttggcggaacgagtgccactgcctctattatctcaattcacacaaacgacgcttgaggcttcgcagtctgccaccattgatgcatcttcccatcatgaggataacaaccaccgtgatttagcaagcgcactgaaacgacaaaatgacacggttgccatacaccagttggcaaaatatgttgaaaccacgattcgtcaagtcttagctatcgacagcgaagattacattgatccgaaaacttcggtgctcgacatggggatggattctttgttgtcggtcgaattacgaaatcgttttgccaccgaattggatttgaaactaccggtttctctcatgtttgataagccgacgattgatggtgtgtcccgcttgcttctggacgaattgcgtcaacaacataaagcgagagctgaagaaacagaaactgtcggtatacactcgattcaccatgcagctcctatagttgctgaagaaagtgcttcgatggaggcagcgcagactctgtcgccaccagtgtcctcatcgccagtaaggatggataatcaagatattgcgattattggtgtcgggtgccgtttacctggcggttcggatgggattgaggctttctggaacaatcttatcggaggtgtggacttagtgagtcctttcgatggaagccgttgggatatcaatcgtttctattctgatgatgtgtcagcgaatgggcgtatgtattcgaatgatggcggacagattacaaatgtacacggatttgacaaccaacttttcggactgagtgatcgtgaagccgaatatatggatccacaacagcgtattgctctggaagttgcctgggagacgattgaatcagcggcctacacgccagaatctctcgcggatcagggaggcatcttcattggccccgggccttctgattttgcagatctatcacaacgacatactaaagcgattacggggttaatgagtcagggccaccatattagtgcgatccctggtcgtattgcgtatcactttgactggaaagggccatgtatggcgattgatacggcttgttcctcatctttagtggcggtgctaaccgcggcacaacatcttaatcagggagattgtcgggttgccttggctgggggagtgaatctgattctttcacccgccaataatatcgttcaatctaaagcaggtatgttgtctccaagcggtcgatgccggacttttgatagtgaggccgatggttatattcgctccgaggggtgtgctatggttttgctcaagcgtttgtccgatgcagagagagatggcgacgtgatttggggggtgataaaaggtggagcgaccaatcagaatggacaaggtcagagtatgactgcgccaagtgcccttcaacaggccaacctgattgagtcggtgctaaccaaagcgaatctacagccgtcaatgatccgttatgttgaggcacatggtactggcaccaagctgggtgatcctatcgaaatgtcagcgctgcaacaaacttatggaagtcaccatgatgctgagaatccactctatgtcggctcagtgaaaagtaacctaggacatacagagagtgctgcgggtgcggtggggctgctgaaagttttgttaatgcttaagcatcgacagattccgcccaacttacatctccatcaattaaacccgttattagaaatagacgaatcagtcgttcgtattccaactacacctgtcgcattaaaagacaaagaaaataatgagttaatgtgtgcagtgagttcattcgggtttagtggcacaaatgcacatttggtcgtggctgcctatgacaatgataaggctcagttatatagtaagaattctgatgatcgaattttctgtctgtcagctcgaacaccaacagcattactggccttgagtcaacgctatatttccgcattggaaagtttaccaacttcagaagacagggacacggttttggccaatagttgttattcatcacttgttggccgttgtcgccatcaacataccatgtgtttatacccagatagttatgaagatctgattgtgcaactgcgagcaatagaaacatctatgaatcaggataagttgccgttacctcagtccatagagcaagtcgctttttgtttaacattgagtacagatattccgccattgaaagcgcttgccatgaaccgacaacagcagtttgttgattcattacacatggcagaacaaagcatcctccagtacgcaagtatcgatcttcacaatgtcagtcaagctgatatcagtccagccttacgactgaaattatcctggttttgtgtgcattacgcgacaatccgatggctgatgtctgtgggtattcaaccggacaaagttttgttcagtgatgatatgtggttggttaccgcggtgttatggcaggaatgcacttgggagagtgcgatcgaacaattgctgcatggcgtttctgaggcaccatctcagttaggacggattccgttggagagaactagtctgagtggtggctctttacttttatcagattttgaaggcgtcggtcaccattgtctgaatgttgtgtcaagcaaagatgacagtttatggcgaaacgcgctagggacactttggtgcggtggacgaacagtgaattgggatgtgtatttcgaacatagtcactgccagcgtcgattgatgccaacttacccatttgaacgacgggactgttcaagaccacaaggtttggttcgtggtgaaaacccggtgaatcgtatgcttgaagagctgctcttgaattaa

SEQ ID NO: 11 - protein, 2228 aa, Vibrio rhizosphaerae MSSRF3
MSESSSWKGSYLKRLNQLSKNQLLALALRQREQLLDVNQQVMQPDDTVAIVGIGCRFPGGVVDTDSYWKLLCSSESAITQMTDERWHMQGIYDPNPEVAGKIHTRSVGLLDQVDKFDASFFSITPREAESMDPQQRLLLEVTWEAIENSGHACSEFEGKSVGLFIGMMNKDYLHLNAPDMMGVNAKHSPYYASGEAFSIGAGRLAYILGLRGPCMTLDTACSSSLVGVHLACQSLLKDECDYAIAGGSSLILSPEASIVSSNARMLSPTGQCWTFDEKADGYVRSEGCAVVALKRLSDALADGDPVVATIAATAVNHNGRSQGLTAPSSTAQAELMQTALKKARIQPSQVRFIEAHGTGTPLGDPIEMNSIQSVYGQERSEDNSLKIGSVKALIGHTEASAGVAGLIKLALCVSKNRIIPQRNFTRLNPHIRLLDGVEIATETQSFSPSESESEPFQLHYGAVNSFGFSGSNAHAIVSNVQLTSPIVTWRSPQLLTISAETPKALLDLMLRYRDYISDESLDLQALAYTSQVGRDHFPVRIALDGEALSTLRESLDRAISSFSHDRYAPKVDHDGIVLILSDLGEDAQRLQQKFSKSSSVFAQHFKTIFEASFADTHVDLHGLIDLDKYALGKEEASYLVLCALCQTLRTAGVAFTAITGTDLCSKLAAASAVGMIQHQDGLRYLRVCLHKEAMIGWQLGIPSVSLIDIWHGGNCSALYQDIESFRGWLEEPNQGTLLNSGVDRKTLFLSVTDGFQTAEGSSVLPLLGEHEDAKDWDTIWATLYEAGLKLHWEGLYSGTRPRRLVLPNYVFQRRHYWPENAKTKQLHEVSPEDCHRFKVLWQEETVTLAAVSDIKPCVILIEGPIDDLPMENLPSEVTLGVLPKGWQERAVIDTVLTEYHLDRSTPVELLVWFRPHDSFSTGWHSDSQSHETIVTAVEQRAKALLNLTQALLERTNYSQFRFGVVTEGVESISERTAVNVVDGLVNGFIQTLCMEQPQLRPRCIDLDPNSLAADKFAQIVTVLYSKDDENHLAFRQGRRYIHRLAKADRVLSKSVTVRHDRAYLITGGLGGIGLSVARWLSRAGAGQILLVSRRGSEAPDVAERLATLKRERMSVTLVQADVQDYDALRGAVESSANFPLAGFIHSAGQLADASLQNLTHTQFQQVFQSKVVGALNLHRLSQEMVSGTDVDLFLLFSSIASLVGSAGQANYTSANGFTAALGRSRRAVGLPATVIHWGPWSDVGMASDERLNHKIERSGLVLIDPDLALSSMLDALTEHQTESVIAQFNWPRINAYLAERVPLPLLSQFTQTTLEASQSATIDASSHHEDNNHRDLASALKRQNDTVAIHQLAKYVETTIRQVLAIDSEDYIDPKTSVLDMGMDSLLSVELRNRFATELDLKLPVSLMFDKPTIDGVSRLLLDELRQQHKARAEETETVGIHSIHHAAPIVAEESASMEAAQTLSPPVSSSPVRMDNQDIAIIGVGCRLPGGSDGIEAFWNNLIGGVDLVSPFDGSRWDINRFYSDDVSANGRMYSNDGGQITNVHGFDNQLFGLSDREAEYMDPQQRIALEVAWETIESAAYTPESLADQGGIFIGPGPSDFADLSQRHTKAITGLMSQGHHISAIPGRIAYHFDWKGPCMAIDTACSSSLVAVLTAAQHLNQGDCRVALAGGVNLILSPANNIVQSKAGMLSPSGRCRTFDSEADGYIRSEGCAMVLLKRLSDAERDGDVIWGVIKGGATNQNGQGQSMTAPSALQQANLIESVLTKANLQPSMIRYVEAHGTGTKLGDPIEMSALQQTYGSHHDAENPLYVGSVKSNLGHTESAAGAVGLLKVLLMLKHRQIPPNLHLHQLNPLLEIDESVVRIPTTPVALKDKENNELMCAVSSFGFSGTNAHLVVAAYDNDKAQLYSKNSDDRIFCLSARTPTALLALSQRYISALESLPTSEDRDTVLANSCYSSLVGRCRHQHTMCLYPDSYEDLIVQLRAIETSMNQDKLPLPQSIEQVAFCLTLSTDIPPLKALAMNRQQQFVDSLHMAEQSILQYASIDLHNVSQADISPALRLKLSWFCVHYATIRWLMSVGIQPDKVLFSDDMWLVTAVLWQECTWESAIEQLLHGVSEAPSQLGRIPLERTSLSGGSLLLSDFEGVGHHCLNVVSSKDDSLWRNALGTLWCGGRTVNWDVYFEHSHCQRRLMPTYPFERRDCSRPQGLVRGENPVNRMLEELLLN

SEQ ID NO: 12 - DNA, 5202 bp, Vibrio rhizosphaerae MSSRF3
atggcgaaggagactgaattgccccattttcgagaccaacttaatcatctgaccaaagctcaattgagtgaattggtcgtagcattacgtgaggaagtgttacagctaaggccagatgaagttagtgaagtgcctatttatcagacccattggcaacaaatatctctaccgcaacttgaagacggagagatatctgctcggttgatgatcattatcccttcccatgagagtttccctgaaatatcgatggaggacagtggcattcaggttgagtacttcgtactggatacaactcaggggtggacgccacaaaacatatcaagacaactagaggggcgtctcgaatatattgatatattaatctttcttttgccaaatacttcatttgatatctcagcaccaaaggctgcgcagacgatgttagatatttggtcggcaacaatggctttggctgctgtgatcgcagagcgggtcagtcctcccagattatggttgttcacgcattgggggcagtcgttacccgaggatgaacatgaaattgatactgtcgcttcccctctgattgctttaggaaaatctttatcattagagtgtcctcggatgtgggggggatgcatcgatttagatacgtcaacattgtctctcacctctgcgatcgaagaagtcttgtgtgatgcgggggatgaagaggtcgcctaccgagcaggaatacgctatatcccggaacttcatcgctcgacgacggctacaaaatcgaccggtgatatggttcgtctaaaaccgaatgccacttatctggtcactgggggaatgggaggcattgggcgtcggttggtgtcgcatctactcaatcatggcgtatctcgagtcgttgtatttggccggagagggaaagaggtaccagagattgaacaaaccttgaatcattggcatcgaatgtacccgcatgctgagattgatttgatttcggtcgatttgggcaacttgtcagcagtgagagaagcgttagatgacttacgtcggcagggacccgcattaaaaggtatttttcatgtcgcgggcagtagccagcaagtcgctttaaatcatctggatatcgactcgattgatagcatgatgcgagcaaaagctgctggcgccttgttcctcgatgtgctgacaagagatgcggcccttgatttccagatttatttttcatccatatctggttgctggggaaccgcatcgttgattccttatgcgatggcgaatcgattcctagatgctttgagtacgtatcgtaatcatcagggacacttcacgaaaagtattgcttgggggccttgggcaaatatcggaatggttattgaccagcagcaggagtctttcagccatttaggattgggtttgattgaaccgaatgtcggtatggcgatgcttggccgtttaatgaatacggttggatctcaggttcaggtggttgatgtcgcgtggcaacgttacgctcagcatctaccgatggacaagcatttgcgttggtttcgacatttgattggtggtgctgagaccatataccaaccagttgtggaaaatacaagtcttgaaaatttctctgacccgcagcaaacgttggaggtcttgtgtagcatcgtgagtgagttactgggtgaggtgttgattgagaatgcggtttacaaaccgacgcaagagattggtttaacatcgattctcggtgttgagctaagtcagagaatccgtcagcggtttgaagtgccatgccgtccaactgtcatttacgattatccgaacctctacgaaatgtcagtcgatatctctgagcaatggtacaagctatatgctcagacgatgaaacaggaagcaaatgctcctaccgttgaacaaacgcattctacccatacaaacgcacagggaaatgaggctatcgctattgtggcaatggcttgtcagttacccagtgctgataacccagaatccttatggacgatgatggagcaagcgctgaatgatggcaaagatgtgattaatcgcacgcctgttcatcgttttgatctgtcccgtttttgttcgatgaatgatgaatcaggtaaggcatttaaccttgctgggggatatttggacgatatttcaggttttgaccatgcatgttttcaattgtctcataatgaagcgaagttcatggatccgcagcaacgactagcgattgaaacaacgtggcgggcctttgaagatgcaggtatagaaccgcatcatttgatcgatggcacatcgagtgagatggccgatacgggggtgttttttgggattggtcagaacgaatatggtccactgtgtcgatcagatataagtagtgagtacgcagggctgatgcctacggggcaatcgatgaacttgattgcagggagaatctctcatctgtttggactcaaggggccggcgattgcatatgatactgcctgttcatcttctcttgttgcgttagatgctgccgtacgtcaccttcgtcgtgaatcagggaaaatcgccattgttggtggtgtgaacacattaattgcaccagaaagttttgtgttgttgtctaaggctggtgctttgtcgaagagaggacgaggatctgcatttgatgcatcggcagatggttatgtcagagccgaaggttgtgttgtgatggtgctcaaacgtttgtctgatgctcaagcggccggagacagaattcatgcggtgattcacggcagtgcggtcaatcatgatgggcggagtagtggactgaccgctcccaacgggagggctcaagagcaggtgattcgtgccgcattaaaagatgctgaggtttcacccgaacaggtgactttggttgaagctcatgggacaggaacgccacttggcgacccgattgaatatcatgccttacgtgctgtctatacggatggaaagtcgaggtctgcgcctctgaaattagggaccatcaagtcattcatcggtcatacagagtctgcttctggtttagcgggattattaaaactggtattgaccctgcaaaagcgaacaatgcctggccagttacattttaatacgatcaatccttatattgatgcgacgcaggggatcgtcattcctcgtcaatctcaggtactagaggatacggggcgattattgggtgcggtcagttcttttgggtttaacgggaccaatgcacatgtaattgtcgagcgtggggaaggggggcattcccgcgccttgccagcccaacctttccatcgtgttcgctgttggtatagtgaccgcccactcaatgaaagtgtcggacttgcacaagcattttctcagcgccaggaacaagcaacccaatggtgtgttcctaccaagtattatgttaagcggtggaaagatgccgaggttgagtgccaccaagcagcgtcggaactgtcaacactttcccatgtgctacttgtctctcagaagggtgccgagcggttgtgtcagcaatggtatcaaacattggttgcacaaggtttgactattacggaagcaagtttcgaacaagttccttctttacaggggcgatttgatagaaccgtcgtttgtttgaatgatggagcagagtcgacagaagatatgtctaccgcttcttggcagaactcggccgcgtctgtttggcaagtcttacaagatcttgtgacagcacgttttgaaagtggccatctgcttgtgtgtgaatccagtcggttgaatcgtgcctccaccgggggcaattcctgctggcaggcagttttatcctgctatcgtaaagagcgagctgatttttcagcgacactggtttcatgcaatgctgaattgtctgactgcttaccacagtctatgacatggttgctcacgacaaaagaaacggagtgttacctctcagcaaatcaagcttatatcccacgactgacagagcttcctccgttttcagcgaattacatgccaattctgtcggcacaacatagttacattgtgacagggggaacggggggagcgggcgcaagtctcatccaatggctgttacgttgtggagcacgacatatcattaatctgaatcgacgtcctcctgatgaggcgcaaacagtattgttgcagcaatggtgtcgcgatttcaatgcaagcattcaaacggtcaggggggatgtctccaattttgctaccttacaaggtgtgctggagaacacactcgcaaaagcgcctccattgaatggtattttccattgtgcgggtgtgctggagcaaaatttgtttgaacagcatacctggtcacaagttgaagaaatgttgcatgcgaagcatcaagggactctcaatcttcatagagcgaccctcgatcaaccattacagtattttgtcgtgttctcttcattggtgtcattgctaggtcagagtggtcagtcaggttatgccttttcgaatgcgatggcagagaaggtcatcctacatcgccgtactcagaatttgcccggactggccattcaatgggggccttgggctggtggtggcatggccagtcgggcgggaaaaggattagcagcacactatcgacaaatgggtgtctcgatgttggaggaggagagctacctccacgcgctagggggggtattggcgagtgctaccgaacctgagtctttgccaccggttattgcgattggtgatattgactggcagcgttatttatcgaccacaatggcaccgctgccattgtgcgaagacttacattcagtcgagaccggactgttgcatgatcaaaccgaaaatgatgaaccggatgaaagtttatcatcttctctatcgaaggttgctcctgaacgccgcatgcgtttgctgaaagagcgccttaagcagattgtaagtgagtgtcttgaccagaatgcccctcaattaattgaggattttgaaggctttagtgcgctcgggatcgactcacttcattctatgattctccataaaaaactagaaaaagaactgggttgtccgttgccccaaaccatcgccttcgattatccgactattactgcactatcaagtttccttgccaaacatcaactgggttcgttgtttgtccatacaccttcaatggcgcaagaagagcatgagactgatgagatggtgaattatagcgaagatgagctggcacggattttacacgaagagattgaacgtctggaggaaacctcgtga

SEQ ID NO: 13 - protein, 1733 aa, Vibrio rhizosphaerae MSSRF3
MAKETELPHFRDQLNHLTKAQLSELVVALREEVLQLRPDEVSEVPIYQTHWQQISLPQLEDGEISARLMIIIPSHESFPEISMEDSGIQVEYFVLDTTQGWTPQNISRQLEGRLEYIDILIFLLPNTSFDISAPKAAQTMLDIWSATMALAAVIAERVSPPRLWLFTHWGQSLPEDEHEIDTVASPLIALGKSLSLECPRMWGGCIDLDTSTLSLTSAIEEVLCDAGDEEVAYRAGIRYIPELHRSTTATKSTGDMVRLKPNATYLVTGGMGGIGRRLVSHLLNHGVSRVVVFGRRGKEVPEIEQTLNHWHRMYPHAEIDLISVDLGNLSAVREALDDLRRQGPALKGIFHVAGSSQQVALNHLDIDSIDSMMRAKAAGALFLDVLTRDAALDFQIYFSSISGCWGTASLIPYAMANRFLDALSTYRNHQGHFTKSIAWGPWANIGMVIDQQQESFSHLGLGLIEPNVGMAMLGRLMNTVGSQVQVVDVAWQRYAQHLPMDKHLRWFRHLIGGAETIYQPVVENTSLENFSDPQQTLEVLCSIVSELLGEVLIENAVYKPTQEIGLTSILGVELSQRIRQRFEVPCRPTVIYDYPNLYEMSVDISEQWYKLYAQTMKQEANAPTVEQTHSTHTNAQGNEAIAIVAMACQLPSADNPESLWTMMEQALNDGKDVINRTPVHRFDLSRFCSMNDESGKAFNLAGGYLDDISGFDHACFQLSHNEAKFMDPQQRLAIETTWRAFEDAGIEPHHLIDGTSSEMADTGVFFGIGQNEYGPLCRSDISSEYAGLMPTGQSMNLIAGRISHLFGLKGPAIAYDTACSSSLVALDAAVRHLRRESGKIAIVGGVNTLIAPESFVLLSKAGALSKRGRGSAFDASADGYVRAEGCVVMVLKRLSDAQAAGDRIHAVIHGSAVNHDGRSSGLTAPNGRAQEQVIRAALKDAEVSPEQVTLVEAHGTGTPLGDPIEYHALRAVYTDGKSRSAPLKLGTIKSFIGHTESASGLAGLLKLVLTLQKRTMPGQLHFNTINPYIDATQGIVIPRQSQVLEDTGRLLGAVSSFGFNGTNAHVIVERGEGGHSRALPAQPFHRVRCWYSDRPLNESVGLAQAFSQRQEQATQWCVPTKYYVKRWKDAEVECHQAASELSTLSHVLLVSQKGAERLCQQWYQTLVAQGLTITEASFEQVPSLQGRFDRTVVCLNDGAESTEDMSTASWQNSAASVWQVLQDLVTARFESGHLLVCESSRLNRASTGGNSCWQAVLSCYRKERADFSATLVSCNAELSDCLPQSMTWLLTTKETECYLSANQAYIPRLTELPPFSANYMPILSAQHSYIVTGGTGGAGASLIQWLLRCGARHIINLNRRPPDEAQTVLLQQWCRDFNASIQTVRGDVSNFATLQGVLENTLAKAPPLNGIFHCAGVLEQNLFEQHTWSQVEEMLHAKHQGTLNLHRATLDQPLQYFVVFSSLVSLLGQSGQSGYAFSNAMAEKVILHRRTQNLPGLAIQWGPWAGGGMASRAGKGLAAHYRQMGVSMLEEESYLHALGGVLASATEPESLPPVIAIGDIDWQRYLSTTMAPLPLCEDLHSVETGLLHDQTENDEPDESLSSSLSKVAPERRMRLLKERLKQIVSECLDQNAPQLIEDFEGFSALGIDSLHSMILHKKLEKELGCPLPQTIAFDYPTITALSSFLAKHQLGSLFVHTPSMAQEEHETDEMVNYSEDELARILHEEIERLEETS

SEQ ID NO: 14 - DNA, 6150 bp, Vibrio rhizosphaerae MSSRF3
gtgacggcaaacaataaagatgtgctgctgcaatcgattaaaacgattaaagcgctcaaaaatcgacttgcacaatatgaaaaaaatcaccaaacagatattgcaatcattggtattggctgccgatttcccggaggggtccgtgatctctcgtcattctggaatctgttagatgccggtaaaagtggtgtcattgaagttggcaacgaccgatggagtaatcagcagtttgttgattcggattatgatgcggtcggtaaattagtctctccctatgccgggttactggaacgcatttatgactttgatgctgaattctttggtttgtcagcggttgaggcagaaaaccttgacccgcagcaacggctgttgctggttcaaagctgggaagcattagaagatgccggttatgatatacatgcattgcgggggagtgatacgggagtttttgtcggtattggtagccaagattatggcatggcactatcggctgatgttcaacatgcgaatgcttatattgcatcgggcaattcgccaagtatggctgccggacgtttatcatatttctatgactttactgggccaacgatgtcaatcgatacggcttgctcttcttctcttgttgctgtgaatgaagcttgtcggcgcttacaggatcatcattgtcaattggcgctggctgcgggcgtgaatgctgttctgaccccacattccggggtgaatttttcacgggcacgaatgttgacttcagaacgcgattgccatgtatttgatgcgcgtgcaaagggttatgttcgaggtgaaggctgcggtgttgtcgtactcaaacgattgtcagatgctttacaagatggagatcgtatccatgcggtgatcaaaagtgtcgcgatcaaccatgatggtcatagtagtggattgactgttccgaatggttcggctcaggaagcggtgattcaatcagcacttgctcaggccaatctcagagcagaggacatccaatatgttgaagctcacggcacaggaaccagtttgggtgacccgatagaggcacgtgcattggcatccgttttttctttaggacatgacgcgcagttgccaattcatgtcgggagtgtcaaagctaacttagggcatttggaagccgcctcgggcattgccagtctgattaaagccgctcttatcgtcagtcgttcagaagcccccccacaacgcggattcgagcaaattaacccgaagatcgactgggatagtgatgtttttcgtattcctctgacaaccgaaaaactgagctgccctgatggggcaagcgtgagtgctggtattagtaactttggttttagtggaaccaatgttcatgccattgtgtctgctgtatcggtcaataaagaggtacatgcggacaagacggatacatcgatgacggggccagtggtacttgccttgagtgcgaaaacatctggagcgctacatcgtcatgtcgaagatatgattcgctaccttgggagccagtcgacacaaaccttacccactgtgagttacacctcgacttgtcgacgggcagctttgtctgaacgaattgctgtttgtggggctgaccccgatgagttgatcaaagcattacaagaagcgatgcgaggaagagtgtcttctatgcaacgcaatcccatcgtgctggttttgtcaggagaggatgatccagaccaattacgtgaactcatttcaacagagaagcatgaagacatcgtgacaggctcttggtctcaggaaactgagcatgttcgattgcatcgtggtttactcgagcaactcgcacattatggtgtttaccctgatcagattttggcggaaggcattgaacctgcgtatattcatgcctggttgacagggacagaagcgcctacacaacaaccttatttgatacggtcgtcagcagcacctgatgtttggctgattgggccgaatgcgactctacaactggagcaaccagaagatatggctgcggcaatgtcgtcatctcttgtcttagggcaaagatgccctgattttctggacaaggataagtgtgagtttcaggtattggcgggtcagaaaacattatctcctgcactggctaaactattttccgcaggtgtggcgattcagtggtcatcttgttattcccaaaagtatgctgtgattgacgattttccgaagcgtcaatttgactataaaacattcaagtcccctcgggttgattgcatactacacaaaaaaaatatgctcaggtctgatattcatccaatggtcacgcataaactcagtcaaccgaatgagatgttgacctatgaattggatgcagagacaccatggctcgatttcattgaacagcatcgtgttcaggggcatcgaattttacctgcctcattattaatcgaactcatgcatcatttagcggcagatgcgctggatgttgctcagttacagttgtcagcaatcgagtttttccatcccgttgatcttgatttagctgataggtcataccttgctcaggttcaacgacacccctctgatgggggaacaccacaagtcgatatcgttttgtggagtcgtggttcgacagttcgagataaagattggattcgtcatgcaagtgcaaaatgtacccctcgaacagaatcagttgtgtccataattgacctagagtccttccacgttgctgatgagtatcgctgggatattctagatgttgatcaattgtatgcgcagcatcatgaagccggggtgatattaggggaacaattccggggcgtaaaacagttgaagtcaaataccaatgtgctggaaggtcaagttgtccttgaaaaagcagatctggccgaatcgacgacattatttaccctgttacttgatagttgtttccagattagcggtggccagccagaaacgaactctgacgttcatttgcttgcgtctctgggaaatgcagtatttgaaacggatttaccatcgagcttaattgtgcggatgttatcgaaaccaagtaatgatgggcgttgttttgatgtattgattacccagcaaaatgggcaagttgtgggggctttttctgatgtcttctttaaacgattgccgaaagcttcaacgcattcaacagcagttcagtcaacctctgaatggttgtctcctgctcaatgtttttattctcagacctggcaacgatcatcttggaaaacagaggggggggctgaacccactagctttttatctccttctgctttatttcagaatgttgaaacagctgatttttgggcagagcgatttggattggaaacttataatgcttatcggcaaggtgttgagcagttctgtttaaaagctgttgtcaaaacattccgagaattgggatatgttgacaacatttctcttccgaaagcattggcccaatgccggattcatgagtctcagcagaaactgttttctcacctttggcacttgttgaaaaagacaggccaagaggcctcggttgctgatgagctagcccatatagattggacacaactttccgtatcttatcccgagtacgactcggaaaccatattcatgcaacactgtgtctctgctttaagaggtgtactgctgggtgaacaagacccattggatatcctattccgggatacatcaatggttggtagtgatgctgtttatttgaattcccccatttctagggcgctcaatgcgcagcttgggcagatggtagcggccttgagtgcaaagcgtccgttaaggattttagaaattggagcaggaactggaggaacaacgaaaagtgtacttgaggctttgcatgacctccctgttgagacttactgctttacggatatctcatcattatttcttaaaagggcggaggagatattccagaaccattccttcatggaattccaaattttagatatcgaacaaccgattgccgagcaattatttgaagctgggacttatgatctcatcattgcagcaaatgtcctgcatgcaacacgctctatctctcagacactgggacacgttcgagagttgctcgcccctgatggatacttgttgttacgggaatgtatttcacaacagttggctgccgatttgagttttgggatgacagaaggttggtggcgttttgaagacacgatgctacgccctgattatgcggtgatgagtgctgaacaatgggagcagcaacttttagaacacgatttttcttacgctcagtctatcctgcctcatccaatgagcgctgaagcgctcattgtggctcaagctgaaaattgttccgaacaggaacaatggcttttaattcaagatggtcaggcgcaaatctgggtcgaagagcttaagcaacaaggcgctttgtgtgaatgtctttcttggaaagatatcatgagtggtgtcaagttacccgcacaatactcgccagattaccttgtttgttttcctccggatactgaagacagtccagacatcgttgaagcgagtatacagtgtgctgaatcaacgatgtcactgtgccgtcatttatttgaagacaaacgtttttatcaaacccgtgtctggggggtgacgacacaagccgaaaaagtgttgccgtcggatccactcacaggacttggccaatcgaccatgacgggagtgatgaaaagtgcagcattagaatacccaggtcggattggcggtgtgattgattgtgaaaaactgtgcggtgatcaagacagagtatcgatgattcaacaaatgttgtcccatattcggcagccaggacatttacgctatatcgcgatcagagctaatcaaccctatcagccattattgttgcctgacatagccaaagaagaaggggctatgtctaattcgatagttcaaagcctctgtgctggaggtgctgtgctcatcacgggtggttttggtggcattggaactgcgttgacgaatgctttaggggcatttatgaagtcaaatggaaattatcctacaggacaaaatacaaaaggtactttggtcttggttagccgtcatatcgaaggcagctctcaacaaactcaggtacatgctttggaaaacatgggttttagagtgctagcgattgaagcggatttgagtattcccgctcaagtcgatgatgtcttcgaacagatagaagcccaatctctccatcttgaacatttaattcataccgcgggtatcggtggtgatcagatgattcgtgaaagtcagtcgggtgatttaagagaggtggtgttatccaagctagcaagcacttggtatctgcatcaacgcgcaccaagggatcttaaaacattcttagttctctcttcgatggttgggctatggggggctaaagggaaagcacactacgtgtttgcgaatcattttgctgatcgtgtaatacagcttcgtcgttcgctagggcttgctggctctgttgtccagttgggacctgtggatgccggaatgttaaatcttgcgggtaaggaagcggcactgcgggttggtgttcgaagtttcaaagtgcaggacatcgcaaagatgctcattggtgaattgcgagctgctgagtgtgcgattttagacattgattggcgcatctttaaacctttctatcgaggaagttggttgcagtcctattttgagaaacttggtttgagaagttatcgcagtgatgcctctgttatgactcatgctgatgaatattcattctcaaatcgttttatggcacaaccagtcgcgaagcgtcgaaaatttgttgaggcagaattgttcagtattttgcgagatgtgttgggattaagcggtgacatcaaaagttatctggaaacagggtttcatgaactcgggatggattcgttattaacgatgtcttttgcagaaaaggttgcaggcatgtctggaaaagaagtgtcgtctattgatgttttggacaatgcaaatcttcaacgcttgagtcagtggttatttgaccaattcaaacatcaactagagaagaccgatgtgacacagaaaaatggacagcatatttcttctgtgttcaacgcgtctcaaccgcaggactccgttacatctggagtcactttggaacagatagagcatgaattaaaagccatgcaagaaacattggaggatatctga

SEQ ID NO: 15 - protein, 2049 aa, Vibrio rhizosphaerae MSSRF3
MTANNKDVLLQSIKTIKALKNRLAQYEKNHQTDIAIIGIGCRFPGGVRDLSSFWNLLDAGKSGVIEVGNDRWSNQQFVDSDYDAVGKLVSPYAGLLERIYDFDAEFFGLSAVEAENLDPQQRLLLVQSWEALEDAGYDIHALRGSDTGVFVGIGSQDYGMALSADVQHANAYIASGNSPSMAAGRLSYFYDFTGPTMSIDTACSSSLVAVNEACRRLQDHHCQLALAAGVNAVLTPHSGVNFSRARMLTSERDCHVFDARAKGYVRGEGCGVVVLKRLSDALQDGDRIHAVIKSVAINHDGHSSGLTVPNGSAQEAVIQSALAQANLRAEDIQYVEAHGTGTSLGDPIEARALASVFSLGHDAQLPIHVGSVKANLGHLEAASGIASLIKAALIVSRSEAPPQRGFEQINPKIDWDSDVFRIPLTTEKLSCPDGASVSAGISNFGFSGTNVHAIVSAVSVNKEVHADKTDTSMTGPVVLALSAKTSGALHRHVEDMIRYLGSQSTQTLPTVSYTSTCRRAALSERIAVCGADPDELIKALQEAMRGRVSSMQRNPIVLVLSGEDDPDQLRELISTEKHEDIVTGSWSQETEHVRLHRGLLEQLAHYGVYPDQILAEGIEPAYIHAWLTGTEAPTQQPYLIRSSAAPDVWLIGPNATLQLEQPEDMAAAMSSSLVLGQRCPDFLDKDKCEFQVLAGQKTLSPALAKLFSAGVAIQWSSCYSQKYAVIDDFPKRQFDYKTFKSPRVDCILHKKNMLRSDIHPMVTHKLSQPNEMLTYELDAETPWLDFIEQHRVQGHRILPASLLIELMHHLAADALDVAQLQLSAIEFFHPVDLDLADRSYLAQVQRHPSDGGTPQVDIVLWSRGSTVRDKDWIRHASAKCTPRTESVVSIIDLESFHVADEYRWDILDVDQLYAQHHEAGVILGEQFRGVKQLKSNTNVLEGQVVLEKADLAESTTLFTLLLDSCFQISGGQPETNSDVHLLASLGNAVFETDLPSSLIVRMLSKPSNDGRCFDVLITQQNGQVVGAFSDVFFKRLPKASTHSTAVQSTSEWLSPAQCFYSQTWQRSSWKTEGGAEPTSFLSPSALFQNVETADFWAERFGLETYNAYRQGVEQFCLKAVVKTFRELGYVDNISLPKALAQCRIHESQQKLFSHLWHLLKKTGQEASVADELAHIDWTQLSVSYPEYDSETIFMQHCVSALRGVLLGEQDPLDILFRDTSMVGSDAVYLNSPISRALNAQLGQMVAALSAKRPLRILEIGAGTGGTTKSVLEALHDLPVETYCFTDISSLFLKRAEEIFQNHSFMEFQILDIEQPIAEQLFEAGTYDLIIAANVLHATRSISQTLGHVRELLAPDGYLLLRECISQQLAADLSFGMTEGWWRFEDTMLRPDYAVMSAEQWEQQLLEHDFSYAQSILPHPMSAEALIVAQAENCSEQEQWLLIQDGQAQIWVEELKQQGALCECLSWKDIMSGVKLPAQYSPDYLVCFPPDTEDSPDIVEASIQCAESTMSLCRHLFEDKRFYQTRVWGVTTQAEKVLPSDPLTGLGQSTMTGVMKSAALEYPGRIGGVIDCEKLCGDQDRVSMIQQMLSHIRQPGHLRYIAIRANQPYQPLLLPDIAKEEGAMSNSIVQSLCAGGAVLITGGFGGIGTALTNALGAFMKSNGNYPTGQNTKGTLVLVSRHIEGSSQQTQVHALENMGFRVLAIEADLSIPAQVDDVFEQIEAQSLHLEHLIHTAGIGGDQMIRESQSGDLREVVLSKLASTWYLHQRAPRDLKTFLVLSSMVGLWGAKGKAHYVFANHFADRVIQLRRSLGLAGSVVQLGPVDAGMLNLAGKEAALRVGVRSFKVQDIAKMLIGELRAAECAILDIDWRIFKPFYRGSWLQSYFEKLGLRSYRSDASVMTHADEYSFSNRFMAQPVAKRRKFVEAELFSILRDVLGLSGDIKSYLETGFHELGMDSLLTMSFAEKVAGMSGKEVSSIDVLDNANLQRLSQWLFDQFKHQLEKTDVTQKNGQHISSVFNASQPQDSVTSGVTLEQIEHELKAMQETLEDI

SEQ ID NO: 16 - DNA, 8853 bp, Vibrio rhizosphaerae MSSRF3
atggaccagacgcatgaattactcttagccagacagaatgcattgataaaacagagtcgtgagaagatccgtgctttgtcagcacagattaatcaaccgattgcgattgtagggatggggtgtcgtgttcccagtgcagataatgttgaagcattatgggaattgcttcaacaaggtgacgaggctatatcggaggtgccattgacgcgttgggacatcgatgattattacgatccgaaccctgggacaccagaaaaaacctatagccgtcgtgctggttatctgtcagatattgagcaatttgacgcgagattttttggtatttctccgcgagaagcccgtcaaatggatccacaacagcgtttattaatggaagtcagttatcatgcacttgagaatgcaatgatatctgctgagcgtttacggaatgaagccgtcggtgtttttgttgggattagctcgagtgaatacggtgcaatgaccttttcaggggatcgtgatgcgagccctgatgcctactcgattacaggtacatcgttgaactcagcggcagggcggctcgcttactactacggttttaatggtccggccttagcaattgatacggcgtgctcttcctctttggttgcaatttatcaggcttgtcgaagtctcgtgaatcaggaatgccatacagctttggctggcggggtgaatactttattgagtcccggaccaacggttgcattagcgcaaaataaagtactgagtgccagtggtcattgcagtccatttagtcatgatgctgatggattggtgcgaggtgaaggatgtggcgtgcttgtgctcaagagacttgaagatgccgtggcagagaaatgccaaatactagctgtcattcgttctgggcatgtcaaccaagatggtgccagtagtggcttaacggttccgaatggttatgcacaacaatctttaatcaagacggcattgaaggttgcccaagtggacccggcggcaattcagtatgttgaaacacatggtactgggacagagctaggggatccgattgaagcgaaggcactacgacaggcactgtgtcaagagcaggtaagacaatcgccacttttgattggctcaattaaagcgaatatcggtcaccttgaggccgcgagtggggtcgctggggtgataaaagtcgtattggccttgaaacatcacatgttgccaaaacaaatcaatgtggcacaaaaatccacattgatttattggggggatgatttagatattgtcaacaattcgaccccattgacctatgaccctgagcacccgttttatgcgggagtgagcagttttggttttagtggcaccaatgcccaccttattcttcaggacccggtcagtgctgttgttgatgtggaggctccgcaaacaatctctcctgttcttgaaaagtcgacctcagaatctcatgcttttcttggtatttcggctaaagatccacaagccttaaaattattgctgcaagactacagtgagtattttgcctcagaaagagactggcaaggggcttgtgctgctgttaacgcagggcgcagtcaatatccgtatcgtgccggttttgttgcgacagatcgtcaaacgctggtatctcaactgaaagctgccagccataatgaggatgtaagggaaccctcactgccaccgcttgttacatggctttttacggggcagggctcgcagtatgttcagatgggaaaggttctgtttgaaacccagccggattttcgacggtggttagaacacgctgacgcggctattcaacctcaccttggtcgttcaattctatctgtaatctggcatgaagctgctcaactcaatgacactcagtatactcagccggccatattctgtttgcagtatgctcttgctcaatttttgtcgacacgtgttgggatgccccaatatgtgttgggacacagtatcggcgaatatgccgcagcggttgtcgctggtatcttcgatttggatgatgctgctcggatgatcgttgctagaggacgattaatggttgaacgttgttcccccgggggaatgatggtcgtttttgcggatcgccaacaagtagaggaattgttaacgcatagccgtcaagatgtgagtttagccgttgtaaatggtcccaaaaaccacgtgctatctggttcaggggaggccattcagcatttggctcagattgctgggaaacgcgacattcggtgtgtttcattgccggtttctcatgcttttcattcatcgatgatgacccctatgttgcgtgaattcgaagccgttgtgcaacagactaattttaatcgtccgcagattagctttatttctactgcgttgggacgttccgtcgatgaagaattattagattcatcctattgggtctctcaggtcagcggggctgttctgtttgaacaggccattgcggccctgacggcagaaccggattggcaggatgcctcttctcgtttgtgtgtagagttagggccacatagacaactgattgatatggcaagaatgttacctgatacgtcagatgctcaatggttgaatatgctccaaagtgggaataaccatcatgtctgctatgaaacgatgcgttctctataccttgctggcctaccgtttcaatggccagaaatttccgcctataagagttcacagcctttgccgcagtatccgtttattcggcaaccatattggttagagggagaacgacgcagaaacgttgcttcagcaaacttagactacgatgtggtatggaatcatgtgttgttcagtcaggcaggagacacatatttaaaatcagagaatgagcagaataatttttgtttagtatgcacggaacaagagtgtcaattggctgcagctttatcgaattcaatggctcagattgggacagaatgtcaaattgtggcaccgaaagctttgattcaagacgggagtgcccttgatcattgtcattcagtgattttatatatgccagacggacaagctccgcctgaatcgcaaacatgttttgaaacattgtcgttgattgtacaaatgactcaagaccgagtggctcgtactcccaggttagtcattgtggttgatagtggcgaacaaggttataggccattggcttctggtcttgccggactttgccgcaccgtgcgtgaagaaacatcattcgatctgacgttgattggtttggatgatcgcttagacgtgacacgccgggctcagatgttagctgccgaaattcatacgcctcgttgtggcgattgggagagacgccttgtggatgatcaggttctggttccccggttggtctctgcatctcaaaccgatacgcatcagcgatcccgggtgaatgttcacccagaacggagctatctcgtgaccgggggaacggggactttaggacgcttatttgcggagatgcttctcgagcaaggtgcgaaagatctaatcctcttggttcgccagacagagcatcatgtactgaatgaattaagaacgaaatcgacgtctttgggggctcgtcttagtgtctatggtgttgatttgactgatggtgatgcaattcagaacgtttttgaagtgttgaagaatactcatctgcctttggccggcatcattcatgctgctgggcaattgtcggatgccgctttcaatcaattgaatgcagaatcgtttgcgcgtgcatttgatgttaaggctgtcggtgggtggtggcttgaccgactgagtgaagatatgccgctggacttttttgtcatgacatcctcgattgcatcggtgttgggggcggttggacaagccaattacgcatccgcgaatggttatcttgatggtctcgcattacaacgatatgagcaaggtaagcctgtcatgagcatccgtctgggggccgttgctgacactggtatgacagcacatgacgatgtgaaacgccagctccgtcgtatggggatcaagccgttgcctttgtctgtcattcgacaccggtgtgcagcatggttgggggcgtcatcaccattgtcgatgttagcagagattgattggcccagagttcgagcccgggaaggggagactgcacgcccattcttgacggagatgttgccgcccataaatggcgcaataacccagtcaatgtcctcatctctatccggaaataaggggaaatgccaacgcgaacagattgaaactatcgtgatcgatacgattgctgcgattctcggtatatctgagtctgaccagatcggaaaaatggacacgctcaatgcgctgggtatggactcagtcactttggttgaattacgtgaacaattgcaattgcaacttggggcggagattccgacaaggttcttgtttgactttccacaagtgggcaagcttattcggcatttacagattgaactccatgatcatgacaatcaacagcgtgcgacagaggggtatgatagtacgtctgagacgctgcccgcagagtctgatgagcaagcaagtcgtcgggatattgcgattgttggtatcgcttgtcgtttccctggtagtgtgaactcccctgaagcgttttggtcagcgttgaaggaagggagagatttagtcggggagattgatgcacttcgttgggatgttcgatcattgatgcgtcaagggcagctagtcacggcacgtgctggtattatcgatcacttggatgctttcgactgtggtttgtttggtattacgccgcgtgaagcgcagtgtatggatccacaacaacgattattgttggaggccgggtgggaagccctggagcgcgcggggtacgatttttcagccaccgatgtgctgggggggatattcattggtcctggtcctgatgattatgcgaagcgatttccgacggatgcagcgaccttatctcatcaccatagcacaggaaatgcattaagtgttactgctgggcgtttagccttcactctggattggcaagggccaacattggcggtggatacagcatgttcttcatcactaatggctgtacatctcgcaactcaggctttacgtcaaggtgaatgcagtattgcgcttgcggggggcgtgaatttgctgttgtcaccagaaacatctgttttactttctaaagggagtatgttagcgcctgatgggcgttgtaaaacatttgatgccaaggcgaatggatatgtccgcagtgaagggtgtggtcttgttgttcttaaatgcttaaaagatgccgttgctgatggggatgatgtcatcgcagtgattcgaggatctgcttccaatcaggatggtcacagtcaaggacttaccgcacccaatggacagtcgcaacaacgtgttttgcgtcgagctttacgggatgctgcggttgaccccacgcaggttgaactgttggaagcgcatgggacgggaacaccgttgggtgatcccattgaaatgtcagcagcgcaagctgtttatgcagagaggctgtctcgtcattccccgttatgggtatcgtctgtcaaaacaaatatgggacatgcggaagcagcggcaggcattgccggacttataaaaacagcgctttgtttgtaccatcagacgattgtgccccatctacatttggcgcaattgaatccagaaattcagctcgacgacacggttcgtatcccagacacaacacagccatggccacatgatggtactgagcgttatgcagccgtgagttcatttggtttcagtgggacaaatgtccacatgatattgcaatctgcacctgaaagagcctctatatctacaaagagggatacaccagatggtttacgaatctcagccgcgacaaaagttgcactcaacgacatgatgcatgcttatcttgatagattggttgtgctacctgcaaaacaatatgaagatttttgccagcagagttggcgtcgtgctgagttggcgcatacaaggatatttgcagctgcgccgcagggggagatgattgctgaacttcgctcaatattggaaagttcacacacggtaacaagtgtgggacaccaaacgtcggctctgtcgatgctgggcggatctttaccgtgttatccattccagcggcaacatttctggcaaccactctcagctgttcgaaaagaacgctccaacgcagttaatctgggcgtgagattgggccccagaagcgcaacgaaggtggtttatgccgttgattacctgaatcagccccctttcaatctcgaagaccatttggtccataacgaatttgtggtaccagccgctgcacatttagccctgattattggcatgctcagggatttgcgcgggagttgttgctgggaagttttagatgttttatgtgagggaacattcatccctgatgcagagacagaagtggccttgtatgagtttacgacgatcactgaagatgaaggcgaagcgttcagcattgaagttctaagtgaacactctgaggggatccggacacacttgcgagctagagcaaaatcgacggagactcaagctgcttctatcgtgattccagtgtcgaatccaccattaactgaaatcaatgcagccactttttatgatgaactttattcttcagaaattgccttgtctcatgaatttcgcagtattgtgtcgatccaacagtttgttggctactcgaagtcagaaatgcgttggtatgcaggtgattcagggccattagtgccgggagagctcgattcactattacaaacgatcgctttggcgacaaaggctgaacagccaggcactagtcatatgagcggggcgacaattcccgttactattgatagattacttgtggcatctctgggagatagagaggcggtcccaaacttgccatttatctgtcagacccgtctcaataaagaagatcgtggcgctgcttttgtccatgacatcgctttgtcggtcgaaggggaacaaccatttctgagtattgagggattatgtactcggtgggtgagtgcgcagcagatcaaaccgatcagagcttcttctgcttatctgaatgaagcgtgggtggctgaacctttggcgacgccattgacaccatcggaagagggggcgccaatttatgttttgttaaacacggtcgaagatgaaattgaccctgatttcgcatctgctttagcaccgctgaacatactgcatcaccctacgactcaggtggttgaacaaacgtgtcgtgaacgacatgaaagtcatgatgttgttttgatttactgtttatctcaatatgatccgcatggcagcgtagcaaacagcgacgggatgcaagggatgatggcgcttgtctctgccgtcagatacgctcataaccttagtgaaaccttgtttgactctggtatagcaacacgttttcaggtgctttcaaaagaacatgcccatccaattggcaaagtaagtggttctcctttgtttgggtttgcatcgggactttgtaaatcactgagccttgaatggtcaaattgctctgtcggaatgctcgatgtggatacgttatcattgcagcaaaacgcaacccttttggaaaatgaatggtgtcaaatgccattgagttgggtctgctatcatcaagaacaccgctatgttcgacggattggggaatttgctggcgaggagattgttgggaagaatgcttttcattgtcgtgacacggggctttacctgttgacaggaggattgggggcactcgctttggagacatgcaaatggcttctaagtcatggcgcaagacgcttcctcttgctaggacgacgtgctgaaaatgagcaaatcagtcaacgattgcaagaacttcatgacacatttaatgctcagttcgagtatagagcctgtgatgtctctgacaggacggctttagagacgttgcttgctgaggaacagatttctgactcgctgtgtggcattttccattgtgctgggacgctgtctgatggggctttctcttccttaactgatgatgacttcgctcatgttatgcgtagcaaggtcattggaagctggaatttacatacattaactcaggacatgtcgcttgattgttttgttctgtattcatcattggcttcgttactgggggcagcaggacaaagtaattatgccgctgcaaacggttttatggatcagcttgcgcattatcgacatgctttaggcttaccagcactggcgctcaattggtctggctgggacgaaatcggtatggcagcaggaaagacatcagagactgggctgcgtttattgaatccagcgagagcgattatggatttggaacgcttgatgtcatctgatgtgattcaagcgggggtcattgatgtggattggtccatctttagcgcgcttgggcatcgaccgatacccgggtttgttcaacattggatagaaagtcatcaggttgaccaaacggataccgacaatgagaaagaagacaatagcgttaatctgttggaagaacttcgagcgcttccatcaatggctcgacttgattctgttaaaagctatttacgtcaaattacaagaaagatcatgaagttggatgaagagagtgaactggatgaccaaaagccatttcaagactttggatttgattctttgatgtccattgaactgaagcaaaagctatataaaaagttgcgtataagagtacctgcaactctgatatttgactatcccaatgtagaaagtctagcaacctatattatgcaaacactagaaccggactgtgagtcaacagagaagccattgccggatacaaataagcatatggatgagctggatgaagagcaactcgcacacatactggaaacactgctgtaa

SEQ ID NO: 17 - protein, 2950 aa, Vibrio rhizosphaerae MSSRF3
MDQTHELLLARQNALIKQSREKIRALSAQINQPIAIVGMGCRVPSADNVEALWELLQQGDEAISEVPLTRWDIDDYYDPNPGTPEKTYSRRAGYLSDIEQFDARFFGISPREARQMDPQQRLLMEVSYHALENAMISAERLRNEAVGVFVGISSSEYGAMTFSGDRDASPDAYSITGTSLNSAAGRLAYYYGFNGPALAIDTACSSSLVAIYQACRSLVNQECHTALAGGVNTLLSPGPTVALAQNKVLSASGHCSPFSHDADGLVRGEGCGVLVLKRLEDAVAEKCQILAVIRSGHVNQDGASSGLTVPNGYAQQSLIKTALKVAQVDPAAIQYVETHGTGTELGDPIEAKALRQALCQEQVRQSPLLIGSIKANIGHLEAASGVAGVIKVVLALKHHMLPKQINVAQKSTLIYWGDDLDIVNNSTPLTYDPEHPFYAGVSSFGFSGTNAHLILQDPVSAVVDVEAPQTISPVLEKSTSESHAFLGISAKDPQALKLLLQDYSEYFASERDWQGACAAVNAGRSQYPYRAGFVATDRQTLVSQLKAASHNEDVREPSLPPLVTWLFTGQGSQYVQMGKVLFETQPDFRRWLEHADAAIQPHLGRSILSVIWHEAAQLNDTQYTQPAIFCLQYALAQFLSTRVGMPQYVLGHSIGEYAAAVVAGIFDLDDAARMIVARGRLMVERCSPGGMMVVFADRQQVEELLTHSRQDVSLAVVNGPKNHVLSGSGEAIQHLAQIAGKRDIRCVSLPVSHAFHSSMMTPMLREFEAVVQQTNFNRPQISFISTALGRSVDEELLDSSYWVSQVSGAVLFEQAIAALTAEPDWQDASSRLCVELGPHRQLIDMARMLPDTSDAQWLNMLQSGNNHHVCYETMRSLYLAGLPFQWPEISAYKSSQPLPQYPFIRQPYWLEGERRRNVASANLDYDVVWNHVLFSQAGDTYLKSENEQNNFCLVCTEQECQLAAALSNSMAQIGTECQIVAPKALIQDGSALDHCHSVILYMPDGQAPPESQTCFETLSLIVQMTQDRVARTPRLVIVVDSGEQGYRPLASGLAGLCRTVREETSFDLTLIGLDDRLDVTRRAQMLAAEIHTPRCGDWERRLVDDQVLVPRLVSASQTDTHQRSRVNVHPERSYLVTGGTGTLGRLFAEMLLEQGAKDLILLVRQTEHHVLNELRTKSTSLGARLSVYGVDLTDGDAIQNVFEVLKNTHLPLAGIIHAAGQLSDAAFNQLNAESFARAFDVKAVGGWWLDRLSEDMPLDFFVMTSSIASVLGAVGQANYASANGYLDGLALQRYEQGKPVMSIRLGAVADTGMTAHDDVKRQLRRMGIKPLPLSVIRHRCAAWLGASSPLSMLAEIDWPRVRAREGETARPFLTEMLPPINGAITQSMSSSLSGNKGKCQREQIETIVIDTIAAILGISESDQIGKMDTLNALGMDSVTLVELREQLQLQLGAEIPTRFLFDFPQVGKLIRHLQIELHDHDNQQRATEGYDSTSETLPAESDEQASRRDIAIVGIACRFPGSVNSPEAFWSALKEGRDLVGEIDALRWDVRSLMRQGQLVTARAGIIDHLDAFDCGLFGITPREAQCMDPQQRLLLEAGWEALERAGYDFSATDVLGGIFIGPGPDDYAKRFPTDAATLSHHHSTGNALSVTAGRLAFTLDWQGPTLAVDTACSSSLMAVHLATQALRQGECSIALAGGVNLLLSPETSVLLSKGSMLAPDGRCKTFDAKANGYVRSEGCGLVVLKCLKDAVADGDDVIAVIRGSASNQDGHSQGLTAPNGQSQQRVLRRALRDAAVDPTQVELLEAHGTGTPLGDPIEMSAAQAVYAERLSRHSPLWVSSVKTNMGHAEAAAGIAGLIKTALCLYHQTIVPHLHLAQLNPEIQLDDTVRIPDTTQPWPHDGTERYAAVSSFGFSGTNVHMILQSAPERASISTKRDTPDGLRISAATKVALNDMMHAYLDRLVVLPAKQYEDFCQQSWRRAELAHTRIFAAAPQGEMIAELRSILESSHTVTSVGHQTSALSMLGGSLPCYPFQRQHFWQPLSAVRKERSNAVNLGVRLGPRSATKVVYAVDYLNQPPFNLEDHLVHNEFVVPAAAHLALIIGMLRDLRGSCCWEVLDVLCEGTFIPDAETEVALYEFTTITEDEGEAFSIEVLSEHSEGIRTHLRARAKSTETQAASIVIPVSNPPLTEINAATFYDELYSSEIALSHEFRSIVSIQQFVGYSKSEMRWYAGDSGPLVPGELDSLLQTIALATKAEQPGTSHMSGATIPVTIDRLLVASLGDREAVPNLPFICQTRLNKEDRGAAFVHDIALSVEGEQPFLSIEGLCTRWVSAQQIKPIRASSAYLNEAWVAEPLATPLTPSEEGAPIYVLLNTVEDEIDPDFASALAPLNILHHPTTQVVEQTCRERHESHDVVLIYCLSQYDPHGSVANSDGMQGMMALVSAVRYAHNLSETLFDSGIATRFQVLSKEHAHPIGKVSGSPLFGFASGLCKSLSLEWSNCSVGMLDVDTLSLQQNATLLENEWCQMPLSWVCYHQEHRYVRRIGEFAGEEIVGKNAFHCRDTGLYLLTGGLGALALETCKWLLSHGARRFLLLGRRAENEQISQRLQELHDTFNAQFEYRACDVSDRTALETLLAEEQISDSLCGIFHCAGTLSDGAFSSLTDDDFAHVMRSKVIGSWNLHTLTQDMSLDCFVLYSSLASLLGAAGQSNYAAANGFMDQLAHYRHALGLPALALNWSGWDEIGMAAGKTSETGLRLLNPARAIMDLERLMSSDVIQAGVIDVDWSIFSALGHRPIPGFVQHWIESHQVDQTDTDNEKEDNSVNLLEELRALPSMARLDSVKSYLRQITRKIMKLDEESELDDQKPFQDFGFDSLMSIELKQKLYKKLRIRVPATLIFDYPNVESLATYIMQTLEPDCESTEKPLPDTNKHMDELDEEQLAHILETLL

SEQ ID NO: 18 - DNA, 11655 bp, Vibrio rhizosphaerae MSSRF3
atgaatcaaacacagaaaagaagtgaaaattcttacaaggatttattgaaaaattccatcctgaagattcaggagaaagatcagcgaatacaagcgcttgaaggtgagcgagataagcctattgcggtgattggtttatcttgccgtttccctggtgcttcgaatccggaatcgttctggcaattgatccgaaatggagagaatgcggtcaccacgatgacagaccagcggtgggatatggctgaatacttttccccacttgtgggggaacccggaaaaataagcacatgtcattttgggttgttgaaagatattgatcaatttgacccttctgcttttggtatctcagaggaagaaacaccttatctggatccgcagcatcggttgctacttgaacaggcatggcattgttttgaacgcgcaggtttcgatatcatggcactgagaggttcagatacgggtgtttttgttggtcaaatgaacagtgattatacaaatttgatcaaaagtactgaggatttgaatgcctatgtaggacaaggtaacgcgttgagcgctgcttcaggtcggttgtcctatgtttttgggctgaatggtccgagtgtatctgtcgatacggcatgttcttcatcactggttgctgtgcatcttgcttgtcaaagtttacggatgggagagtgctctacggctttagccggaggggtaaatttattattaagccccgaagctgcgattggtgcttctgttgctcacatgctttcagtgcgtgggcgctgcaatacatttggtgacggggcggatggttatgttcgctctgaagggtgtggcctcgtcttactcaaaaccttggagcaagcggaaaaagacggtgacaagatactggcggtaattcgaggttcagcagtgaatcaggatggacgcagtcatggattgaatgctcccaatggacccgcccagatggacgttatccgtcaggctttgtctcaggcaaagctggatcctgccgaggtaaattatctcgaagcgcatgggactggcaccgccctcggagaccctgttgaggttcaggctgtggatcaggtctatggacaagcaaaagggcgtaccaaaccgttagtgatgggggcggtgaaagccaatatcggtcattgtgaatcggctgcaggggttgctgggttgattaagttaatccagatggtccaaggcgatgtactgccgccgattacgcatttggagcaactcaacccgcactttgatggtttgagcgagtcgcttgtttttccgaagggcgcgtcgaaagatgggacgtcttcgttcacatggcatgatgagcagtctcgtatcgctgctgtgagctcgtttggttataccggtacgaatgttcatatgatagtctctcagcactcgactgagaaagatgcgctgaacgttcaagcccatgctgaatcaagtaagacacagtcccaagctgtttatccattctatttttctgcgcatagcattacgtcattgcgccaacaactctctcaattccgtgattttttgaaagaaacgcctcacctttcactgcctcttatttccggagcggtgaatagtattcgtaatcattttgcttatcgttttggttgtcatgcggcaacgaggcaagaacttttatcttgtcttgtgtcagggattcaaggtgaatactcagagccgcttggaaaaccgactatggtcgcctgttttggtccttccaatagtcaatatcccataccacctcagatctttgcctctgacacatggtgctcgtattttcggtgtattgaaacccgagtcacacaagcgactgaattcagtatcgaagcgttgatgtcgtctcaggcatatcccaccctgcgccacttgatttcacaactttcttatgtcgagcacctcgtggaagaaggggccgagataagaaaggttcaggcatctggtacaggcacgattgctgccctcgtttttgctaaatctttgacacttgagcaagcagcagatttatgtgtgcgcttggatcaaatctcaggagctgatttaacagagcaagagcaacgagttgaagcggatatccgatatctgattcaggacatacacctaaaagaagcgcagcgtcagatctttgtcaaaggacatgactcgacgcgtcaggatgtcgattgggtaaaatggccttatcacaatgtctcagaagcaaagcaaagactgcttgaggcgttgctctcacaacagcatccgtatcactggtcaatgctagactcagagattggcattgatgtttcagctttgcagatttttcctcagaagactatcgaatccgagactcagccctcatggattaatttacgcatcaatttgtttaatcgtggggcgacgctgcgatgggactatacgacgataccgcaggataaatctcaatatcccgtcttgccggattatgtctttgcacgacgtcgctattggttacccccacaagtgacagagcgagtggcgtcactcccatcaatgttctcaactgtggggcacagtatctttacaacagttttagccgtgcctgatggtggaagcttatatgcgggtgaacttgccttatcacggttaccatttttaagagatcacctcgttgcaggagagattgttctacctgcaagtgtttatataaacttaattgcagaggtgtgtgtcggtcccgatggactgcctgcgaacattgttcaaatacagatcagccaagcgtgtattttggattctcaacccattggtgtgtattgtcgagtcgacccgactgagaatggcatagcgaaggttgaaatttatacaacatcagagcgtgggaaatggacgaaacatgtcagtgcgcaagtaaatgttggttttcgtgccgagttaaatatcaagccgcttgctgatgttcggaaacggtgctctcaggtcatctctgttgaacaacacttactcagagccaaacagtcgggtattgagtatggcccttcttttcaggccattcgtcagttatatagcggttcaggtgttgcattggccaaaatcgaatggccatctgatttgccgaattactggtcaggttgtggcttacatccggtgatgttggatgcttgctttcaggtcatcagtggggcaatggggacgcaaaattcagacagaaaactacctttgtatgtgccgacagagcttcatgaaatgcatcttcagggaaatagtgctgagagcttatggtgttttgtacaaattcttgcccctgacgaagtgtttcacaatgaagcaacgatgtatgactacttgagccaacaagaaaagctttccgttgctttatatgtatatgatgaacagggaaaagcgattctctcaattaagcgttttgaggcatcgaagtatcggccggagcttaaaggggcattatggcaagaatggttgctagaaaagcaatggataccctgtcggttttcttccgagaaattagatgtcgatgtgtctgcattgctgacgcaggcacaatctcggtttagtactgtaccttatacgatgtctgtggacatgatgcaggatctggatgcgctttctggacatttcattcatcgagcattgacgcagtttggcttcgatgagcagttttttacatcattacttactgctcatacgctgcccgatgagatgctcgaacaatacgggattctgccagattatcagcgattactaaaaaaattagtttccctgagtgagcacctccctgtttcagcactgcctgctgatgaaatcgaatcaaaactgagatctaagctaggggaagaaacgcatgagttggacttattcgttcgctgtggtaacgcactagctgaggtactgcaagggcaaacgaaagcggttgatctgctattcgaggcgggccattcacaagggaccgaggcgctttatcaagatagccctgggagtttgttgttaaatcaacggatagcaacactggttgctgaagctgtaagtcaaataccttcaggcagacgattaaggattttagaagttggtgcagggactggtgctacaacgcaacaagttttggagcaagtcgatggtaaaaacatcgattatgtctttaccgacatttcgccccattttttgatgcgtgcaaaagaaaaatttggtgcattcgagtcggtagactatcggatatttgatttgcagcaaaatccactagaacaagaattcactgctggggcatttgatgttgtgattgcagtaaatgtgctgcatgcaaccgctgatctgactcagaccctgaatcatttatctctctgtctagccgatggcggcctcttgttattacgtgaggtaacagagcagcaagcctggcttgacctcagctttgggatgacacctggctggtggagttatacggacacatcgttacgtcaaaatggaccattattaaataccgaagagtggcagactttgttacatgattcggggtttgagtcaacgcttgtgacggatgagctagagcgaaccgaaagtatatttattgcagagaaaattgcgagcaccactgatgtggtatctaagggatgctacttagttcttggtgatgaagacaccatcgaaacccaagggctaaacgccctgaaaaatgagttggcgcaacgtggattggattgggtgtttatttccctgaatgaattcatgcagcatcctcgctttcaagaattagatgcattttcaacattactgatgtcggtggaagcgcagcaaggcccgattatcggtttagtctatgcgtggtctatgagtccgtcagacctaaactgtgacgacctattggagaagtctgagcgttatctgaaatatccattactgctttgtcaaacgctgttggatgcgcgctggcgacacctttctcctagctttttgaccgcaggtgcccagccacgggatcacaaagttgcacagcctttacaggcgatggtttggggacacgtgttcacttatatcaacgaaaatgcagcatttgcacggcttattgatttagatggtacttctgtttcaggtgaatgcttgtatgaagcgttgacacaaaaagaagaatgtcagttggttgttcgagaggggcagttgtgtgctgcacgtcttaaacgggcaacgttatcggttccggaagcgccagcacagcggtgtaacattgtggctgatgggacctatgtgatcaccggtggatttggcgatttaggactgcaaacggcacaaaaacttgttgaacaaggggcccaaaacttgattctcattggccgtcgtgagcgtcgagatgtcatgaaaacattggatatgctccgtgaacaaggtgtgaatgtcgttcctctttatgttgatgtcagtgatggtgaggcactcaatactgcactgaaccaagtgttagatgagttacccatgattcgaggggttgtccattcggtgggggtcttagatgatggcgtaatagaaaaacaacgctgggaaagatatctgaaggtactaaaacccaaggtctttggtgcaattcacctctatgaggcagtatctggttacgacctggatttttttgtgatctattcttcagcagcagcggtaatgggaaatccaggccaggctaatcatgccgctgccaatgcatttttagacgcattcgcttggtatctgcgtggacaaggctgccctgggcttgcgattggctggggggcttggtctgaaataggtgctgcggctgcaagagggattacagcgcgtttgactcaaaatcaatccattgcaggtaccatctctcctgagcagggtgctgatttgattgcaaaacagtttgccagtaaaaatgtgcagttcactgtattacctatagacttcgaccgaaagatagatacacagtatttaccacaggttcaacgtttgttatcgagcctactgaagagtaacaaatcgggagtccagcaagcaggaacagcacctgataatcgagaaaaggaacatgcagatcagtttgtgcaagacttgctacaaatggatgaagctcaacgacaacgtcaagtcgaaaattatctacaaggtaccttagtcaaattactcaagcaacatgggaacattgaacatcaaaccagtttatttgaattggggcttgattctctgcttgccattgacttgcgaagcttgttggaaaatcaattccggcagaaatttgagtcaaccctattgtatgactatcccaccattcattcattgactgagtttcttctccaatgtgtcagtgacaatcatgggcagcaaaaaactccgttcactcatcatgcagaaacgctttccagtgatcctgcaacgaaaactgctgatgtcgaactgcaaggagatgatgcgattgcggtggttgggatgtcctgccgcttccctgggggggcaaattcgattggagagtattgggaattactgaaaaatggtgtagatgcggttcagccgattcctgaaacacgttgggatcattcgcgatattatgacccggacaaaacgcaatcggggaaaatctatgttgcagaaggatgttttattgatgaagtcgctcagttttgtccagagagatttggtattgctggtattgaagcagaccttatggacccgcaacagcggctacttttagatgtttgttatgaagctttagagagtgcaggacagaacccaatgggattaggcggttctgaaacgggcgtcttcatgggggttatgacgcaggactacttacatttgacgcaacatgtgcgtgagaatgcgttttatgtcggaactggttctgcgaatagtgtggtcgcaggacgagtgtctcatgtcttcggtctgatggggccgacaatgactcttgatacagcctgttcatcttccttagtcggggttcagatggcatgcactaatttgcggtctggtgcatgtgatatggcgatagcaggtggtgtaagtttacagctttctccagaacctctggttatcgagtgtgcaggtggcatgctttcaccaagcgggcactgtcgcacatttgatgccagtgctgatggttttgttcgtggggaaggttgtggtgtagtggtcttaaaacgcttggcggatgctaagcgggatcgggacccaatcgtgggtgtgattcgtggcggtgcagtgaaccatgatgggcgtgccggcggtttgaccgtaccgagtgggttatcgcagcaaaaggtgttggaaaaagcgcttcatgatgcccagttatccccttctgatatctcctatattgaagcacatggcacgggaactcacctcggggaccctctggaactaaatgcattacaggcggttttcagtgaatcacgcgataaggcgccattatatgtcggttcggttaaaaccaatattggtcatgctgaggctgcagcgggtgttgccgggttgatcaaggttctactctgtttgaaacacgaaaccttcgtccctcatttgcattttgaacagcctaaccccaattttgattgggaacagagcaatatacaggtaacgacttctcttcatgagtggacgtcgaaaacaaagcgtcgagctggagtgagttcttttgggttaagtggaacaaatgcgcatcttgtagtcgaagagtttcaggaaatgatgctccctcgaaaacaaccctcagagtttgtacctttggtggcgctctctcacatcaacccgacacaactaaagcgtgatgcggcccgataccatcagttactgactgaggatacgccattgctaatggatgcagcttatactttctgtatatctcgtactgggcaaagtgtgcaggcagtctttccggcgcaaacacaagaagcattgctcgctgggttaaagagattagccgatggtgagctggatattgtagatagaaagattaaacggccacggttggagtggcagatatcggtgaatcaaagagctgcatggatgaatcaggcctgtacttactatgacttgtatcatctgtttcgagggacgattgatgccagtgttgaagcgctcaagcagcgaggtttcgatgtattgtcctcgatagaactatgtacattgaaagacggttctgtagacgaaaatatcgcgctacctgagcaagttcgtttggccatcttaaatgtagcgtatggacgtttgctcttgtcactaggcattgcacccgagtgtatccaagttgaagggttatccttgctatgtgttgcaaccctcgtcggtgtgtcggatatcgaacagatgatcgatggtatgcttgcggacagtgatactgagcttaaagctattttagccgggatccatttttctcatggtcatatgaggatgagctttaaagctgatcgtcgtttgtcctctgctgtcaatgaacgctgcgactctatttgtccaaaaacgtccgttgattatgtttggggggacagtgatatttctctggatcttgttcgaagtgctgtcaatgggaaagcgtttactgaactccaccagttactgagcatgtgctcttctctgggattggatattgactggtatacttatttttctcccgttgaaccgcatcggatagccttgccttctagtcacttcccaacgcgacggtactgggttaatgaatcactcgagcaagaacaaacacgccaagtgccactgatcagtcaggacatcactgcagctgtagatgggtgtcgatatgttgacttccacttatctgttgaacatcaggttttcctcgacgaacatcgtttgagtaatacacggatattgcctgccgctgggagtctggcttttatccttcatgctcatcaatgttcgtatcctcttgaaatgaaaggggttcgcttggttcgccccgttcattttgaatcacagttgtccgttcagcttgcgatgaagccgacaggacggacggagctattttattgtgaaccaaccatgaaagagtggcatgtctttgcaacgatggaagccattgacatgagtgaatcatcgactgagagagctctcatcacggatgataatcaacttttgtcgacattccacgctgtactgaatgcgcctaggtacaccatggataaagagaccttttatcaggcgtctatccctcgagaactgaatcttggagagtcatatcgactgattgaggaagtgcaaggtgatggtctgtgtttggttgctagaattcggaatatttcatctgaatttgtattagatccgcgtgttttggatgcctgtatacagtctgtcaacggggtgggggacaagttagagagacaatcagacggactctttttgccttatgcgatacaaagtatttctattcatgactggccgtcaggctcacatttttggtgcatgacgcagtttttacccgagaacagtcatgctgatgaattggtctacgatattaccgttgtggatgaacatgaacgtatttgtgcgcggtttgaacatgccagcttcagaaaagtcaatgtccctatggttgacagcgccaagccagagacatcgattatccatcaattgcgatggcaaccctcgcctttacgaaatacagagtccgtgaattcaatcgcaattgatgcagttgaaaacttacaaaatgtattactaattggtaaagcaaatgctgttcaacaacggttgcaagagcaattaagtgattgtgcttgtcaccaagtggatgccgcagaagctgcacggcaatcagtcgatgaacttcgccttctattggatgaatgttcaaaggctggcgcgttcatctatactgagttgcttgaagcatctgatcgatcaccacacgatgttgaaatattgactcaagatatttggtatttgcagcaatggttagttgctgcaagcaaacaaggtaagccatttatcgttctgacccgaaccggacagtctgtctgtctgaatgatgtgcccttatctgcggcaagtgtcgcggctgcggctctggtgcaaactgctgttcaagaatttccgtcattcccggttctactgatcgatttggatgacctttcgacagaaagcgaggatggtatcggatggttagcggaaatcacaccgtcggtacaagactctgtggtcgcttaccgtgagggaaaacgttaccgaagcgttttggaagccgtacattctatagaagacgtgtcttgtgggaattccgtctctcctgtcaaaccgaaccggacttatataattacaggtgggttaggtgatattggcctactcacggctgaaaagctagtctcggaaggggcgacctcattggtactcatgagtcggaacgagcataccaatacgaatgaacgtattgaagcgctgcgaaatgttggatgtcaggtcgagatccgactgggtgatgtgtccgatatgacggtcctgcaagcacatattgagcagatgacgtcttgcatgccccctgttgctggtattttccatacggccgctatttcagccaatgccctcatgacacaacagacggcggccgactgggcggatgtgttgtcggttaaagtacagggggctctgaatttacttgaaatgagtcgagagatggaactggactgttttgtcctgttctcttccattgcatctattgggggatcaatcggtcaatctaattacgcagttgcgaatgcctttctagattatttcgcacaatcttggtcgcagttaggtgttccggtgatgagtatcaactggggcgcctgggaaaatacggggatggctggacgcgctgaagcgaaaggtattggattcgaacagcgtttgagtccagagcaggctctttcatctctctgcttcctgatgcaaaacccaagacctcaggtggtatgttttagagaaaatagggctttgaatacatctggtttggagctgtcgggtggtacttctctcaatgaagagctcaaacatcgtttgtttggagaggcgttcttcaatttatcggcagatgaacaaaacaatactgtcagccagacgattcggagcatattgagcgattttcttaaggttgatcccgaccacattaaggatgagcagccattttttgattgtgggatggattcaattaccgcagtcgatttcagtcaccaagtcggtgaagtctttggggttgagcttcacgttgatattgttttcgactatcccaatttgctgagccttgcagattatgtgctgcaactgttgttacaggatgtgccttcattcgatcctgttatcagcggggaagacggagcactcgtggatactttgtcccgtttatctgttgatgagaagcaacatcatatcaaacggacgattcggaacattttgtgtgagtttctaaaggttgatggaagccatattaaggatgaacagccattttttgacttagggatggactcaattactgcggttgagttcagtcgtcatgtgggagaagcatttgcactcgaactacatgtcgacattattttcgattaccccaatttatcaacgctcactacacaggtaatggaactattgtcattcgatgtatcacctcagccaatctcagaggacgtgacaattgagcagctctctaaaatgctggaacaggaattagaaaatgacgcatag

SEQ ID NO: 19 - protein, 3884 aa, Vibrio rhizosphaerae MSSRF3
MNQTQKRSENSYKDLLKNSILKIQEKDQRIQALEGERDKPIAVIGLSCRFPGASNPESFWQLIRNGENAVTTMTDQRWDMAEYFSPLVGEPGKISTCHFGLLKDIDQFDPSAFGISEEETPYLDPQHRLLLEQAWHCFERAGFDIMALRGSDTGVFVGQMNSDYTNLIKSTEDLNAYVGQGNALSAASGRLSYVFGLNGPSVSVDTACSSSLVAVHLACQSLRMGECSTALAGGVNLLLSPEAAIGASVAHMLSVRGRCNTFGDGADGYVRSEGCGLVLLKTLEQAEKDGDKILAVIRGSAVNQDGRSHGLNAPNGPAQMDVIRQALSQAKLDPAEVNYLEAHGTGTALGDPVEVQAVDQVYGQAKGRTKPLVMGAVKANIGHCESAAGVAGLIKLIQMVQGDVLPPITHLEQLNPHFDGLSESLVFPKGASKDGTSSFTWHDEQSRIAAVSSFGYTGTNVHMIVSQHSTEKDALNVQAHAESSKTQSQAVYPFYFSAHSITSLRQQLSQFRDFLKETPHLSLPLISGAVNSIRNHFAYRFGCHAATRQELLSCLVSGIQGEYSEPLGKPTMVACFGPSNSQYPIPPQIFASDTWCSYFRCIETRVTQATEFSIEALMSSQAYPTLRHLISQLSYVEHLVEEGAEIRKVQASGTGTIAALVFAKSLTLEQAADLCVRLDQISGADLTEQEQRVEADIRYLIQDIHLKEAQRQIFVKGHDSTRQDVDWVKWPYHNVSEAKQRLLEALLSQQHPYHWSMLDSEIGIDVSALQIFPQKTIESETQPSWINLRINLFNRGATLRWDYTTIPQDKSQYPVLPDYVFARRRYWLPPQVTERVASLPSMFSTVGHSIFTTVLAVPDGGSLYAGELALSRLPFLRDHLVAGEIVLPASVYINLIAEVCVGPDGLPANIVQIQISQACILDSQPIGVYCRVDPTENGIAKVEIYTTSERGKWTKHVSAQVNVGFRAELNIKPLADVRKRCSQVISVEQHLLRAKQSGIEYGPSFQAIRQLYSGSGVALAKIEWPSDLPNYWSGCGLHPVMLDACFQVISGAMGTQNSDRKLPLYVPTELHEMHLQGNSAESLWCFVQILAPDEVFHNEATMYDYLSQQEKLSVALYVYDEQGKAILSIKRFEASKYRPELKGALWQEWLLEKQWIPCRFSSEKLDVDVSALLTQAQSRFSTVPYTMSVDMMQDLDALSGHFIHRALTQFGFDEQFFTSLLTAHTLPDEMLEQYGILPDYQRLLKKLVSLSEHLPVSALPADEIESKLRSKLGEETHELDLFVRCGNALAEVLQGQTKAVDLLFEAGHSQGTEALYQDSPGSLLLNQRIATLVAEAVSQIPSGRRLRILEVGAGTGATTQQVLEQVDGKNIDYVFTDISPHFLMRAKEKFGAFESVDYRIFDLQQNPLEQEFTAGAFDVVIAVNVLHATADLTQTLNHLSLCLADGGLLLLREVTEQQAWLDLSFGMTPGWWSYTDTSLRQNGPLLNTEEWQTLLHDSGFESTLVTDELERTESIFIAEKIASTTDVVSKGCYLVLGDEDTIETQGLNALKNELAQRGLDWVFISLNEFMQHPRFQELDAFSTLLMSVEAQQGPIIGLVYAWSMSPSDLNCDDLLEKSERYLKYPLLLCQTLLDARWRHLSPSFLTAGAQPRDHKVAQPLQAMVWGHVFTYINENAAFARLIDLDGTSVSGECLYEALTQKEECQLVVREGQLCAARLKRATLSVPEAPAQRCNIVADGTYVITGGFGDLGLQTAQKLVEQGAQNLILIGRRERRDVMKTLDMLREQGVNVVPLYVDVSDGEALNTALNQVLDELPMIRGVVHSVGVLDDGVIEKQRWERYLKVLKPKVFGAIHLYEAVSGYDLDFFVIYSSAAAVMGNPGQANHAAANAFLDAFAWYLRGQGCPGLAIGWGAWSEIGAAAARGITARLTQNQSIAGTISPEQGADLIAKQFASKNVQFTVLPIDFDRKIDTQYLPQVQRLLSSLLKSNKSGVQQAGTAPDNREKEHADQFVQDLLQMDEAQRQRQVENYLQGTLVKLLKQHGNIEHQTSLFELGLDSLLAIDLRSLLENQFRQKFESTLLYDYPTIHSLTEFLLQCVSDNHGQQKTPFTHHAETLSSDPATKTADVELQGDDAIAVVGMSCRFPGGANSIGEYWELLKNGVDAVQPIPETRWDHSRYYDPDKTQSGKIYVAEGCFIDEVAQFCPERFGIAGIEADLMDPQQRLLLDVCYEALESAGQNPMGLGGSETGVFMGVMTQDYLHLTQHVRENAFYVGTGSANSVVAGRVSHVFGLMGPTMTLDTACSSSLVGVQMACTNLRSGACDMAIAGGVSLQLSPEPLVIECAGGMLSPSGHCRTFDASADGFVRGEGCGVVVLKRLADAKRDRDPIVGVIRGGAVNHDGRAGGLTVPSGLSQQKVLEKALHDAQLSPSDISYIEAHGTGTHLGDPLELNALQAVFSESRDKAPLYVGSVKTNIGHAEAAAGVAGLIKVLLCLKHETFVPHLHFEQPNPNFDWEQSNIQVTTSLHEWTSKTKRRAGVSSFGLSGTNAHLVVEEFQEMMLPRKQPSEFVPLVALSHINPTQLKRDAARYHQLLTEDTPLLMDAAYTFCISRTGQSVQAVFPAQTQEALLAGLKRLADGELDIVDRKIKRPRLEWQISVNQRAAWMNQACTYYDLYHLFRGTIDASVEALKQRGFDVLSSIELCTLKDGSVDENIALPEQVRLAILNVAYGRLLLSLGIAPECIQVEGLSLLCVATLVGVSDIEQMIDGMLADSDTELKAILAGIHFSHGHMRMSFKADRRLSSAVNERCDSICPKTSVDYVWGDSDISLDLVRSAVNGKAFTELHQLLSMCSSLGLDIDWYTYFSPVEPHRIALPSSHFPTRRYWVNESLEQEQTRQVPLISQDITAAVDGCRYVDFHLSVEHQVFLDEHRLSNTRILPAAGSLAFILHAHQCSYPLEMKGVRLVRPVHFESQLSVQLAMKPTGRTELFYCEPTMKEWHVFATMEAIDMSESSTERALITDDNQLLSTFHAVLNAPRYTMDKETFYQASIPRELNLGESYRLIEEVQGDGLCLVARIRNISSEFVLDPRVLDACIQSVNGVGDKLERQSDGLFLPYAIQSISIHDWPSGSHFWCMTQFLPENSHADELVYDITVVDEHERICARFEHASFRKVNVPMVDSAKPETSIIHQLRWQPSPLRNTESVNSIAIDAVENLQNVLLIGKANAVQQRLQEQLSDCACHQVDAAEAARQSVDELRLLLDECSKAGAFIYTELLEASDRSPHDVEILTQDIWYLQQWLVAASKQGKPFIVLTRTGQSVCLNDVPLSAASVAAAALVQTAVQEFPSFPVLLIDLDDLSTESEDGIGWLAEITPSVQDSVVAYREGKRYRSVLEAVHSIEDVSCGNSVSPVKPNRTYIITGGLGDIGLLTAEKLVSEGATSLVLMSRNEHTNTNERIEALRNVGCQVEIRLGDVSDMTVLQAHIEQMTSCMPPVAGIFHTAAISANALMTQQTAADWADVLSVKVQGALNLLEMSREMELDCFVLFSSIASIGGSIGQSNYAVANAFLDYFAQSWSQLGVPVMSINWGAWENTGMAGRAEAKGIGFEQRLSPEQALSSLCFLMQNPRPQVVCFRENRALNTSGLELSGGTSLNEELKHRLFGEAFFNLSADEQNNTVSQTIRSILSDFLKVDPDHIKDEQPFFDCGMDSITAVDFSHQVGEVFGVELHVDIVFDYPNLLSLADYVLQLLLQDVPSFDPVISGEDGALVDTLSRLSVDEKQHHIKRTIRNILCEFLKVDGSHIKDEQPFFDLGMDSITAVEFSRHVGEAFALELHVDIIFDYPNLSTLTTQVMELLSFDVSPQPISEDVTIEQLSKMLEQELENDA

SEQ ID NO: 20 - DNA, 7341 bp, Vibrio rhizosphaerae MSSRF3
atgacgcatagaaatttgcaggaaacgacaccgatgcaagatacaaaggaattgttcagtaagagtctgaatgaaattaaacgactgaaagcgattaaccatcaactggaaacgatgcataaggagcccattgccattgtcggagcggcttgtcggtatccggggggaattggatcattggaacagttctgggaagctctggagcaaggacgtgattgcattgaaagaatgcaagatcagcgttggccgatgtcccgttttcttactgataatcctcataccgaaggtggtatttatagtgatgcgatggggttgcttgagaatgttgaccgttttgatcctgcacactttggtttatcggttgaagaagcaatacatttggatcctcagcatcggctcttgatggaaatggtttgggaaacgattgaagatgcaggatatgctgtagacagtttttcgggcacccggaccggtgtctatattgggttaatgagcgatgactatggtcagttgcagggccctttggaaaaagcaagctactatgtggggtcggggacggcacgtagttgtgctgctggtcgactctcttatacctatgggctggaagggcctgcgatgacgattgatacggcttgctcttcatccttagtgagtgtgcacctcgctgtgcaggcgatccgccgcggtgagtgtgataaggcaattgcgggcggagcgaatcttattctctcaccacaaggtagtgttgttgcttgtcgttctcaaatgttatcacgatcaggacactgtcagacttttgatatcaatgcggatgggtatgttcgttcagaggggtgcggtacggtcttgttgaaacgtctcagcagtgcgttgcaagatggggatcaaatttatgcattgattcgtgggtcagcgatcaatcacgatggccgtagccaagggttaacggcaccgagtggtcaagcccaacgcaaagtcattgctgccgctttagatgatgcaggggtttcacctgataaaattgaatttgttgaatgtcatggcacgggaacagcacttggtgatccaatcgaagttcgtgcgcttgaatcggcttatatcaagcaatcgcatcatcagcgaaaggcagatcagcctgtggataggctgcccttaaaactcggtgcactaaaatcgaatcttggtcatatggagtctgcggctggtattggtggattacacaaagccatggaagtgattcgtcaccgtaaggtgcctcaaaatcttcacttcaaaacattgaatccacaaatcaaagtggatcaaagtatgcttcatattgtcgatgaaccgacgcctttggctactgaagggcctgtatttgctggcgtgagctcatttggctttagtgggacgaatgcccacattattttggaatcatatacagaagcgaaagaaccgaaggatgaaaatgaagggaaaatatttcggttgtctgctcgttcccgcgctgctctgaatgattatgcaaaacaatatctcgaattcctgaatcagcattctgacatcgacttgtctgccttgtgttacaccgcggcaaccgggcgtgatgatagcgcgtatcgaatcgcgtttgcggttaacgatgtcaaagacctagaagcttgtctgaaagaatatattgaggtcacgggaggcgatgaatttttgccagaagcaggttccttccccttactttgggtgatcagtggccaaaccaatgtggactggcgcttagcgcatcacctataccagacgcgaagcacttatcgatcttcaattgagaaagcttacaaatatctagaagaaatggggtatccacactcgctcgctgactttgaatcaattttgacagggaccgagcagacagcggatgtattacctcatgcaatccatcgctgggcactggctgaactgttggttcatctaggcttatctccgacgtgtattacggggattggggttggggaatatgttgctgcgagtgtcgctggcttgattggttggcagtcgctgatggatattctatccacagggacggtcaaaaaaaccattgctttgggtcgcaagcgatatgactttgtcagtagtgccggcaaaaatgcagtattacccgaatcttggtttgggcatgagcctctggcacagaatgtgacactggaaaccctcggagccacatttagtatcgaatacgacacggtagattattgttgtatttccttggacgatacattgtcactgacgatgacggatacgcccgaggaaaatctgttccaatgggtatatggtcagacaaatcgggatgcaacaatgccgctccaaggatttctggcacagtgttatatgagtggttataccttgcattgggagcgagtctatgaggatagtcaatgtctgaaaatgagtttaccgacctatcctttccaacggcaaagcatatggactaagtggggttactcgtttgatgagacgcttccatcgacggttggtcgttccggaaaaatgttacagatgtcatcacgccatgagcgtcagggtgaagaggtcattgaacacccaatcttacagacggtctttgcctgtccttcagggattcgtaacttccaaggcgaactctcactggctcgaatgccttacctgggcgcacaccagatactcggtgaatccattgtgccggttagtttgtttgttgatattgttctcacggcaggtcggtggtgttggccagagcaaaacctatggattgaagacttgcagttgctgcataagtgtcctttaggtgaagcaccgattgacatttattgccatgtcaacccagatgctggaactgtttcagtgtatagcaaattacagcacgcaaaagagtggcagcaacatgtcagaggcagactatcgactagtgctcaggcgacggcaatgacgccacaggtcagtttgtctgaatttcagcatcaatgccatgaggcgatagctattcgtcatttctatcagggaattgcattaagtggcatgacttatggggaagactttcagggtatcttcgagatatatcggggcccccatacagcactggcaaaaattgctttaccaccgacggtcgagcagtctcttgttggatatagcacccatccaattctattggatgggtgcttgcaggcgattgtcgctgcatcagtgcagcgtcatgagcagggggttcgggtgccttcacaggttcgaggcattcgtctcttcaaaccgctcccggaaattttatggtgtgctgttgaggtctcttctgtgaactcgtctgatacggaggaaccggaggccggaaatagttacgcttctctgactgtgtttgattcgctcggtgaggtggtgatgaccattgagaggtttgaaacaacgcattatacggacactgaatctgttaaaactgccgcttatcatgaatggttgtatgacaagcaatggttacctgatgaaacggacggatccattcaggcaagagcggaaaacccagcatctgttgaacgtcattggttgctcttttctgatcagggcagtgtttgcaaggcactggaaacagcattgattgaccgtggtgatcgtgtttccgttctctgcctgaaccaacccgaggaacctcattatcggcaagtttcttttcaaagtatggcagatctaaatgaggtattcgacgcagtggagcatgatgtacatgctctaaccggtgtgatttatggctggtcagtcaccccatttgaagcggagagtcttggacatgagcaattatcgatttatgccaagtacccactttgggtatgtcaatcaattttggatcctcggcgaagacggttggcattgagctttctgacgtgtgacagtcagcctgtttatggacaagcgccttcacaaccgttggctgcgatgttatggggccacgtgacgagctacatcaatgaaaatgtagttcatgcaaatttgattgacttgttgtcaacagatcttatcaataatcatgaccttgaccatgtgctccacatactggatcaagtgaatgaatgtcaattcgctattcggaatggccaacgtttgattgctcgaattctcccaagtcagacagatttgtctcacaatgttcaattggatactacaggcacctatctgattactggtggattaggtgcacttggcttagaaacagcgaaagcgttaattcgtcaaggggctcgtcatctcattttagtcggccgtcgggctggtctcacagatgacagtcaagccgcgatacagtctcttgaggcgttgggcgcttctgtctatccattagttgcagatatttcgaaggcgcatgcttttgtcaacagcttaactgaacggttgattggcctacctcctttaaaaggagtcgtccactcagcgggtgtcttggccgatggcgttgtagcacagcagtcatgggaggattacctcaaagtatttgcgcctaaggttgcagggactttgtcgctctatcaagcggtgcgtgaacaagcactcgatttctttgtgatctactcgtctgcagcatcaatattaggaaaccctgggcaagcaaattacgcagctgcaaattcgtttgtggatagctttgtttggtatttaagaggtcaaggtgtcgctgcgacatcaattaattgggggggctggtcagaaattggcttggcagccaatatggcgcagcaagtacctgatagagaaaatgcgctacttggcttgattccacccagacagggcattcaggtgatagaggagcagctcatcagtgcgcatcctcaatttgctgtcttgcctttgaatcgtcaaatgacccttgatggcgataaaatgccgtatctacgtcaattattaagcaatgttctggacgatgatcgtaagcacctttcgacatccgcgatgcagaaacctgaagagcaatccacagctctgagcgttctggcaagtttaggacgtgttcatggtaatgatcgatgccgtctgctgaagcaatatttaaatcaagtgatcggtgagatgttaaaaattccgggtcagcttgatgagcgagccagtctgtttgatcttgggctcgattctttattaggtgttgatttgagactacggatagaaaaagatctggattgtgtcttggcatcgacacttttccatgactattcgacgatcgaaagtttaacaacctatctgttaaacgacgtgatcaaagacattgaagttgaagcgacgcaaaaggagcattcgccaacggctaatccgatgcatcatttgttaagcagtgctgatgtgaaggtgacaatctcacaaccagagaaaccacaagaaacgccacaggatcaagttgctggttcaggaaaaaatgaaagtcatagaaaatacacttctaacgctaagcagggtgatattgcgatcatcggtttatcgggacgttatcctggcgcatctgatctggagacgttttgggaaaatttacgggaaggttacgatggcatcactcgtgtaccagaagaacggtgggatgctgctgcttattatgatgaacgtaaaaatgtatccggtaaaagctatggggatatcgggggattcattgaaggtgtagatcagtttgaccctgagtttttcaatatcccacagcatatggcagcctacattgaccccaaagagcgcctgttccttgaaaccgtgtggaatttgttggaagacgcagcttatacccgggataaactgaaacagaattatcattctcgggttggtgtttttgtgggggcgatgtatcaactctatagtgcgtgtgccgggaatgttcatgagcagacggcaacgatgctgtcttcttataatgctattgctcaccgtgtgtcctatttcttcaatcttaaggggccgagtgttgccattgacaccatgtgttcatcttctcttacctccgtgcatttggcttgccagagtctatataacggagattgtgagattgcgattgcaggcggggtcaatctctctcttcatcctttgaaatatgtcggattaagtcaggctcacatcatgggcagtcatgcagggagccgaagtttcagcgatggagatgggtatctaccgtctgaaggggttggtgctgtactactcaaacctttagagagagctatcgaagatggtgaccgaatcgaagcggtgattaaagcttcaacgatcaatcatggcggtcattcaactgggttttatgcgccgaatcctgaagctcagaccgagttgatcgagacaaactttcagcgcgcgcagcttactcctgacagtattcagtatgttgaggcggcagccaatggggccagccttggagactccattgaatttaaagtattgaaccaagtattcatgaatgccggtgtgaaaaaaggcagttgtccgatggggacggttaagtcgaatatcggtcatgctgaagcggcttctggtatggcgcaaattgcgaaagttattttgcagatgaagcatcagacaatgatcccaacggtgaaagccgagcctctgaatccaaacattccactgttagagtctcccttccgtctgttgcatcagtgtcagcgttgggaacagccagagaatgatcaacgacgtgcgacggtcagttcatttggtgccggtggcgccaatgcgcacctgatcctagaagagtatgctccgaccgagagaagtacggcactgaaatcatcaattgttcctcatggagacgagatgatcgtgctttctgcgcgctcaaaaactcagttgcaacagatggtgaagaatatgctgatgtatttggacaaacatcaatcaaatgaggttgagacacaatcgaaatggttggctgatattgcacatactttgcagactggcagggaagaaatggattgtcgtttgtcattgctggttagcagtattgatgagttacgtcaggggttatctcattatctttcttctgaatctgttcttgatacggtttcagggaaggatatcgctcctgttcttatacaaacggggaacattcaagaccagctcgagctgcgaaacctccttttaggagtggccggagaagcgatggctcagtctttgattgctgaacggcagttggagaaactgatgctgcactgggtacagggagggcgtatctgttgggataaacttcgacaggggcagccggtgaagtgtatcagtttgccaacttatccctttgagcgacaacgttattggctctcaggtccgacaacgggcgaacagagacgacgggaaccccagatgggagaagaaataggtacaacctatgagtaa

SEQ ID NO: 21 - protein, 2446 aa, Vibrio rhizosphaerae MSSRF3
MTHRNLQETTPMQDTKELFSKSLNEIKRLKAINHQLETMHKEPIAIVGAACRYPGGIGSLEQFWEALEQGRDCIERMQDQRWPMSRFLTDNPHTEGGIYSDAMGLLENVDRFDPAHFGLSVEEAIHLDPQHRLLMEMVWETIEDAGYAVDSFSGTRTGVYIGLMSDDYGQLQGPLEKASYYVGSGTARSCAAGRLSYTYGLEGPAMTIDTACSSSLVSVHLAVQAIRRGECDKAIAGGANLILSPQGSVVACRSQMLSRSGHCQTFDINADGYVRSEGCGTVLLKRLSSALQDGDQIYALIRGSAINHDGRSQGLTAPSGQAQRKVIAAALDDAGVSPDKIEFVECHGTGTALGDPIEVRALESAYIKQSHHQRKADQPVDRLPLKLGALKSNLGHMESAAGIGGLHKAMEVIRHRKVPQNLHFKTLNPQIKVDQSMLHIVDEPTPLATEGPVFAGVSSFGFSGTNAHIILESYTEAKEPKDENEGKIFRLSARSRAALNDYAKQYLEFLNQHSDIDLSALCYTAATGRDDSAYRIAFAVNDVKDLEACLKEYIEVTGGDEFLPEAGSFPLLWVISGQTNVDWRLAHHLYQTRSTYRSSIEKAYKYLEEMGYPHSLADFESILTGTEQTADVLPHAIHRWALAELLVHLGLSPTCITGIGVGEYVAASVAGLIGWQSLMDILSTGTVKKTIALGRKRYDFVSSAGKNAVLPESWFGHEPLAQNVTLETLGATFSIEYDTVDYCCISLDDTLSLTMTDTPEENLFQWVYGQTNRDATMPLQGFLAQCYMSGYTLHWERVYEDSQCLKMSLPTYPFQRQSIWTKWGYSFDETLPSTVGRSGKMLQMSSRHERQGEEVIEHPILQTVFACPSGIRNFQGELSLARMPYLGAHQILGESIVPVSLFVDIVLTAGRWCWPEQNLWIEDLQLLHKCPLGEAPIDIYCHVNPDAGTVSVYSKLQHAKEWQQHVRGRLSTSAQATAMTPQVSLSEFQHQCHEAIAIRHFYQGIALSGMTYGEDFQGIFEIYRGPHTALAKIALPPTVEQSLVGYSTHPILLDGCLQAIVAASVQRHEQGVRVPSQVRGIRLFKPLPEILWCAVEVSSVNSSDTEEPEAGNSYASLTVFDSLGEVVMTIERFETTHYTDTESVKTAAYHEWLYDKQWLPDETDGSIQARAENPASVERHWLLFSDQGSVCKALETALIDRGDRVSVLCLNQPEEPHYRQVSFQSMADLNEVFDAVEHDVHALTGVIYGWSVTPFEAESLGHEQLSIYAKYPLWVCQSILDPRRRRLALSFLTCDSQPVYGQAPSQPLAAMLWGHVTSYINENVVHANLIDLLSTDLINNHDLDHVLHILDQVNECQFAIRNGQRLIARILPSQTDLSHNVQLDTTGTYLITGGLGALGLETAKALIRQGARHLILVGRRAGLTDDSQAAIQSLEALGASVYPLVADISKAHAFVNSLTERLIGLPPLKGVVHSAGVLADGVVAQQSWEDYLKVFAPKVAGTLSLYQAVREQALDFFVIYSSAASILGNPGQANYAAANSFVDSFVWYLRGQGVAATSINWGGWSEIGLAANMAQQVPDRENALLGLIPPRQGIQVIEEQLISAHPQFAVLPLNRQMTLDGDKMPYLRQLLSNVLDDDRKHLSTSAMQKPEEQSTALSVLASLGRVHGNDRCRLLKQYLNQVIGEMLKIPGQLDERASLFDLGLDSLLGVDLRLRIEKDLDCVLASTLFHDYSTIESLTTYLLNDVIKDIEVEATQKEHSPTANPMHHLLSSADVKVTISQPEKPQETPQDQVAGSGKNESHRKYTSNAKQGDIAIIGLSGRYPGASDLETFWENLREGYDGITRVPEERWDAAAYYDERKNVSGKSYGDIGGFIEGVDQFDPEFFNIPQHMAAYIDPKERLFLETVWNLLEDAAYTRDKLKQNYHSRVGVFVGAMYQLYSACAGNVHEQTATMLSSYNAIAHRVSYFFNLKGPSVAIDTMCSSSLTSVHLACQSLYNGDCEIAIAGGVNLSLHPLKYVGLSQAHIMGSHAGSRSFSDGDGYLPSEGVGAVLLKPLERAIEDGDRIEAVIKASTINHGGHSTGFYAPNPEAQTELIETNFQRAQLTPDSIQYVEAAANGASLGDSIEFKVLNQVFMNAGVKKGSCPMGTVKSNIGHAEAASGMAQIAKVILQMKHQTMIPTVKAEPLNPNIPLLESPFRLLHQCQRWEQPENDQRRATVSSFGAGGANAHLILEEYAPTERSTALKSSIVPHGDEMIVLSARSKTQLQQMVKNMLMYLDKHQSNEVETQSKWLADIAHTLQTGREEMDCRLSLLVSSIDELRQGLSHYLSSESVLDTVSGKDIAPVLIQTGNIQDQLELRNLLLGVAGEAMAQSLIAERQLEKLMLHWVQGGRICWDKLRQGQPVKCISLPTYPFERQRYWLSGPTTGEQRRREPQMGEEIGTTYE

SEQ ID NO: 22 - DNA, 1071 bp, Vibrio rhizosphaerae MSSRF3
gtgtcgcttttttcatcatttgaacttggtaatgccacattaaaaaatcgaatagctatctcacccatgtgtatgtattgcgcgaaagatggcataccagatgattggcatttagttcatctcgggagtcgggcagtcggtggtgctggcttaatatttacagaagctacagcagtttcacctgagggacgtattacacatggttgtacagggttatggaatgaccagcaggtatctgcatgggcgcggattgtggactttttacatgcgcaagggactgtcccgggcattcagctcgctcatgccggacgtaaggcaagtaccgatttaccttggttaggtggtcatccacttcccccggatgcggggggatggacctcttatgcaccaagtccactggcgtttaatcaggggtacaatgtgccagttacattggataaaggcggtattgctaaagtgatagaagactttgcctcagcggctcggagggcgaagtctgccggctttagcgtgattgaaattcatgcagcgcatggctatttgttccatgaattcctttcccccttatcaaaccaaagagaagatgattatggcggctcccttgagaaccgggcgcggttgctgcgttcagtcattgcctctgtcagaagcgagtggccagctccctttccattggttgttcgtttatcggcaacagattgggcacccggtggctgggatattgacgagtgtgttcaattggcgatttggctgaaagaagatggtgtggatctgatagatacatcttctggcttgaatattgcggatgcaaaacctccttttgcacccgaatatcaagttaaattttctgcccagattcgtcgtgaggcaggcattgcaacaggtacagtcggcatgattactcctggtaaggaagcagatgacatcatttatcgaggtgatgcggatcttgtattgtttgccagagagtctttgcgtgacccttattttccttttcgtgcggctacggcgtgtggtgctgaggtttgtgtccccaaacaatatttacgggcttggtcgtctgccacttag

SEQ ID NO: 23 - protein, 356 aa, Vibrio rhizosphaerae MSSRF3
MSLFSSFELGNATLKNRIAISPMCMYCAKDGIPDDWHLVHLGSRAVGGAGLIFTEATAVSPEGRITHGCTGLWNDQQVSAWARIVDFLHAQGTVPGIQLAHAGRKASTDLPWLGGHPLPPDAGGWTSYAPSPLAFNQGYNVPVTLDKGGIAKVIEDFASAARRAKSAGFSVIEIHAAHGYLFHEFLSPLSNQREDDYGGSLENRARLLRSVIASVRSEWPAPFPLVVRLSATDWAPGGWDIDECVQLAIWLKEDGVDLIDTSSGLNIADAKPPFAPEYQVKFSAQIRREAGIATGTVGMITPGKEADDIIYRGDADLVLFARESLRDPYFPFRAATACGAEVCVPKQYLRAWSSAT

SEQ ID NO: 24 - DNA, 831 bp, Vibrio rhizosphaerae MSSRF3
atgaatttatcaaatataccgagtttggcacttgctcatttgtcggttttaaatgtgccaccactcgaattggtcagcctggcagcaaaggtcggtttttctgatattggcttgcggttatatcctgctttttcgggttcaattttttatgaattacctgaaggttctgcccaatgtcgtgatatgcaacgtcgtttgagtgatgaaggcattggcgtcaacgatattgaatttattggtattggtgaaaattttaacccttcagatttgcaaggcctccttgataccgctggcgctctcggtgccaaacgtttatatgtttgtggcgatgatcctgatcaaactcgcttaattgataattatgcgcgtttgtgtgagttggcagcaccttatgatctttatgtggaactggaatatatggcttggcgggcggtgaaaaattttgaggatgcgtttgacgttgtaaccgccgctgggcaatcgaatggcggtatgttaattgacgcgcttcatttatttcgcactggagggacagtgcaagacatattacgtgcacctgacttcattcattctattcagttgtgtgatgcgatagctgaacaatgtccggcaacatctgaagatttactgcaagaagctcgttcagggcgtttagctccgggacaaggctctttacctttgaaatctttattaacaacattggctgatgggactgcaatctctttggaagtcccgacaaatagtgataagcctgctgcgcagcatgctcaagaaatctttcaatccacaataaatttgatccatcgggtagaggaaagtctatgtctttaa

SEQ ID NO: 25 - protein, 276 aa, Vibrio rhizosphaerae MSSRF3
MNLSNIPSLALAHLSVLNVPPLELVSLAAKVGFSDIGLRLYPAFSGSIFYELPEGSAQCRDMQRRLSDEGIGVNDIEFIGIGENFNPSDLQGLLDTAGALGAKRLYVCGDDPDQTRLIDNYARLCELAAPYDLYVELEYMAWRAVKNFEDAFDVVTAAGQSNGGMLIDALHLFRTGGTVQDILRAPDFIHSIQLCDAIAEQCPATSEDLLQEARSGRLAPGQGSLPLKSLLTTLADGTAISLEVPTNSDKPAAQHAQEIFQSTINLIHRVEESLCL

SEQ ID NO: 26 - DNA, 906 bp, Vibrio rhizosphaerae MSSRF3
atgtctttaaatgatttgcaagttcctgtaagcggcaatgtgatcgacactgagcagtcacctgtgtgtcaattagggttgattggttcagggattggacattcattgtcgccagcgctttataagtctgaagccactgcgcatcaaattcagtgtacttatacgctgtttgatttagatgagccagaaaccggagatttatcagaactattggcgcaggcagaacgctcccttttttcggggctcaacattacgattccctgtaagcaatccgtgattcccttgttagatacactgagtctggaagcaaaagcgattggagcagtcaataccgtttgttttcaaggccacaaacgggtgggatataacacggatgctgagggattccgtgaaagctttctcgcacagttgcagcatgtgaatctagagcgagtcatacagtttggagccggtggtgtcggtgcagcaaccgcatttagtttgttagagcttggggctcagaaactgacaattgtggatgtcatcgatgaccgcgcgcaccaactggttgatagacttaaggcctatttcccagagcgcgaaattacaatgacccatccttcagatgccgagataaaagcaacgatgggtatcgttaatgcgacgcctatcggctcagataagtattcaggttcagtagttgaactgaacctgctttcctcccatatgtgggttgcagatgtcgtctattccccagatgaaacagtgcttttaaaagcagcccgatcattaggatgtccaacattggatggtttgagcatgctgatccatcaggcggtgagggcatttgaattgtttacggggcagcaggctgatgtcactcgcatgttgaaacgttttagacaatcgcaagcaaatcatgcatag

SEQ ID NO: 27 - protein, 301 aa, Vibrio rhizosphaerae MSSRF3
MSLNDLQVPVSGNVIDTEQSPVCQLGLIGSGIGHSLSPALYKSEATAHQIQCTYTLFDLDEPETGDLSELLAQAERSLFSGLNITIPCKQSVIPLLDTLSLEAKAIGAVNTVCFQGHKRVGYNTDAEGFRESFLAQLQHVNLERVIQFGAGGVGAATAFSLLELGAQKLTIVDVIDDRAHQLVDRLKAYFPEREITMTHPSDAEIKATMGIVNATPIGSDKYSGSVVELNLLSSHMWVADVVYSPDETVLLKAARSLGCPTLDGLSMLIHQAVRAFELFTGQQADVTRMLKRFRQSQANHA

SEQ ID NO: 28 - DNA, 1011 bp, Vibrio rhizosphaerae MSSRF3
atgagagctttactaagtaaaacaacgggaggaccagacagccttgaactgtgtgaagtgccagaccccgaattggcgccgaatgaagtgctgatcaaggttagatcttgtgcaatcaattacccggatatcttgattattcaagatttatatcaggataaaccacaacgccccttcattccaggctctgaaatagcaggcattgtcgaagcgataggtacccaagttaaaactttgcaaatcggtgaccatgtatttgcggccacaggtaatcaaggtggcctcgcagagaaaattacattaagcgaaaaagactgttatttgtttcctcatgaacagtcttttgaagaagcttcgagtctactgctcacttatgcaacagcctattacactttaaagaaccttgctgatctccaagcaggggaaacactcctagtccttggcgcagcaggaggcgttgggcttgctgctgtagagctaggtaaggtcatgggagcgcatgttattgctgtcgcctcttcagaagaaaagctctctctggcttatcgctatggggcacatgctgggatcatcggtccagaagatatatcaactcaagaagcatcaaagtattttaaaaatcaacttcaggccacctgcggtcaacaccttccaaatgtcatcgttgatcctgttggtggttgctattgcgagccagcattacggactatagctcatcgaggacgctacttagtgataggatttacagccggtattcctaaaatcccaatgaatctgattctactcaaagcttgccaagtatttggtgtcctttgggggaaatttgttaacgatgaaacagtatccaatcgtaaaaatgtgcaagaactcatcacactttggcaagcaggtaagattaaacccttcatttcagagactttccctctggctcagtcaggaacagctatgaaacgcttacaggatcgtaaggtcatgggcaaagttgttgtcacaatggaatctatagattaa

SEQ ID NO: 29 - protein, 336 aa, Vibrio rhizosphaerae MSSRF3
MRALLSKTTGGPDSLELCEVPDPELAPNEVLIKVRSCAINYPDILIIQDLYQDKPQRPFIPGSEIAGIVEAIGTQVKTLQIGDHVFAATGNQGGLAEKITLSEKDCYLFPHEQSFEEASSLLLTYATAYYTLKNLADLQAGETLLVLGAAGGVGLAAVELGKVMGAHVIAVASSEEKLSLAYRYGAHAGIIGPEDISTQEASKYFKNQLQATCGQHLPNVIVDPVGGCYCEPALRTIAHRGRYLVIGFTAGIPKIPMNLILLKACQVFGVLWGKFVNDETVSNRKNVQELITLWQAGKIKPFISETFPLAQSGTAMKRLQDRKVMGKVVVTMESID

SEQ ID NO: 30 - DNA, 1509 bp, Vibrio rhizosphaerae MSSRF3
atggaaagtattccccatttaattttcaatttccaaaatagaggaatagccttaacgctgaaggatggtgcgctcgcttatagagcgccaaaaggcaccctcacaaatcaagataaaaccacgctcagtgaacaccgagatgagatcatcacttatttatcagcgctagaaactcaacttgaaacacctgtgacttttgaagaatcgaccgaaatagaaccctccgcgttacagaaactttggtggttttggtatggcgtaccagaacgtcagctagaacaggaacgacttccattaattaagccttatcggaataccagcaaagccagagtcgaagctgcaattagacaactcatctcccgacataacatacttagagcatcattttatgaggatgacggttatttaaagatcaccttaaatgatgcgggttcgtttccgattgaatatcagacctatacacccacaggctcagaactggaaacggaaaaagaactgaaagaattagcaaaagcattttcagaaaagcaacttccactcaatggtcagtggctgctccgagccaaaattgtgagcctttccaaagaagactttttgttattgttcgttttcaatcatatcattgtcgatgcaggatcaatcgtattaattatgtctgaattggatactctgatttccaacgctcttccacaaacactcccgcctgccatacagtttactgattatgtcatatgggaaaaacaatggctgaacagcgcagaccgacagcccttattcaattactggaatcagcgtctgcaaaatttatcgccactgaaagccccagttagcaaggaaattctcagttggcaatctggcacgaaagtagactataaatttatcataaaaggctactttctacagcaaatcagagcatatgctgttgcacacaaaacctcattatttaatgtatttcttaccacttttgcacttgctctgtctcggtggtctggtatttcaaaatttccgatccgttgtgtggggaatttacgtactgccccctcagtcactcccatcatcggttatcttgtttgcagtgaccttgtagaagttgacataccatccgattacgatttcgaatccatactgaaatacaatgagattgaataccacagtgctataaaactcagaattcctaccatgctgagagttccgttaccaagtggtaccacaggacaaggcgttgaagatccaagacatattgcaacagggataaatatgttcattattcgccaatccgacgcgaaacaaacgtctgagacggaaacatatgaatggccacccaaagtaacgcgttcctcaggggaaccttggcctatccttttgccatcgatttatttccgactgctcgattttggcgattcaatcgatgtttctctagagctcaatgatgaacagttatccgctcaagaacaacaagctttgctcgatacgtttttcgcaacaacagcagaatttttgtttcaataa

SEQ ID NO: 31 - protein, 502 aa, Vibrio rhizosphaerae MSSRF3
MESIPHLIFNFQNRGIALTLKDGALAYRAPKGTLTNQDKTTLSEHRDEIITYLSALETQLETPVTFEESTEIEPSALQKLWWFWYGVPERQLEQERLPLIKPYRNTSKARVEAAIRQLISRHNILRASFYEDDGYLKITLNDAGSFPIEYQTYTPTGSELETEKELKELAKAFSEKQLPLNGQWLLRAKIVSLSKEDFLLLFVFNHIIVDAGSIVLIMSELDTLISNALPQTLPPAIQFTDYVIWEKQWLNSADRQPLFNYWNQRLQNLSPLKAPVSKEILSWQSGTKVDYKFIIKGYFLQQIRAYAVAHKTSLFNVFLTTFALALSRWSGISKFPIRCVGNLRTAPSVTPIIGYLVCSDLVEVDIPSDYDFESILKYNEIEYHSAIKLRIPTMLRVPLPSGTTGQGVEDPRHIATGINMFIIRQSDAKQTSETETYEWPPKVTRSSGEPWPILLPSIYFRLLDFGDSIDVSLELNDEQLSAQEQQALLDTFFATTAEFLFQ

SEQ ID NO: 32 - DNA, 1230 bp, Vibrio rhizosphaerae MSSRF3
atgctaaaacaaccgccccctctaaaagggatccgagtcattgattacagtcactttctcgcaggtccttatgtttcacgctgtttagccgcgttaggtgccgaagtcattaaggttgagcgtcccaaagcaggcgacgcaggacgaaaccatccctattttatcaaaggacacagtgggtattttctacagcaaaatatggggaaaaaaggactatgtgtaaacgccaaagatcctcgtggtctggaattcatcaaaaagctgacgaaaacggctgatgtctttatcgaaaactacagacccggcgctctaaaaaaactaggattgggatacgacacactagcagaagagaatccgggcttaatctactgctctatctcagcctacggacaaactggacctgattccgagcgtgcaggttttggtttaattgcagaggcaaaaagtggtgcaatggatctcattggccaaccaaatgatgtgccaccactgatgagaatgcctgtcgctgatatgtatgcgggtagtcatggtgtttctgcggtatgtgcagctttatttagtcgccatacaaccggtattggccaacatattgatattgcattatatgactgtatggtttcaatgcatgattttgcagtacagttccataccctgagcgatggtaccgatgtggtgaagcgaagtggtaaccatttgcctcaatccaccctttatggtgtatttcctgcttctgatggtgctatcgtcattgctgcacaagtgaatgaagtttggcaacaattggcgcacctcattgggggtgaagaactcgccaacgatactcgctttcagacagcagaaaatcgtaatacaaatagtcaagctgctgttgacttggtcaaatcctgggcaaaacaccgaacagtcgcaaactgtattgagcttcttgaaaaagcgaatgtgcctaacgccccagtccagacaatcaaacaagtattggcagacccacagattcatgcacgtcgaatgatcattgaacaggaacatccagaattaggtaccattttgatgcccaatgttccttttaatttctccaatattgaacctcaacggttagatatcgcaccaaatcttggtcaacataatcaggatattgccgaaagtatcggctacaccgatgaagaaattaaggcattggaaaatgatggtgttttatatcaagtcccccttaaagatgaaactaactga

SEQ ID NO: 33 - protein, 409 aa, Vibrio rhizosphaerae MSSRF3
MLKQPPPLKGIRVIDYSHFLAGPYVSRCLAALGAEVIKVERPKAGDAGRNHPYFIKGHSGYFLQQNMGKKGLCVNAKDPRGLEFIKKLTKTADVFIENYRPGALKKLGLGYDTLAEENPGLIYCSISAYGQTGPDSERAGFGLIAEAKSGAMDLIGQPNDVPPLMRMPVADMYAGSHGVSAVCAALFSRHTTGIGQHIDIALYDCMVSMHDFAVQFHTLSDGTDVVKRSGNHLPQSTLYGVFPASDGAIVIAAQVNEVWQQLAHLIGGEELANDTRFQTAENRNTNSQAAVDLVKSWAKHRTVANCIELLEKANVPNAPVQTIKQVLADPQIHARRMIIEQEHPELGTILMPNVPFNFSNIEPQRLDIAPNLGQHNQDIAESIGYTDEEIKALENDGVLYQVPLKDETN

SEQ ID NO: 34 - DNA, 1674 bp, Vibrio rhizosphaerae MSSRF3
atgactgaatcggaattaagtaagagcaaaccgtcagagattgccattatcgctgctatccaatcctcaaaagcacctttaagaccaagccattttcacaacgatagcaagccattcttatttgatacaaacgcttcaactgatattaagtcgaataaaaatcacaacactacgtctctggatacgaatttttttgagacgatttatcaaccagaattataccttgatgagcaggttaaatgttatctagaaatgatctggcagctacttgaatcagcaggcgtcacaagagacgttattaataaaaacaatagtgacaacattgtcgtttatatcaatcaagaaccaaattttgcgatagatacaacctttagtgacactagactcagtaattatcaggaatatcaaacaatatatgaaagcatcaccaaccatctctccaaattattgggtttaccctcttcctgttttttaaacgaacgagctttacctacctctatccattcatttggtgagttatcccattatttattggttggagatactcagattgctataatacttggcgtccccttatctaaaacatccaatcaattacaattgagtacaatgcccccggatgcaacaccttggggcgtcttacttggtaaggatcccagaagcagccagcatcatgaactccctattttggctatggtgtcacctgattctgaaagacctaataaattcaataattattcctctcatatcctgtctccagtagaaataccagccacagcccccgagctgaaccaccctgaacttattgttctatcagccaacacatctcaagcactacaaataaaagctgccgaactattcaatcatatccttgattttattccccttgaaaagatcaataatgccagttcaatgctcacgaactccggaacgctgtccttacaagatctagcttatactctgcaatgtggtagagaggcgatgggctatagattagcaatggttgtcaaccgcttagatgaacttctcttcggtctcgctcaatttctcacaaacgagggagaggccaccccccccgtgcaaaatgtattaaatagggttcaacctcaagtgccagtgaatctctatcgtggtcatccaaatcgtaacaaccaatttcaaaaccttttctccggtagtagtggggaaacgatgctcaacacgctcattgaaacaggagaactcagtaatctcgcgttatattggagccaaggcggtgatatttcatggacaagaataaaaagatcggtcataccgaataaaattcacctgcctgtttatcctttttatcagccatccacacatacacagcaaaatgccgaagaaaataccgccttgtcttcgagtatcaccatcagtggcatcgaatccgacttaaccactatctggcaagagctcttagggagaccgaaaatagggcgacatcaaaacttttttgaaatcggcggggattcacagttaggaatgcatatgctctccattatacgtggcaccataggcgctgatttaccactcaactgtctctatgaagcacaaaccattgccaagatgagtgaacaaatcgtactcactcttgcgctctcatcatccgaatactctcaagacgacgaatatgaagaaggcttcatcgtctag

SEQ ID NO: 35 - protein, 557 aa, Vibrio rhizosphaerae MSSRF3
MTESELSKSKPSEIAIIAAIQSSKAPLRPSHFHNDSKPFLFDTNASTDIKSNKNHNTTSLDTNFFETIYQPELYLDEQVKCYLEMIWQLLESAGVTRDVINKNNSDNIVVYINQEPNFAIDTTFSDTRLSNYQEYQTIYESITNHLSKLLGLPSSCFLNERALPTSIHSFGELSHYLLVGDTQIAIILGVPLSKTSNQLQLSTMPPDATPWGVLLGKDPRSSQHHELPILAMVSPDSERPNKFNNYSSHILSPVEIPATAPELNHPELIVLSANTSQALQIKAAELFNHILDFIPLEKINNASSMLTNSGTLSLQDLAYTLQCGREAMGYRLAMVVNRLDELLFGLAQFLTNEGEATPPVQNVLNRVQPQVPVNLYRGHPNRNNQFQNLFSGSSGETMLNTLIETGELSNLALYWSQGGDISWTRIKRSVIPNKIHLPVYPFYQPSTHTQQNAEENTALSSSITISGIESDLTTIWQELLGRPKIGRHQNFFEIGGDSQLGMHMLSIIRGTIGADLPLNCLYEAQTIAKMSEQIVLTLALSSSEYSQDDEYEEGFIV

SEQ ID NO: 36 - DNA, 354 bp, Vibrio rhizosphaerae MSSRF3
atggatagaagaaaaaaactaaagaaacagtataaagaaacaccacgaccgatgggtgcttatcgggtttacaatcaagaagatcgtctatcactgataggaacctgtaaagacgttcaagcgagactgaaccgacataagaccgaacttaaacttggtagccatcgaaataaagcaattcaggcggactggaaccgtttaggcgaagaagcatttactttcgaaatcattgaactcatcgaacctttggacgacccagactataacccagacgatgatttagaagagctcatggagataatatcggcacaagatgcctatacgcctgacaaactctacaacaataaaaagtaa

SEQ ID NO: 37 - protein, 117 aa, Vibrio rhizosphaerae MSSRF3
MDRRKKLKKQYKETPRPMGAYRVYNQEDRLSLIGTCKDVQARLNRHKTELKLGSHRNKAIQADWNRLGEEAFTFEIIELIEPLDDPDYNPDDDLEELMEIISAQDAYTPDKLYNNKK

SEQ ID NO: 38 - DNA, 597 bp, Vibrio rhizosphaerae MSSRF3
atggaagctataagtagaatcgataagtgtagagttcaggcaaatcgcctaagaaaaatggtcaagttaggcttagaaaaagagagcgtcgaagaggatatcaaggcgatacttgaaagatttaaaacatcaggaattttgaaaagccgatcagatgatgaatcaatacagttaaagcgtcttaatactttttccataaacaagtatttggagctacttgcaaaagaagcaggatatatcgattggtcaacaatggcacaggtgatcaaagtggaagatcacattgatgatactgaagccactgagttatatcgttcaggtgtcagtgagcccaacttaaatgtctggtgcccaacttatgatgatgcaaaggcgtacttagatactcacaagggtttctacttactacaatttaaagggcaatgttttctggctcaagcaccacacattgaaggtttaggtctcaatcctcaagatcctgactgggaaaaaataggtcgagactgggtaaaaccgaaagatccagaggcgaaatcacgcttgcgagaaaaattacaacaagcgcgaaaacaaagggagggcgttcaaaatagctga

SEQ ID NO: 39 - protein, 198 aa, Vibrio rhizosphaerae MSSRF3
MEAISRIDKCRVQANRLRKMVKLGLEKESVEEDIKAILERFKTSGILKSRSDDESIQLKRLNTFSINKYLELLAKEAGYIDWSTMAQVIKVEDHIDDTEATELYRSGVSEPNLNVWCPTYDDAKAYLDTHKGFYLLQFKGQCFLAQAPHIEGLGLNPQDPDWEKIGRDWVKPKDPEAKSRLREKLQQARKQREGVQNS

SEQ ID NO: 40 - DNA, 1383 bp, Vibrio rhizosphaerae MSSRF3
atgtctacagccccgcctctagccaataccagcccgagcgctcacaaaagttattttaaagaagcacttaaactgtcattaccattagttatttcacagtttctcatcatggcaaatggatttatttgtaatctaatgttgtctcgggtcagtgaatcagcttttgcagctggtttatttattaatacgatccaaatgattcttgtcaccgttgtttttggactgctttcatctctcagcccattgattggtattgtcacaggtcagggaaagcctgaacgtgtaggccagttatttatcgctgggtgcattatttcactgatactgagtttctctagcattgctatattgatctatattgagccaattatgttgctgttagggcagcctgagaatttggctcatctctgcgcagagtattttcgaatttacttatggtctgtacctgctgctggtttaattacagtttttatacagctttttttaggaacatttaaacaggttgttgtatttctatatagcataggaaatttactcttatcatctgtcctcagttatatccttatatttggtaaatttgggtttccagcgatggggttggaggggctagcttgggctatctcaataacttcttgcgtggcagtgcttagtctaggttgctttatatttagaagctctgattataaagataagaaattaactaatctgaaatggcagtttctcaaacaagattcaggtcgtattattcgacttggtttccctatttcgttacatgtagggaatgagatgttttcattcctattcattattataatggttggttggattagtatagaagcactcaatatgcagcaggttgtcactagatacttaatgatgctagccatccctatatttggcttatctcaggcggttactgttgcaattagcaagcagtttggagaggaaaagtttggtgaagtcataaatatagggcgagcttatatccgaatgggagttatctattcttctgttgttctattattatttgcaaccattcctgatgtgtttattcgagtgtttattgataacaccccccaaaatacagctatttataaaactctctccattatattaattttagttgcaattgggcaggttttcgatgcgataaaaaatattgttacagggtcgcttagagggcttcatgatactaaatttccgatgctacttagttttattactgtgtggccgataggcgtgcctcttgcttatttaatgggatttacctttgaatggggattgattggcattacgattgcccatagtttggcaatcgcattttcttgtatcttcttgtattggcgttggattcagaaatccagaaatatggccaaaaggtag

SEQ ID NO: 41 - protein, 460 aa, Vibrio rhizosphaerae MSSRF3
MSTAPPLANTSPSAHKSYFKEALKLSLPLVISQFLIMANGFICNLMLSRVSESAFAAGLFINTIQMILVTVVFGLLSSLSPLIGIVTGQGKPERVGQLFIAGCIISLILSFSSIAILIYIEPIMLLLGQPENLAHLCAEYFRIYLWSVPAAGLITVFIQLFLGTFKQVVVFLYSIGNLLLSSVLSYILIFGKFGFPAMGLEGLAWAISITSCVAVLSLGCFIFRSSDYKDKKLTNLKWQFLKQDSGRIIRLGFPISLHVGNEMFSFLFIIIMVGWISIEALNMQQVVTRYLMMLAIPIFGLSQAVTVAISKQFGEEKFGEVINIGRAYIRMGVIYSSVVLLLFATIPDVFIRVFIDNTPQNTAIYKTLSIILILVAIGQVFDAIKNIVTGSLRGLHDTKFPMLLSFITVWPIGVPLAYLMGFTFEWGLIGITIAHSLAIAFSCIFLYWRWIQKSRNMAKR